(19) United States
(12) Patent Application Publication
(10) Pub. No.: US 2025/0236873 A1
(43) Pub. Date: Jul. 24, 2025

(54) Methods Of Treating Decreased Bone Mineral Density With Cluster Of Differentiation 109 (CD109) Inhibitors

(71) Applicants: Regeneron Pharmaceuticals, Inc., Tarrytown, NY (US); The Royal Institution for the Advancement of Learning / McGill University, Montreal (CA)

(72) Inventors: Jonas Bovijn, Tarrytown, NY (US); Olukayode Sosina, Tarrytown, NY (US); Sirui Zhou, Montreal (CA); Luca Andrea Lotta, Tarrytown, NY (US); Aris Baras, Tarrytown, NY (US); John Brent Richards, Montreal (CA)

(21) Appl. No.: 19/033,694

(22) Filed: Jan. 22, 2025

Related U.S. Application Data
(63) Continuation of application No. 18/392,181, filed on Dec. 21, 2023, now Pat. No. 12,234,460.
Application No. 18/392,181 is a continuation of application No. 17/816,084, filed on Jul. 29, 2022, now Pat. No. 11,891,607.
(60) Provisional application No. 63/230,707, filed on Aug. 7, 2021.

(51) Int. Cl. C12N 15/113; A61K 31/4535; A61K 31/565; A61K 31/57; A61K 31/663; A61K 31/675; A61K 38/23; A61K 38/29; C07K 16/28; C12Q 1/6883

(52) U.S. Cl. CPC C12N 15/1138; A61K 31/4535; A61K 31/565; A61K 31/57; A61K 31/663; A61K 31/675; A61K 38/23; A61K 38/29; C07K 16/2875; C12Q 1/6883; C12N 2310/11; C12N 2310/14; C12N 2310/531; C12N 2320/30; C12Q 2600/156

(57) ABSTRACT

The present disclosure provides methods of treating a subject having decreased bone mineral density or at risk of developing decreased bone mineral density, and methods of identifying subjects having an increased risk of developing decreased bone mineral density.

### REFERENCE TO SEQUENCE LISTING

This application includes a Sequence Listing filed electronically as an XML file named 381204340SEQ, created on Dec. 11, 2024, with a size of 363,730 bytes. The Sequence Listing is incorporated herein by reference.

### FIELD

The present disclosure relates generally to the treatment of subjects having decreased bone mineral density with Cluster of Differentiation 109 (CD109) inhibitors, and methods of identifying subjects having an increased risk of developing decreased bone mineral density.

### BACKGROUND

Degenerative conditions of the bone can make individuals susceptible to bone fractures, bone pain, and other complications. Two significant degenerative conditions of the bone are osteopenia and osteoporosis. Decreased bone mineral density (osteopenia) is a condition of the bone that is less severe than osteoporosis and is characterized by a reduction in bone mass due to the loss of bone at a rate greater than new bone growth. Osteopenia manifests in bone having a mineral density lower than normal peak bone mineral density, but not as low as found in osteoporosis. Osteopenia can arise from a decrease in muscle activity, which may occur as the result of a bone fracture, bed rest, fracture immobilization, joint reconstruction, arthritis, and the like. Osteoporosis is a disease characterized by a gradual bone weakening due to demineralization of the bone and/or problems with its architecture. Osteoporosis manifests in bones by making them more susceptible to breaking. Hormone deficiencies related to menopause in women, and hormone deficiencies due to aging in both sexes contribute to degenerative conditions of the bone. In addition, insufficient dietary uptake of minerals essential to bone growth and maintenance are potential causes of bone loss. Genetic influences on osteoporosis have also been reported (Morris et al., Nature Genet., 2019, 51, 258-266).

The effects of osteopenia can be slowed, stopped, and even reversed by reproducing some of the effects of muscle use on the bone. This typically involves some application or simulation of the effects of mechanical stress on the bone. Compounds for the treatment of osteopenia or osteoporosis include pharmaceutical preparations that induce bone growth or retard bone demineralization, or mineral complexes that supplement the diet in an effort to replenish lost bone minerals. Low levels of estrogen in women, and low levels of androgen in men are the primary hormonal deficiencies that cause osteoporosis in the respective sexes. Other hormones such as the thyroid hormones, progesterone, and testosterone contribute to bone health. As such, the aforementioned hormonal compounds have been developed synthetically, or extracted from non-mammalian sources, and compounded into therapies for treating osteoporosis. Mineral supplement preparations containing iodine, zinc, manganese, boron, strontium, vitamin D3, calcium, magnesium, vitamin K, phosphorous, and copper have also been used to supplement insufficient dietary uptake of such minerals. However, long-term hormonal therapies have undesirable side effects such as increased cancer risk. In addition, it is uncertain if many of the mineral and hormonal supplements proposed can actually reduce the risk of fracture. Moreover, therapies using many synthetic or non-mammalian hormones have additional undesirable side effects, such as an increased risk of cardiovascular disorders, neurological disorders, or the exacerbation of pre-existing conditions.

Cluster Of Differentiation 109 (CD109) is a member of alpha2-macroglobulin/complement (AMCOM) family of thioester containing proteins. This glycosyl phosphatidylinositol (GPI)-linked glycoprotein localizes to the surface of platelets, activated T-cells, and endothelial cells. In addition, CD109 is expressed in hematopoietic cell lineages and some epithelial cells. CD109 protein binds to and negatively regulates signaling by transforming growth factor beta (TGF-β) in keratinocytes, and also associates with TGF-βRI and TGF-βRII. Moreover, CR109 could function as protease inhibitors like most members of AMCOM family.

### SUMMARY

The present disclosure provides methods of treating a subject having decreased bone mineral density or at risk of developing decreased bone mineral density, the methods comprising administering a CD109 inhibitor to the subject.

The present disclosure also provides methods of treating a subject having osteopenia or at risk of developing osteopenia, the methods comprising administering a CD109 inhibitor to the subject.

The present disclosure also provides methods of treating a subject having Type I osteoporosis or at risk of developing Type I osteoporosis, the methods comprising administering a CD109 inhibitor to the subject.

The present disclosure also provides methods of treating a subject having Type II osteoporosis or at risk of developing Type II osteoporosis, the methods comprising administering a CD109 to the subject.

The present disclosure also provides methods of treating a subject having secondary osteoporosis or at risk of developing secondary osteoporosis, the methods comprising administering a CD109 inhibitor to the subject.

The present disclosure also provides methods of treating a subject with a therapeutic agent that treats or inhibits decreased bone mineral density, wherein the subject has decreased bone mineral density or is at risk of developing decreased bone mineral density, the methods comprising the steps of: determining whether the subject has a CD109 missense variant nucleic acid molecule encoding a CD109 predicted loss-of-function polypeptide by: obtaining or having obtained a biological sample from the subject; and performing or having performed a sequence analysis on the biological sample to determine if the subject has a genotype comprising the CD109 missense variant nucleic acid molecule encoding a CD109 predicted loss-of-function polypeptide; and: i) administering or continuing to administer the therapeutic agent that treats or inhibits decreased bone mineral density in a standard dosage amount to a subject that is CD109 reference, and/or administering a CD109 inhibitor to the subject; ii) administering or continuing to administer the therapeutic agent that treats or inhibits decreased bone mineral density in an amount that is the same as or less than a standard dosage amount to a subject that is heterozygous for the CD109 missense variant nucleic acid molecule, and/or administering a CD109 inhibitor to the subject; or iii) administering or continuing to administer the therapeutic agent that treats or inhibits decreased bone mineral density in an amount that is the same as or less than a standard dosage amount to a subject that is homozygous for the CD109 missense variant nucleic acid molecule; wherein the presence of a genotype having the CD109 missense variant nucleic acid molecule encoding the CD109 predicted loss-of-function polypeptide indicates the subject has a decreased risk of developing decreased bone mineral density.

The present disclosure also provides methods of identifying a subject having an increased risk of developing decreased bone mineral density, the methods comprising: determining or having determined the presence or absence of a CD109 missense variant nucleic acid molecule encoding a CD109 predicted loss-of-function polypeptide in a biological sample obtained from the subject; when the subject is CD109 reference, then the subject has an increased risk of developing decreased bone mineral density; and when the subject is heterozygous or homozygous for the CD109 missense variant nucleic acid molecule encoding the CD109 predicted loss-of-function polypeptide, then the subject has a decreased risk of developing decreased bone mineral density.

The present disclosure also provides therapeutic agents that treat or inhibit decreased bone mineral density for use in the treatment of decreased bone mineral density in a subject having: a CD109 missense variant genomic nucleic acid molecule encoding a CD109 predicted loss-of-function polypeptide; a CD109 missense variant mRNA molecule encoding a CD109 predicted loss-of-function polypeptide; or a CD109 missense variant cDNA molecule encoding a CD109 predicted loss-of-function polypeptide.

The present disclosure also provides CD109 inhibitors for use in the treatment of decreased bone mineral density in a subject that: a) is reference for a CD109 genomic nucleic acid molecule, a CD109 mRNA molecule, or a CD109 cDNA molecule; or b) is heterozygous for: i) a CD109 missense variant genomic nucleic acid molecule encoding a CD109 predicted loss-of-function polypeptide; ii) a CD109 missense variant mRNA molecule encoding a CD109 predicted loss-of-function polypeptide; or iii) a CD109 missense variant cDNA molecule encoding a CD109 predicted loss-of-function polypeptide.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the term “isolated”, in regard to a nucleic acid molecule or a polypeptide, means that the nucleic acid molecule or polypeptide is in a condition other than its native environment, such as apart from blood and/or animal tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or Alternately phosphorylated or derivatized forms.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates. In some embodiments, the subject is a human. In some embodiments, the human is a patient under the care of a physician.

It has been observed in accordance with the present disclosure that CD109 missense variant nucleic acid molecules encoding a CD109 predicted loss-of-function polypeptide (whether these variations are homozygous or heterozygous in a particular subject) associate with a decreased risk of developing decreased bone mineral density. The details or directionality of CD109's involvement in bone mineral density is unclear. The data presented herein is the first to show that rare, nonsynonymous/loss-of-unction variants in CD109 are associated with a decreased risk of developing decreased bone mineral density. Therefore, subjects that are CD109 reference or heterozygous for CD109 missense variant nucleic acid molecules encoding CD109 predicted loss-of-function polypeptides may be treated with a CD109 inhibitor such that decreased bone mineral density is inhibited, the symptoms thereof are reduced, and/or development of symptoms is repressed. It is also believed that such subjects having decreased bone mineral density may further be treated with therapeutic agents that treat or inhibit decreased bone mineral density.

For purposes of the present disclosure, any particular subject, such as a human, can be categorized as having one of three CD109 genotypes: i) CD109 reference; ii) heterozygous for a CD109 missense variant nucleic acid molecule encoding a CD109 predicted loss-of-function polypeptide; or iii) homozygous for a CD109 missense variant nucleic acid molecules encoding a CD109 predicted loss-of-function polypeptide. A subject is CD109 reference when the subject does not have a copy of a CD109 missense variant nucleic acid molecules encoding a CD109 predicted loss-of-function polypeptide. A subject is heterozygous for a CD109 missense variant nucleic acid molecules encoding a CD109 predicted loss-of-function polypeptide when the subject has a single copy of a CD109 missense variant nucleic acid molecule encoding a CD109 predicted loss-of-function polypeptide. A CD109 missense variant nucleic acid molecule encoding a CD109 predicted loss-of-function polypeptide is any nucleic acid molecule (such as, a genomic nucleic acid molecule, an mRNA molecule, or a cDNA molecule) encoding a variant CD109 polypeptide having a partial loss-of-function, a complete loss-of-function, a predicted partial loss-of-function, or a predicted complete loss-of-function. A subject who has a CD109 polypeptide having a partial loss-of-function (or predicted partial loss-of-function) is hypomorphic for CD109. A subject is homozygous for a CD109 missense variant nucleic acid molecule encoding a CD109 predicted loss-of-function polypeptide when the subject has two copies (same or different) of a CD109 missense variant nucleic acid molecules encoding a CD109 predicted loss-of-function polypeptide.

For subjects that are genotyped or determined to be CD109 reference, such subjects have an increased risk of developing decreased bone mineral density, such as osteopenia, Type I osteoporosis, Type II osteoporosis, and/or secondary osteoporosis. For subjects that are genotyped or determined to be either CD109 reference or heterozygous for a CD109 missense variant nucleic acid molecules encoding a CD109 predicted loss-of-function polypeptide, such subjects or subjects can be treated with a CD109 inhibitor.

In any of the embodiments described herein, the CD109 missense variant nucleic acid molecules encoding a CD109 predicted loss-of-function polypeptide can be any nucleic acid molecule (such as, for example, genomic nucleic acid molecule, mRNA molecule, or cDNA molecule) encoding a CD109 variant polypeptide having a partial loss-of-function, a complete loss-of-function, a predicted partial loss-of-function, or a predicted complete loss-of-function. In some embodiments, the CD109 missense variant nucleic acid molecules encoding a CD109 predicted loss-of-function polypeptide is associated with a reduced in vitro response to CD109 ligands compared with reference CD109. In some embodiments, the CD109 missense variant nucleic acid molecules encoding a CD109 predicted loss-of-function polypeptide is a CD109 variant that results or is predicted to result in a premature truncation of a CD109 polypeptide compared to the human reference genome sequence. In some embodiments, the CD109 missense variant nucleic acid molecules encoding a CD109 predicted loss-of-function polypeptide is a variant that is predicted to be damaging by in vitro prediction algorithms such as Polyphen, SIFT, or similar algorithms. In some embodiments, the CD109 missense variant nucleic acid molecules encoding a CD109 predicted loss-of-function polypeptide is a variant that causes or is predicted to cause a nonsynonymous amino-acid substitution in CD109 and whose allele frequency is less than 1/100 alleles in the population from which the subject is selected. In some embodiments, the CD109 missense variant nucleic acid molecules encoding a CD109 predicted loss-of-function polypeptide is any rare missense variant (allele frequency <0.1%; or 1 in 1,000 alleles), or any splice-site, stop-gain, start-loss, stop-loss, frameshift, or in-frame indel, or other frameshift CD109 variant.

In any of the embodiments described herein, the CD109 predicted loss-of-function polypeptide can be any CD109 polypeptide having a partial loss-of-function, a complete loss-of-function, a predicted partial loss-of-function, or a predicted complete loss-of-function.

In any of the embodiments described herein, the CD109 missense variant nucleic acid molecules encoding a CD109 predicted loss-of-function polypeptide can include variations at positions of chromosome 6 using the nucleotide sequence of the CD109 reference genomic nucleic acid molecule (SEQ ID NO:1; ENSG00000156535.15 chr6:73,695,785-73,828,316 in the GRCh38/hg38 human genome assembly) as a reference sequence.

Numerous genetic variants in CD109 exist which cause subsequent changes in the CD109 polypeptide sequence including, but not limited to: 6:73730573:A:G, 6:73823473:GA:G (p.Ser1394fs, p.Ser1317fs, p. Ser1377fs), 6:73763607:C:A (p.Phe343Leu, p. Phe266Leu, p. Phe343Leu), 6:73803256:G:T (p.Gly972Val, p. Gly895Val, p. Gly972Val), 6:73818486:T:C (p.Val1337Ala, p. Val1260Ala, p. Val1320Ala), 6:73787379:G:A (p.Gly828Glu, p. Gly751Glu, p. Gly828Glu), 6:73771510:A:G (p.Ile586Val, p. Ile509Val, p. Ile586Val), 6:73806987:A:T (p.His1035Leu, p. His958Leu, p. His1035Leu), 6:73758991:A:G (p.Met241Val, p. Met164Val, p. Met241Val), 6:73823456:A:G 6:73762778:A:C (p.Glu298Ala, p. Glu221Ala, p. Glu298Ala), 6:73763660:A:G (p.Lys361Arg, p. Lys284Arg, p. Lys361Arg), 6:73730573:A:G (p.Lys169Arg, p. Lys169Arg), 6:73806956:G:A (p.Glyl025Ser, p. Gly948Ser, p. Glyl025Ser), 6:73792628:G:C (p.Asp902His, p. Asp825His, p. Asp902His), 6:73806926:A:T (p.Thr1015Ser, p. Thr938Ser, p. Thr1015Ser), 6:73771576:G:A (p.Glu608Lys, p. Glu531Lys, p. Glu608Lys), 6:73815026:C:T (p.Arg1272*, p. Arg1195*, p. Arg1255*), 6:73765952:G:A (p.Gly377Asp, p. Gly300Asp, p. Gly377Asp).

Any one or more (i.e., any combination) of the CD109 missense variant nucleic acid molecules encoding a CD109 predicted loss-of-function polypeptide can be used within any of the methods described herein to determine whether a subject has an increased risk of developing decreased bone mineral density. The combinations of particular variants can form a mask used for statistical analysis of the particular correlation of CD109 and increased risk of developing decreased bone mineral density.

In any of the embodiments described herein, the decreased bone mineral density is osteopenia, Type I osteoporosis, Type II osteoporosis, and/or secondary osteoporosis. In some embodiments, the decreased bone mineral density is osteopenia. In some embodiments, the decreased bone mineral density is Type I osteoporosis. In some embodiments, the decreased bone mineral density is Type II osteoporosis. In some embodiments, the decreased bone mineral density is secondary osteoporosis.

Symptoms of a decreased bone mineral density include, but are not limited to, increased bone fragility (manifesting as bone fracture as a result of a mild to moderate trauma), reduced bone density, localized bone pain and weakness in an area of a broken bone, loss of height or change in posture, such as stooping over, high levels of serum calcium or alkaline phosphatase on a blood test, vitamin D deficiency, and joint or muscle aches, or any combination thereof.

The present disclosure provides methods of treating a subject having decreased bone mineral density or at risk of developing decreased bone mineral density, the methods comprising administering a CD109 inhibitor to the subject.

The present disclosure also provides methods of treating a subject having osteopenia or at risk of developing osteopenia, the methods comprising administering a CD109 inhibitor to the subject.

The present disclosure also provides methods of treating a subject having Type I osteoporosis or at risk of developing Type I osteoporosis, the methods comprising administering a CD109 inhibitor to the subject.

The present disclosure also provides methods of treating a subject having Type II osteoporosis or at risk of developing Type II osteoporosis, the methods comprising administering a CD109 to the subject.

The present disclosure also provides methods of treating a subject having secondary osteoporosis or at risk of developing secondary osteoporosis, the methods comprising administering a CD109 inhibitor to the subject.

In some embodiments, the CD109 inhibitor comprises an inhibitory nucleic acid molecule. Examples of inhibitory nucleic acid molecules include, but are not limited to, antisense nucleic acid molecules, small interfering RNAs (siRNAs), and short hairpin RNAs (shRNAs). Such inhibitory nucleic acid molecules can be designed to target any region of a CD109 nucleic acid molecule. In some embodiments, the antisense RNA, siRNA, or shRNA hybridizes to a sequence within a CD109 genomic nucleic acid molecule or mRNA molecule and decreases expression of the CD109 polypeptide in a cell in the subject. In some embodiments, the CD109 inhibitor comprises an antisense molecule that hybridizes to a CD109 genomic nucleic acid molecule or mRNA molecule and decreases expression of the CD109 polypeptide in a cell in the subject. In some embodiments, the CD109 inhibitor comprises an siRNA that hybridizes to a CD109 genomic nucleic acid molecule or mRNA molecule and decreases expression of the CD109 polypeptide in a cell in the subject. In some embodiments, the CD109 inhibitor comprises an shRNA that hybridizes to a CD109 genomic nucleic acid molecule or mRNA molecule and decreases expression of the CD109 polypeptide in a cell in the subject.

The inhibitory nucleic acid molecules can comprise RNA, DNA, or both RNA and DNA. The inhibitory nucleic acid molecules can also be linked or fused to a heterologous nucleic acid sequence, such as in a vector, or a heterologous label. For example, the inhibitory nucleic acid molecules can be within a vector or as an exogenous donor sequence comprising the inhibitory nucleic acid molecule and a heterologous nucleic acid sequence. The inhibitory nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3XFLAG, 6XHis or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The inhibitory nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The inhibitory nucleic acid molecules can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O—, S—, or N-alkyl; O—, S—, or N-alkenyl; O—, S— or N-alkynyl; or O-alkyl-O-alkyl, wherein the alkyl, alkenyl, and alkynyl may be substituted or unsubstituted C₁-10alkyl or C₂-10alkenyl, and C₂-10alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

In some embodiments, the antisense nucleic acid molecules are gapmers, whereby the first one to seven nucleotides at the 5′ and 3′ ends each have 2′-methoxyethyl (2′-MOE) modifications. In some embodiments, the first five nucleotides at the 5′ and 3′ ends each have 2′-MOE modifications. In some embodiments, the first one to seven nucleotides at the 5′ and 3′ ends are RNA nucleotides. In some embodiments, the first five nucleotides at the 5′ and 3′ ends are RNA nucleotides. In some embodiments, each of the backbone linkages between the nucleotides is a phosphorothioate linkage.

In some embodiments, the siRNA molecules have termini modifications. In some embodiments, the 5′ end of the antisense strand is phosphorylated. In some embodiments, 5′-phosphate analogs that cannot be hydrolyzed, such as 5′-(E)-vinyl-phosphonate are used.

In some embodiments, the siRNA molecules have backbone modifications. In some embodiments, the modified phosphodiester groups that link consecutive ribose nucleosides have been shown to enhance the stability and in vivo bioavailability of siRNAs The non-ester groups (—OH, ═O) of the phosphodiester linkage can be replaced with sulfur, boron, or acetate to give phosphorothioate, boranophosphate, and phosphonoacetate linkages. In addition, substituting the phosphodiester group with a phosphotriester can facilitate cellular uptake of siRNAs and retention on serum components by eliminating their negative charge. In some embodiments, the siRNA molecules have sugar modifications. In some embodiments, the sugars are deprotonated (reaction catalyzed by exo- and endonucleases) whereby the 2′-hydroxyl can act as a nucleophile and attack the adjacent phosphorous in the phosphodiester bond. Such alternatives include 2′-O-methyl, 2′-O-methoxyethyl, and 2′-fluoro modifications.

In some embodiments, the siRNA molecules have base modifications. In some embodiments, the bases can be substituted with modified bases such as pseudouridine, 5′-methylcytidine, N6-methyladenosine, inosine, and N7-methylguanosine.

In some embodiments, the siRNA molecules are conjugated to lipids. Lipids can be conjugated to the 5′ or 3′ termini of siRNA to improve their in vivo bioavailability by allowing them to associate with serum lipoproteins. Representative lipids include, but are not limited to, cholesterol and vitamin E, and fatty acids, such as palmitate and tocopherol.

In some embodiments, a representative siRNA has the following formula:

Sense: mN*mN*/i2FN/mN/i2FN/mN/i2FN/mN/i2FN/mN/i2FN/mN/i2FN/mN/i2FN/mN/i2FN/*mN*/32FN/

Antisense:/52FN/*/i2FN/*mN/i2FN/mN/i2FN/mN/i2FN/mN/i2FN/mN/i2FN/mN/i2FN/mN/12FN/mN/i2FN/mN*N*N

wherein: “N” is the base; “2F” is a 2′-F modification; “m” is a 2′-O-methyl modification, “I” is an internal base; and “*” is a phosphorothioate backbone linkage.

The present disclosure also provides vectors comprising any one or more of the inhibitory nucleic acid molecules. In some embodiments, the vectors comprise any one or more of the inhibitory nucleic acid molecules and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

The present disclosure also provides compositions comprising any one or more of the inhibitory nucleic acid molecules. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

In some embodiments, the CD109 inhibitor is or comprises LY294002, which is a PI3K inhibitor that suppresses CD109 expression.

In some embodiments, the CD109 inhibitor comprises a nuclease agent that induces one or more nicks or double-strand breaks at a recognition sequence(s) or a DNA-binding protein that binds to a recognition sequence within a CD109 genomic nucleic acid molecule. The recognition sequence can be located within a coding region of the CD109 gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the CD109 gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

Suitable nuclease agents and DNA-binding proteins for use herein include, but are not limited to, zinc finger protein or zinc finger nuclease (ZFN) pair, Transcription Activator-Like Effector (TALE) protein or Transcription Activator-Like Effector Nuclease (TALEN), or Clustered Regularly Interspersed Short Palindromic Repeats (CRISPR)/CRISPR-associated (Cas) systems. The length of the recognition sequence can vary, and includes, for example, recognition sequences that are about 30-36 bp for a zinc finger protein or ZFN pair, about 15-18 bp for each ZFN, about 36 bp for a TALE protein or TALEN, and about 20 bp for a CRISPR/Cas guide RNA.

In some embodiments, CRISPR/Cas systems can be used to modify a CD109 genomic nucleic acid molecule within a cell. The methods and compositions disclosed herein can employ CRISPR-Cas systems by utilizing CRISPR complexes (comprising a guide RNA (gRNA) complexed with a Cas protein) for site-directed cleavage of CD109 nucleic acid molecules.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in a CD109 genomic nucleic acid molecule or it can be a nickase that creates a single-strand break in a CD109 genomic nucleic acid molecule. Additional examples of Cas proteins include, but are not limited to, Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. Cas proteins can also be operably linked to heterologous polypeptides as fusion proteins. For example, a Cas protein can be fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA.

In some embodiments, targeted genetic modifications of CD109 genomic nucleic acid molecules can be generated by contacting a cell with a Cas protein and one or more gRNAs that hybridize to one or more gRNA recognition sequences within a target genomic locus in the CD109 genomic nucleic acid molecule. For example, a gRNA recognition sequence can be located within a region of SEQ ID NO:1. The gRNA recognition sequence can include or be proximate to the start codon of a CD109 genomic nucleic acid molecule or the stop codon of a CD109 genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

The gRNA recognition sequences within a target genomic locus in a CD109 genomic nucleic acid molecule are located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes PAM. In addition, 5′-NGA-3′ can be a highly efficient non-canonical PAM for human cells. Generally, the PAM is about 2-6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10, about 2 to about 5 base pairs, or three base pairs upstream or downstream of the PAM sequence. In some embodiments (such as when Cas9 from S. pyogenes or a closely related Cas9 is used), the PAM sequence of the non-complementary strand can be 5′-NGG-3′, where N is any DNA nucleotide and is immediately 3′ of the gRNA recognition sequence of the non-complementary strand of the target DNA. As such, the PAM sequence of the complementary strand would be 5′-CCN-31, where N is any DNA nucleotide and is immediately 5′ of the gRNA recognition sequence of the complementary strand of the target DNA.

A gRNA is an RNA molecule that binds to a Cas protein and targets the Cas protein to a specific location within a CD109 genomic nucleic acid molecule. An exemplary gRNA is a gRNA effective to direct a Cas enzyme to bind to or cleave a CD109 genomic nucleic acid molecule, wherein the gRNA comprises a DNA-targeting segment that hybridizes to a gRNA recognition sequence within the CD109 genomic nucleic acid molecule. Exemplary gRNAs comprise a DNA-targeting segment that hybridizes to a gRNA recognition sequence present within a CD109 genomic nucleic acid molecule that includes or is proximate to the start codon or the stop codon. For example, a gRNA can be selected such that it hybridizes to a gRNA recognition sequence that is located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the start codon or located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs can comprise 20 nucleotides.

Examples of suitable gRNA recognition sequences located within the human CD109 reference gene are set forth in Table 1 as SEQ ID NOs:37-58.

TABLE 1
Guide RNA Recognition Sequences Near CD109

| Strand | gRNA Recognition Sequence | SEQ ID NO: |
|---|---|---|
| + | GCCTCCAAGTCCTGTCTCAAT | 37 |
| + | GGTACCATCACGGCAAAGTAT | 38 |
| + | GTACCATCACGGCAAAGTATA | 39 |
| + | GCTACAGTTGAAGGCCTATTT | 40 |
| + | GATTGAAGGAGTTAAGCTATA | 41 |
| + | GGTCTTGGACTAACAACTACT | 42 |
| + | GAAAGATGCCACTGAGGTTAA | 43 |
| + | GCATCTACTCAGGATACCACT | 44 |
| + | GGTACAGCCAACGGCAGTTAA | 45 |
| + | GGCTCTTATGGAAGTTAACCT | 46 |
| + | GACAGGCGGTGAGAAGTTACA | 47 |
| + | GCCCAGTGGTCTCAGTAGATA | 48 |
| + | GCCGATCCTTACATAGATA | 49 |
| + | CCTAGATTCTTAAGCATTA | 50 |
| + | AAGCCTGTGTAATTGTGTA | 51 |
| + | AGAGTTCAGATCACTGCAA | 52 |
| + | AGGAGACGTAACGCTTACA | 53 |
| + | TGTAAGCACTAATGTGTTC | 54 |
| + | CTGTACCTGATTCTATCAC | 55 |
| + | ACTAAGAAGTAAGTGTAAC | 56 |
| + | TGCAGAATATGCTGAGAGG | 57 |
| = | TTGGAGATGTTCTTGGTCC | 58 |

The Cas protein and the gRNA form a complex, and the Cas protein cleaves the target CD109 genomic nucleic acid molecule. The Cas protein can cleave the nucleic acid molecule at a site within or outside of the nucleic acid sequence present in the target CD109 genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the CD109 genomic nucleic acid molecule to which a DNA-targeting segment of a gRNA will bind.

Such methods can result, for example, in a CD109 genomic nucleic acid molecule in which a region of SEQ ID NO:1 is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the CD109 genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

In some embodiments, the methods of treatment further comprise detecting the presence or absence of a CD109 missense variant nucleic acid molecule encoding a CD109 predicted loss-of-function polypeptide in a biological sample from the subject. As used throughout the present disclosure, a “CD109 missense variant nucleic acid molecule encoding a CD109 predicted loss-of-function polypeptide” is any CD109 nucleic acid molecule (such as, for example, genomic nucleic acid molecule, mRNA molecule, or cDNA molecule) encoding a CD109 polypeptide having a partial loss-of-function, a complete loss-of-function, a predicted partial loss-of-function, or a predicted complete loss-of-function.

The present disclosure also provides methods of treating a subject with a therapeutic agent that treats or inhibits decreased bone mineral density, wherein the subject has decreased bone mineral density or is at risk of developing decreased bone mineral density. In some embodiments, the subject has decreased bone mineral density. In some embodiments, the subject is at risk of developing decreased bone mineral density. In some embodiments, the methods comprise determining whether the subject has a CD109 missense variant nucleic acid molecule encoding a CD109 predicted loss-of-function polypeptide by obtaining or having obtained a biological sample from the subject, and performing or having performed a sequence analysis on the biological sample to determine if the subject has a genotype comprising the CD109 missense variant nucleic acid molecule encoding a CD109 predicted loss-of-function polypeptide. In some embodiments, the methods further comprise administering or continuing to administer the therapeutic agent that treats or inhibits decreased bone mineral density in a standard dosage amount to a subject that is CD109 reference, and/or administering a CD109 inhibitor to the subject. In some embodiments, the methods further comprise administering or continuing to administer the therapeutic agent that treats or inhibits decreased bone mineral density in an amount that is the same as or less than a standard dosage amount to a subject that is heterozygous for the CD109 missense variant nucleic acid molecule, and/or administering a CD109 inhibitor to the subject. In some embodiments, the methods further comprise administering or continuing to administer the therapeutic agent that treats or inhibits decreased bone mineral density in an amount that is the same as or less than a standard dosage amount to a subject that is homozygous for the CD109 missense variant nucleic acid molecule. The presence of a genotype having the CD109 missense variant nucleic acid molecule encoding the CD109 predicted loss-of-function polypeptide indicates the subject has a decreased risk of developing decreased bone mineral density. In some embodiments, the subject is CD109 reference. In some embodiments, the subject is heterozygous for a CD109 missense variant nucleic acid molecule encoding a CD109 predicted loss-of-function polypeptide.

For subjects that are genotyped or determined to be either CD109 reference or heterozygous for a CD109 missense variant nucleic acid molecule encoding a CD109 predicted loss-of-function polypeptide, such subjects can be treated with a CD109 inhibitor, as described herein.

Detecting the presence or absence of a CD109 missense variant nucleic acid molecule encoding a CD109 predicted loss-of-function polypeptide in a biological sample from a subject and/or determining whether a subject has a CD109 missense variant nucleic acid molecule encoding a CD109 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when the subject is CD109 reference, the subject is administered a therapeutic agent that treats or inhibits decreased bone mineral density in a standard dosage amount. In some embodiments, when the subject is heterozygous for a CD109 missense variant nucleic acid molecule encoding a CD109 predicted loss-of-function polypeptide, the subject is administered a therapeutic agent that treats or inhibits decreased bone mineral density in a dosage amount that is the same as or less than a standard dosage amount.

In some embodiments, the treatment methods further comprise detecting the presence or absence of a CD109 predicted loss-of-function polypeptide in a biological sample from the subject. In some embodiments, when the subject does not have a CD109 predicted loss-of-function polypeptide, the subject is administered a therapeutic agent that treats or inhibits decreased bone mineral density in a standard dosage amount. In some embodiments, when the subject has a CD109 predicted loss-of-function polypeptide, the subject is administered a therapeutic agent that treats or inhibits decreased bone mineral density in a dosage amount that is the same as or less than a standard dosage amount.

The present disclosure also provides methods of treating a subject with a therapeutic agent that treats or inhibits decreased bone mineral density, wherein the subject has decreased bone mineral density or is at risk of developing decreased bone mineral density. In some embodiments, the subject has decreased bone mineral density. In some embodiments, the subject is at risk of developing decreased bone mineral density. In some embodiments, the method comprises determining whether the subject has a CD109 predicted loss-of-function polypeptide by obtaining or having obtained a biological sample from the subject, and performing or having performed an assay on the biological sample to determine if the subject has a CD109 predicted loss-of-function polypeptide. When the subject does not have a CD109 predicted loss-of-function polypeptide, the therapeutic agent that treats or inhibits decreased bone mineral density is administered or continued to be administered to the subject in a standard dosage amount, and/or a CD109 inhibitor is administered to the subject. When the subject has a CD109 predicted loss-of-function polypeptide, the therapeutic agent that treats or inhibits decreased bone mineral density is administered or continued to be administered to the subject in an amount that is the same as or less than a standard dosage amount, and/or a CD109 inhibitor is administered to the subject. The presence of a CD109 predicted loss-of-function polypeptide indicates the subject has a decreased risk of developing decreased bone mineral density. In some embodiments, the subject has a CD109 predicted loss-of-function polypeptide. In some embodiments, the subject does not have a CD109 predicted loss-of-function polypeptide.

Detecting the presence or absence of a CD109 predicted loss-of-function polypeptide in a biological sample from a subject and/or determining whether a subject has a CD109 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the subject.

Examples of therapeutic agents that treat or inhibit decreased bone mineral density include, but are not limited to: calcium and vitamin D supplementation (vitamin D2, vitamin D3, and cholecalciferol), bisphosphonate medications, such as FOSAMAX®, (alendronate), BONIVA® (ibandronate), RECLAST® (zoledronate), ACTONEL® (risedronate), MIACALCIN®, FORTICAL®, and CALCIMAR® (calcitonin), FORTEO® (teriparatide), PROLIA® (denosumab), hormone replacement therapy with estrogen and progesterone as well as EVISTA® (raloxifene), and EVENITY® (romosozumab). In some embodiments, the therapeutic agent that treats or inhibits decreased bone mineral density is vitamin D2, vitamin D3, cholecalciferol, alendronate, ibandronate, zoledronate, risedronate, calcitonin, teriparatide, denosumab, or raloxifene. In some embodiments, the therapeutic agent that treats or inhibits decreased bone mineral density is vitamin D2. In some embodiments, the therapeutic agent that treats or inhibits decreased bone mineral density is vitamin D3. In some embodiments, the therapeutic agent that treats or inhibits decreased bone mineral density is cholecalciferol. In some embodiments, the therapeutic agent that treats or inhibits decreased bone mineral density is alendronate. In some embodiments, the therapeutic agent that treats or inhibits decreased bone mineral density is ibandronate. In some embodiments, the therapeutic agent that treats or inhibits decreased bone mineral density is zoledronate. In some embodiments, the therapeutic agent that treats or inhibits decreased bone mineral density is risedronate. In some embodiments, the therapeutic agent that treats or inhibits decreased bone mineral density is calcitonin. In some embodiments, the therapeutic agent that treats or inhibits decreased bone mineral density is teriparatide. In some embodiments, the therapeutic agent that treats or inhibits decreased bone mineral density is denosumab. In some embodiments, the therapeutic agent that treats or inhibits decreased bone mineral density is raloxifene.

In some embodiments, the dose of the therapeutic agents that treat or inhibit decreased bone mineral density can be decreased by about 10%, by about 20%, by about 30%, by about 40%, by about 50%, by about 60%, by about 70%, by about 80%, or by about 90% for subjects that are heterozygous for a CD109 missense variant nucleic acid molecule encoding a CD109 predicted loss-of-function polypeptide (i.e., a less than the standard dosage amount) compared to subjects that are CD109 reference (who may receive a standard dosage amount). In some embodiments, the dose of the therapeutic agents that treat or inhibit decreased bone mineral density can be decreased by about 10%, by about 20%, by about 30%, by about 40%, or by about 50%. In addition, the subjects that are heterozygous for a CD109 missense variant nucleic acid molecule encoding a CD109 predicted loss-of-function polypeptide can be administered less frequently compared to subjects that are CD109 reference.

In some embodiments, the dose of the therapeutic agents that treat or inhibit decreased bone mineral density can be decreased by about 10%, by about 20%, by about 30%, by about 40%, by about 50%, for subjects that are homozygous for a CD109 missense variant nucleic acid molecule encoding a CD109 predicted loss-of-function polypeptide compared to subjects that are heterozygous for a CD109 missense variant nucleic acid molecule encoding a CD109 predicted loss-of-function polypeptide. In some embodiments, the dose of the therapeutic agents that treat or inhibit decreased bone mineral density can be decreased by about 10%, by about 20%, by about 30%, by about 40%, or by about 50%. In addition, the dose of therapeutic agents that treat or inhibit decreased bone mineral density in subjects that are homozygous for a CD109 missense variant nucleic acid molecule encoding a CD109 predicted loss-of-function polypeptide can be administered less frequently compared to subjects that are heterozygous for a CD109 missense variant nucleic acid molecule encoding a CD109 predicted loss-of-function polypeptide.

Administration of the therapeutic agents that treat or inhibit decreased bone mineral density and/or CD109 inhibitors can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more.

Administration of the therapeutic agents that treat or inhibit decreased bone mineral density and/or CD109 inhibitors can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in decreased bone mineral density, a decrease/reduction in the severity of decreased bone mineral density (such as, for example, a reduction or inhibition of development of decreased bone mineral density), a decrease/reduction in symptoms and decreased bone mineral density-related effects, delaying the onset of symptoms and decreased bone mineral density-related effects, reducing the severity of symptoms of decreased bone mineral density-related effects, reducing the number of symptoms and decreased bone mineral density-related effects, reducing the latency of symptoms and decreased bone mineral density-related effects, an amelioration of symptoms and decreased bone mineral density-related effects, reducing secondary symptoms, reducing secondary infections, preventing relapse to decreased bone mineral density, decreasing the number or frequency of relapse episodes, increasing latency between symptomatic episodes, increasing time to sustained progression, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of decreased bone mineral density development/progression (such as, for example, a complete or partial avoidance/inhibition or a delay), and an increased survival time of the affected host animal, following administration of a therapeutic protocol. Treatment of decreased bone mineral density encompasses the treatment of a subject already diagnosed as having any form of decreased bone mineral density at any clinical stage or manifestation, the delay of the onset or evolution or aggravation or deterioration of the symptoms or signs of decreased bone mineral density, and/or preventing and/or reducing the severity of decreased bone mineral density.

The present disclosure also provides methods of identifying a subject having an increased risk of developing decreased bone mineral density. In some embodiments, the method comprises determining or having determined in a biological sample obtained from the subject the presence or absence of a CD109 missense variant nucleic acid molecule (such as a genomic nucleic acid molecule, mRNA molecule, and/or cDNA molecule) encoding a CD109 predicted loss-of-function polypeptide encoding a CD109 polypeptide. When the subject lacks a CD109 missense variant nucleic acid molecule encoding a CD109 predicted loss-of-function polypeptide (i.e., the subject is genotypically categorized as a CD109 reference), then the subject has an increased risk of developing decreased bone mineral density. When the subject has a CD109 missense variant nucleic acid molecule encoding a CD109 predicted loss-of-function polypeptide (i.e., the subject is heterozygous or homozygous for a CD109 missense variant nucleic acid molecule encoding a CD109 predicted loss-of-function polypeptide), then the subject has a decreased risk of developing decreased bone mineral density.

Having a single copy of a CD109 missense variant nucleic acid molecule encoding a CD109 predicted loss-of-function polypeptide is more protective of a subject from developing decreased bone mineral density than having no copies of a CD109 missense variant nucleic acid molecule encoding a CD109 predicted loss-of-function polypeptide. Without intending to be limited to any particular theory or mechanism of action, it is believed that a single copy of a CD109 missense variant nucleic acid molecule encoding a CD109 predicted loss-of-function polypeptide (i.e., heterozygous for a CD109 missense variant nucleic acid molecule encoding a CD109 predicted loss-of-function polypeptide) is protective of a subject from developing decreased bone mineral density, and it is also believed that having two copies of a CD109 missense variant nucleic acid molecule encoding a CD109 predicted loss-of-function polypeptide (i.e., homozygous for a CD109 missense variant nucleic acid molecule encoding a CD109 predicted loss-of-function polypeptide) may be more protective of a subject from developing decreased bone mineral density, relative to a subject with a single copy. Thus, in some embodiments, a single copy of a CD109 missense variant nucleic acid molecule encoding a CD109 predicted loss-of-function polypeptide may not be completely protective, but instead, may be partially or incompletely protective of a subject from developing decreased bone mineral density. While not desiring to be bound by any particular theory, there may be additional factors or molecules involved in the development of decreased bone mineral density that are still present in a subject having a single copy of a CD109 missense variant nucleic acid molecule encoding a CD109 predicted loss-of-function polypeptide, thus resulting in less than complete protection from the development of decreased bone mineral density.

Determining whether a subject has a CD109 missense variant nucleic acid molecule encoding a CD109 predicted loss-of-function polypeptide in a biological sample from a subject and/or determining whether a subject has a CD109 missense variant nucleic acid molecule encoding a CD109 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when a subject is identified as having an increased risk of developing decreased bone mineral density, the subject is treated with a therapeutic agent that treats or inhibits decreased bone mineral density, and/or a CD109 inhibitor, as described herein. For example, when the subject is CD109 reference, and therefore has an increased risk of developing decreased bone mineral density, the subject is administered a CD109 inhibitor. In some embodiments, such a subject is also administered a therapeutic agent that treats or inhibits decreased bone mineral density. In some embodiments, when the subject is heterozygous for a CD109 missense variant nucleic acid molecule encoding a CD109 predicted loss-of-function polypeptide, the subject is administered the therapeutic agent that treats or inhibits decreased bone mineral density in a dosage amount that is the same as or less than a standard dosage amount, and is also administered a CD109 inhibitor. In some embodiments, such a subject is also administered a therapeutic agent that treats or inhibits decreased bone mineral density. In some embodiments, when the subject is homozygous for a CD109 missense variant nucleic acid molecule encoding a CD109 predicted loss-of-function polypeptide, the subject is administered the therapeutic agent that treats or inhibits decreased bone mineral density in a dosage amount that is the same as or less than a standard dosage amount. In some embodiments, the subject is CD109 reference. In some embodiments, the subject is heterozygous for a CD109 missense variant nucleic acid molecule encoding a CD109 predicted loss-of-function polypeptide. In some embodiments, the subject is homozygous for a CD109 missense variant nucleic acid molecule encoding a CD109 predicted loss-of-function polypeptide.

In some embodiments, any of the methods described herein can further comprise determining the subject's gene burden of having a CD109 missense variant nucleic acid molecule encoding a CD109 predicted loss-of-function polypeptide, and/or a CD109 predicted loss-of-function variant polypeptide associated with a decreased risk of developing decreased bone mineral density. The gene burden is the aggregate of all variants in the CD109 gene, which can be carried out in an association analysis with decreased bone mineral density. In some embodiments, the subject is homozygous for one or more CD109 missense variant nucleic acid molecules encoding a CD109 predicted loss-of-function polypeptide associated with a decreased risk of developing decreased bone mineral density. In some embodiments, the subject is heterozygous for one or more CD109 missense variant nucleic acid molecules encoding a CD109 predicted loss-of-function polypeptide associated with a decreased risk of developing decreased bone mineral density. The result of the association analysis suggests that CD109 missense variant nucleic acid molecules encoding a CD109 predicted loss-of-function polypeptide are associated with decreased risk of developing decreased bone mineral density. When the subject has a lower gene burden, the subject is at a higher risk of developing decreased bone mineral density and the subject is administered or continued to be administered the therapeutic agent that treats or inhibits decreased bone mineral density in a standard dosage amount. When the subject has a greater gene burden, the subject is at a lower risk of developing decreased bone mineral density and the subject is administered or continued to be administered the therapeutic agent that treats or inhibits decreased bone mineral density in an amount that is the same as or less than the standard dosage amount. The greater the gene burden, the lower the risk of developing decreased bone mineral density.

The CD109 missense variant nucleic acid molecule encoding a CD109 predicted loss-of-function polypeptide, and/or a CD109 predicted loss-of-function variant polypeptide used for determining the subject's gene burden include, but are not limited to 6:73730573:A:G, 6:73823473:GA:G (p.Ser1394fs, p.Ser1317fs, p. Ser1377fs), 6:73763607:C:A (p.Phe343Leu, p. Phe266Leu, p. Phe343Leu), 6:73803256:G:T (p.Gly972Val, p. Gly895Val, p. Gly972Val), 6:73818486:T:C (p.Val1337Ala, p. Val1260Ala, p. Val1320Ala), 6:73787379:G:A (p.Gly828Glu, p. Gly751Glu, p. Gly828Glu), 6:73771510:A:G (p.Ile586Val, p. Ile509Val, p. Ile586Val), 6:73806987:A:T (p.His1035Leu, p. His958Leu, p. His1035Leu), 6:73758991:A:G (p.Met241Val, p. Met164Val, p. Met241Val), 6:73823456:A:G 6:73762778:A:C (p.Glu298Ala, p. Glu221Ala, p. Glu298Ala), 6:73763660:A:G (p.Lys361Arg, p. Lys284Arg, p. Lys361Arg), 6:73730573:A:G (p.Lys169Arg, p. Lys169Arg), 6:73806956:G:A (p.Glyl025Ser, p. Gly948Ser, p. Glyl025Ser), 6:73792628:G:C (p.Asp902His, p. Asp825His, p. Asp902His), 6:73806926:A:T (p.Thr1015Ser, p. Thr938Ser, p. Thr1015Ser), 6:73771576:G:A (p.Glu608Lys, p. Glu531Lys, p. Glu608Lys), 6:73815026:C:T (p.Arg1272*, p. Arg1195*, p. Arg1255*), 6:73765952:G:A (p.Gly377Asp, p. Gly300Asp, p. Gly377Asp).

In some embodiments, the subject's gene burden of having any one or more CD109 missense variant nucleic acid molecules encoding a CD109 predicted loss-of-function polypeptide represents a weighted sum of a plurality of any of the CD109 missense variant nucleic acid molecules encoding a CD109 predicted loss-of-function polypeptide. In some embodiments, the gene burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 10,000, at least about 100,000, or at least about or more than 1,000,000 genetic variants present in or around (up to 10 Mb) the CD109 gene where the genetic burden is the number of alleles multiplied by the association estimate with decreased bone mineral density or related outcome for each allele (e.g., a weighted burden score). This can include any genetic variants, regardless of their genomic annotation, in proximity to the CD109 gene (up to 10 Mb around the gene) that show a non-zero association with decreased bone mineral density-related traits in a genetic association analysis. In some embodiments, when the subject has a gene burden above a desired threshold score, the subject has a decreased risk of developing decreased bone mineral density. In some embodiments, when the subject has an gene burden below a desired threshold score, the subject has an increased risk of developing decreased bone mineral density.

In some embodiments, the gene burden may be divided into quintiles, e.g., top quintile, intermediate quintile, and bottom quintile, wherein the top quintile of gene burden corresponds to the lowest risk group and the bottom quintile of gene burden corresponds to the highest risk group. In some embodiments, a subject having a greater gene burden comprises the highest weighted gene burdens, including, but not limited to the top 10%, top 20%, top 30%, top 40%, or top 50% of gene burdens from a subject population. In some embodiments, the genetic variants comprise the genetic variants having association with decreased bone mineral density in the top 10%, top 20%, top 30%, top 40%, or top 50% of p-value range for the association. In some embodiments, each of the identified genetic variants comprise the genetic variants having association with decreased bone mineral density with p-value of no more than about 10⁻², about 10⁻³, about 10⁻⁴, about 10⁻⁵, about 10⁻⁶, about 10⁻⁷, about 10⁻⁸, about 10⁻⁹, about 10⁻¹⁰, about 10⁻¹¹, about 10⁻¹², about 10⁻¹³, about 10⁻¹⁴, about or 10⁻¹⁵. In some embodiments, the identified genetic variants comprise the genetic variants having association with decreased bone mineral density with p-value of less than 5×10⁻⁸. In some embodiments, the identified genetic variants comprise genetic variants having association with decreased bone mineral density in high-risk subjects as compared to the rest of the reference population with odds ratio (OR) about 1.5 or greater, about 1.75 or greater, about 2.0 or greater, or about 2.25 or greater for the top 20% of the distribution; or about 1.5 or greater, about 1.75 or greater, about 2.0 or greater, about 2.25 or greater, about 2.5 or greater, or about 2.75 or greater. In some embodiments, the odds ratio (OR) may range from about 1.0 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, from about 4.5 to about 5.0, from about 5.0 to about 5.5, from about 5.5 to about 6.0, from about 6.0 to about 6.5, from about 6.5 to about 7.0, or greater than 7.0. In some embodiments, high-risk subjects comprise subjects having gene burdens in the bottom decile, quintile, or tertile in a reference population. The threshold of the gene burden is determined on the basis of the nature of the intended practical application and the risk difference that would be considered meaningful for that practical application.

In some embodiments, when a subject is identified as having an increased risk of developing decreased bone mineral density, the subject is treated with a therapeutic agent that treats or inhibits decreased bone mineral density, and/or a CD109 inhibitor, as described herein. For example, when the subject is CD109 reference, and therefore has an increased risk of developing decreased bone mineral density, the subject is administered a CD109 inhibitor. In some embodiments, such a subject is administered a therapeutic agent that treats or inhibits decreased bone mineral density. In some embodiments, when the subject is heterozygous for a CD109 missense variant nucleic acid molecule encoding a

CD109 predicted loss-of-function polypeptide, the subject is administered the therapeutic agent that treats or inhibits decreased bone mineral density in a dosage amount that is the same as or less than a standard dosage amount, and is also administered a CD109 inhibitor. In some embodiments, the subject is CD109 reference. In some embodiments, the subject is heterozygous for a CD109 missense variant nucleic acid molecule encoding a CD109 predicted loss-of-function polypeptide. Furthermore, when the subject has a lower gene burden for having a CD109 missense variant nucleic acid molecule encoding a CD109 predicted loss-of-function polypeptide, and therefore has an increased risk of developing decreased bone mineral density, the subject is administered a therapeutic agent that treats or inhibits decreased bone mineral density. In some embodiments, when the subject has a lower gene burden for having a CD109 missense variant nucleic acid molecule encoding a CD109 predicted loss-of-function polypeptide, the subject is administered the therapeutic agent that treats or inhibits decreased bone mineral density in a dosage amount that is the same as or greater than the standard dosage amount administered to a subject who has a greater gene burden for having a CD109 missense variant nucleic acid molecule encoding a CD109 predicted loss-of-function polypeptide.

CD109 variants that can be used in the gene burden analysis include any one or more, or any combination, of the following (the Variant column indicates the chromosome, physical genomic position in base pairs, reference allele, and alternative allele for each variant, according to build 38 of the Human Genome sequence by the Human Genome Reference Consortium; coding DNA and protein changes are provided according to the Human Genome Variation Society nomenclature, and refer to three CD109 transcripts annotated in the in the Ensembl database (URL: world wide at “useast.ensembl.org/index.html”); annotations on these three transcripts are reported in the table in the following order: ENST00000287097:ENST00000422508:ENST00000437994):

| Variant | Coding DNA change | Protein change |
|---|---|---|
| 6:73696217:T:C | c.2T > C:c.2T > C:c.2T > C | p.Met1?:p.Met1?:p.Met1? |
| 6:73696249:C:T | c.34C > T:c.34C > T:c.34C > T | p.Leu12Phe:p.Leu12Phe:p.Leu12Phe |
| 6:73696250:T:G | c.35T > G:c.35T > G:c.35T > G | p.Leu12Arg:p.Leu12Arg:p.Leu12Arg |
| 6:73696250:TC:T | c.37delC:c.37delC:c.37delC | p.Leu13fs:p.Leu13fs:p.Leu13fs |
| 6:73697404:C:CG | c.81dupG:c.81dupG:c.81dupG | p.Phe28fs:p.Phe28fs:p.Phe28fs |
| 6:73697413:G:GT | c.89dupT:c.89dupT:c.89dupT | p.Thr31fs:p.Thr31fs:p.Thr31fs |
| 6:73697434:A:G | c.109A > G:c.109A > G:c.109A > G | p.Arg37Gly:p.Arg37Gly:p.Arg37Gly |
| 6:73697459:G:T | c.134G > T:c.134G > T:c.134G > T | p.Gly45Val:p.Gly45Val:p.Gly45Val |
| 6:73697504:C:T | c.179C > T:c.179C > T:c.179C > T | p.Ala60Val:p.Ala60Val:p.Ala60Val |
| 6:73697506:G:T | c.181G > T:c.181G > T:c.181G > T | p.Glu61*:p.Glu61*:p.Glu61* |
| 6:73697515:A:T | c.190A > T:c.190A > T:c.190A > T | p.Lys64*:p.Lys64*:p.Lys64* |
| 6:73697534:CTG:C | c.211_212delGT:c.211_212delGT:c.211_212delGT | p.Val71fs:p.Val71fs:p.Val71fs |
| 6:73723265:C:T | c.262C > T:c.262C > T:c.262C > T | p.Leu88Phe:p.Leu88Phe:p.Leu88Phe |
| 6:73723278:C:G | c.275C > G:c.275C > G:c.275C > G | p.Ser92*:p.Ser92*:p.Ser92* |
| 6:73730377:C:G | c.310C > G:c.310C > G | p.Leu104Val:p.Leu104Val |
| 6:73730398:C:T | c.331C > T:c.331C > T | p.Gln111*:p.Gln111* |
| 6:73730430:CT:C | c.365delT:c.365delT | p.Leu122fs:p.Leu122fs |
| 6:73730456:C:CT | c.390dupT:c.390dupT | p.Val131fs:p.Val131fs |
| 6:73730474:A:G | c.407A > G:c.407A > G | p.Asp136Gly:p.Asp136Gly |
| 6:73730475:C:G | c.408C > G:c.408C > G | p.Asp136Glu:p.Asp136Glu |
| 6:73730512:C:T | c.445C > T:c.445C > T | p.Arg149Cys:p.Arg149Cys |
| 6:73730513:G:A | c.446G > A:c.446G > A | p.Arg149His:p.Arg149His |
| 6:73730513:G:T | c.446G > T:c.446G > T | p.Arg149Leu:p.Arg149Leu |
| 6:73730528:T:TA | c.461_462insA:c.461_462insA | p.Phe154fs:p.Phe154fs |
| 6:73736444:C:G | c.569C > G:c.338C > G:c.569C > G | p.Ser190Cys:p.Ser113Cys:p.Ser190Cys |
| 6:73736450:CT:C | c.579delT:c.348delT:c.579delT | p.Gln194fs:p.Gln117fs:p.Gln194fs |
| 6:73736460:A:AT | c.586dupT:c.355dupT:c.586dupT | p.Ser196fs:p.Ser119fs:p.Ser196fs |
| 6:73736462:C:G | c.587C > G:c.356C > G:c.587C > G | p.Ser196Cys:p.Ser119Cys:p.Ser196Cys |
| 6:73736471:C:T | c.596C > T:c.365C > T:c.596C > T | p.Pro199Leu:p.Pro122Leu:p.Pro199Leu |
| 6:73736491:A:G | c.616A > G:c.385A > G:c.616A > G | p.Ile206Val:p.Ile129Val:p.Ile206Val |
| 6:73756670:G:A | c.661G > A:c.430G > A:c.661G > A | p.Val221Ile:p.Val144Ile:p.Val221Ile |
| 6:73756683:G:T | c.673 + 1G > T:c.442 + 1G > T:c.673 + 1G > T | |
| 6:73758949:C:T | c.679C > T:c.448C > T:c.679C > T | p.Pro227Ser:p.Pro150Ser:p.Pro227Ser |
| 6:73758966:T:TTG | c.697_698insGT:c.466_467insGT:c.697_698insGT | p.Leu233fs:p.Leu156fs:p.Leu233fs |
| 6:73759007:TA:T | c.740delA:c.509delA:c.740delA | p.Asn247fs:p.Asn170fs:p.Asn247fs |
| 6:73759013:G:A | c.743G > A:c.512G > A:c.743G > A | p.Gly248Asp:p.Gly171Asp:p.Gly248Asp |
| 6:73759024:G:T | c.754G > T:c.523G > T:c.754G > T | p.Ala252Ser:p.Ala175Ser:p.Ala252Ser |
| 6:73759025:C:A | c.755C > A:c.524C > A:c.755C > A | p.Ala252Glu:p.Ala175Glu:p.Ala252Glu |
| 6:73759026:AAAGT:A | c.757_758 + 2delAAGT:c.526_527 + | p.Lys253fs:p.Lys176fs:p.Lys253fs |
| | 2delAAGT:c.757_758 + 2delAAGT | |
| 6:73759029:G:T | c.758 + 1G > T:c.527 + 1G > T:c.758 + 1G > T | |
| 6:73762393:TG:T | c.771delG:c.540delG:c.771delG | p.Lys258fs:p.Lys181fs:p.Lys258fs |
| 6:73762396:GA:G | c.773delA:c.542delA:c.773delA | p.Lys258fs:p.Lys181fs:p.Lys258fs |
| 6:73762400:C:T | c.775C > T:c.544C > T:c.775C > T | p.Pro259Ser:p.Pro182Ser:p.Pro259Ser |
| 6:73762403:G:A | c.778G > A:c.547G > A:c.778G > A | p.Val260Met:p.Val183Met:p.Val260Met |
| 6:73762409:G:A | c.784G > A:c.553G > A:c.784G > A | p.Gly262Arg:p.Gly185Arg:p.Gly262Arg |
| 6:73762479:AG:A | c.844delG:c.613delG:c.844delG | p.Thr283fs:p.Thr206fs:p.Thr283fs |
| 6:73762481:G:A | c.855 + 1G > A:c.624 + 1G > A:c.855 + 1G > A | |
| 6:73762796:AT:A | c.913delT:c.682delT:c.913delT | p.Ser305fs:p.Ser228fs:p.Ser305fs |
| 6:73762870:GA:G | c.987delA:c.756delA:c.987delA | p.Glu329fs:p.Glu252fs:p.Glu329fs |
| 6:73762882:G:A | c.997G > A:c.766G > A:c.997G > A | p.Gly333Ser:p.Gly256Ser:p.Gly333Ser |
| 6:73762882:G:T | c.997G > T:c.766G > T:c.997G > T | p.Gly333Cys:p.Gly256Cys:p.Gly333Cys |
| 6:73763574:AG:A | c.998-1delG:c.767-ldelG:c.998-1delG | |
| 6:73763575:G:A | c.998-1G > A:c.767-1G > A:c.998-1G > A | |
| 6:73763575:G:C | c.998-1G > C:c.767-1G > C:c.998-1G > C | |
| 6:73763576:G:A | c.998G > A:c.767G > A:c.998G > A | p.Gly333Asp:p.Gly256Asp:p.Gly333Asp |
| 6:73763624:A:T | c.1046A > T:c.815A > T:c.1046A > T | p.Tyr349Phe:p.Tyr272Phe:p.Tyr349Phe |
| 6:73763629:A:AT | c.1053dupT:c.822dupT:c.1053dupT | p.Glu352fs:p.Glu275fs:p.Glu352fs |
| 6:73763662:C:A | c.1084C > A:c.853C > A:c.1084C > A | p.Pro362Thr:p.Pro285Thr:p.Pro362Thr |
| 6:73763664:ATC:A | c.1091_1092delTC:c.860_861delTC:c.1091_1092delTC | p.Leu364fs:p.Leu287fs:p.Leu364fs |
| 6:73763666:C:A | c.1088C > A:c.857C > A:c.1088C > A | p.Ser363Tyr:p.Ser286Tyr:p.Ser363Tyr |
| 6:73765929:G:T | c.1108-1G > T:c.877-1G > T:c.1108-1G > T | |
| 6:73765949:A:G | c.1127A > G:c.896A > G:c.1127A > G | p.Asp376Gly:p.Asp299Gly:p.Asp376Gly |
| 6:73765957:C:T | c.1135C > T:c.904C > T:c.1135C > T | p.Gln379*:p.Gln302*:p.Gln379* |
| 6:73765969:G:T | c.1147G > T:c.916G > T:c.1147G > T | p.Glu383*:p.Glu306*:p.Glu383* |
| 6:73766146:C:T | c.1324C > T:c.1093C > T:c.1324C > T | p.Gln442*:p.Gln365*:p.Gln442* |
| 6:73766155:G:A | c.1332 + 1G > A:c.1101 + 1G > A:c.1332 + 1G > A | |
| 6:73766825:T:C | c.1399T > C:c.1168T > C:c.1399T > C | p.Tyr467His:p.Tyr390His:p.Tyr467His |
| 6:73766830:C:G | c.1404C > G:c.1173C > G:c.1404C > G | p.Ile468Met:p.Ile391Met:p.Ile468Met |
| 6:73766841:CAA:C | c.1416_1417delAA:c.1185_1186delAA:c.1416_1417delAA | p.Asp474fs:p.Asp397fs:p.Asp474fs |
| 6:73766861:G:A | c.1434 + 1G > A:c.1203 + 1G > A:c.1434 + 1G > A | |
| 6:73766946:A:G | c.1435-2A > G:c.1204-2A > G:c.1435-2A > G | |
| 6:73766951:G:A | c.1438G > A:c.1207G > A:c.1438G > A | p.Gly480Arg:p.Gly403Arg:p.Gly480Arg |
| 6:73766987:C:T | c.1474C > T:c.1243C > T:c.1474C > T | p.Arg492*:p.Arg415*:p.Arg492* |
| 6:73767006:A:C | c.1493A > C:c.1262A > C:c.1493A > C | p.Tyr498Ser:p.Tyr421Ser:p.Tyr498Ser |
| 6:73767006:A:G | c.1493A > G:c.1262A > G:c.1493A > G | p.Tyr498Cys:p.Tyr421Cys:p.Tyr498Cys |
| 6:73767012:T:C | c.1497 + 2T > C:c.1266 + 2T > C:c.1497 + 2T > C | |
| 6:73768070:C:T | c.1513C > T:c.1282C > T:c.1513C > T | p.Gln505*:p.Gln428*:p.Gln505* |
| 6:73768071:A:C | c.1514A > C:c.1283A > C:c.1514A > C | p.Gln505Pro:p.Gln428Pro:p.Gln505Pro |
| 6:73768110:C:CT | c.1556dupT:c.1325dupT:c.1556dupT | p.Leu519fs:p.Leu442fs:p.Leu519fs |
| 6:73768136:C:T | c.1579C > T:c.1348C > T:c.1579C > T | p.Pro527Ser:p.Pro450Ser:p.Pro527Ser |
| 6:73768137:C:T | c.1580C > T:c.1349C > T:c.1580C > T | p.Pro527Leu:p.Pro450Leu:p.Pro527Leu |
| 6:73768146:G:A | c.1589G > A:c.1358G > A:c.1589G > A | p.Cys530Tyr:p.Cys453Tyr:p.Cys530Tyr |
| 6:73768176:G:C | c.1619G > C:c.1388G > C:c.1619G > C | p.Gly540Ala:p.Gly463Ala:p.Gly540Ala |
| 6:73768176:G:T | c.1619G > T:c.1388G > T:c.1619G > T | p.Gly540Val:p.Gly463Val:p.Gly540Val |
| 6:73768178:G:A | c.1621G > A:c.1390G > A:c.1621G > A | p.Glu541Lys:p.Glu464Lys:p.Glu541Lys |
| 6:73768190:G:A | c.1633G > A:c.1402G > A:c.1633G > A | p.Asp545Asn:p.Asp468Asn:p.Asp545Asn |
| 6:73768197:T:TA | c.1645dupA:c.1414dupA:c.1645dupA | p.Ile549fs:p.Ile472fs:p.Ile549fs |
| 6:73768206:C:G | c.1649C > Gx.1418C > G:c.1649C > G | p.Pro550Arg:p.Pro473Arg:p.Pro550Arg |
| 6:73768232:G:C | c.1674 + 1G > C:c.1443 + 1G > C:c.1674 + 1G > C | |
| 6:73771427:A:G | c.1675-2A > G:c.1444-2A > G:c.1675-2A > G | |
| 6:73771428:G:T | c.1675-1G > T:c.1444-1G > T:c.1675-1G > T | |
| 6:73771463:C:T | c.1709C > T:c.1478C > T:c.1709C > T | p.Pro570Leu:p.Pro493Leu:p.Pro570Leu |
| 6:73771481:T:C | c.1727T > C:c.1496T > C:c.1727T > C | p.Leu576Pro:p.Leu499Pro:p.Leu576Pro |
| 6:73771502:C:T | c.1748C > T:c.1517C > T:c.1748C > T | p.Pro583Leu:p.Pro506Leu:p.Pro583Leu |
| 6:73771508:C:T | c.1754C > T:c.1523C > T:c.1754C > T | p.Ser585Phe:p.Ser508Phe:p.Ser585Phe |
| 6:73771526:C:G | c.1772C > G:c.1541C > G:c.1772C > G | p.Ala591Gly:p.Ala514Gly:p.Ala591Gly |
| 6:73771538:G:A | c.1784G > A:c.1553G > A:c.1784G > A | p.Ser595Asn:p.Ser518Asn: p.Ser595Asn |
| 6:73771564:G:T | c.1810G > T:c.1579G > T:c.1810G > T | p.Asp604Tyr:p.Asp527Tyr:p.Asp604Tyr |
| 6:73780421:TA:T | c.1828-2delA:c.1597-2delA:c.1828-2delA | |
| 6:73780423:G:T | c.1828-1G > T:c.1597-1G > T:c.1828-1G > T | |
| 6:73780425:TG:T | c.1831delG:c.1600delG:c.1831delG | p.Val611fs:p.Val534fs:p.Val611fs |
| 6:73780447:TA:T | c.1853delA:c.1622delA:c.1853delA | p.Asn618fs:p.Asn541fs:p.Asn618fs |
| 6:73780458:ATTAT:A | c.1866_1869delTTTA:c.1635_1638delTTTA: | p.Tyr622fs:p.Tyr545fs:p.Tyr622fs |
| | c.1866_1869delTTTA | |
| 6:73780492:C:CT | c.1899dupT:c.1668dupT:c.1899dupT | p.Gln634fs:p.Gln557fs:p.Gln634fs |
| 6:73781262:T:C | c.1906T > C:c.1675T > C:c.1906T > C | p.Cys636Arg:p.Cys559Arg:p.Cys636Arg |
| 6:73781268:C:T | c.1912C > T:c.1681C > T:c.1912C > T | p.Leu638Phe:p.Leu561Phe:p.Leu638Phe |
| 6:73781269:T:A | c.1913T > A:c.1682T > A:c.1913T > A | p.Leu638His:p.Leu561His:p.Leu638His |
| 6:73781273:G:A | c.1917G > A:c.1686G > A:c.1917G > A | p.Trp639*:p.Trp562*:p.Trp639* |
| 6:73781320:G:A | c.1963 + 1G > A:c.1732 + 1G > A:c.1963 + 1G > A | |
| 6:73781320:GT:G | c.1963 + 2delT:c.1732 + 2delT:c.1963 + 2delT | |
| 6:73782612:A:C | c.1964-2A > C:c.1733-2A > C:c.1964-2A > C | |
| 6:73782612:AG:A | c.1964-1delG:c.1733-1delG:c.1964-1delG | |
| 6:73782618:CAA:C | c.1969_1970delAA:c.1738_1739delAA: | p.Asn657fs:p.Asn580fs:p.Asn657fs |
| | c.1969_1970delAA | |
| 6:73782625:G:T | c.1975G > T:c.1744G > T:c.1975G > T | p.Glu659*:p.Glu582*:p.Glu659* |
| 6:73782696:C:CA | c.2047dupA:c.1816dupA:c.2047dupA | p.Ser683fs:p.Ser606fs:p.Ser683fs |
| 6:73782709:C:G | c.2059C > G:c.1828C > G:c.2059C > G | p.Arg687Gly:p.Arg610Gly:p.Arg687Gly |
| 6:73782709:C:T | c.2059C > T:c.1828C > T:c.2059C > T | p.Arg687*:p.Arg610*:p.Arg687* |
| 6:73782716:A:AT | c.2070dupT:c.1839dupT:c.2070dupT | p.Pro691fs:p.Pro614fs:p.Pro691fs |
| 6:73782718:T:C | c.2068T > C:c.1837T > C:c.2068T > C | p.Phe690Leu:p.Phe613Leu:p.Phe690Leu |
| 6:73783720:C:T | c.2119C > T:c.1888C > T:c.2119C > T | p.Gln707*:p.Gln630*:p.Gln707* |
| 6:73783723:G:T | c.2122G > T:c.1891G > T:c.2122G > T | p.Glu708*:p.Glu631*:p.Glu708* |
| 6:73783760:G:A | c.2159G > A:c.1928G > A:c.2159G > A | p.Trp720*:p.Trp643*:p.Trp720* |
| 6:73783761:G:C | c.2160G > C:c.1929G > C:c.2160G > C | p.Trp720Cys:p.Trp643Cys:p.Trp720Cys |
| 6:73783761:G:T | c.2160G > T:c.1929G > T:c.2160G > T | p.Trp720Cys:p.Trp643Cys:p.Trp720Cys |
| 6:73783765:G:T | c.2164G > T:c.1933G > T:c.2164G > T | p.Ala722Ser:p.Ala645Ser:p.Ala722Ser |
| 6:73783771:G:T | c.2170G > T:c.1939G > T:c.2170G > T | p.Gly724Cys:p.Gly647Cys:p.Gly724Cys |
| 6:73783772:GT:G | c.2175delT:c.1944delT:c.2175delT | p.Phe725fs:p.Phe648fs:p.Phe725fs |
| 6:73783783:T:C | c.2182T > C:c.1951T > C:c.2182T > C | p.Ser728Pro:p.Ser651Pro:p.Ser728Pro |
| 6:73783784:C:CT | c.2184dupT:c.1953dupT:c.2184dupT | p.Glu729fs:p.Glu652fs:p.Glu729fs |
| 6:73783795:6:C | c.2194G > C:c.1963G > C:c.2194G > C | p.Gly732Arg:p.Gly655Arg:p.Gly732Arg |
| 6:73783796:G:A | c.2195G > A:c.1964G > A:c.2195G > A | p.Gly732Asp:p.Gly655Asp:p.Gly732Asp |
| 6:73783810:AC:A | c.2210delC:c.1979delC:c.2210delC | p.Thr737fs:p.Thr660fs:p.Thr737fs |
| 6:73783817:C:T | c.2216C > T:c.1985C > T:c.2216C > T | p.Pro739Leu:p.Pro662Leu:p.Pro739Leu |
| 6:73785363:G:A | c.2224-1G > A:c.1993-1G > A:c.2224-1G > A | |
| 6:73785364:C:A | c.2224C > A:c.1993C > A:c.2224C > A | p.Leu742Ile:p.Leu665Ile:p.Leu742Ile |
| 6:73785364:CT:C | c.2225delT:c.1994delT:c.2225delT | p.Leu742fs:p.Leu665fs:p.Leu742fs |
| 6:73785365:T:C | c.2225T > C:c.1994T > C:c.2225T > C | p.Leu742Pro:p.Leu665Pro:p.Leu742Pro |
| 6:73785382:T:C | c.2242T > C:c.2011T > C:c.2242T > C | p.Phe748Leu:p.Phe671Leu:p.Phe748Leu |
| 6:73785385:T:C | c.2245T > C:c.2014T > C:c.2245T > C | p.Phe749Leu:p.Phe672Leu:p.Phe749Leu |
| 6:73785408:C:A | c.2268C > A:c.2037C > A:c.2268C > A | p.Tyr756*:p.Tyr679*:p.Tyr756* |
| 6:73785408:C:G | c.2268C > G:c.2037C > G:c.2268C > G | p.Tyr756*:p.Tyr679*:p.Tyr756* |
| 6:73785410:C:G | c.2270C > G:c.2039C > G:c.2270C > G | p.Ser757Cys:p.Ser680Cys:p.Ser757Cys |
| 6:73785419:G:A | c.2279G > A:c.2048G > A:c.2279G > A | p.Arg760Lys:p.Arg683Lys:p.Arg760Lys |
| 6:73785454:A:T | c.2314A > T:c.2083A > T:c.2314A > T | p.Asn772Tyr:p.Asn695Tyr:p.Asn772Tyr |
| 6:73785458:A:G | c.2318A > G:c.2087A > G:c.2318A > G | p.Tyr773Cys:p.Tyr696Cys:p.Tyr773Cys |
| 6:73787237:A:T | c.2341A > T:c.2110A > T:c.2341A > T | p.Lys781*:p.Lys704*:p.Lys781* |
| 6:73787241:T:A | c.2345T > A:c.2114T > A:c.2345T > A | p.Val782Glu:p.Val705Glu:p.Val782Glu |
| 6:73787264:T:G | c.2368T > G:c.2137T > G:c.2368T > G | p.Phe790Val:p.Phe713Val:p.Phe790Val |
| 6:73787271:T:G | c.2375T > G:c.2144T > G:c.2375T > G | p.Ile792Ser:p.Ile715Ser:p.Ile792Ser |
| 6:73787330:A:T | c.2434A > T:c.2203A > T:c.2434A > T | p.Ser812Cys:p.Ser735Cys:p.Ser812Cys |
| 6:73787338:TG:T | c.2446delG:c.2215delG:c.2446delG | p.Ala816fs:p.Ala739fs:p.Ala816fs |
| 6:73787366:C:T | c.2470C > T:c.2239C > T:c.2470C > T | p.Pro824Ser:p.Pro747Ser:p.Pro824Ser |
| 6:73787378:G:T | c.2482G > T:c.2251G > T:c.2482G > T | p.Gly828*:p.Gly751*:p.Gly828* |
| 6:73787379:G:A | c.2483G > A:c.2252G > A:c.2483G > A | p.Gly828Glu:p.Gly751Glu:p.Gly828Glu |
| 6:73787394:C:T | c.2498C > T:c.2267C > T:c.2498C > T | p.Thr833Ile:p.Thr756Ile:p.Thr833Ile |
| 6:73787447:G:A | c.2551G > A:c.2320G > A:c.2551G > A | p.Val851Ile:p.Val774Ile:p.Val851Ile |
| 6:73787448:T:TA | c.2555dupA:c.2324dupA:c.2555dupA | p.Ala853fs:p.Ala776fs:p.Ala853fs |
| 6:73787452:G:T | c.2556G > T:c.2325G > T:c.2556G > T | p.Lys852Asn:p.Lys775Asn:p.Lys852Asn |
| 6:73788469:C:G | c.2558C > G:c.2327C > G:c.2558C > G | p.Ala853Gly:p.Ala776Gly:p.Ala853Gly |
| 6:73788503:CT:C | c.2594delT:c.2363delT:c.2594delT | p.Leu865fs:p.Leu788fs:p.Leu865fs |
| 6:73788564:C:T | c.2653C > T:c.2422C > T:c.2653C > T | p.Pro885Ser:p.Pro808Ser:p.Pro885Ser |
| 6:73788565:C:T | c.2654C > T:c.2423C > T:c.2654C > T | p.Pro885Leu:p.Pro808Leu:p.Pro885Leu |
| 6:73788582:G:A | c.2671G > A:c.2440G > A:c.2671G > A | p.Gly891Ser:p.Gly814Ser:p.Gly891Ser |
| 6:73788588:G:T | c.2677G > T:c.2446G > T:c.2677G > T | p.Glu893*:p.Glu816*:p.Glu893* |
| 6:73788601:T:TC | c.2691dupC:c.2460dupC:c.2691dupC | p.Thr898fs:p.Thr821fs:p.Thr898fs |
| 6:73792622:CCAGGAG:C | c.2702-3_2704delCAGGAG:c.2471- | p.Gly901del:p.Gly824del:p.Gly901del |
| | 3_2473delCAGGAG:c.2702- | |
| | 3_2704delCAGGAG | |
| 6:73792628:G:C | c.2704G > C:c.2473G > C:c.2704G > C | p.Asp902His:p.Asp825His:p.Asp902His |
| 6:73792629:A:T | c.2705A > T:c.2474A > T:c.2705A > T | p.Asp902Val:p.Asp825Val:p.Asp902Val |
| 6:73792637:G:T | c.2713G > T:c.2482G > T:c.2713G > T | p.Gly905Cys:p.Gly828Cys:p.Gly905Cys |
| 6:73792686:G:T | c.2762G > T:c.2531G > T:c.2762G > T | p.Cys921Phe:p.Cys844Phe:p.Cys921Phe |
| 6:73792688:G:A | c.2764G > A:c.2533G > A:c.2764G > A | p.Gly922Ser:p.Gly845Ser:p.Gly922Ser |
| 6:73792703:A:C | c.2779A > C:c.2548A > C:c.2779A > C | p.Ile927Leu:p.Ile850Leu:p.Ile927Leu |
| 6:73792712:G:T | c.2788G > T:c.2557G > T:c.2788G > T | p.Ala930Ser:p.Ala853Ser:p.Ala930Ser |
| 6:73792718:A:G | c.2794A > G:c.2563A > G:c.2794A > G | p.Asn932Asp:p.Asn855Asp:p.Asn932Asp |
| 6:73792736:T:A | c.2812T > A:c.2581T > A:c.2812T > A | p.Tyr938Asn:p.Tyr861Asn:p.Tyr938Asn |
| 6:73792754:C:T | c.2830C > T:c.2599C > T:c.2830C > T | p.Gln944*:p.Gln867*:p.Gln944* |
| 6:73792775:G:GA | c.2856dupA:c.2625dupA:c.2856dupA | p.Ala953fs:p.Ala876fs:p.Ala953fs |
| 6:73792780:A:C | c.2856A > C:c.2625A > C:c.2856A > C | p.Lys952Asn:p.Lys875Asn:p.Lys952Asn |
| 6:73792782:C:A | c.2858C > A:c.2627C > A:c.2858C > A | p.Ala953Asp:p.Ala876Asp:p.Ala953Asp |
| 6:73792784:C:CT | c.2863dupT:c.2632dupT:c.2863dupT | p.Ser955fs:p.Ser878fs:p.Ser955fs |
| 6:73792799:C:T | c.2875C > T:c.2644C > T:c.2875C > T | p.Gln959*:p.Gln882*:p.Gln959* |
| 6:73792802:G:A | c.2878G > A:c.2647G > A:c.2878G > A | p.Gly960Ser:p.Gly883Ser:p.Gly960Ser |
| 6:73803231:G:A | c.2890G > A:c.2659G > A:c.2890G > A | p.Glu964Lys:p.Glu887Lys:p.Glu964Lys |
| 6:73803253:A:G | c.2912A > G:c.2681A > G:c.2912A > G | p.Asp971Gly:p.Asp894Gly:p.Asp971Gly |
| 6:73803256:G:A | c.2915G > A:c.2684G > A:c.2915G > A | p.Gly972Asp:p.Gly895Asp:p.Gly972Asp |
| 6:73803256:G:T | c.2915G > T:c.2684G > T:c.2915G > T | p.Gly972Val:p.Gly895Val:p.Gly972Val |
| 6:73803264:A:G | c.2923A > G:c.2692A > G:c.2923A > G | p.Ser975Gly:p.Ser898Gly:p.Ser975Gly |
| 6:73803298:C:T | c.2957C > T:c.2726C > T:c.2957C > T | p.Thr986Ile:p.Thr909Ile:p.Thr986Ile |
| 6:73803301:G:A | c.2960G > A:c.2729G > A:c.2960G > A | p.Trp987*:p.Trp910*:p.Trp987* |
| 6:73806844:G:T | c.2961G > T:c.2730G > T:c.2961G > T | p.Trp987Cys:p.Trp910Cys:p.Trp987Cys |
| 6:73806855:T:C | c.2972T > C:c.2741T > C:c.2972T > C | p.Phe991Ser:p.Phe914Ser:p.Phe991Ser |
| 6:73806870:T:G | c.2987T > G:c.2756T > G:c.2987T > G | p.Phe996Cys:p.Phe919Cys:p.Phe996Cys |
| 6:73806897:T:C | c.3014T > C:c.2783T > C:c.3014T > C | p.Ile1005Thr:p.Ile928Thr:p.Ile1005Thr |
| 6:73806902:C:T | c.3019C > T:c.2788C > T:c.3019C > T | p.Gln1007*:p.Gln930*:p.Gln1007* |
| 6:73806908:G:A | c.3025G > A:c.2794G > A:c.3025G > A | p.Val1009Met:p.Val932Met:p.Val1009Met |
| 6:73806921:C:CAT | c.3040_3041dupTA:c.2809_2810dupTA: | p.Trp1016fs:p.Trp939fs:p.Trp1016fs |
| | c.3040_3041dupTA | |
| 6:73806956:G:A | c.3073G > A:c.2842G > A:c.3073G > A | p.Gly1025Ser:p.Gly948Ser:p.Gly1025Ser |
| 6:73806956:G:C | c.3073G > C:c.2842G > C:c.3073G > C | p.Gly1025Arg:p.Gly948Arg:p.Gly1025Arg |
| 6:73806981:T:A | c.3098T > A:c.2867T > A:c.3098T > A | p.Val1033Glu:p.Val956Glu:p.Val1033Glu |
| 6:73806984:T:G | c.3101T > G:c.2870T > G:c.3101T > G | p.Ile1034Ser:p.Ile957Ser:p.Ile1034Ser |
| 6:73806987:A:T | c.3104A > T:c.2873A > T:c.3104A > T | p.His1035Leu:p.His958Leu:p.His1035Leu |
| 6:73807002:G:A | c.3119G > A:c.2888G > A:c.3119G > A | p.Gly1040Asp:p.Gly963Asp:p.Gly1040Asp |
| 6:73807005:G:A | c.3122G > A:c.2891G > A:c.3122G > A | p.Gly1041Asp:p.Gly964Asp:p.Gly1041Asp |
| 6:73807029:C:G | c.3146C > G:c.2915C > G:c.3146C > G | p.Thr1049Arg:p.Thr972Arg:p.Thr1049Arg |
| 6:73807034:T:A | c.3151T > A:c.2920T > A:c.3151T > A | p.Tyr1051Asn:p.Tyr974Asn:p.Tyr1051Asn |
| 6:73807035:A:G | c.3152A > G:c.2921A > G:c.3152A > G | p.Tyr1051Cys:p.Tyr974Cys:p.Tyr1051Cys |
| 6:73807035:A:T | c.3152A > T:c.2921A > T:c.3152A > T | p.Tyr1051Phe:p.Tyr974Phe:p.Tyr1051Phe |
| 6:73808113:T:G | c.3220T > G:c.2989T > G:c.3220T > G | p.Phe1074Val:p.Phe997Val:p.Phe1074Val |
| 6:73808146:G:A | c.3253G > A:c.3022G > A:c.3253G > A | p.Asp1085Asn:p.Asp1008Asn:p.Asp1085Asn |
| 6:73808153:A:T | c.3260A > T:c.3029A > T:c.3260A > T | p.Tyr1087Phe:p.Tyr1010Phe:p.Tyr1087Phe |
| 6:73808156:C:T | c.3263C > T:c.3032C > T:c.3263C > T | p.Thr1088Ile:p.Thr1011Ile:p.Thr1088Ile |
| 6:73808159:T:C | c.3266T > C:c.3035T > C:c.3266T > C | p.Leu1089Pro:p.Leu1012Pro:p.Leu1089Pro |
| 6:73808176:G:T | c.3283G > T:c.3052G > T:c.3283G > T | p.Ala1095Ser:p.Ala1018Ser:p.Ala1095Ser |
| 6:73808244:AGAAGGTAAT:A | c.3352_3355 + 5delGAAGGTAAT:c.3121_3124 + | p.Glu1118fs:p.Glu1041fs:p.Glu1118fs |
| | 5delGAAGGTAAT:c.3352_3355 + 5delGAAGGTAAT | |
| 6:73808245:GA:G | c.3354delA:c.3123delA:c.3354delA | p.Gly1119fs:p.Gly1042fs:p.Gly1119fs |
| 6:73809981:CA:C | c.3356-2delA:c.3125-2delA:c.3356-2delA | |
| 6:73809992:C:T | c.3364C > T:c.3133C > T:c.3364C > T | p.Gln112*:p.Gln1045*:p.Gln1122* |
| 6:73809995:T:A | c.3367T > A:c.3136T > A:c.3367T > A | p.Phe1123Ile:p.Phe1046Ile:p.Phe1123Ile |
| 6:73810034:C:A | c.3406C > A:c.3175C > A:c.3406C > A | p.Gln1136Lys:p.Gln1059Lys:p.Gln1136Lys |
| 6:73810044:C:T | c.3416C > T:c.3185C > T:c.3416C > T | p.Ser1139Phe:p.Ser1062Phe:p.Ser1139Phe |
| 6:73810054:T:G | c.3426T > G:c.3195T > G:c.3426T > G | p.Ile1142Met:p.Ile1065Met:p.Ile1142Met |
| 6:73810089:T:TA | c.3462dupA:c.3231dupA:c.3462dupA | p.Gln1155fs:p.Gln1078fs:p.Gln1155fs |
| 6:73810106:G:A | c.3478G > A:c.3247G > A:c.3478G > A | p.Glu1160Lys:p.Glu1083Lys:p.Glu1160Lys |
| 6:73810143:G:T | c.3515G > T:c.3284G > T:c.3515G > T | p.Arg1172Ile:p.Arg1095Ile:p.Arg1172Ile |
| 6:73810154:G:A | c.3526G > A:c.3295G > A:c.3526G > A | p.Gly1176Ser:p.Gly1099Ser:p.Gly1176Ser |
| 6:73810155:G:A | c.3527G > A:c.3296G > A:c.3527G > A | p.Gly1176Asp:p.Gly1099Asp:p.Gly1176Asp |
| 6:73810155:G:C | c.3527G > C:c.3296G > C:c.3527G > C | p.Gly1176Ala:p.Gly1099Ala:p.Gly1176Ala |
| 6:73810155:G:T | c.3527G > T:c.3296G > T:c.3527G > T | p.Gly1176Val:p.Gly1099Val:p.Gly1176Val |
| 6:73810157:G:T | c.3529G > T:c.3298G > T:c.3529G > T | p.Gly1177Cys:p.Gly1100Cys:p.Gly1177Cys |
| 6:73810158:G:A | c.3530G > A:c.3299G > A:c.3530G > A | p.Gly1177Asp:p.Gly1100Asp:p.Gly1177Asp |
| 6:73810172:C:T | c.3544C > T:c.3313C > T:c.3544C > T | p.Gln1182*:p.Gln1105*:p.Gln1182* |
| 6:73810990:A:G | c.3547-2A > G:c.3316-2A > G:c.3547-2A > G | |
| 6:73810993:A:C | c.3548A > C:c.3317A > C:c.3548A > C | p.Asp1183Ala:p.Asp1106Ala:p.Asp1183Ala |
| 6:73811013:G:C | c.3568G > C:c.3337G > C:c.3568G > C | p.Ala1190Pro:p.Ala1113Pro:p.Ala1190Pro |
| 6:73811013:G:T | c.3568G > T:c.3337G > T:c.3568G > T | p.Ala1190Ser:p.Ala1113Ser:p.Ala1190Ser |
| 6:73811029:C:A | c.3584C > A:c.3353C > A:c.3584C > A | p.Ala1195Glu:p.Ala1118Glu:p.Ala1195Glu |
| 6:73811051:GA:G | c.3607delA:c.3376delA:c.3607delA | p.Thr1203fs:p.Thr1126fs:p.Thr1203fs |
| 6:73811110:T:C | c.3665T > C:c.3434T > C | p.Ile1222Thr:p.Ile1145Thr |
| 6:73811120:CA:C | c.3677delA:c.3446delA | p.Asn1226fs:p.Asn1149fs |
| 6:73811121:A:T | c.3676A > T:c.3445A > T | p.Asn1226Tyr:p.Asn1149Tyr |
| 6:73812271:G:A | c.3768 + 1G > A:c.3537 + 1G > A:c.3717 + 1G > A | |
| 6:73812271:G:C | c.3768 + 1G > C:c.3537 + 1G > C:c.3717 + 1G > C | |
| 6:73812271:G:T | c.3768 + 1G > T:c.3537 + 1G > T:c.3717 + 1G > T | |
| 6:73812272:T:A | c.3768 + 2T > A:c.3537 + 2T > A:c.3717 + 2T > A | |
| 6:73814990:GTA:G | c.3783_3784delTA:c.3552_3553delTA:c.3732_3733delTA | p.Tyr1261fs:p.Tyr1184fs:p.Tyr1244fs |
| 6:73814994:A:G | c.3782A > G:c.3551A > G:c.3731A > G | p.Tyr1261Cys:p.Tyr1184Cys:p.Tyr1244Cys |
| 6:73815026:C:T | c.3814C > T:c.3583C > T:c.3763C > T | p.Arg1272*:p.Arg1195*:p.Arg1255* |
| 6:73815098:CAT:C | c.3887_3888delAT:c.3656_3657delAT:c.3836_3837delAT | p.His1296fs:p.His1219fs:p.His1279fs |
| 6:73815113:G:A | c.3901G > A:c.3670G > A:c.3850G > A | p.Val1301Met:p.Val1224Met:p.Val1284Met |
| 6:73815117:G:A | c.3905G > A:c.3674G > A:c.3854G > A | p.Cys1302Tyr:p.Cys1225Tyr:p.Cys1285Tyr |
| 6:73818386:A:G | c.3912-2A > G:c.3681-2A > G:c.3861-2A > G | |
| 6:73818413:A:G | c.3937A > G:c.3706A > G:c.3886A > G | p.Met1313Val:p.Met1236Val:p.Met1296Val |
| 6:73818415:G:C | c.3939G > C:c.3708G > C:c.3888G > C | p.Met1313Ile:p.Met1236Ile:p.Met1296Ile |
| 6:73818417:C:A | c.3941C > A:c.3710C > A:c.3890C > A | p.Ala1314Asp:p.Ala1237Asp:p.Ala1297Asp |
| 6:73818423:T:G | c.3947T > G:c.3716T > G:c.3896T > G | p.Met1316Arg:p.Met1239Arg:p.Met1299Arg |
| 6:73818425:G:A | c.3949G > A:c.3718G > A:c.3898G > A | p.Glu1317Lys:p.Glu1240Lys:p.Glu1300Lys |
| 6:73818443:G:T | c.3967G > T:c.3736G > T:c.3916G > T | p.Gly1323Cys:p.Gly1246Cys:p.Gly1306Cys |
| 6:73818450:TG:T | c.3976delG:c.3745delG:c.3925delG | p.Val1326fs:p.Val1249fs:p.Val1309fs |
| 6:73818456:CT:C | c.3982delT:c.3751delT:c.3931delT | p.Ser1328fs:p.Ser1251fs:p.Ser1311fs |
| 6:73818479:G:T | c.4003G > T:c.3772G > T:c.3952G > T | p.Glul335*:p.Glu1258*:p.Glu1318* |
| 6:73820459:A:G | c.4060-2A > G:c.3829-2A > G:c.4009-2A > G | |
| 6:73820480:G:C | c.4079G > C:c.3848G > C:c.4028G > C | p.Cys1360Ser:p.Cys1283Ser:p.Cys1343Ser |
| 6:73820528:A:T | c.4127A > T:c.3896A > T:c.4076A > T | p.Asp1376Val:p.Asp1299Val:p.Asp1359Val |
| 6:73820537:T:C | c.4136T > C:c.3905T > C:c.4085T > C | p.Val1379Ala:p.Val1302Ala:p.Val1362Ala |
| 6:73820549:A:C | c.4148A > C:c.3917A > C:c.4097A > C | p.Asp1383Ala:p.Asp1306Ala:p.Asp1366Ala |
| 6:73820549:A:G | c.4148A > G:c.3917A > G:c.4097A > G | p.Asp1383Gly:p.Asp1306Gly:p.Asp1366Gly |
| 6:73820549:AT:A | c.4150delT:c.3919delT:c.4099delT | p.Tyr1384fs:p.Tyr1307fs:p.Tyr1367fs |
| 6:73823456:A:G | c.4163-2A > G:c.3932-2A > G:c.4112-2A > G | |
| 6:73823473:GA:G | c.4180delA:c.3949delA:c.4129delA | p.Ser1394fs:p.Ser1317fs:p.Ser1377fs |
| 6:73823481:A:G | c.4186A > G:c.3955A > G:c.4135A > G | p.Asn1396Asp:p.Asn1319Asp:p.Asn1379Asp |
| 6:73823485:C:G | c.4190C > G:c.3959C > G:c.4139C > G | p.Ser1397Cys:p.Ser1320Cys:p.Ser1380Cys |
| 6:73823530:GC:G | c.4236delC:c.4005delC:c.4185delC | p.Cys1413fs:p.Cys1336fs:p.Cys1396fs |
| 6:73823534:C:A | c.4239C > A:c.4008C > A:c.4188C > A | p.Cys1413*:p.Cys1336*:p.Cys1396* |
| 6:73823583:A:AT | c.4293dupT:c.4062dupT:c.4242dupT | p.Ile1432fs:p.Ile1355fs:p.Ile1415fs |
| 6:73823592:TTC:T | c.4299_4300delCT:c.4068_4069delCT:c.4248_4249delCT | p.Phe1433fs:p.Phe1356fs:p.Phe1416fs |
| 6:73823612:C:A | c.4317C > A:c.4086C > A:c.4266C > A | p.Tyr1439*:p.Tyr1362*:p.Tyr1422* |

The present disclosure also provides methods of detecting the presence or absence of a CD109 missense variant nucleic acid molecule (i.e., a genomic nucleic acid molecule, an mRNA molecule, or a cDNA molecule produced from an mRNA molecule) encoding a CD109 predicted loss-of-function polypeptide in a biological sample from a subject. It is understood that gene sequences within a population and mRNA molecules encoded by such genes can vary due to polymorphisms such as single-nucleotide polymorphisms. The sequences provided herein for the CD109 variant genomic nucleic acid molecule, CD109 variant mRNA molecule, and CD109 variant cDNA molecule are only exemplary sequences. Other sequences for the CD109 variant genomic nucleic acid molecule, variant mRNA molecule, and variant cDNA molecule are also possible.

The biological sample can be derived from any cell, tissue, or biological fluid from the subject. The biological sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting any CD109 missense variant nucleic acid molecule encoding a CD109 predicted loss-of-function polypeptide, preliminary processing designed to isolate or enrich the biological sample for the genomic DNA can be employed. A variety of techniques may be used for this purpose. When detecting the level of any CD109 variant mRNA molecule, different techniques can be used enrich the biological sample with mRNA molecules. Various methods to detect the presence or level of an mRNA molecule or the presence of a particular variant genomic DNA locus can be used.

In some embodiments, detecting a CD109 missense variant nucleic acid molecule encoding a CD109 predicted loss-of-function polypeptide in a subject comprises performing a sequence analysis on a biological sample obtained from the subject to determine whether a CD109 genomic nucleic acid molecule in the biological sample, and/or a CD109 mRNA molecule in the biological sample, and/or a CD109 cDNA molecule produced from an mRNA molecule in the biological sample, comprises one or more variations that cause a loss-of-function (partial or complete) or are predicted to cause a loss-of-function (partial or complete).

In some embodiments, the methods of detecting the presence or absence of a CD109 missense variant nucleic acid molecule encoding a CD109 predicted loss-of-function polypeptide (such as, for example, a genomic nucleic acid molecule, an mRNA molecule, and/or a cDNA molecule produced from an mRNA molecule) in a subject, comprise performing an assay on a biological sample obtained from the subject. The assay determines whether a nucleic acid molecule in the biological sample comprises a particular nucleotide sequence.

In some embodiments, the biological sample comprises a cell or cell lysate. Such methods can further comprise, for example, obtaining a biological sample from the subject comprising a CD109 genomic nucleic acid molecule or mRNA molecule, and if mRNA, optionally reverse transcribing the mRNA into cDNA. Such assays can comprise, for example determining the identity of these positions of the particular CD109 nucleic acid molecule. In some embodiments, the method is an in vitro method.

In some embodiments, the determining step, detecting step, or sequence analysis comprises sequencing at least a portion of the nucleotide sequence of the CD109 genomic nucleic acid molecule, the CD109 mRNA molecule, or the CD109 cDNA molecule in the biological sample, wherein the sequenced portion comprises one or more variations that cause a loss-of-function (partial or complete) or are predicted to cause a loss-of-function (partial or complete).

In some embodiments, the assay comprises sequencing the entire nucleic acid molecule. In some embodiments, only a CD109 genomic nucleic acid molecule is analyzed. In some embodiments, only a CD109 mRNA is analyzed. In some embodiments, only a CD109 cDNA obtained from CD109 mRNA is analyzed.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse-transcribed into a cDNA prior to the amplifying step. In some embodiments, the nucleic acid molecule is present within a cell obtained from the subject.

In some embodiments, the assay comprises contacting the biological sample with a primer or probe, such as an alteration-specific primer or alteration-specific probe, that specifically hybridizes to a CD109 variant genomic sequence, variant mRNA sequence, or variant cDNA sequence and not the corresponding CD109 reference sequence under stringent conditions, and determining whether hybridization has occurred.

In some embodiments, the determining step, detecting step, or sequence analysis comprises: a) amplifying at least a portion of the nucleic acid molecule that encodes the CD109 polypeptide; b) labeling the amplified nucleic acid molecule with a detectable label; c) contacting the labeled nucleic acid molecule with a support comprising an alteration-specific probe; and d) detecting the detectable label.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising a CD109 variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2× SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, such isolated nucleic acid molecules hybridize to CD109 missense variant nucleic acid molecules (such as genomic nucleic acid molecules, mRNA molecules, and/or cDNA molecules) under stringent conditions. Such nucleic acid molecules can be used, for example, as probes, primers, alteration-specific probes, or alteration-specific primers as described or exemplified herein, and include, without limitation primers, probes, antisense RNAs, shRNAs, and siRNAs, each of which is described in more detail elsewhere herein, and can be used in any of the methods described herein.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to CD109 missense variant genomic nucleic acid molecules, CD109 missense variant mRNA molecules, and/or CD109 missense variant cDNA molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the CD109 variant missense genomic nucleic acid molecules, CD109 missense variant mRNA molecules, and/or CD109 missense variant cDNA molecules disclosed herein. The primers described herein can be used to amplify CD109 missense variant genomic nucleic acid molecules, CD109 missense variant mRNA molecules, or CD109 missense variant cDNA molecules, or a fragment thereof.

In the context of the disclosure “specifically hybridizes” means that the probe or primer (such as, for example, the alteration-specific probe or alteration-specific primer) does not hybridize to a nucleic acid sequence encoding a CD109 reference genomic nucleic acid molecule, a CD109 reference mRNA molecule, and/or a CD109 reference cDNA molecule.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

The nucleotide sequence of a CD109 reference genomic nucleic acid molecule is set forth in SEQ ID NO:1 (ENSG00000156535.15 chr6:73,695,785-73,828,316 in the GRCh38/hg38 human genome assembly).

The nucleotide sequence of a CD109 reference mRNA molecule is set forth in SEQ ID NO:2. The nucleotide sequence of another CD109 reference mRNA molecule is set forth in SEQ ID NO:3. The nucleotide sequence of another CD109 reference mRNA molecule is set forth in SEQ ID NO:4. The nucleotide sequence of another CD109 reference mRNA molecule is set forth in SEQ ID NO:5. The nucleotide sequence of another CD109 reference mRNA molecule is set forth in SEQ ID NO:6. The nucleotide sequence of another CD109 reference mRNA molecule is set forth in SEQ ID NO:7. The nucleotide sequence of another CD109 reference mRNA molecule is set forth in SEQ ID NO:8. The nucleotide sequence of another CD109 reference mRNA molecule is set forth in SEQ ID NO:9. The nucleotide sequence of another CD109 reference mRNA molecule is set forth in SEQ ID NO:10. The nucleotide sequence of another CD109 reference mRNA molecule is set forth in SEQ ID NO:11. The nucleotide sequence of another CD109 reference mRNA molecule is set forth in SEQ ID NO:12. The nucleotide sequence of another CD109 reference mRNA molecule is set forth in SEQ ID NO:13. The nucleotide sequence of another CD109 reference mRNA molecule is set forth in SEQ ID NO:14. The nucleotide sequence of another CD109 reference mRNA molecule is set forth in SEQ ID NO:15. The nucleotide sequence of another CD109 reference mRNA molecule is set forth in SEQ ID NO:16.

The nucleotide sequence of a CD109 reference cDNA molecule is set forth in SEQ ID NO:17. The nucleotide sequence of another CD109 reference cDNA molecule is set forth in SEQ ID NO:18. The nucleotide sequence of another CD109 reference cDNA molecule is set forth in SEQ ID NO:19. The nucleotide sequence of another CD109 reference cDNA molecule is set forth in SEQ ID NO:20. The nucleotide sequence of another CD109 reference cDNA molecule is set forth in SEQ ID NO:21. The nucleotide sequence of another CD109 reference cDNA molecule is set forth in SEQ ID NO:22. The nucleotide sequence of another CD109 reference cDNA molecule is set forth in SEQ ID NO:23. The nucleotide sequence of another CD109 reference cDNA molecule is set forth in SEQ ID NO:24. The nucleotide sequence of another CD109 reference cDNA molecule is set forth in SEQ ID NO:25. The nucleotide sequence of another CD109 reference cDNA molecule is set forth in SEQ ID NO:26. The nucleotide sequence of another CD109 reference cDNA molecule is set forth in SEQ ID NO:27. The nucleotide sequence of another CD109 reference cDNA molecule is set forth in SEQ ID NO:28. The nucleotide sequence of another CD109 reference cDNA molecule is set forth in SEQ ID NO:29.

The amino acid sequence of a CD109 reference polypeptide is set forth in SEQ ID NO:30, and is 1,428 amino acids in length. The nucleotide sequence of another CD109 reference polypeptide is set forth in SEQ ID NO:31, and is 1,368 amino acids in length. The nucleotide sequence of another CD109 reference polypeptide is set forth in SEQ ID NO:32, and is 1,445 amino acids in length. The nucleotide sequence of another CD109 reference polypeptide is set forth in SEQ ID NO:33, and is 665 amino acids in length. The nucleotide sequence of another CD109 reference polypeptide is set forth in SEQ ID NO:34, and is 1,374 amino acids in length. The nucleotide sequence of another CD109 reference polypeptide is set forth in SEQ ID NO:35, and is 854 amino acids in length. The nucleotide sequence of another CD109 reference polypeptide is set forth in SEQ ID NO:36, and is 847 amino acids in length.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms. The examples provided herein are only exemplary sequences. Other sequences are also possible.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×his or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

As used herein, the phrase “corresponding to” or grammatical variations thereof when used in the context of the numbering of a particular nucleotide or nucleotide sequence or position refers to the numbering of a specified reference sequence when the particular nucleotide or nucleotide sequence is compared to a reference sequence (such as, for example, SEQ ID NO:1). In other words, the residue (such as, for example, nucleotide or amino acid) number or residue (such as, for example, nucleotide or amino acid) position of a particular polymer is designated with respect to the reference sequence rather than by the actual numerical position of the residue within the particular nucleotide or nucleotide sequence. For example, a particular nucleotide sequence can be aligned to a reference sequence by introducing gaps to optimize residue matches between the two sequences. In these cases, although the gaps are present, the numbering of the residue in the particular nucleotide or nucleotide sequence is made with respect to the reference sequence to which it has been aligned.

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequence follows the standard convention of beginning at the amino terminus of the sequence and proceeding forward (i.e., from left to right in each line) to the carboxy terminus.

The present disclosure also provides therapeutic agents that treat or inhibit decreased bone mineral density for use in the treatment of decreased bone mineral density in a subject having: a CD109 missense variant genomic nucleic acid molecule encoding a CD109 predicted loss-of-function polypeptide; a CD109 missense variant mRNA molecule encoding a CD109 predicted loss-of-function polypeptide; or a CD109 missense variant cDNA molecule encoding a CD109 predicted loss-of-function polypeptide. Any of the therapeutic agents that treat or inhibit decreased bone mineral density described herein can be used in these methods. The subject can have or have a risk of developing decreased bone mineral density, osteopenia, Type I osteoporosis, Type II osteoporosis, or secondary osteoporosis.

The present disclosure also provides uses of therapeutic agents that treat or inhibit decreased bone mineral density for use in the preparation of a medicament for treating decreased bone mineral density in a subject having: a CD109 missense variant genomic nucleic acid molecule encoding a CD109 predicted loss-of-function polypeptide; a CD109 missense variant mRNA molecule encoding a CD109 predicted loss-of-function polypeptide; or a CD109 missense variant cDNA molecule encoding a CD109 predicted loss-of-function polypeptide. Any of the therapeutic agents that treat or inhibit decreased bone mineral density described herein can be used in these methods. The subject can have or have a risk of developing decreased bone mineral density, osteopenia, Type I osteoporosis, Type II osteoporosis, or secondary osteoporosis.

The present disclosure also provides CD109 inhibitors for use in the treatment of decreased bone mineral density in a subject that: a) is reference for a CD109 genomic nucleic acid molecule, a CD109 mRNA molecule, or a CD109 cDNA molecule; or b) is heterozygous for: i) a CD109 missense variant genomic nucleic acid molecule encoding a CD109 predicted loss-of-function polypeptide; ii) a CD109 missense variant mRNA molecule encoding a CD109 predicted loss-of-function polypeptide; or iii) a CD109 missense variant cDNA molecule encoding a CD109 predicted loss-of-function polypeptide. Any of the CD109 inhibitors described herein can be used in these methods. The subject can have or have a risk of developing decreased bone mineral density, osteopenia, Type I osteoporosis, Type II osteoporosis, or secondary osteoporosis.

The present disclosure also provides uses of CD109 inhibitors in the preparation of a medicament for treating decreased bone mineral density in a subject that: a) is reference for a CD109 genomic nucleic acid molecule, a CD109 mRNA molecule, or a CD109 cDNA molecule; or b) is heterozygous for: i) a CD109 missense variant genomic nucleic acid molecule encoding a CD109 predicted loss-of-function polypeptide; ii) a CD109 missense variant mRNA molecule encoding a CD109 predicted loss-of-function polypeptide; or iii) a CD109 missense variant cDNA molecule encoding a CD109 predicted loss-of-function polypeptide. Any of the CD109 inhibitors described herein can be used in these methods. The subject can have or have a risk of developing decreased bone mineral density, osteopenia, Type I osteoporosis, Type II osteoporosis, or secondary osteoporosis.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### General Methodology

### UK Biobank Cohort Description

Genetic associations were examined in the United Kingdom (UK) Biobank (UKB). The UKB is a population-based cohort of individuals aged between 40 to 69 years and recruited via 22 testing centers in the UK between 2006-2010. Genetic and phenotypic information from close to 300,000 European-ancestry participants in UKB were used.

### Phenotype Definition

Data pertaining to quantitative ultrasound of the heel were extracted from UKB. eBMD trait values (in g/cm²) were derived using a combination of speed of sound (SOS) and bone ultrasound attenuation (BUA; eBMD=0.002592×(BUA+SOS)−3.687). Sex-specific quality control measures were implemented for SOS (Subjects were excluded if SOS≤1,450 or ≥1,700 m/s for men, ≤1,455 or ≥1,700 m/s for women), BUA (exclude if BUA≤27 or ≥138 dB/MHz for men, ≤22 or ≥138 dB/MHz for women), and eBMD (exclude if ≤0.18 or ≥1.06 g/cm² for men, ≤0.12 or ≥1.025 g/cm² for women). Phenotypic values for eBMD were first transformed using rank-based inverse normal transformation, applied within each ancestry group and separately in men and women, and adjusted for fine-mapped common genetic variants associated with eBMD.

### Genotype Data

High coverage whole exome sequencing was performed as previously described (Dewey et al., Science, 2016, 354, aaf6814; and Van Hout et al., Nature, 2020, 586, 749-756) and as summarized below. A modified version of the xGen design available from Integrated DNA Technologies (IDT) was used for target sequence capture of the exome. A unique 10 bp barcode (IDT) was added to each DNA fragment during library preparation to facilitate multiplexed exome capture and sequencing. Equal amounts of sample were pooled prior to exome capture. Sequencing was performed using 75 bp paired-end reads on Illumina NovaSeq instruments. Sequencing had a coverage depth (i.e., number of sequence-reads covering each nucleotide in the target areas of the genome) sufficient to provide greater than 20× coverage over 90% of targeted bases in 99% of IDT samples. Data processing steps included sample de-multiplexing using Illumina software, alignment to the GRCh38 Human Genome reference sequence including generation of binary alignment and mapping files (BAM), processing of BAM files (e.g., marking of duplicate reads and other read mapping evaluations). Variant calling was performed using the GLNexus system (Lin et al., 2018, bioRxiv: 343970). Variant mapping and annotation were based on the GRCh38 Human Genome reference sequence and Ensembl v85 gene definitions using the snpEff software. The snpEff predictions that involve protein-coding transcripts with an annotated start and stop were then combined into a single functional impact prediction by selecting the most deleterious functional effect class for each gene. The hierarchy (from most to least deleterious) for these annotations was frameshift, stop-gain, stop-loss, splice acceptor, splice donor, stop-lost, in-frame indel, missense, other annotations. Predicted LoF genetic variants included: a) insertions or deletions resulting in a frameshift, b) insertions, deletions or single nucleotide variants resulting in the introduction of a premature stop codon or in the loss of the transcription start site or stop site, and c) variants in donor or acceptor splice sites. Missense variants were classified for likely functional impact according to the number of in silico prediction algorithms that predicted deleteriousness using SIFT (Vaser et al., Nature Protocols, 2016, 11, 1-9), Polyphen2_HDIV and Polyphen2_HVAR (Adzhubei et al., Nat. Methods, 2010, 7, 248-249), LRT (Chun et al., Genome Res., 2009, 19, 1553-1561) and MutationTaster (Schwarz et al., Nat. Methods, 2010, 7, 575-576). For each gene, the alternative allele frequency (AAF) and functional annotation of each variant determined inclusion into 7 gene burden exposures: 1) pLOF variants with AAF<1%; 2) pLOF or missense variants predicted deleterious by 5/5 algorithms with AAF<1%; 3) pLOF or missense variants predicted deleterious by 5/5 algorithms with AAF<0.1%; 4) pLOF or missense variants predicted deleterious by at least 1/5 algorithms with AAF<1%; 5) pLOF or missense variants predicted deleterious by at least 1/5 algorithms with AAF<0.1%; 6) pLOF or any missense with AAF<1%; 7) pLOF or any missense variants with AAF<0.1%.

Association Analysis of Gene Burden of Rare pLOF and Missense Variation in CD109

Association between the burden of rare predicted loss-of-function or missense variants in CD109 and eBMD was examined by fitting a linear regression model, including adjustment for a polygenic score that approximates a genomic kinship matrix, using REGENIE v1.0 (Mbatchou et al., Nature Genetics, 2021). Analyses were adjusted for age, age², sex, age-by-sex and age²-by-sex interaction terms, experimental batch-related covariates, ten common variant-derived principal components, and twenty rare variant-derived principal components. Association analyses were performed using single variants, and using gene burden tests. In gene burden tests, all individuals are labelled as heterozygotes if they carry one or more qualifying rare variant (as described above based on frequency and functional annotation) and as homozygotes if they carry any qualifying variant in the homozygous state. This “composite genotype” is then used to test for association.

Effector Index for eBMD Causal Genes

Effector Index, a novel machine-learning algorithm, has been described elsewhere in the literature (Forgetta et al., bioRxiv: 2021, 2020.2006.2028.171561). Training data were generated by performing GWAS analysis for eleven diseases and traits (type 2 diabetes, low density lipoprotein cholesterol level, adult height, calcium level, hypothyroidism, triglyceride level, glucose level, red blood cell count systolic blood pressure, diastolic blood pressure and direct bilirubin level). Fine-mapping was performed for each GWAS dataset, and genomic annotations were used as features to predict positive control genes at fine-mapped GWAS loci, using a gradient boosted trees algorithm (XGBoost). This trained algorithm was then tested on fine-mapped and annotated eBMD associations data at the CD109 locus to test the probability that the CD109 gene is the gene at this locus that influences eBMD.

### Mendelian Randomization Analysis of Circulating CD109

Two-sample Mendelian randomization (MR) was used to examine the association between genetically-predicted circulating CD109 and eBMD. This approach uses common genetic variants associated with CD109 protein concentration (termed protein quantitative trait loci, or pQTLs) as instrumental variables. The lead CD109 cis-pQTL was identified in two previously published studies performed in the INTERVAL (N=3,301) and AGES (N=3,200) cohorts (Sun et al., Nature, 2018, 558, 73-79; and Emilsson et al., Science, 2018, eaaq1327). The pQTL-outcome associations for this analysis were extracted from a previously published GWAS of eBMD in UKB, and the TwoSampleMR R package was used to perform MR analysis using the Wald ratio method. Colocalization analyses were performed to interrogate the influence of confounding by linkage disequilibrium. This entailed assessing the whether the genetic association signal for CD109 protein concentration is likely to share the same causal variant with the eBMD genetic association signal at CD109. These colocalization analyses were implemented using two previously published algorithms, Coloc (Giambartolomei et al., PLOS Genetics, 2014, 10, e1004383) and eCAVIAR (Hormozdiari et al., Am. J. Hum. Genet., 2016, 99, 1245-1260).

### Example 1: Loss-of-function of CD109 is Associated with Higher Estimated Bone Mineral Density

Whole exome sequencing of 278,807 European-ancestry individuals in the UK Biobank (UKB) was performed to identify predicted loss-of-function (pLoF) and missense genetic variants in each gene in the genome. The association of each sequenced gene and genetic variant in UKB with estimated bone mineral density (eBMD, measured using ultrasound of the heel) was examined. eBMD is a commonly-used biomarker of bone density and strength, and is highly correlated with bone mineral density as measured using dual-energy X-ray absorptiometry (DXA) technology. Lower levels of bone density are strongly associated with a higher risk of osteoporotic fractures.

The exome-wide analysis in UKB found that the burden of rare (alternative allele frequency [AAF]<1%) pLoF variants in the CD109 gene is associated with 0.18 standard deviation units higher eBMD (P-value=1.20×10⁻⁰⁹, meeting a Bonferroni-corrected, exome-wide statistical significance threshold of P<3.6×10⁻⁷ (corrected for 20,000 genes and seven variant aggregation models)) (Table 2; estimates of association pertain to the burden of CD109 pLoF variants with AAF<1%, and were derived in UKB).

TABLE 2
Association of rare pLOF variants in CD109 with higher eBMD.

| Beta, per allele, | Beta, per allele, | | | |
|---|---|---|---|---|
| SD units of | g/cm^{z} units of | | Genotype counts, | |
| eBMD (95% CI) | eBMD (95% CI) | P-value | RR\|RA\|AA genotypes | AAF |
| 0.18 | 0.022 | 1.20 × 10⁻⁰⁹ | 277945\|862\|0 | 0.0015 |
| (0.12, 0.24) | (0.015, 0.029) | | | |

Genotype counts indicates the number of individuals in each of three genotype categories: RR indicates individuals carrying no rare pLoF variants in CD109; RA indicates individuals carrying a rare pLoF variant in a single CD109 allele; AA indicates individuals carrying rare pLoF variants in both CD109 alleles. AAF indicates the alternative allele frequency of pLoF variants included in this analysis. g/cm², grams per centimeter squared; SD, standard deviation; CI, confidence interval.

The association of CD109 variants with higher eBMD was also significant when examining the gene burden of rare pLoF or predicted-damaging missense variants in CD109 (Table 3; estimates of association pertain to the burden of CD109 pLoF or predicted-damaging missense variants with AAF<1% or <0.1% and were derived in UKB (see Genotype Data below for description of in silico algorithms used to identify predicted damaging missense variants)). These genetic data suggest that loss-of-function of CD109 leads to a higher eBMD in humans.

TABLE 3
Association of rare pLOF or missense variants in CD109 with higher eBMD

| | Beta, per | Beta, per | | Genotype | |
|---|---|---|---|---|---|
| | allele, SD | allele, g/cm² | | counts, | |
| Genetic | units of eBMD | units of eBMD | | RR\|RA\|AA | |
| exposure | (95% CI) | (95% CI) | P-value | genotypes | AAF |
| pLoF or | 0.07 | 0.008 | 7.0 × 10⁻⁰⁸ | 273953\|4846\|8 | 0.0087 |
| damaging | (0.04, 0.09) | (0.005, 0.011) | | | |
| missense | | | | | |
| variants, | | | | | |
| AAF < 1% | | | | | |
| pLoF or | 0.11 | 0.014 | 3.2 × 10⁻⁰⁹ | 276693\|2114\|0 | 0.0038 |
| damaging | (0.08, 0.15) | (0.009, 0.018) | | | |
| missense | | | | | |
| variants, | | | | | |
| AAF < 0.1% | | | | | |

Genotype counts indicates the number of individuals in each of three genotype categories: RR indicates individuals carrying no rare pLoF variants in CD109; RA indicates individuals carrying a rare pLoF or damaging missense variant in a single CD109 allele; AA indicates individuals carrying rare pLoF variants or damaging missense variants in both CD109 alleles. AAF, alternative allele frequency of variants included in this analysis. g/cm², grams per centimeter squared; SD, standard deviation; CI, confidence interval.

### Example 2: Variants Associated with Lower CD109 Protein Concentration in Blood are also Associated with Higher eBMD

Using Mendelian randomization, it was discovered that lower circulating CD109 protein (encoded by the CD109 gene) due to common genetic variants in the CD109 locus was associated with higher eBMD (Table 4; the lead cis protein quantitative trait locus (pQTL) for CD109 was obtained in two independent cohorts: INTERVAL and AGES. A two-sample Mendelian randomization analysis was performed using eBMD GWAS data from UKB as the outcome dataset). This relationship was further supported by a colocalization analysis, performed using two distinct algorithms (Coloc and eCAVIAR; Coloc posterior probability of H3=0.042, Coloc posterior probability of H4=0.958; eCAVIAR CLPP C1=0.024, CLPP C2=0.002). These results provide complementary evidence for the results reported in Table 2, which showed that loss-of-function of CD109 is associated with a higher eBMD. Several individual rare pLoF and missense variants in CD109 showed nominal evidence of association with eBMD in UKB (P-value for association<0.05; Table 4).

TABLE 4
Mendelian randomization analysis supports a causal
influence of circulating CD109 concentration on eBMD

| | | Effect on | |
|---|---|---|---|
| | | eBMD, in SD | |
| | | units, per SD | |
| | | unit decrease | |
| | | in circulating | |
| | | CD109 | |
| pQTL source | PQTL | concentration | P-value |
| INTERVAL | rs6903575 | 0.056 (SE = 0.004) | 6.4 × 10⁻³⁷ |
| AGES | rs6909201 | 0.043 (SE = 0.003) | 3.2 × 10⁻⁰⁹ |

pQTL, protein quantitative trait locus;
SD, standard deviation;
SE, standard error.

### Example 3: Machine-learning Algorithm Applied to Common Genetic Variation at CD109 Identifies Further Evidence Implicating CD109 as the Causal Gene Mediating the Association with eBMD

A machine-learning algorithm (Effector Index) was applied to eBMD genome-wide association data and strong evidence was observed to suggest that CD109 is the causal gene mediating the eBMD GWAS association in this genomic region (Effector index=0.96, which indicates that the probability that CD109 is the causal gene at this locus is high).

1. A method of treating a subject having decreased bone mineral density, the method comprising administering a Cluster of Differentiation 109 (CD109) inhibitor to the subject.

2. The method of claim 1, wherein the decreased bone mineral density is an osteopenia.

3. The method of claim 1, wherein the decreased bone mineral density is Type I osteoporosis.

4. The method of claim 1, wherein the decreased bone mineral density is Type II osteoporosis.

5. The method of claim 1, wherein the decreased bone mineral density is secondary osteoporosis.

6. The method according to claim 1, wherein the CD109 inhibitor comprises an inhibitory nucleic acid molecule that hybridizes to a CD109 nucleic acid molecule.

7. The method according to claim 6, wherein the inhibitory nucleic acid molecule comprises an antisense nucleic acid molecule, a small interfering RNA (siRNA), or a short hairpin RNA (shRNA).

8-14. (canceled)

15. The method according to claim 7, further comprising administering a therapeutic agent that treats decreased bone mineral density in a standard dosage amount to the subject wherein the subject does not have any CD109 missense variant nucleic acid molecule encoding a CD109 predicted loss-of-function polypeptide.

16. The method according to claim 7, further comprising administering a therapeutic agent that treats decreased bone mineral density in a dosage amount that is the same as or less than a standard dosage amount to the subject wherein the subject is heterozygous for a CD109 missense variant nucleic acid molecule encoding a CD109 predicted loss-of-function polypeptide.

17. (canceled)

18. The method according to claim 15, wherein the CD109 predicted loss-of-function variant nucleic acid molecule is 6:73730573: A:G, 6:73823473:GA:G, 6:73763607:C:A, 6:73803256:G:T, 6:73818486: T:C, 6:73787379:G:A, 6:73771510:A:G, 6:73806987: A:T, 6:73758991: A:G, 6:73823456: A:G 6:73762778: A:C, 6:73763660:A:G, 6:73730573: A:G, 6:73806956:G:A, 6:73792628: G:C, 6:73806926: A:T, 6:73771576:G:A, 6:73815026:C:T, 6:73765952:G:A, or an mRNA molecule produced therefrom, or a cDNA molecule produced from the mRNA molecule.

19. The method according to claim 16, wherein the CD109 predicted loss-of-function variant nucleic acid molecule comprises the variation in 6:73730573: A:G, 6:73823473:GA:G, 6:73763607:C:A, 6:73803256:G:T, 6:73818486: T:C, 6:73787379:G:A, 6:73771510:A:G, 6:73806987:A:T, 6:73758991:A:G, 6:73823456: A:G 6:73762778: A:C, 6:73763660: A:G, 6:73730573: A:G, 6:73806956:G:A, 6:73792628:G:C, 6:73806926: A:T, 6:73771576:G:A, 6:73815026:C:T, or 6:73765952:G:A, or an mRNA molecule produced therefrom, or a cDNA molecule produced from the mRNA molecule.

20. A method of treating a subject with a therapeutic agent that treats or inhibits decreased bone mineral density, wherein the subject has decreased bone mineral density or is at risk of developing decreased bone mineral density, the method comprising the steps of:
determining whether the subject has a Cluster of Differentiation 109 (CD109) missense variant nucleic acid molecule encoding a CD109 predicted loss-of-function polypeptide by:
obtaining or having obtained a biological sample from the subject; and
performing or having performed a sequence analysis on the biological sample to determine if the subject has a genotype comprising the CD109 missense variant nucleic acid molecule; and
administering or continuing to administer the therapeutic agent that treats or inhibits decreased bone mineral density in a standard dosage amount to a subject that is CD109 reference, and/or administering a CD109 inhibitor to the subject;
administering or continuing to administer the therapeutic agent that treats or inhibits decreased bone mineral density in an amount that is the same as or less than a standard dosage amount to a subject that is heterozygous for the CD109 missense variant nucleic acid molecule, and/or administering a CD109 inhibitor to the subject; or
administering or continuing to administer the therapeutic agent that treats or inhibits decreased bone mineral density in an amount that is the same as or less than a standard dosage amount to a subject that is homozygous for the CD109 missense variant nucleic acid molecule;
wherein the presence of a genotype having the CD109 missense variant nucleic acid molecule encoding the CD109 predicted loss-of-function polypeptide indicates the subject has a decreased risk of developing decreased bone mineral density.

21-38. (canceled)

39. A method of identifying a subject having an increased risk of developing decreased bone mineral density, the method comprising:
determining or having determined the presence or absence of a Cluster of Differentiation 109 (CD109) missense variant nucleic acid molecule encoding a CD109 predicted loss-of-function polypeptide in a biological sample obtained from the subject;
wherein:
when the subject is CD109 reference, then the subject has an increased risk of developing decreased bone mineral density; and
when the subject is heterozygous or homozygous for the CD109 missense variant nucleic acid molecule encoding the CD109 predicted loss-of-function polypeptide, then the subject has a decreased risk of developing decreased bone mineral density.

40-78. (canceled)

79. The method according to claim 7, wherein the antisense nucleic acid molecule, small interfering RNA (siRNA), or short hairpin RNA (shRNA) comprises non-natural or modified nucleotides.

80. The method according to claim 7, wherein the antisense nucleic acid molecule, small interfering RNA (siRNA), or short hairpin RNA (shRNA) is administered parenteral, intravenous, subcutaneous, intra-arterial, or intramuscular.

81. The method according to claim 1, wherein the decreased bone mineral density is osteopenia or osteoporosis, wherein the inhibitor is an antisense nucleic acid molecule, a small interfering RNA (siRNA), or a short hairpin RNA (shRNA) administered parenteral, intravenous, subcutaneous, intra-arterial, or intramuscular.

82. The method according to claim 1, wherein the decreased bone mineral density is osteopenia or osteoporosis, wherein the inhibitor is a small interfering RNA (siRNA) administered parenteral, intravenous, subcutaneous, intra-arterial, or intramuscular.

83. The method according to claim 1, wherein the decreased bone mineral density is osteopenia or osteoporosis, wherein the inhibitor is a small interfering RNA (siRNA) administered parenteral, intravenous, subcutaneous, intra-arterial, or intramuscular, and wherein the subject is heterozygous for a CD109 missense variant nucleic acid molecule encoding a CD109 predicted loss-of-function polypeptide.

84. The method according to claim 83, wherein the CD109 predicted loss-of-function variant nucleic acid molecule is 6:73730573: A:G, 6:73823473: GA:G, 6:73763607:C:A, 6:73803256:G:T, 6:73818486:T:C, 6:73787379:G:A, 6:73771510:A:G, 6:73806987:A:T, 6:73758991:A:G, 6:73823456:A:G 6:73762778: A:C, 6:73763660: A:G, 6:73730573: A:G, 6:73806956:G:A, 6:73792628:G:C, 6:73806926:A:T, 6:73771576:G:A, 6:73815026:C:T, 6:73765952:G:A, or an mRNA molecule produced therefrom, or a cDNA molecule produced from the mRNA molecule.

85. The method according to claim 1, wherein the decreased bone mineral density is osteopenia or osteoporosis, wherein the inhibitor is a small interfering RNA (siRNA) administered parenteral, intravenous, subcutaneous, intra-arterial, or intramuscular, and wherein the subject is wildtype for a CD109 missense variant nucleic acid molecule encoding a CD109 predicted loss-of-function polypeptide.

86. The method according to claim 85, wherein the CD109 predicted loss-of-function variant nucleic acid molecule is 6:73730573:A:G, 6:73823473:GA:G, 6:73763607:C:A, 6:73803256:G:T, 6:73818486:T:C, 6:73787379:G:A, 6:73771510:A:G, 6:73806987:A:T, 6:73758991:A:G, 6:73823456:A:G 6:73762778:A:C, 6:73763660:A:G, 6:73730573:A:G, 6:73806956:G:A, 6:73792628:G:C, 6:73806926: A:T, 6:73771576:G:A, 6:73815026:C:T, 6:73765952:G:A, or an mRNA molecule produced therefrom, or a cDNA molecule produced from the mRNA molecule.


SEQUENCE LISTING (58 sequences)

SEQ ID NO: 1 - DNA, 132532 bp, Homo sapiens
  source 1..132532: mol_type = genomic DNA
atagttgctatgaatttctcctcacaaggatgggcctgtttactcaccctgggcatcgttggtagagcgctagtgtaaacagcctgaggaacccggtgggccggggaagtgggcgcgctctgttctccgcggccagctgggacgccgggccaggtggggccgcctgcgtttagcaactgctttctcaccccctggatttgcgatgtttgccacagcagcgagaagcgccattgtaatggggatgggaggggtggagcctccaagtcctgtctcaatttagatctctcactctgctgttaggcgcgcccatttcagattactaaactcgaattaagagggaaaaaaaatcagggaggaggtggcaagccacaccccacggtgcccgcgaacttccccggcagcggactgtagcccaggcagacgccgtcgagatgcagggcccaccgctcctgaccgccgcccacctcctctgcgtgtgcaccgccgcgctggccgtggctcccgggtaggaacgtgggcgcgcggggggcgcgcgggcgcgcgggcctgggccgctctgcggctctgggccagggcttcggggaggtggcggctgctgtgcagcagcgggtgggaaatgccctcgcggctgcagtccccagcctggtactggcctggaggtttgaccatatgtagcttcagcgtggctctccatgggacagttaactttctccactcatgaagttgtttaagcgtctccccggccataacaactttctgaagagagtcattttatttttagtccacattgcctctgccttttgcttctcaactttttgctcccagattgcgattcttctctcaaagacatgatatattttttctaaccaagatgtctcaaccttagcattgccaaagtgtggcggaggggactgatgtatgggttcaggggcagacattataggaagaaaacaacagcacccaataatggcaagccccattcattcctaaagatttctgtagttgagccctaacccctagttatagcagagaaagtgctgctttagtgacttcttttatgattcgctatacctggaattttcaccagttgtagtttattttcaaatggtatatttcaatcaatggtattggttaaataattttttaccctcgttgggcagctactcaaggataaaggtttgaataagaaacaggcaaaatcttagttaaaaaataacaacaacaacaagaaaataatcacgctgggataaggtcctctgtaaaggagaagcttagagacttttgctttgccaataactccattatgccccggtgcaagccatttacatcatggttccttctgtgcggttcgtggtttataaaatggaaataataacacaggccttcctctagtagtgatgcatgaattactgcattaaaattgatttatgggaattattgttgtttcagtagcatttcaattcagttgccaaatagagcagtgggcaatgttaacggaaacaactgcaattggcgcagtatggagtgcctatcgcactaggaaatctgagggtcacaaaagaaaggagatgtgaggataagaaactttgtttttcccttgttgggaactctttaggcctcggtttctggtgacagccccagggatcatcaggcccggaggaaatgtgactattggggtggagcttctggaacactgcccttcacaggtgactgtgaaggcggagctgctcaagacagcatcaaacctcactgtctctgtcctggaagcagaaggagtctttgaaaaaggtaagataaacagcataaagtcttacccttctgcagtaataactggaatatgttaataaggtcatgtgttaggtagtatagcagagaaaccccaaatttgcagtatcttacctaatatacttttaattctcact[450 bp omitted]tttgtagcagaaccaagtttggttcttgtttcaagtttgggttctgacatggcccaagattaaattgggccaatgaatgtcttctttttcagtgctaagggaaaaataattttgggaggaagaggtaaatacttaaatacttaaaacatttttcattaaaatgaacttttattgcatttttttctttttttagattcttataattatttttagagacaagctctcaccatgttgcccaggctggtctcgaactcctaagttcaagtgatccacccaccttggcctcccaaagtgctgggattacagtcctaagctactgctcctggccgagacatacatttttataatgttaagagttatttaaaaaaaaaaatttaaggcacgtaatgaagggtggttggtattcattacataagtgtttattctctacaagcgtagagaaatgaacaccatcatgaaatgaaataaaagtaaaagtttatctgcatctgtggttctttccccactggaagttcactgggtaggtatatttggaataggagtcaggaagagatggtgtgtgtaggggcaagtcacttttgtccctcatagaagcaatgtcagggaagtgggaggtcttatttccctggaggggaagtataggttagcccttaagatctgagtttgaatcctggtactactttgctactagttgtatgatcttaggtaggttacttaactgctttgagccacagtttccttgtctataaaatggaaataatgaaactgatttcccagggcagttataaagttaaaatctgtatatagtacctcttaggcatgcaataaacaacaattgctaatattattagtgtaataatactgcagaagaggatgcagaaagtccatttcctttttacatgggggcagacttcaactttcgttattggactatcatttctgaaaatgaatgacccctgatcttatgatagtcgctaaagaaaatattgaaatattaaatcacaggacataatacttaaggtagttccacattcattatttattcattaagaatttatgagtgggaagagggagggtattaaaaaaccacctattgggtaccacacttatcacctggatgatgaaataacatgtacactaaagccctatgacaggcattttacctatataacaaacctgcacgtgtatccctgaaactaaaataaaagttaaaaaaaaaagaatttataatattggctccactagtgtggcagttttcctgcaatgcagagatccagaatatatatggatgtttttgataaatatttgagctttgcttggacatttacatactctaattctagaatttcacctgttaggtcttttctgtgatctctgttatcagtctctctttttttttgcaacaaatagtacctacattggggtgcatgttgttgtggcttggttttcagctttattcttagatcttcttttgggagaagggttccttgtatgcccataggaaagttcaagtactcaagactggggcgggcaaagctgtcctgggagcagaatggatctgtgggaaggaaggagggaaaacgctgcttctttcatgggctgtttttgactctattgacatagagtttcattgaaaatgccattgctaggagcactctcctgtttcaaaggagaatgttactgtattatcagaggctgggagttattgttctcagagcagatgcttcctgtaatgtgaggacttgaggatcaagacacttctccctgcttgtcttcacatcctttgctttatctggcttctccagttgtaacaatatgtagataaaccaggacttctaaatcaggggtctggatcttcaagtatctggatgtggattcatgttttgttttttctccatcacataaacaccaaacaccaacaaataaataccttttatttttaagggacagagtctcactctgttgcccaggccagagtgcagtggtgaaatcatagctcactgcaacttggaattcttgggctcaagtgatcctgctgtctcagcctcctgaatcagctgggactacaggcaagtgccagcatgctaggtttaaaattttttttttttttgtagagatggggtcttgctatgttgcccagactggtcttgaactcctgggctcaagcgatcttcctgcctcagcctcccgaagtgctgggattatgggtatgagcctgattgtttcattttcctatgagccacccccagccccccaacagaaggttggttttaataaagatttatcttctagtaaagttgggtaaggcagtagactgtgtcttagtagcttatttccagcagttttggatttcttagaaatcctcaggatagggaggtgttttacccaaaccacaatagatttctggctcaatttgtgttggaaatagtaataactaaattgttcaaatggagaatgtatttaataaaggtgaaaaagtatggcatttcttcagggaagagttcatttgtttgtttgacagatatgggggagattttatttacatttccacttttatggccaggagtgtaggttatattactgttattcatttaaaaatacattaattctacatttattggggattgattgtagtttttttttttttttttttttttttttttgagacacagtctcactctgtcacccaggctggagtgcagtggtgcgatctcggctcactgcaacctctgcctccctggttcaagcgattctcctgcctcagccttccgagtaactgggactacaggcatgtgccaccatgcctggctaattttttgtatttttagtagagacgaggtttcactgtgttagccaggttggtcttgaactcctgacctcgtgacctgcctgcctcggcctcccaaagtgctaggattataggcgtgagccaccgtgcccagcgattatgggctttttttttttttttggtgaataatagacctttagaaaaagtatttctgctacactttgcagagttctgggaaggtgtatcaatgcctttctagtagtgagattcaaaagattgcttgtcactgttaccactgtcactgcctctaccactatcagcatcattgccatcagctccatagtgattagaactatgttcctaatcttatcttcctttgcataaagagttagttaaataaacataactggcaaaacgtagtaaaagttcagcaggtaataattaccagaggaaagaagatggcacagctattggcatttaaaatcaagtcagattactgttttgggtggagagtgagtctgtccctttgtctcttcccatattttttttctctttccttactgtgaaaggagttccttgccaaatcaagaaaaaagaaattaagaacattttgagaactgtcctctcatctgttaaggcatataaaataatttaattttcgtgatgtccttgaaaccataatttctgttttattttcccttctcttgctacaatttgacattttcatttgtaaactttcagtagttttcctgaacaagagtcacttcaagtaataaagaatataacctttctccctcattaatgaattcatcatcattagtttcatgtaaacctgatttaagaaaaaatgctagaggctgagtgcagtggctcacaaccgtaatcccagagcttgggaggctgaggtgggaggattgcttgagcccaggagtttgagaccagcctggacaacacagtgaaattctatctcaaaaacaaaaacaaaagaacccttaaaaccaaaaactcccaaaaacctaaactgaaaatgcctactgaaattttgggaactctgcatgggcgtctccaggtgagtgcctgctggtttgggggcaccattttttaaatgctgccaatcctacttccatcttcccattgagttactggggtggaatgtgctctataaatgtgtctgtaactcgctttttctttttgccctaaaatcctggagagttaatggagtgttaacctttttttaaagattttttaaaaaatctattttttcctttgttgtctttctcacattgactatgatatattttattttccatcttggctcagtaagggtgggaaatttttaaaaattgagatattagctgaaaaacttaaaaaatcatccaatttggtttggatggttttctggtggaaacaaagctcctgaacatgcatttcatgcctcgtagataaaaatcagtcccatggtctttatgaaagtagattaattagtgtagtgtgtgggcagatctcccagagcaggttttcacaggcagctttgccaacataactcagtctgaacctgcagttatgttagaattatttttaaagggtgtaggcttttcattcacacatcgtctttgttcttgtgtgtgatgtatattaacaggtaggtggtacctgagaataaaggaccacagcataatcacgatgtgcctgggctctggagcctggctacctgggcacaggtgctggccattccacttagtggttgtgtggctgtgggcagtcatgatctcactaggcttcagtttcatcatctgaaaaatagtggtttctgccttatacaattatatggatattgtgaggattaaatgaggtaacatattcaaggcacttaacacaaaggaattattcaagaaaatggtagctattgttactagaagagagtagcaacagtttatgtcaaaggcaattattaatcacctattgtgacaatcttgaaccgggaactctataaaagacacatccctaggaggggattggaagggatttaacatattaaatatccagtttcctcatagtgccaaggactttatgcactatatttaattttaacaacttgatgaagtagatactcttgtcttcattttggaggtgaggaaattgaggcaaagagagaagaattagtttttctgagttaacccagttagtaaatggtgaagcctgaatttgaagttagttgtatctgaggtccagagaccataaggttttctattgctgggaagagagtgaagtatttttatttccttgattttgggatattgatgcaatgttgcagttgtcttccactaaaatcattttcacatttgcttgcagtagatagacctggatctaatgaaatctaatttgaatcgaaagaattacaaaatgaaatgccagctctcctcttaatttactttcagcctcatagagttttggagctggaggggacctcaaagataagcaaattcaatagcctaatatatggctgggagactgaggtgaggcatgggatctggggattttcccaaggtcaaatcacttattggtggcagagctggaaacagattgcagtttcccaaatttgcagtccagtgctatttccagtatactctacgaaagtttcctaacatatggagagctgagcttaagacctaagatatatttacaaatgctgattcttctgatatggaagaaaataaggctaactaaggagtttaaaaatgcttacttgcatactgtgaaagtttcttattttagctcaaggtggagttgctctttatgcactatttgttctgtataaggggtgggggctgggttagaagcttcatagaccttttaaaaactaaattctgttgctttgtttctcagagtccaaattgtatttattaaaaaggctgattcatggcctccctcaagacatgggtaagaatcaagaatctcagctgggtgtggtggctcacgcctataatcccagcactttgggaagccaaggtaggtggatcacttgaggccagaagttcgagaccagcctggccaacatggtgaaaccccgtctctaccaaaaacacaaaaattagccaggtgtggtggtgggcgcctatagtcccagttacttgggaggctgaggcaggagaatcgcttgaacccaggaggcagaggctgcagtgagattgtaccagtgcactccagcctgggtgacagagcaagactccatctcaaaaaaaaaaaaaaaaagagaatctaacctttatgttccaccatgaccacaggtgattcttagtcacattagaatgagaacaactgccctttggtatctcacaaattgtactcgaccaaatactggaaagctcgagggtgagtagcatgcccctgcaagcctgtgatgattcatggcggcggagggaaggctcctcgtggactttcatgtttgcttttaagcaaccctcaaccctaagcgctggtgttcattttcagccgcagagaaacatgacttggggtttgattgtgaatcaacgattgggtgaagttaaaagtgaacacactgattctttaaactccaagttaaacatgtgctgctcctcttctagcactggatgagtcactgtcctagtgactcactgaaggattgtggtggaaatagaaaacagatcccataagaatttgggctgggttactgttgatcctttatgtcctgtgaagagggctttataatgctgaaactagttgtttccccacagacatttcttctcattaatgtattatttattgattatttgctgtatgtgaaacatgaatgaatggggagtggggaataagagaaaggaaggcactggaggacattcagaggtcccagaaggataatataatattaaaagtgcagaaggctagaggagtttttattttttgtataaaaaatacaatgcggtttgtaaatagtgcttcttgtatgtgagcctgtgcctggtactattatcttttacttccagagctatacagatgtccattcataaatgtcccacctctgctgacatatttggcctttggagggaacagttacttcacgtactagagacgggaagggtgtgccggaggaaagagctgctggctggtgtggcagttatgaaaaggagataggggtttgtgtatatattttctaaaaagatgtttcattctgctttggatatgagtttggggagacatccaggtggagatgtccagcagatagttttgaaattgcagctggggagagagatttaggcattattttcatagaggcagacaatattttgaagtcctggaaatacataagatgcacaagagagacagagctcatggaaagaacaagtcttttgagagaagcttggaaagaggaaaataagttaatgaagggaaaaagaccagacacagtggctcatgcctgtaatcccagcaacttgggaggctgaggtgagcagatcgcttgagctcaagagtttgaaatcagcctggacaacatattggaacctcatctctactaaaaataaaaaaaaggtagctggacgtggtgacatgtgcttgtagtcccagctactcaggaggctcaggcaggaggatttcttgagctacagttatagtgccgttgtactctagcctgggtaacaaagcatggccctgtctcaagaaaaagaaaagaaaagaaaagaaaagaaaagaatagaatagacaacgagcaatcagagaagggtaaattgaaaatgtctcagaagccaagggaagaagaagttccaagaaggaaggtttaatagttaataatcaattctctcaggtcagagaagaggaggacagaggaaagaacattggtgacgcttaagtaaatggctttaagttgaatgttggaagcaaaagcaaatttgagggaataaatggatggtgaaaaatataatcaaaatttattctttccagaagtttggtagttgaaaggagagaggtggacggtctctcaaaaggcacattagaaaggtgaaagtggttaaggcaaaggagagggtggataattcctgggttagaagtggtgagatcagtattggggcagaggcaagtggattatccttaaaaaaccctcatactttcttctttgaagaaagaaagaaatagaagagaatcattaaggaaagaaatattttgaggtatgaaattgagatgaagaataaaattgaaatacagttttaggaaaacttatttaccgacttgtaaattgcacagatctggccaccactttataggaaatgggggtaaaagttaaaagtagtggcagaccagaatgagaaacacaggactggagatcttccctggtggtgccctaaactgggtcaccgctggattgtaggaaattagaatacaggggcaggaagtcatatttgtatctgggagagctaacacagggaggagagagaagaggaaagggctgtttacgatgaagatatgcctcagttgaagctcgagaggactgggtggagttactttattactgccatggttgtcagtgaatttgtaattcactcttagtgtgggccaagatgaccaaggtgcaatgataaaacttgtctaggaggattaaagtggatcttggtcttgctctatctctttcactcttcacttacctcggagaattctatgaggagactttcttccgttgcaaggctcagggtgaaattggtcaaagatttggtggagggggtagcagtaggtaggtagatgacaggtctataggttgttagttctgaagaaacgattcataaattaaaatccgaaaaccagactggtaaagagcttggaaataaatgaagaggaaaatttttaaagaacacctagtagctcccttttggcagggctgtgttaggaatttacatttatgaaaatatagctggcagaaatgatttagtattacaaacttaagtttcactgcaatatatgttaaagctcctcaggaatatagataagacagggaatttattattctgagcaattcaaatttattcagcaccaaattgcctatggcacaatgctgggtgacataaatatttggtgaccacatgaggttcctgtgcctgtgctcttcgcagggaagcagagtaggcttcatttgatgtgtcctcccacgttgattatagaggcttgttcgggtgccgtgttgacagtttcaaagtcaagcgtgggtttactggactagagaactggatgaattagctgtgtctgccatggccatgggagttggggagtgtggtatgaatgctgcgaaggattctgtagcccatccataaactttatttatggatcagttgaagaggaatggtctaagaggaacggttatctacgtagctacataggggcttgaggtagtaacaacatggataagagagaacagacgaaagagtgggtgtatccaaatagtaaatgagagtggagtaagctacagaggaggaacaaggagggaagggatatgaggttatgtttgcactactgttttggaaggaatagctgggtctgtcctgcactgtttgggaggcacagtgggttagaatgggtgctcaggaaccacactgaaattgaaccctagcatcacctaatagtagtacccacctcgtagcattgttgagaggattaaatgagataatacttaagagctgtatcatccaatatggtagctactaaccacatgtggtttttgagcacttgaaatgtggctcatacaaattgagatgtgccaggagtataaaatacataatggattttgaatacttagttccaagaaatgcaataaaacttatgattttaaaataatgatttcatgttcaatgttaatattttggacatattgagttaaataaaatatgttattaaaagtaatgtcacccaattttttctttttttggcacataaaagacacctaatttaataaatattagttattattttctcttttattatgcagttaagaactttgatttaaaattgttatctttatatatatatatatatatatatatatatatatatatatatatatatttattatactttatgtcctagggtacatgtgcacaacatgcaggtttgttacatatgtatacatgtgccatgttggtgtgctgcacccattaacgcgtcacttacattaggtgtatctcctaatactatcccttcccccttcccccatcccacgacaggccccggtgtgtgatgttccccgtcctgtgtccaagtgttctcattgttcaattcccacctatgagtgagaacatgcggtgtttggttttttgtccttgcgatagtttgctgagaatgatggtttccagcttcatccatgtccctacaaaggacatgaactcatcattttttatggctgcattgtattccatggtgtatatgtgccacaatttcttaatccagtctgttgttgttggacatttgggttggttccaagtctttgctattgtgaataatgcctcaataaacatacgtgtgcatgtgtctttatagcagcatgatttatagtcctttgggtatatacccagtaatgggatggctgggtcaaatggtatttctagttatagatccctgaggaatcaccaccctgtcttccacaatggttgaactagtttacagtcccaccaacagtgtaaaagtgttcctatttctccacatcctctccagtacctgttgtttcctgactttttaatgatcgccattctaactggtgtgagatggtatctcattgtggttttgatttgcatttctctgatggccagtgatgatgagcatttttccatgtgttttttggctgcacaaatgtcttcttttgagaagtgtgtgttcatatcctttgcccacttgttgatggggttgtttgttttttttcttgtaaatttgtttgacttctttgtagattctggatattagccctttgtcagatgagtaggttgcaaaaatgttctcccattctgtaggctgcctgttcactctgatggtagtttcttttgctgtgcagaagctctttagtttaattagatcccatttgtcaattttggcttttgttgctattgcttttggtgttttagacatgaagtccttccccatgcctgtgtcctgaatggtattgcctaggttttcttctagggtttttatggttttaggtctaacatttaagtctttaatccatcttgaattaatttttgtataaggtgtaaggaagggatccagtttcagctttctacatatggctagccagttttcccagcaccatttattaaatagggaatcctttccccatttcttgtttgtgtcaggtttgtcaaagatcagatagttgtagatgtgtggcattatttctgagggctctattgtgttccattggtctatatctctgttttggtaccagtaccatgctgttttggttactgtagcctcgtagtatagtttgaagtcaggtagcatgatgcctccagctttgttcttttggcttaggattgacttggcaatgtgagttgttttttggttccgtatgaactttaaagtagttttttccagttctgtgaagaaagtcattggtagcttgatggggatggcattgaatctataaattaccttgggcagtatggccattttcctgataccaaagcctggcagagacacaacaaaaaaagagaattttaggccaatatccctgatgaacatcgatgcaaaaatcttcaataaaataccggcaaaccaaatccagcagcacatcaaaaagcttatccaccatgatcaagtgggcttcatccctgggatgcaaggctggttcaacatatgaaaatcaataaacgtaatccagcatataaacagaaccaaagacaaaaaccacatgattatctcaacagatgcagaaaaggcctttgacaaaattcaacagcgcttcatgctaaaaactctcaataaattaggtattgatgggacatatctaaaaataataagagctattcatgacaaacccacagccaatatcatactgaatgggcaaaaactggaagcattccctttgaaaactggcacaagacagggatgtcctctctcaccactcctattcaacatagtgttggaggttctggctagggcaatcaggcaggagaaagaaataaaaggtattcaattaggaaaagaggaagtcaaattgtccctgtttgtagatgacatgattgtatatctagaaaaccccattgtctcagcccaaaatctccttaagctgataagcaacttcagcaaagtctcaggatacaaaatcaatgtgcaaaaatcaccagcattcttatacaccaataacagacaaacagagagccaaatcatgagtgaactcccattcacaattgcttcaaagagaataaaatacctaggaatccaacttacaagggatgtgaaggacctcttcaaggagaactacaaaccactgctcaacgaaataaaggaggacacaaacaaatggaagaacattccatgctcatgggtaggaagaatcaatatcttgaaaatggccatactgcccaattttttctattttttttttagtgcacatctttaaaacatttatcacaatgcttggtacatagaagacaccgagggaatgttaactatgttgtacctggtgtttctcagtatcagatattaatactgatgcctgaccacaagcctgtggcatgagtccaagtctccctgctgatgactcattttggcttaggtagatcttcacccttatcccttatcccttgaggaagaagaaaatctccctaaagagagccacaggggtcggcaaaatttctgtaaagggccagatgttaagcatgtaggttttgccaaccttcaggtctttattacagctgctcaactctgcctttatagctcggaagcagctgtagacggttcataaacaaaagagcatggttgtgtaccagcagaactttatttatggatgctgaaattttgagttcatataatttttacttatcatgaaatattcttcttcttttgatattccccccagctatttagaaatgcaaacccattcttagttcatgaactgtacaaaaaacaggcgtgggccagactggatctgtaggctgtggattgctgaccctgccattaaaaatgccctgtacatttagcagccaagaaccttgcttgactctgggtttttcatttgtacttaacctaacatttgctttatagtgtaccattttatttattttacttttagtaggtttgtgtttgacctgcttttttttttactcaatgtttgtgtttaacctgcttttttttagacaatggaacaaaactttgtttatgaaactcaaatttattaagatatttgtaagcaaagggaaataagagagaaaaatataaaaggagtatgtaagggaccaaaggagattttttgtttggttttgttctgctcagaatacttgtgttttctgtgtagactgaatttacccaatagtttttttgccttaaagtgacattaaactgtgaagcagaaacattgtatttttaaaatcgtactttaagtttttttttttttttttgagacggagtctcgctctgtcgcccaggctggagtgcagtggcgggatctcggctcactgcaagctccgcctcccgggttcacgccattctcctgcctcagcctcccaagtagctgggactacaggcgcccgccactacgcccggctaattttttttttgtatttttagtagagacggggtttcaccgttttagccgggatggtctcgatctcttgacctcgtgatccgcccgcctcggcctcccaaagtgctgggattacaggcgtgagccaccgcgcccggcctaagtttttaaaatattttatttttatagatttagggggtacatgtgcagttttcttacatggatattttgggtagtggtggagtctgggcttttagtataaacatcagccaaatagtgtatgttgtatccattaagtcatttcttatcccttacctccctgccatatgaatctccaatgtctattatttcactctctaaaaaatattgtattttatacaaaaattagccgggtatggtggcgcatgcctctaatcccagctactggggggctgaggcaggaggattgcttgaacctggaaagcggaggttgtagtgagccgagatcatgccactgcactccagcctgggcaacgaagcgagactccgtctcaaaaaaaaaaaaattgtattttaaagttttgttttccatgaatttgtggggggaaaacccttatttcttaccttatctgttgatcaaagaaactgctgaagaatgaggaaaaaaatttagtaacttgataaagagatcaaaattttcagctaaaaaagtgataataaatttattattgcataagactgagtagcaaaaggctgccaaaaataattaagcagcaaacactaagtcaggcatatatatatatctagtggaaaatccactttttttttcatagagagtatttctgtacctagttggatttccggttacacactactccttcccttcaatgattttccaaagttatttggtgaggacagatgtcagaagaggcagcatataatggtattacagagtatgtttaaaaagttaaggaaaggttagtttttgtgtatgcttattacacaatttaaaaatagctttgactctatttgtaaatcaaatggccagttctcaggctgtgccaaatgcgaagccttcaaacaaggtttcttctgagttgcttaagcagccctgggaaggagaaagcatctgaaagggttttcagattgattgtctctggttatacacagaaatgattggagttttataaagttatctataaaaacactaagaagaaccttacataacagagtgtgtctttagttagtgatttaaaagagtgccatactgggtttcaagaggtggtgaccatttcccgtgtctgaatggagcctcgccaaaagggagaaaaatgttcacaacagtttgcaaatactatctatttaattctcaacctagcaacagcagagaaagagattattattcctctctcatctatgctgcccccactctcctccccgctgcccccgattttatgttttggttgtgtaaattattgctaaatgaatagaactttcaaataagtcctatgctaggacttcataagtgcctaggaaattccaagctgtatttgtaaatttaaagggaagatcagcagaaaaaaatgtcaagtacttaaaatcgactttttattaaggacgcatgagaccagtgagtaggccttagattggatgtgtccacatatcctctgagtcgctccctggatccagcaagaaataggggtgggtgggacatctttgtccctacaccatagcgttggggaagagtctttgtattttgtatttttattttttttttaatagagatggggtcacacgatgttgcccaggctggtcttgaactccttgcctcaagggatccttctgcctcagccctcccaaagtgctggtattataggcgtgagctgccacacctggcctgggtaggacttctgtttctagctctgtatctttcttgcatctgtgaacagataaggtcatgtgtctgagcagtaggacccaggcatgcagttgaactgctcgtccaacttctctctggtgagagaattcctcaaagatttgacttcattcagagctgcattaaaaacaaaagacaaaacaaaaaacacttctactatgaaatgagatttcctgcacttcaaatctgtttagcttgttttgaagtgccagaatgcttggcctccaattagtcaatctccgagagcctcatttggattgtagactcccaactgtttttttactttctccttgctgatgtcattggataaaaataaattaccacccctcccccgtcgttcttcatctttgtaaatttgctgccggtgggtggtagctggtatcagaccagggcaggaaggaagcacattgcttcctccagttatggagctagcataatagtcaacccctgatgatttatcttaatacaatgaaaaataagtaactctcaataatggagggtgctggtttgttggggcgaagctcactgcagtttgtttctattttattccaatgtgggacattgtggtggatgctgtgttttagggcccagatcgctctttagggatgaaggactttttcccgaaagctgctaggagtgttgtgcccgactgccttcagagccagttgtctctgatgatcgtatcagctagagaattgccttgcccatggtggcctccctcctggggatggcctgtattatgtgactgattgacgtggggtatgaaggtctggccctttcgccccagttcaggacaactctcactggccctgactgcttctgagctgccctggtggatgcctctttggagcctgcattgcagccccactgctctctcccctagtgcagtttccccctttcccttctgcaggtgctgatccaccgaatactccttagtaaacctcctgcattgtaattaccgtctcagagttggctactgtttaactttcctttgaagaggaatgtttcagatcttggaatcaacttcaaacatgtcaaaactgtgtagtgtgaagaattgacatgagttgggaaagtctttcgtttttatatccttttcagagttcttgtatgacacaaaatattttagagttagctccgctactgcagtgcttggagtataactctaagtttcttctaagagaaacttcatggaagaagaatccacaggttttggtgagtgataaacttagagttgaggagagaaaagagttcaagatgattgagattttgaacatccatgattgaggaagtggtgatagcaacatatacaaacaggctatacaggcagtggaacttttactataatcaaagatatgtataatgaaaacaatggtgaggcacaaggctgggtccagtggctcacgcctgtaatcccagcactttgggaggctgaggcgggcagatcacttgaggtcaggagttcgagaccagcctggccaacatggcacaaccctgtctctactaaaaacacaaaaaattagccagctgtgatggtgcttgcctgtagtcccagctacatgggaggctgaggcatgagaatctcttgaacccgggaggtggaggttgcagtgagctgagatcgtgccactgcactccagcctgggtgacagagcaagactctgtctcaaaaacaaacaaaaaaccagtgatgaggcacggtgactcacatctgtaatcctagggccttgggaagtcaaggtgggaggatcactcgagcctaggagtttgagagcagcctgggcaacataatgagcccccatttctacaaaaaagtaaaaaaaattagccaggcatggtggcctgtgcttgtagtcctagttactggggaggctgaggcaggaagatcgcttgagtccaggagtttggggtggcagtgagctatgattgcaccactgtactccagcctgggtgacagagtgagaccctgtctccaaaaaaaaaaaaaaaaaaggtatgtataataaaaccaactatacaactgattctttttttaatttttaattgttgtggatacatagaagatgtatatatttacagggtacatgagatactttgaaacaggcatgcaatgtcaacaaccacatcatagtaactggggtatccgttccctcaagcatttgtcctttgtgttacaaacaatccagttatactattttagttatttaaaaatgtacaatcaaattgttattgactacagtcctcctgttgtgctatcaaatactaggtcttattcatttttctaactattttttgtgcccattaaccatcctcacttgctccctaacccttcactactctccctagcctctggtaaccatccttctacactcttatctccattattagttaaattgttttaatttttagctaccacaaataagtgagaacatgcaaagtttgtctttctgtgtttgggttatttcacttaacataatgaccttcagttccatccatgttgttgcaaatgacaggatctcattcatttttatggctgagtagtacttaattgtgtacatgtaccacattttctttatccattcatttgttgatggacacttaggttgcttccaaatcttagctattatgaacagtggtgtaacaaacatgggagtgcagatagctctttgatatactgatttcctttcttttgggtatatactcagcagtgggattgctagattgtatggtagctctatttttagttttatgaggaacctccaaactgttctccatagtggttgtgctaattttcatttccatcaacagtgtatgagggttccctttcctccacatcctcatcagcatttgttactgcctgtcttttggataaaagccattttaactggggcgagatgatatctcattgtagttttgatttgcatttctctaatgatcaatggtgttgagagggccttttcatatacctgtttgccatttgtatgtcttctttcgagaaatgtctattcagatcgttttcccatttttaattggattattagattttttcctgtagagttatttgagttccttatgtgttttggttattaatcccttgtgagatgagtagttacaaagattttctctcattctgagttgtatcttcattttgttgattgtttcctttattgcgcagaagctttttaacttgacgtgatcccatttgtcgatttttgctttggttgcctgtgcttgtgggatattgctcaagaaatctttgcccagttcagtgtcctggagagtccctccaaagttttcttttagtagtttcacagttttaggttttaggtttaagtctttaatccattttgatttgattttagtgtatggtgagagacagggtctagtttcattcttctgcatatgtacatccagatttcctagcaccatttattgaagagactatcttttcctcaatatatgttcttggcacctttgtcaaaaatgagttcactgtaggtgtatggatttgtttctggcttctctattgtgttccattggtcgtgtgtctgtttttatgccaatatcatactgttttggttactatagctctgtagcataatttgaagtcaggtaatgtgattcctcctttttttttttctttctcagtatggctttggctattctgggtctttggtggttccataaaaattttaggattttttttctatttctgtgaagaatgccattggtattttgatagagattgcattaaatctatagattgctttaggcagcatggacattttaacaatattgataatttcaatccgtgaacatggaatatcttttcatttttttggtgtcatcttcaatttttttcatcagtgttttatacttttcattgtacagatttttcacttctttgatcaagttaatgcctgggtatttaactttatttgctgctattgtaaatgggattactttcttgatttctttttcagattgttcactgctggcatatagaaattctactgattcttttttttttttttttttttttttttgagatggagtctcgctctgtcacccaggctggagtgcagtggcgcaatctcggctcactgcaaactccgcctcccgggttcagggcattctcctacctcagacttccaagtagctgggactacaggcgcctgcaaccacgcccggctaattttttgtatttttagtagagacggggtttcaccgtgttaaccaggacggtctcgatctcctgacctcgtgatccgcccacctcggcctcccaaagtgctgggattacaggcgtgagccaccgcgcctggcttctactgattcttgtatgttgattttgtatcctgcaactagactgagtttatcagttctaatcgtgttttggtggcatctttaggtttttccaaatataagatcatattggctgggcgtggtgactcatgcctgtaatcccagcactttgggaggtggaggagggccagatcacttgagcctgggagtctgagaccagcctgggcaacatagggagaccctgtgtctatttatctttaaaaagtaaattaattaaaaatattatatcatctgcgaacaaaaataatttgacttcttcctttccaatttgcctgcccttcatttctttctcttgtctgattgccctagctaggacttccagtactatgttgagtaacagtggtgaaagtgggcattcttgtcgtcttcctgatcttagaggaaaggctttcagtttttccccatttagtatcatactagctgtgggtctgtcatatatggctttttttatgttgaggtgtgttccttctatatgcagtttttttgagggtttttatcatgaagaggtgctgaattttatcaaatttttttaaattatcaattgaaatgatcatgtagtttttgcccttcattctgttgatatgatgtatcacattgattgatgtgcatacaactttttttttttttatattcagagtcagtgcagagcagtggtttagagctcaggcccatgagtcaggtggttagggtcagaagcttggccccactactttttgtctgtgtgaccctgggaaagtcatttcacatgtctatgtctcagtgttctcatttgtaaaataagggtaataatacttaatagtattgttgtgagctttaaactcaatgtatgtaatgctttagaacactctgtaagtggtatgtactattattattattttattcatataactaaaaaattgtattctgtctgatctgttggttgtttcattgttgctaaacacaaatttaagtcttctatccccgtgaccagttctgtccaatacaagtataagtcaactacataagttattggcaattttctagtagccgcactaaaaaagaaagagaaacaggtgaaattaattgtaataatatattttatttaacccaataatttacaagtattattagttcagcatttaatcactataaaattattgacatgacattttacatttttttggttgtaagtctcagaaatatggggtgtattttacacttgctgcttatttcagtggctcaatagccacatgtatttagaggctcctgtatgtgactgagcagctttaaagagttgctagttaatcttgctcatttctgaagtgttccagtcaactgttgctgtgaaataagccgcccccagatttagtagattaaaacaacaatggtttattatttctcacaattctcatttagcctatacatgctagattgatgattattgccattatttttgtgatagttgtgaattccatgatagtttttgaattccacgattccttctacatttattagttggaattctgtggtaaggaataactctttcttctctttcactcagtcatatatttaattgtacatcagcatgaactcatagattcttgttgtattcagtaggttgtaacccattatttattttattattcaagtgatctgaccataatattttaaattaagaaaacaccctctccacacatgctcaggtacctgatgagatcagaacaaattctgctacatggatggtattctcaattccatcccccaacacccccgtattgcaatgtttaaatgtttcagcccaaatatttcacagggtctgcttgcagagccccatgcttctcttcaacaaatgtttttacaggacaaaatttgtccagtaaattgtctctatttatggttcctttgaaagttttaccaagtttacttgctgaaatatcctagggcttttcaatttcacttcaattcagtatagaatataataagtctttcccattaaaagtaagcttaatgaacagaagtttcttcctacaaggtaagatactcccaaagtataactggtgtatatccaaagatatgaaatcagtatgttgaagagatagctgcacccccacatccactgcagcattattcccaatatctaagatatggaatcgatgtcagtgtccatcaatggataaatggataaagaaagtgtggtatatatacgtaatggaacactattcagccattttttttaacaaaaaaaatgtaaaaagaaggaggtcttgtcattttcagtgacatggatgaacttggaggacattatgttaagtgaaataagccaagcacagaaaaacaaatattgcatgatctcacttatatgtggaaggtaaaaaaagatgaactcatagaaacaaagggtaaaatggcagttaccagaagtgaaaggaatggggagatgttggtcaatagacacaaaatttcagttatacgggaggaataatttcaagagatctagtgtacagcatggtgactatggttaatatcaacatattgtatacttaaaaattgcttagagtagattttaattgttcctttccacaagtagttatgtgaggtaaggcacatgttaaatagtgtggtttagccattccataagttatacctcattccataaggtatacatctatcatctatatgtcgcataccataaatatattcaattattacttgtcaattaaaaaaagaataaaacattttcagagttaatttatgtgcacattaaacccatcctagaataatattttgctaaccaaaaaaagatccaggacaaatgctaaatcaggatgctggaataacagcgtcaactgggataatcctggtgaggcctcatcttgtccaaagacccccttttgattgggccaggcctcctggctactgctgtctttctggctgtggtctttgctcacccagctctttggtttggtagactcaggttgatctccactgctttgcatttgctgctcttttgcctgtaacattgtacccctccttgctcatatggctaatttctccatatcctacaaccttagacactggcatcgttttctctaggtgccttcccttcatccctccaatgtgctccttgtcctacctgtactatgtatatctgttccacgatacttccttcttcacctttttgaaacaccatgttcaatatcctgttatttaagagtcttgctctttgcttcctatgataacagttattagaggaactgatctgtgctaggtggtaagtgatggtgatgaattacttatctgttcctgaaaacatacttgtatttcatggaggcccactggggtggtagtcttcaaagtgagtgtgcacacgtctaagagtatatgagaccatttataaggatggaagtagaaaatgtagtaatttttatttcttttttttttttttttgagacagtatctccctctgtagcccaggctggagtgcagtggcacgatcttggctcgctgcaagctctgcctcctgggttcacaccattctcctgcctcagcctcccgagtagctaggactacaggcgcctgccaccacacccggctaatttttggtatttttagtagagatggggtttcaccatgttagccaggatggtctcaatctcctgacctcatgatccacccgcctcggcctcccaaagtgctgggattacaggcatgagccacccacgcccggccagtaatttttatttctattattttattaagcaaagctaagaagttaaagtgtttctctttttctttttattgatttatttatttatttttgagacagagtcttgctctgttgcccaggctgtagtgcagtgatgtgatctcagctcactgcaacctccacctcctgggttcaagcaatcctccttcctcagcttcccaagtagctgggactacaggcgtgccactatgcctggctaatttttgtagtttttagtggagatggggttttgccatgttggccaggctggtctctaactcctgacttcaggtgttctgtctgcctcagtctcccaaagtgctgggattataggtgtgagccactgtgcctggctataaagtttttctaatgtttaatattttggttgagaaaatacatgtatataattttataaatctaatttgacattggtaaggtatactaagtatttttattgatggagtactctatcataaaaatttgaagactacgtctataggggcatatgacatcctcaatggaaaactcttcaagctttttgacactataatagctaacatatttcaagtgttttctatgtgtcacgcactgttctatatgctttacaggtatcaacatacgtattccttataaccacctcatgagattgatatgattattagcatcctcatcttatggatgaaaaaaattgagctagagaatcaggtaatttgttcaaggacagggccagtacatatcaaattcttgatgtggtctgattccagatgctgctagcttaacccctgctgtgtactgtgctgtgctctggtgcctctgctctcacatgatagacccatttggcttcctggaggagagggaccacatctgtcttgcacattgtgctagtttttacggggcctattatttccttctctctctttaaaatcatcttttgctccattttatttcccaagggcctgccatcttcgataggaactttggacaagttctcttttatttttaacatgaaaccatttttccccttgaaataaatggatctgatgagaaattggcatgagtttaattagcagtcctatgtatagaaactttacacgaaaaaagtctgcatcaccccagtactcacgagtcagttatgagtcccagctcacccctgacatgactaatgccttcctttcttcctttatcacgagggtatgaggacaagaaataaacctcagactacttctgtacagcgtgtgaagtgtcccgcaagcacgtataaccttaaaatagcaacaagggcagcagcaacaacgacaacaaaaatgataatggcaaagagaagcagaacctaatgttttttgggtttgcccactgatacccttttaatcctgatttttaattgtttggcacaaagatatgtaataatttcttgggtcttctgggcatttaaatgtcattttggtttcaccaaatgtatttatgattccacctacacatgtaagagtattgggagagttttggaaggtttgtattctatcatcatattcaaattgtgctaactctgttttctttcctgttttccttgtaggctcttttaagacacttactcttccatcagtaagtatccatttaaaaaatttggtttcagaaatatattgtatcagtgaactaaataaattaatattttattggattttatgtcaattcacacatttcacgtttcatgtaagtttggcttgagtttagttcagccttcagattatttatgaataatattatttcaaggagattgattatgaggctcttcaaactcttggcagagattttcctcttgtaaaatagaagacttcactgaaggaaactggcctggactattagcacagaaatctggactggtcataaaaaggaacgagaccatgtcctttgcaggaacatggatggagctggaggccattatctttagcaaactaaaataggaacagaaaactaaacaccacatgttctcacttataagtgggagctgaatgatgagaacacatggacacatagaggggaacaacacacactggggcctactggagggtagagggagggaggagggagaggagcaggaaaaatagctaatgggttctaggtttagcaactgggtgatgaaataatctgtgcgacaacccccatgacacaagtttacctatgtaacaaacctgcacttgtacccctgaacttaaaataaaagttaaaaaaaaacaaggcatctgggctggtttgctcctgcttctgtcgtaatgagccttctagtgtggaactctgctcctcagtttccttgtgcttgaaaagagcagtgccatgctgagacatactagaacttgaggcaaaaggaaaaatcagtaatattgaccctgtctttgtttaaaacttcgacttctttgtttataattttttgcattaattttgatattttaaaatattgtattaaaatattattttcttgattgctaaattttttggtacccccttaaattttgcaccttagacaagtgcctcactcacctcaccatagccctggccttggaaaagagaatgaagataccatctttatctcacagcatgtcctggagtgaaatgagatgtggaagtgttgtagcacttgctatagaaatggaaaggagttattattttgttgagagattcataaaaccttattattaaatgtttgtggatgaatcttcctatagctttgatttggaagtttatactttcattactatgctttgttttggggctttacatgcttaatgtacagtatagtaaaccacagcatccaacatcaggtgcagtttgatatatgtacttttctgagaacagaggactaatgacttaggccctgacacactgtatacctgattagtatcagcattacctcataagccaggttcgctcattaccttaaatttttttttttttttttttgagacaggtcttggtctgttgcccgggctggagttcagtggcacaatcatggctcactgcagcctcaatctcccaggttcaagcaatcttctcacctcagcctcccgactactggcatgtgccaccatgcctggctgattttttattttttgtagaaacaggtttccctatgttgcccaggcttgtctcaaactcctgggcccaagcatccctcctgccttggtcccccaaagtgctgggattacaggagtgaaccaccatgcctggcccacttagtaccttttgaaagttacttgtcaagtacattcagaaaccacatttattttggggggttcatagagaatattatactctcaaacaatactttataaaaattacttagtatgctatatctattctgtttgttacatattatatttgtactttctttcctaagaaatatactgctgctaataatagaaagtggaagaaaaaaggcataagcaggcagaagtgggggaaaaaaaccctgcaatctttgtggcaataaaacattagattgcattaaaataacctaaaacattagatactggccccagtgcctgggcgcagaactttgttgaaagggtattccagtggtaagatgaaaataaatcctgtgtcgtatatttgccctatagcagaaggagggtggggtttaagctcaggaatcacagcctaaagcacaataattgtctggtttcctcaaaggagtttgcagtttcacataccggaagtgtagaaaggctgaaaggagaaggcggggcaagggcatgagagtttctttcaatgatactagtataaactgtacgtgtctgagctaatattaactagaaatttgtctactacacttctttttttttttttttttttttgagacagagtttctctcttgttgcccaggctggagagcagtggcgcgatctcggctcactgcaacctccgtttcccggattcaagagattctcctgcctcagcctcccgagtagctgggattacaggcatgcaccaccatgcccagctaatttttttttgtatttttagtagggacagagtttcgccatgttggtctggctggtcttgaacttccaacctcaagtaatccgcctgcctcggcctcccaaagtcctgggattacaggcgcaagccactgcgcccagcctactatacttcttttaagtcttaagtatatgaaatactaactctgacatccagtatttgtctttaatattatgtcatttcaattgtatataaatatagctattatctattgaaagctttattagagattatattttggagtgtatctctttttagtaaagcacagtatgaaagacttagaaataaagtgtatggacaaatttgataatgcttgacaaaaaaagaaggctctgcattttaatgagttttcaattaaagcagaaccaactttctttttccccagtgagaacatacctgtgaatagaaccaccttttaaagtgcacttgcattctgacacttgtaagtgtaatattttaaattatttttcaggtctttatggctttttagtgtaaaatataaatatttcaagaatgtgttaacacaaatgtgttttttatttctgatttactcattgtacttcaagacactaaaataacccccaatatgaagtaaataaaaaacataaattgagacaatataaatattgttttgtacacaatatggcagaaaaacttcttttgttctctttgtcaaatgaaggagtggttttatttgattagaaatgtttttaatgtgtaagtataattactaatatatttttaatggaaactatcatcttagatttctcaattttattttgaaaaattgtctccgttatgtttggtaactggcattattcctgaagtatatgaggataaatactattgtgttgtctcagtttccttacagcagtgaatctttcagagaaggtgaaatggctttagacagcccagattagaataagtaatccttatttattgtctagcaagaatgtttttgaatgtgcttaccttatgtatcctaggcagagagctatcatctcctcagagagtttgcatttatgtatacacgaaatagatgtgcctaagtgagagtcaacatagccaatccagaggctgattgatattctgttggtcaaagctttgggatattgtcaattcccagactcttgtcctgctttcagacagcctctcactgtgggaactgcaccttcctccagccctgtgccctctggtcaaggagtccaaaatctctgtgggtaaataattgactcctgtttaagagccatggaagtcacttatcaaggaacagctttttttccccctttcccccaccaatttcgggaacctttttcagtaactgtggccccaagacggcagaaggcaatttaccagtaaagatgcaggagcttctgttacctcatagtgtccactctgctgtgggcaggttataacccactccagatctggcacagagagagaaacttttataaaaattgcttaaagtcaataatcttttcttcagtctcattatgcgcccccattgttaaccatggagcaccgaggtctgcattgacaccacaagactctggagcaccacccagagtacgtggtgacaccccgagacatgtggtgatgccccatgcttcatacaggcacccaggtctaatgattcaccaaggcccatattgtcatctctgggtggcttgccctgggactaacactgagtctttgaggatcccaaattacctaaattttatatatggcaaggtggggtttttaacggggaaaagttaaaaaagcaaattagttaaggtaacccaaaggaacaggaagatagtcagtaatgtttcccctgagtcagtgtcagctccctgaaggaggatagagtaggaggtagaataaaatgttttcgggcatatttgcatttattttaattctctgaccctttaggctgcttgtggttaataggcttctttagctctaggcctctgtgtcattaatttgacatcagtcttcaatctagcatctgttctggggcctctgtgccactaaaaatgataaacatatcaaatcaggaagagcaatcccatcaaatatatttttaaacatgaaatatttagaaatcgatgccatttataagcatataatattctggttcctggtgtatatattttcttgtttatagtattaggaaatctgattcaaagagtctgaggactgaagtatcacttgagaaaatagaatttaagattgcaagttccaggctagactctgctctaccagggcacaggtctttggttcatgttgtatcccaagtacctagggcgtcctggtacacaaagggtgctcaataaatacttctttaggctgggcgcagtggctcacatgtataatcccatcactttgggaggccgaggtgggtggatcacttgagttcaggaattcgaaaccagcctggccaacatggtaaaaccttttctctactaaaaatacaaaaattagctaggcatggtggcgcacacctgtagtcccagctacttgggaggctgaggcaggagaatcgcttgaacccaagaggtggggttgagattgggccactgcactccagcctgggcaacagagcaagagttcgtctcaaaacaaaacaaaacaaaacaaaacacttctttattgaataaaagcaaaaattgacaccagaagtcatttgaaggtgacatactgattgtttttcctaacattttattattaacttttcaaatatacaaaaaacttgaaaatgtttcataatgaacactcctatacttgccacttagggtctaccattaacattttactatacttgctctatcatacatctatccatctacccatctttctatccatccattagttcatcttatttttaaatgtatttccaagtaaattgcagacatcaatactcttctccctaaatattgctgctgaccattttattgttgacagtggtttcttgcactttcttcatattcttaaccacattgatagcattctgatgtctgctattattggaaaatcccctttgagctatctgtagacaaaacttatttgcaaaaatgagtcaggatatagtttcgggacctcagttttaatatgaaaatcaactaaaagtcaagcatgctagttatctattgctaagtaactctaaaactttgcttcaaacaataataaattaaacttttattacttctcacagtctttgtgggtgaagaattgggaacaatatggttgggcatttctgatttggagtctcacgaggttgcaatcaagatatcagctagggctgcagtcatctgaaggcttcatcagggctaacacattctgtgaggcagctcattcactaggctgacacgtggttgttggtggttggtgggaggccctggttcctcccaatgtgggcctctccacagacttctttagtgtccttaccacaccactctggcttcctccagagtgagtgatccaagcgagaaagagagacaacagggaagaaggtattctttttctgacatagcctaattctaggatttggaaggaaatcactaagtctggggaattaggcttcaccttttgaagggactaaagtcagagaatttttggacatattttaaaattaccacatcaaagtgtgtgtgtgtgtgtgtgtgtgtagtgtgtgtgtctgaaatgattttaataagaatatccacctgagggattgttgtgaattaaaatgatatagtgcaggtaaactgctcagtatggctctggtctgctaatgctagaaggtaggacttctattgttattattattattattattattattattattattattattattttgagacaaattctcgctctgttgcccaggctggagtgcagtggcacgatctcggctcactgcaacctccatgttcaagcgattctcctgcctcagcctcccggagtagctgggactacaggcgcatgccaccacacctgggtgatttttgtagttttagtagagatggggtttcaccatgttggccaggctggtctcaaactcctgacctcaggtgatctgcctgccttggtctcccaaagtgctgggattacaggtgtgagttaacgtgcctgggaaggatttctgttattaatagtagtagtagaagtagcagcatcatcatccctaccaagactatgacacatatgcgcaagatgcattaggagaatgcatctcagaaaacaggttgagagaatatagcaagaaacaaattttgtgcagaagagcctttcatcagctgactgcctcaaatagggtgagcgtggaaaagaaggttgaaggatgttagatattctgacctgcaataactgttagggaggatgaatttgcacttggaataggaaatggcaaggtgactcctgctgagggagagcagtgggctatgtgtcccccattccaggagccctgtgatgtaactggagagcaaactgcaggaagttaaaagagggaaaacaaattaactctgatgcctgggaggggcaaattttcaacatcgcagttacttttgataatactgtgtatttactttttttattctaacttgcaattaaaacatttttgaggtaaaataatgagaccttgatgtgtgatctctttttccccccagctacctctgaacagtgcagatgagatttatgagctacgtgtaaccggacgtacccaggatgagattttattctctaatagtacccgcttatcatttgagaccaagagaatatctgtcttcattcaaacagacaaggccttatacaagccaaagcaagaagtgaagtttcgcattgttacactcttctcagattttaagccttacaaaacctctttaaacattctcattaaggtaagtgccagacagaaatgaagcaaggaattctagccatcttactaaacccctctgggaagttctgcttgtgttactgacactggaatattttagaattcacctaagataagcttccctcccaacatggaactcctctaataggaaagaaggtggtgcatgcactgggttaagagctcagcctctagagtcaacctgcttgagttccaggcctggggtcttcattgatttatacagttgtcaaacatttattaaacttctgtgttccaggcacagcgatagggcagtgaacagaagagattaagtccctgctgacactgagtttatttttcagtgggtagtaggagacagacataataaataaacatacaaatatatatttataaagggacagatatgtatgtatttgtgtgtgtgtatgagagagaaaaagggagtgagagacagagagtttgtgtttgctattttctttctttcttttttttttttgagatggagtttcactcttgttgcccaggctggagtgcaatggcacgatcttggctcactgcaacctccgcctccttggttcaagcgattctcctgcctcagcctcctgagtagctgggattataggcatgtgccaccacgtctgcctaaatttgaatttttagtagagacggggtttcaccatgttggtcaggctgacctcgtgatccacccacctcagcctcccaaagatgctgggattataggcgtgagccactgcgcccagcatgtttgttgctttctctagggttgtctgggaagactgatttgagcagagagccaaaggcagtaaaagaaaataagcccatacagagggggaagcacatggaaaggccctgagggggagcttgtttggcgcattggaggaacaacaagctggcagtgtgctggagccccggggcgaggggacaggtgctaggacatgaggtcctatacaagtaggtgggttagggtggggctcatgcagggccttattggccatgatagggactttgaaacttatgctaagtgaagtgagaattcactggagaattctgaggcaagacttgatgtaatctaacttaacatcttaaaaggatcactgtcagctctaagaagaatagatgctagatgctagctgtgtgatcctgggtaaatttattaaccttctagaacctcagtttccttatctagaaaatatgtaataataaaacctcaaaaggtttttatgaggcttaagtgagataattcaagtaaaccatttagaactgtgtctggcacatagtacatgctcactaaacgttagccattattattattttaggaaccccttttgaaaggtagtagttttgtctctgctgcttaatactctgatgatttggaggcactgtggttagcacactgagtgctttcctgtgagaatcaagttgaagacaactgagatccagctgggcctggttttctgtatttaaacggagaagaacactggacatttgatcattacttggaaatagggtgactaacatcccagtctgcctgggatgcctctgattttaaaactgaaaagtctctggtcctgggaacctccttagtctcaggacattggttactccagatattctaatgaataacatagtgtggaaggtaaagtaggccttttagtttggtatcttcctattcttagtttatttgttgttataaattaaaacaacaaaacccaatcaaataaacatgtatacatttaactttgagccaagatatatttgtagccagtatcacatttgttgccatggtgttcaatgcatattgcttgaattgaactagcattatttcagagtttgcctttggtatgtaatggaatgtttataaagcaggttgacatagtgaatagtatttctaaattcatgtcacccccctgagcagagttgaactgactccattcataccatctttcatcatttatccacatatatggagagcttactgtatgtcagaaaggatgtgtacaatagatggtttaataaatgtctggctatctctgggtgtttacattcaatgcttccttctactgcaggcaaataaaagttagcactgcataaattgcctgttctagacatcaacccagaactaaccctgctggttctggataattagtctgtgcattttcatagttctgcctgtctactctgctctagtatcttcctttgaatctttgatttgcagattctttgggctcttctgtgcagtttgggacttagcagaccacgtttggctttctggaaagggctcagagagactttcctcttattagctctggtactgatatgactctcttggccaggggaggggagtttaaactaaaattctacctggaacccaaagtgttttggaactatgagtgaggggtggccatgctcctgtctctgtacctgggaagtgatgctcaaatgtggacttaggtctagctgccttcctccttgtaacttgcagaggccaggtgggagcagtaatgtgaactctttcagctgaaaacagacatttgggccaggtttacaagtctatgatattttctacatctttcagtgtggaataattggtgtcttggcaaaacttttcagaacaaattgggacttagcctgaactggagcccctgactgagacagttgtactccttggcacagcaccacctgcaggctgatgtggagaagtgcacctgagagttaacggggcccatcttgcccagctagaaatggctgcaggatgtagctaagatactgactgtcacagcaccttcagggtgttcattgttgttcttggggactgcaggccatctttgggtgttattcattttgcatgccccaataactggtcttctcaggagggaaaggagcttcgaggccttttggcagtagaaaaaacataacctatgtctcccacgcttctggcctcctctagccaatttggcctgacacaatggaattgagtgcctctgacactgcgatggcagctggcatggctggcttctgctctgacctctccatccagaagttgttggtgctagtacgggtgaatgatcttgtaggggaacaggtgttattggcttgggggtatatcctgttattgtgccctgacactcagttttggagcagtggtggctaactcagaacctgaccgctggccaggtgaagaagctaccttatttgagtgatttgaattctctaattctacaggctggacagacatgttattcacacataatccagaaatagtcctcacagacatattctccagccagggagcttactgcttgagacaatcaggtactgtgtgctgtgttgaggcatctcagagatccagagcgatctaaaggtgactcgaagggcatgtggggccactattccaggatctctggcctgcagcttttctggctgtgacgggggtattgctcatactgtacatggctgtgtcgtagcagcagatggccgcatgcaaatgctgggctccgctgtgacatcaccatgcttggctggttcaaatcctgatgtgtcttgtagacttctggtgtttctgcattctctccctctgccttattggtaacagttagacctaggtccaagacaggcttgggaaagcagaggaattatctcattctcccccaaccctccagatcccccaggtacaaagccatcaatgaggtcagttcagacttcattgtccaggagaaatgtatgccctcactaccataatgtttcagtagaatatggacattccattttcctacacttcaggtaacaccaagaattattgttattattatttttatctttgtcagactgacagaagaaacacaatattgttttcatttgtatttccttaattactagacagcatatatttattggctatttgtatttcttcctttgttcctgagcctttgaacttcatagccctcccatgcgtcatgctgtggcaggcacagtagggccaactgcccattacaatgcctggcccaggagtttaccaaggatacagtgacctgcttgttcttgacttgcttttggtctctggttttcacaattggtttctggcttattagactgctttctgcacagttctcagcattgtacttacccctaactgtcatatctccactctgggagagaggtttcttctgttctaataactggccccactcactacttctaggcaaagtttagaggataagtctaagggaaaatattcaacccctgctctcagtcacagtctagttgggaataataaaatatagatttttggcaaaataaaatatgtaaaataaacatattttactgcctttaacaaaaatatgtattcaagagagataaactccagatatactagggactttccaggaagaagtcacctctgcctattggaggcatgtataggaggcaagaaactgaaactgatttcaccagtggacaaggaagggagggatttccatgcagcagttgcagagacctacatgcatgggctttttgggaataaactgcaaacaaatccgtatgtttggaggaagatgagcctagaaatgtagatggagtagtattagtaagggccttgtaaactaggcaaaggagtgtagacttcctgtaggcaaacaaaagccatgggtacctagtggtctagtataatcatcatatactataattagggactataaaaagtgtatagccactgctttttatatgccagggactgtgccaagtatttcaaccactgtaactcattctcagatttgcgtttcagatggatcacactgttggcagtggggaagatgtattgtagagggcaagactagagggggaagattagttgcattagttagaaaataggacatggtggctggatgtgaggatggaaagaaggagacaaattagagatatattaacctaacaaagttaaaatgtcaggaaatgtgagagaggaatctaggatgactgctggaatggcttggataaatgttgcatgaagggattgttgtttgggaggttataggaagaatgaagtgttttccatgtatatccgtgattaaacatctctctaatatgcttttgttgagttaaaaggcagacatgagtttggattctgaatttcatcatgcactggtgctgtaatattgagtcatgccagtcacttgaatgcgtacctccctctccaggcatcagttttctcaactataaagtgaggataaccatgtcctaaaggagaattgtaatgattagagataaggcagagtgcctgacacatattgggcctttaataaatggggcctgttatctctacagttaaattttcttttaacactactaccaccaccgccgccaccaggggtttggtgacgtacatgttgtagtgtttcatgtagaaggaaatagggtgccaggaattaaaaggtggagcagaatcaagatgaacttgggagaaatatgagaaaagaataaactttgatttgtcttctttagaaatatagagcctacgttttccaaaatgttcatgcagcattcatccaagccattccagcagtcatcctagattcctctctcacatttccagaccttttaactttgttaggttaatatatctctaatgtgtctccttctttccatcctcacatcctgccatccagccatatttgtgccgtattttggagtaaagttctggacctgggtgggaatgctgcaaggcattattcctaacctgataggatacacatgcacatgggcagcctctacatacttacatgtctggttttcattttaggaccccaaatcaaatttgatccaacagtggttgtcacaacaaagtgatcttggagtcatttccaaaacttttcagctatcttcccatccaatacttggtgactggtctattcaagttcaagtgaatgtgagtataaatatattttttggggggaatgttaaagtgatttaattaggtcaggtgggggaaaatctccaaagtcatttatacaatgaagagaaaaaaattaatgtgaacatttagacaatttaaacggtttatttttaatttatgatgtttatcaagggtttatttagctttcattctaacatattagaattttcataactgttgtctttttctatgatatgatgtttaaaggttaaaacattttgacattttagtacacaatgtttacttatttgtttcatgcattattttaaaactaagtaatgaacacatagctattatttggcaaattttatgccttagaataatgactcctaatgagtgattagattaacctacttttatctttgttttaagaaacagtgacttaaatagctacaccgactgctattccccaatcctttttccctagcttctacacctattgcctttacttgctgccaacttgtatagctctgaaatctaaatggagggcattaaatggcatataaatctaaatggggggcattcattggaatgaatgagacataaacacaatgggatggaaacaattttaaaaagcaatgagacttaactgttttatgtgatataaattggtcatttagtatataacatgtaaacctttatatttttctatatttatgtgacatataaacatataagatataatcaactttttctgtatctaaaggggaagatatgtaatatgaaatgcatgaaattgtagcttattctaagcattgttgctgagccttgagtaggtgggactcgtgtgtgaattgacctgtatgaatgagaacagtgctggaggcacaatggactctcaatatttgcttcatggccaccctgcatctttatgggtgtttgtatgatgtgtttaccaatattattctttttaaggtagcagaagctctaccgaatgaatgtatgatgtggttttaaagaacatttgaggcaatattatgaaaggggctcatactgttagagaacaatgtaatgcataaccattaaagtcatggacaattttcagtgaggtctttgcttatttccttacttataacagtgtaataataaccactgttataaatggcagcctagagcaggatgtgatgagttctaggggaagtggaattctttatatctgagcttgtagagaaagtggaaagagtaggcatttgtgaaacttagaaaatagggaaaaatttgtgtgggagtagggaaaatggaagagaacgttatgtgtctggggatttgggagaagttgatgttactggagcatgtggagaatgatggcggtgtagattaggaagcacgttgtgagtttaaagtagtttgaatattattttcagttcccacttaaattcttaagaatcactttttttggtccatccatccatcgatccttctatccacccacccacccaccaacccagtatgtgttaaggactttatggctgtgatgaatacaaagaaggtaaacatatgacctccattttcaaaggtcattgtcatggtgacatgaacaatatgataaaaattaaatgacataagggggacagacaaacacagggcagtgcaatagcagaggaattgtaaggtgttcagtccagcttggtgataaagagggtttattgttagcacaagcactgatgttttggggccagctgctccttctgtctcccatggtctgctctgttctcctttcactcttctgccaacctttgaggtgttcttcagttcttctgattccattagcttagctaaataaccatcatgcccagaatctttaaagcaagcctctgcacaggtggctggcagcctgtgggtgggccgccctgggtcaaggtgtccatttttggtccattcagatgccacctatgcacaatgaccagcaagagcagttttagctggataacaggcgctgagggattaatttgttcattgaatggtggacagaataagtaccagaagagagggggatagaatagcagagggagactttgcagaagatgtgagattatactttgaaagatttataggatttatcatgttgtggtttgtggacttggcttgtcatattgcttttatagaaaacaaatgctcttaaggaaatttaggtgcttaaaaaatgaaaagcatttgcaggatctggcccttgcccaacttttcagtcttgtaaagtgccgcccttcctcttgaactctatgctttagacatcttatgtcccttaagtttcttaaactcaccgatctgtctcattctagagcctttcatcatgctgttccctctgctgagaatgttttttcttccttattctcgctacttgcttttaacctgcagccattctctaaagtggcctggctgttccagacattcccatagtactgtgtgcttcccttaattcattcatctcacttttaattagtggtccatgatagacagtagcctttgtaaagctggtgaccttctggtcttgctcacctctctattcctgggaccaagttcagtgcttttgcatgtaagtagaaagcagtattttcttaaaacgaatgtgtgaaaactatatagcaataatttgtcttgaaccaaatgcaattttcccttttgtatattctataaagcatctttatctttttgaagacaagtatactgatcaggtacttgtcatttgaggtatgttaatttgaggcattcagctaagaaaaaataagccccttaaaaaaaagagcccttttagaaccaccaggggtcagtataaccaaggaaatgttatgagaagcaactgtggccttggtctctggcttttgtttttctttgactactgcgcagcattaaaggactgtatatttcttgttattaaaccccttttcttttagtagtgaataataacatagaggaaatgtatttattatttaaatagcccaccattagagactatatttttacttttcggtggcagtcaccattctcaatataaaactcagggaaggaaaatatttattttcaagaaagtattggctgggcgtggtggctcacacctgtaatcccagcactttgggaagccgaggcgggcggatcacgaggtcaggagaccgagaccatcttggctaatacagtgaaaccccgtctctactaaaaatacgaaaaattagctgggtgtggtggcgggcgcctgtagtcccagctactcgggaggctgaggcaggagaatggcgtgaacctgggaggcggagcttgcagtgagccaagatcatgccaccgctctccagcctgggcgacagagtgatactctggctaaaaaaaaaggaagtattttttaaattttttaatttgtaagagacagggttgtcgtctgtcactcaggctagagtgaagtggcacaatcatagttcactgcagcctcgaactcctgggctcaagtgatcctcctgcctcagcctcctgagtagcagggactacaggtatgtgccactacgcctgcctagttaaaaaatttttttttccttttcatagagacagggtcttgttatgttgcccaggctgatcttgagctcctggactcaagcaatccttctgccttggcctcacaaagtgctgggattacatgtgtgagccatcatacacagcctttgggagagttttgaaattccattttgtctgtagaaaaaatatggaatgatatgagtattcccactgatggacaatctctttaagaattaccggagttatcagagctttgatttgtaatgactatacaggtaatcagaaagtggtattcttcagttaccctttgtcctaggtcaggaaaaggatcctttaagtaaaacatacttgacggctccttggcatttctccacttgtattaataacatgtttgtttgtagagtaagagtgaataagtcctttatgataccgtgttaacctttctggattaatctaaattgtttgcccttcaagtattgtattgcttttcaccttcaaggttttttatgatagtaatggtgagtaattattttggtctagtatgatttctagttttcttttctttctttcttttttttttttttcgagacagagtctcgctcttttgcccaggccgtggtgtgatctcggctcactgaaacctccacatcccaggttcaagcgattctcctgcctcagcctcctgagtagctgggattacaggcatgcaccaccatgcccagctaatttttgtatttttagtagagacggtttcatcatgttggccagggtggtcttgaactccagacctcaagtgatgcaccctactcggcctcccaaagtgctgggattacaggcatgagccactgtgcctggccaagaaaggaaacaaatgttttgggggattagttaagtgatttgcccaatgtcccacaacttaccaaactaggattagaactcaggccttttgacaccaaatccaaggttctctctaccctgccaccttggttttccatcttatgttttatctctgtcattgttcagcatatgatggtgggacatcataagttctcaatatatacatagactttttttttttttacaatgctagtttaatcaaattgcaaagaaactcttgatgattataatgtttaagacctcttaaaatcagtaaagtttagtaagagccctggctctttttgtgctacttatacgtgtgcttaattgacacacatattctagtgtgaatagttctttgctactttggggattgtacttcttctcttgagaatgtgaccaagtcttctacttctgccatactttgattgccctgctctttttcatcatttctttgtctagagcaccaagccctcctctcagagggcagaggtatattttagctgtaacatcttttcatagaagtgtgttttatagggccgttatactttaggtacattataagttgagtgtgggaatatgttctgatttaataattgacttgaaaatacaccttaggaagataatgtgttttaaaagttagacattcctttattttcatttctttgcaatatagttctctaggtaccttatctctaggtatgttatctcttgattggtgttgacagttttctcaaataattgcacatggtttttttgttttgttttgttttgttttttgagaaagagtctcactctgttgaccagtctggagtgcagtgtgcaatctcagctcattgcaacctccacctcccaggttgaagtgattttcctgcctcagcctcctgagtagctgggattacaggtgtgcaccatcatgttcagctaatttttgtatttttagtagagatggggtttcaccatgttggtcaggctgggctcgaactcctgacctcaggtgattcacttgccttgggtttccaaagtgctaggattacaggcatgagccactgtgcctggcctgcaaatggtttttccctccattttttattttctgtagtaaaatacacataacgtaaaatttgccattgtaaccatttttaagtgtacagttcagtggtattaagtacattaatattgttgtgcattcatcaccaccatccatctctgtaactcttttcatcttgcaaaactgaaaatctacacctattaaacaataactctccattccctcccaccccccagcccctggcagccactgtacattctatctctgtggtgttgattagtcttatatcagcagaatcatacaggatctctttttgagactggcttttttctcttagcataatgttattatggctcacccatgttgtagcatatatcagaattttctctcttttaaaggctgaataatattccattgtgtgaatataccacatttggttgcgaatgtttttgcatctatgatttaaaaattatattcgaggttattagaaggtgattttaggggttctttaaaagaaaggctaatgttggtgtctgagaaccaggcttctgggagccaggaggcatctggccagagcagtgcagcctgcagcccagagctccaggtctttgcgtgacccacgcactggtgctcatgttcctccaagagcccgaaagaagagcctgagtcactgagagattcctggtcatttgatgacggcagccctgtgctgtctttcctcattgtcagatggaagtctcttcttctttctagagtggcttaggcatttgagtttaccataatggccacctgaggctgcagctggttctgaaacatcagtgtgccacagaatcaatggactgggagcttgtttaaagatgcatagtccaggtcccacccccagatcacaatttactaattttggcatgggagctaggagtttgaattttaaataagcactcaaggaaattctactagttttttgaaccacattttgagaagtgctcataaatgcccgcagctaggcatctttagaatccatcttttccattgagaggcagtgttgggcagtgttgggcagtcaacaaggtatggaactggggacaggccatctgatttattctttgaattcctcagcagtgctactttctgctatttactttacctttttaggtaatcagctcagtagagctccccagcaccttagacatactaagtcagaatttctgggacaatgtatatcctttacaacagcttctctgatgcttcagatgcacagccagcttgggaaccactgaacttgataatcatcttttctatctgtcactaactacaatttcgtaaccgttgtctcaatcttaagccattgttagaggtggggaacaagaaatagcaagcagaaaactcaacagggttaaggcttgaagtcactcttctggagccctgtataatgaaacgggttatcaaattattatgtaactcagaagcaatctgcattgagttatttctttgcatctaacagttatatctagtttctcttgagttacttgttttttctagattaggtgagggttgccaacaccttactgctttttataacctttcaggatattccttattttttctggacttgttgtccagcctctgcctgggtcagcttgctgcttctgttaagtatctctggagccgtttttcttttgcaaaagagcacatctttgtttggacagttcttgtccctaaagcactctaccaggagcatctgcttaacccgcctggcttgtgacgacaacttaacagaaagcatggagcaaaactttgacaggtcagaaaacagtggcattctctctgaaatgtggttgtggcttccaaaatactttcactagtctttgcttataagaagaatgctttgtttgggaaggatctcaaactcttacttccttgttcagtgagaaagggagggaggagatggttttagaatattgatttcttagagcattatctggcagagctcagcactttctcctccattcttccaaaggccccgataaacacctctagaatagcatttattaatcagtgtagcgttattttcgttttttaaaaactgtggtaagatatatatgtaaaatttaccattttaacaatttaaaagttaagtagcatcaggtgcattcacattgtggtgcaaccatctgccaccatccatctccagaacctattattatcttcagatatttaaaaaaatgtctgcctcatttcttaaactgtgaactccttgaagacaggaattctattttattaattttcatatttgttctgtctaatataatatctgacctgttatgggaactcattcatgcttgtgggatgaaagaatgagtgaatagagcttcttctagatgattgctgttattatcctttgtgatgttctttgacttctagtctcactctgctatatcatcttgcaacttagtagaaatatctttgtatattttcattttgagaatatatatggttgctgtatttgatgaacctgtgagctaatggaaaacaggacttttggattaatgccctggactttctcattttgaaaattaaaaaaaatctcaggcaagagctgaatattctagtttgttataaagtctaaaatagacttataattttggtgccatattattcttgtgtctttattcagccatttgatctgctagtgccagtcctttggcagtgtgcattttaatttctaggccccatatgcaaattttcatgaggagttctttgttttgttctttctctacagaagccaccctaggaggacccctcctttcaaatgtgtcttctgtacccctctctgaagagacatgtttttggcatcagttagaaaactttggggccagactttaggatgttgtgttcatcaatgaaatggagggtctgagtatgtagcatcatcagtacataggatagggactcagtgctatagatgatcttagaggtcgcctagatccatctccccactttatggaggaagaatctgagataagtgacgtgacctggttaggctcacacagtgtgttgtgggagggtcaggactagcaactagtttactcatactttcagttctgtaaaattaaattatttaatatgtgcagcaattgctgttttatttatttatttatttttgagacagagtcttgctctgtcacccaggctggagtgcagtggtgctgtctcagcttactgcaacttctgcctcccaggttcaagtgattctcctgccttagcctcctgagtagctgggatttacaggcatgtgccaccatgggcctggctaatttttgtatttttagtagagacagggtttcaccatgttggccaggctggtcttgaactcttgacctcaggtgatccgcccgccctggcctcccaaagtgctgggattacaggcatgagtcatcatgcctggccagcaattgctgttttaatatggcctccttttaaggttgaatttagatgtcaacagggcttgctgtatgcattggtggagaagatacctacttcttttatatgacttgtggatgccacgggatcaccttgtttgcacgttggattgttcccaacgtgtcccagattctgaccacagcagccatgtgtttagattgaagatttgagtccagcatgtaggttacttggttcatctatttaatatccattaatatactattaaggattaggctgctgtcacctcacagcctctataaccttggtcatatgaggcttacaaagattaattttttgttgtttgtttttgagacagagtctcgctgtgtcacccaggctggagtgcagtggcacaatcttggcttactgtaacctccgcctcccaggttcaagcgattcacctgcctcagcctcccgagtaactgggattacaggcatccaccaccacgcctggctaaatttttgtatttttagtagagacaaggtttcgccacattggccaggctggtcttgaacttctgacctcaagtgatccgcctgcctctgccttccaaagggctgggattataggcataagccactgtgctcagccaagaagattaattttaaaaataactgtttacctttaggtagattcacagaaagttgcgaagatagcacaaagcggccctatattatcttaacgcagtgcacccagtggttatatcttatgttattatagtacaatataaaaagcaagaatttgacgtttgtgtggtatgtgtgtatggttctgtatgatgctttattttcagtggcatagagggagttttggaaaatgataactcactgggctctgaccagtggtggcagccctgggtgagggggaagggggcactgaaatgagtaccaagtccctgggtggagtgaggaaagggagaaagagcaagaaatggagttattttccctcaagggtaactttcctattcagtacaaacctgtagctatgctgctgcttttgctcccttgcttgagataggtgcccaagaaaggtggtcatgttacatgtctttaaaaaccatagttccttctactttgagtaggaacagagagggttaaatgggtgatttttgtttattgtttgtttttaaagagaatatgtttctagttagtactttttttttttcaacaccaaggaaacaagtttgtcccttgtactgcataatgtatttgtactgcagatggtaacactgttttaggagtatgaatgtgcttgtgatccctggatacctggccactattctgctaactggaggcttgtatggtctatgtcttggtcattggttagatccattattcccaagtcctgtcactatgagtgactgtcaggtatagaccagcttcagttcttcaggtgtttagttccagccttgataagaaccagaatatttgtaatcctttctaatgagagaagggatccaggaacaaaactgtctcctggggaggttctgattcctccctcctcacactcagtagattatcttcctttccacttcacagagaacacagtcatcagatgggaatttctttcactttttctgccagatttgcatgtgttgcctgtgcccatctcatgcttattctcttctttttagaggaaaggtcctattcttcacatctaagaccaatatcccgatttctgccttggcttctttcttcctctactttgtcagaaaacttgcagtgttgacagtctctttcttctcagtcttcaaactggatctctttctgtcttagatctttttataaaatctaatatgtttaattctttctcattgaaaaaacaaaaaatatagagaagtactgtactttaactctacttcctcttcagtgacttcacattcccattctagccagtcttctaaaaaaaaatgtctgatctctctgcctccattttcccaactcactccagtttgactgagatttctttctacttgtttggattcttcttcttggtcactttcgtcatttactctctattacatattggcacattgcaagtttggttttaagtttctttcatttctcactctagggaagttcacccgaaggcatggtttcagtttccatctatacaccaatgactctcaaattggcctctctgagagaacctatacctctgagttctagacctttaaaactatctacctttaaaactatcaacatctgtatttggatgtttcaaagcaccccaaactcttcatgtccagtatcattcttaacaatctttccccaccacacctggctcctctgcagcatttcctaccttatttaattctctatccattcggctgtgcaggctggtaaccttagagttcttgacacctcatgctgcctgaccacccatattcaggccttcatcaattcccacagctttttcccttaaataaatatatttctttctttcttttttttttttcctaccctgagacagagtcttgctctgtcacccaggcaatctcagctcactgcaacctccacctcccgagttcaagtgatcttcctgcctccatctcccgagtagctgggagtacaggcgtgtgccaccatgcccggctaatttttgtattttttgtagagatggggtttcaccatgttgcccaggctggtctctaactccttagctcaagtgatctgcctgcctcaaccttaaatatatttcaaatccacctacttctctatttccacctctccctcctttgtccaagccactattatattgttcctagatattacagtgtctcttaatctctctgcatttggtttttttcccctttgcaaccaattatctacacagcagtaaaaataatctcaacattagctggatcatgttattccccagcttaaaactgtccaaagactttccactggcctcagaacaaaattcagatgttttaccatggcctacacagggtcttacctgatctgactctgtttttcccatctcatctccagatttcataactctctgtaaatgttaattcacagcttttatcataatttgtaatacatactcacatatgtggttatttgataaatatccttctttccactagatcagaaggtccatgcggggttgtattcccattacacctgccactcaggcgcaatgaatatctcttgaaggaatggatgaatgaatgcatttgttgtgtttgggcctcagccctggtaattctgattcagtagctctggggtaggaactggacacctctatttattaaacccaccacagctgattctgatgctgagtttgggttgaaccccatagagaaatatgttgcaactacagtttagaccacaggtgtttagactctaaacaggaaagggaaggaaaggatgatgtaaatgttaataaacattttctctcattttgcaaaccctattgactctgtcattcctctaagtaggacagtgtacatcagctgacacttttacaggatgatatttccattgtgatttcttttttatttttaagctctcccaattttatggtaaaaataaaagaggggaaaaatagggtattacatgattgctattacgtaggcaataggtttaggcatagtcaggttagtagggatcggaataaagctaaaaatatgaagcttttgtgttgtatgcgagggtgggaagaaacaccagtcagtacacatctaaaggtgacaagacacagacctggctttcaaggagcttacaatcagaaaagcttcgtgtaacaaatgcaaagagcattagattaggagtcctaagacctgggtactgtgctctagttaagtgatggtgggcatggcatttcagctgtgggtctccgtttcctcagctgtaaaataaggggtttggatcaagggatttttaaggatcttcacaactttacaagtccaagatagggctggaggggactgtaggatttggatgggaagtggtgggctctgagctggctgacagggctgtggggctgggagcaggcactggctggcatggagggcttgtagagagtagtagttgaagagaaaggtggggaagtttgtgggccgagctgtggaggatattggctatcaggctgaaattttgtatttcattttgtagacaacaggcagctctaaaaacttttgtgtattttactttgaatttcaatcagtgatgcaaatttcaatataggacttctgaggacaaaggaaacaaaatgtgtgtgaatgggtctctcaaattataaaagttataggatcataccttcataattttcctctggttataaaagtaatgcatgagtaaggtaaaaacatccaaacatgatagtgtagaaagtatgacacccttatggtaaacttccaaacaaacaaaaatcatcagtatgtgttgtcttcttattgctttagtagaaagtttctatgtaaaagtatgacatgattaaagttgtattttaggaagatcactttggtggcagcatgtagggtgattagagggagagaaactaaagtccaggagactaccagggggttagtattattgtttaacaaatatacgaataccttcaatgttagcttataatcgaataccttcagtgttagtaagttcaaaggtaatgggggactgcggtaagagagcagaaaaaattaaagaggaatggatgaattcaagagacacttaaaggcagaatcattaggattagtgactggatttcgattggagcagagaagaaagaggagcaaaaggtacctgggaatttcaagttgggtctttgggagaatcacccatatcagttctagggaagttagggatggaagctcagtttgggtgagtggtatgagctagatgtatttggactgagccatcagaaatcacatttaggagcaaagcagagaagccatccaaagcaatagagaaagaacagccagttttgggaagagaaaagcaagcaagttcaggcacagagcagagggcaagagattcagagagagtggctttccaggtgctgcagaggcctcaaagggagtgaggcctgagaaaggtgacaggcggttagaggaaagcaacctggcataccgagcatcttctggggtcagtgaaatgggagaggcagaatcgagatttcaagagcaataaagcgcataaatagctatacataacatagctatgtaatgtagggtaatgtatataatagtctacaacttctgttcctaagaaggagagaattaatatgattatctggtggaggcagcagcaatataatttcatttatgttatggtattcctattcttacttaacatattcatgcccagttatttatgtgtaactgtgaggttgaacatttattacttgacaggtgaagtaaaaaagcacattatttttgtgccattttgtcctgttctttgggacgaattcttccctactccttggataaaatgtagggaaaatctcatcatttttatggttactctttggtaacaaaaggtcctgtcatgttcggttactttcatatacacagtctacattcagtaattaatatgtccagtctaataaaataaaatgctcccttctccagctcgggtcttctttggtctggatgcctgcctccaactcactaggctgcctttgactcctccagactgaattgggtggaggaaagagaagtgaggggtaacaaaattctcatctgatgtataaggatgagtcatgtgaacttgctaatatttgtcctgcaaaaatgtcaatttaatatggttcaatgtaatactgttgtcatctgcatgagacctgtctgggctaagacggcatttaaaactacatttttcctttttagaactcctttctgtattcctaggaggacccttaagattgtggggaggtctttcatccaaagtaccctggatgcttttgaatctgtctcctccaaatggcagctcataactccatctttagctgctgggcatctagatcacacacctcagcttccctcttctgagggcttgccttattcctactaaggctgttttgatcaggatagaaaacagttccatgctgacttgctcttttggcatatctagttgagggaaaatgctcatgctttctctgatcctgaacattcaggagatgcagaccactcctagcatagcacactcttagctccactgccagcacaaatattataccctttagagttttcaggtgtgtcaacagcctgctcactgacagcttgaggatgctcccagggacacatacctagttgtctgcattgtctatggaagcccgtttccctaggcttgaaatgagggccaagcaactctacctgatgccccagggtggggtgagtgccactcctagctcatcgaaagctctctcctgaaattcccttctagttttatttttatcttgacctttccaccttggtgatgggttaagagttacaaaaccagtttttggaaacctcctttacaggtcctgcatccttcacctgtggaaggtggacatagttgacatctattgtcttgggggctaaacgcaaaactgagtctgaaatagttcccttttaacctcccgttatgtgcagatttaacaaattcagtgcctttcttaaagtaaaaggtacatccagacactggagagtagtgctaaaacaaactattagggaattgaaatgaccataaaagaacataatgaacaaacagaaataatccaaccagcaccttagttttgattctggcattgctttttactccagaagcaagagttgaaagaaactgacctgcattttaaaatatattttttaatggtcatgtaatatgctatatttcttcaaatctgttaaaaatagaaactcaagagttggatggcatcatggttagggaagaaaaattctgtaactggaatagatggttgtagtaattcacatgtctcaatttgtattctagtcataacatacttcttaaaagggtggtaaaaataatgtgttttctctttcccacttcatttgtctaatttctgtaacatacaccctcacaatgaattattgactcagggacaagaggtagagaaattacagaggttgctaagaatacctgctgactcttattttctacttctgctttagaagttgtttagaaaatctaatcaaggccgtcttacatccagtagaaacagagagtttctactggatgtgctttggagcaggtttttaagagagagctttccccatggaagagtgaccatattggggaaagaagagctcaggagacatgagttatgcttctcttgcctggggaggtgtttgcttaaaagacatgctgcccaagtatttaagccttcaggttagggtcttttttccagttgttggcagagtgaatgagcatgaactgtatttgatgaactacagttaacacaaatactcattttgaagcagataaaattattttgaaagtgaaggaaaattgtttcataagagaaacttgtacttcctacgaaaagactgtgtttttttgctccatgtttttagtaaagtttagtcaaatttagtttagttcacctagaacgaacactgaacaatttggaaatacattttattcctttttttaaactagaggttagcaaaccctggcagaaaaaccaaatgtagcctgacacctgtttttgtgaataaagatttattggaacatagtcgtgctcgtttgtttacaaattgtgtctggctacatttgcattgcaacagcattgttaagtagttgggacagagaccatatggcctgcaaagcctaaaatatttactatcccactctttacaggaaaagtttgcagatccctgttctagattaatgaaagtactcagtatcagtgtttttgtggttatatatggcataaatggctattaattctctgaattattattaattttaaatatttcagtatcttttgtacttgattatatatctcattaaaataattgaattaattctgaaaattggtatggtctgtttgcctaatggacaatacactggacattacaaaactaatttctgcttttcaaatcccttggacagtaaaataatttatttcttgatttgcattttaccacatatgagcaccttgggaagacattgtgtagtagaaagcatactgtcagcacccacttccaaaatgttttctagatttattggaacaatttaaaatgtcagtgaaaagaagtacttttcttcctttgtgttcttcatttttctatgatggctggatgcattcactccttggggcattcatgttccttccttaaagggcagagttaaggttactctgtgggtcatttgcctcttcagggttgtttgatccttctcaagagaagattattttaatgtattgcaggagatgctgcctgataactgaggctgatctgttgagcactgtaggaaccagactggggacaagaattgactcatttattcatttatacttatgtttattcaacaaacgttgaaggcatgctttactaggttctgtgctctcaaggatttcatagtctcttacaggagatggacacaaagcaaatgattatcatatactgtgttaggcaagatgattgagttatagtcaggatattataggagagaaggggcatctaatccaccatggggagagggcaatcaagaaaagctatttgaaagaggctaaatccagatcaataaatccaggacattctgtcagaaattcagatggagatgtctggcaggtggatttgagtctcaatctgggatagagataaggatttaggtgtctttattctaggaatggcagttaaaaccctgagagctgatgagagcctgaagagtgggaagagggctcagcatgggaaaggccaacatttaaggatcagaaagagatgcataaaggaaacaaggaaggggtcagagagataattaggaataccaagagaacaaaagacaatggtgtttagagactgagtgaggggaaggagacggtcaatggaaagatggaaaatccaggattgatcagggactggcaatgtaggaggcagaggggagatggaattcatttcaaatattctaatgtaaactacttatgtgttaaacaaatgatgacctaaaacttagtttcaggctgttgtttaaagtaggctcaaataagtaagttgatttcctatatttgtaaaagctaataatttttatttaatcaaacacacatttattgagcacttattttgtactaggtgctataacaggttggcaccctaagggataaactatgcgggatataaaaatgatcaaaaagtgaagtagtaatatttaagctaagccctggaaaatggggagggatggaagtacatataagtcaagaataggcaagagttagagcaaaaacacagtgcatgaaaccccaggacactgtcagtcttgaattattgttcttgactatgtctgtctccaccactagactctctaatgctgaggactgtgatttatagagcctcacatggtgttttgcatgttgtatttactcaattaatgtttgctatatgaatgaaacttgagaaacagaaacatcataattgttacttattccatagtcttgtctgtgctggcctcagtaataaagcaaaaactaacttaaagactagcattcttatgttcattagtaaaacaggtaatagataaattaccttcttgatttgacttttgagaatgacgtgttagaacttataaatgaatcaaatacgtgtaatttataatgttactatatgatatgaaaacaatttgagatagaagtcataaatatgaagtttcttaagactttgatcaaaacaacaaaatttctttgagattttctttttttaaaacttttatttttcaacaacagctaaccaaagatgggatcttctttacatgacaatgttgaagccaattgatacacaatttatttgttagcgtgttagtggttctatttgttttatattttgaagtgaactatggccttcttttaggactgaaaaataacttcgtggtgtacagatattttagattaaccaagggtacttgttaacaaaagctattattttatagctgcatctccgtgtttgatttgataaatttgggaattaaatttacaatgcaaatttcattagtggaattacatagccaatttggagaggttctatttaatgtgctaccttgatttagaatgagcgatttagaaatatgaagagttgactgggcatggtgtctcatgcctgtaatcctaggactttgggaggctgaggtgtgattgcttgagctcaggagtttgagaccagcctgggcaacatggcaaaatcctgttctacttaaaaaaaaaaaaaattagctgggcatagtgacttgcccctgtggtcccagctagttggtgggctgaggcaggagaatcgcttgagtccaggaggttgaggcttcagtgagctgtgtttgtgccactatactccagcctgggtgacaaagtgagaccctgtctgaaaaaacaaaaacaaaaacaaaagcaacaaaaaaaggagaaatatgaagagttagataaatgatacgataatagcccacgttttcttgttgattgtggaaaaggaaataattaacagtatctattataaccacataggatgtattgatttaagaattacagtagaaaataattcaaggaattggaaaagtgaggcatgttgttctatgaagattttgcatccttaattatttataagtatttgagttatgtgtaacttcattttctccattaatttttgataatgatatccaaaacaaatctgcagttatttgttagtttgacctgtgttatctttgcttcataaattcctcaattatctagtttaatctagttcttatttcacaaatgatggaagtcagattaagcagtttgtttaaatgacccgcaatgaattatagagataggtcaccattttatgctaacgggttggcaagaaaaattcatgaaattttacaggcttcatgttgcagttgtctacaaatacacttccataaatgaaaataaacaagtttgttcattgcatcattataaatgtaatttaaaaattaattttctaaattgttttatgcaagtaagtaagcaagcaaaataaataagaactcatatactttcctgtcttttttttctcccctgcccaataggaccagacatactatcaatcatttcaggtttcagaatatggtaagagttcctcaatctattttggaagaaaaaaacatttgttactttcagcagagattagagaaatttttaagagatgtattatttgaagtaaaaatctcaatggagttaaaattctggtgttttttaaaggcctaaccatattttttttcctttgtaaaaattctaaaatctcattatacagaaaaaagtagaaaaaaatcacctatagttgcattgtgctagtctttttcttatccacaacatgatttttctatttttctactttgttgtgataaaaaaatatttaaagtgttgcacttaaaaatacttaacaaattctcttccatatttttcatttgattgacattggtaaagatggtctgtttctcaggtaaatcctcttcttgcaggtgccatacttacaatatctgttagaaacccatagataaaaacagtgggacccatctattttgttggtgaatggagaggaatgagagatgagactgaaagcataggatcttagtattagttataatttgaatagttattttgaggttgcacatgcagtttttcatttgatttgagcttcttttttgagattgggttttatttaggcagtagaaattataatatacaggtagtaatacctcaggctctgtgggccatatgtctctgttgttactactcaattttgccgtggtagcatgagagcagccaaagataatatgtacatgaatgttgctgtggttcaataaactttattttatgggcatgaaaatttgaatttcatataattttcatatgtcatgaaatattcttcttcttttgatttttttcaaccagttaaaaattgtaaaaaatgttcttggttcatgctggtcatagtttgctgaactctgatctgtaactgtgctctccagtagcggggccacttgccatgtgtggctgttgagcacttcaaatgtggctagtccagatggagacagcagatgtcaaagacttagtacaagaaaaatgaaagaaagaaaaatagttcattaataaccatttttaaatttttattatttgttgaaaagatactattttagatatatagggttaagtaaaatataaaaattaatttcacctgcttatttttactttaaaaatttgactactagaaatgaaaaattacacatatggctcccattttatgtcttttagacagccctgatctagaagaataagtcataggggaccaacatttttataggctatctttatatcccattgtgatggtatgctgggtggttgtatgttgttgaatgaggtatagtggaggtggggaattgattgcaaacctatgatgtttgaggaagaggcatttggattttcttggtggtgggggttgcgggggcggggtcatagttgactcacgtgactttttggtctccctgtcctagactgatctagagtctaggagtgtcttggtacattcagggccattagccttgattggagctgaaggtctgccctatattgttgactatttggcttttttgatacttctcttatgttaattgaatggtgaggtgccttgttatgagagtggcctgtttctttggtcagagaactatagaaaaaaagttttatcgactgtaaagaaacacacattttttactctggtggagtgctttatatcaattatagtattagtacttataaaatagtttataccttaattattattattatttttcgagacggagtctcactgtgtcactcaggctggagtgcagtggcatgatctcgggtcactgcaaccactgcctcctgggttcaagcgattctcctgcctcagcctcctgagtagctgggattacaggcatgcaccaccatgcccaacaatttttgtatttttagtagagataggattcaccatgctggccaggctggtcttgaactcctgacctcaggtgatccacctgcctctgcctcccgagttctagaattacaggcgtgagccactactcccagcctcataccttaattatataaagctgaccaaattagggatttctagttttgggtgaaacaaagggcttttcatctgtcttaatatgtatttggattctaataaatttaagttcaaatagtacaaattattaactccttcacaacaattttatttttcattttattcagtggaagatgatagtgaagtagattctttaaaagatttacccttgcttcttcgtctcaaaaagaaaaaaagatttacccttgcttttcttttccagtattaccaaaatttgaagtgactttgcagacaccattatattgttctatgaattctaagcatttaaatggtaccatcacggcaaagtaagtgtcatttttcttttgatatgactcaaaaccattataaaactgtgtgaccaaaagctgattgtatacttcagaatattagggcaatttttgcttattaaaataccttagtggacagtaaatgactatttataataaataattaagcatgggacagagctccacatttcactgcatctatcactctgcttgcaagccgacccagtcttaaaatatttcccatatttcttagacttattaagggatttatattatagatagcactgtttttttttcagatgacatggacttgaagccaccacgcctggctaattttttttcctttatagagatggggtcttgccattttctccaggctggtcttgaactcctggcctcacatgatactcctgccttggtctcctaaagtgctgagactacaggcatgttccaccactcctgggctgatttgctttttaaaaattagtcaagcttttattttctgagtgattaacttatcccaaatgcttcccggttctatttgggacaataattgtatctttgtttcacaattattataaccagcttggtgtttggaaaactaaatctgcttcacattaaagtcaaaattaaaaatttttatgtctgagagtactttaaattatatttggattttaaagatccttttactaccatgtgatataactctgttaaaatacaatatcctggcattctagttttgcttttatttaatctttagcacgatacaagtagagtcaggtttcgaagactcttgaatgagtgaatttgatgctcaggttgctaaccagggtttccactgactttactgtttttgtttggaagtgtggtgcatttgaacttggatttttttttttttcttcttagtttggaatatgctatattttaagatttgcttttgagttgagttgatccagatgaggggctgtttctgtgttcagttaatggattcaggaagccgccgtttactgctgtgatttatagactgcaaagcaaaattgacttccatgtctggctcagcatttcacaatagagatagaagcaacccaggaaatgtacttaaaaaaggctttctgggccgggcgcagtggctcacgcctgtaatcccagcactttgggaggccgaggcgggcgggtcacaaggtcaggagatcgagaccatcttggctaacacggtgaaaccccgtctctactgaaaatacaaaaaattagccgggcgcggtggcgggcgcctgtagtcccagctactcgggaggctgaggcaggagaatggcgtgaacctgggaggcggagcttgcagtgagccgagattgtgccactgcagtccggcctgggctaaagagcgggaccccgtctcaaaaaaaaaaaaaaaaaaaaaaaaaaaaaagcctttctggcagtaaaatgtattgcctcttcttcacatgaagagactttcctataatgagagaggataaaattaatacatagaaagaaaagtagcagagaatatttgacttacgaagctaagtttgggccaggtgaggtggctcgtgcctataatcccagcactttggggggccaaactgggtggatcacttgaggtcaggagtttgagaccagcctggccaatatggtgaaactccatttctactgaaaatacaaaaattagctgggtgtagtggcagacgcctataatcccagctattcgggaggctgaagcagagaattgattgaacctgggaggcagaggttgcagtgaccctagatcgcaccactgcactccagcctgggtgacagagcgagactctgtctcaaaaaataaaataaaataaaattcgctggacgtggtggtgtgcgcttgtcatcccagctactcaggaggctgaggcaggagaatctcttgaacccctgaggtggaggttgcagtgagccgagatcatgccactgcatccagcctgggtgacagagtgagactgtgtctaaacacacacacacacacacacacacacacacacacacacacacacacacacacacacaaacccagaaactaagtttggtactacagattttaaaaattattcttattttaaattttttggcaaaaaatcaatttttgacagccatgagcacatttggtgcttcagaatctatggtgtaataaaccttcctcgctaatattggaacatgtagcaatagaccatggtgattgcactatcagctgacatttaaaaatcttctaaagctgcgaagtttcgtgggcattcagttaatgggccagtccagatttctaggagtgaaagatttatgtaatctttaaaaaatctttggacaggaaactgtcacttccaattatgatcaggtattttctggagtctttcagatggtcagaggttgggcagagtgtgtgttaagaactgttttaagctactatagttaaaagtcttcccattttggaaacttaatttcaaggatcaccatttattttttatttatttatttattttttgagttggagtctcactctgttgcccaggctggagtgcagtggcacgatcttggctcactgcagcctccacctcctgggttcaaacagttctctgcctcagcctccaagtagctgggattacaggcacccaccaccacgcctggctaatttttttgcatttttagtagagatggggtttcaccatcttggccaggctggtcttgaactcctgacctcgtgatccaccagcctcggcctcccaaagtgctgggattacaggcatgagccaccgtgcccatccaggatcaccgtttattaaagacatttttgttgggtttagtttaaaaaattatagtaggagtatgtttttaatgtaacttttttctttttaaaattttctctttttagtgttttgccatcccataatgtatttttttttaatttatttattttgagacagagtcttgctgtcttgctgtgttgtccaggctggagtgcagtgatgcgatctcggctcactgcaacctccacttcctgggttcaagcagttctcctgcctcagcctccaaagtagctgggagtacaggtgcctgccaccatgcccagctgatttttgtatttttagtagagacagggtttcactatgttggccaggctggtcttgcactcctgacctcgtgatccacccgcctcggcctcccaaagtgctgggattacaggcgtgagccaccgctcccagcccaatgtaattttttaaaatgttagattgctaattgctaatgtatgctattgaaagtagtatttttattgtgttctgacaatatcaagttgatacataaaaagactatgtttataattattcaggtatacatatgggaagccagtgaaaggagacgtaacgcttacatttttacctttatccttttggggaaagaagaaaaatattacaaaaacatttaaggtaacttttgcagacactttataacttgtgatgggtaaaatatgtgtatttttctagaaataagatttagtactgaattaacaaaaagttaagtgtaagaaaatcaagagctttccagcattgaacaaatgagaatttatgcaattatagcgattaaaatggtaccagagctatcatattgttgtgacttagtttgagttttatttctaaaatttgaattgctaaaatggtaaataatactgaacttttaaacaaaacagataaatggatctgcaaacttctcttttaatgatgaagagatgaaaaatgtaatggattcttcaaatggactttctgaatacctggatctatcttcccctggaccagtagaaattttaaccacagtgacagaatcagttacaggtttgtagactttaaagtggaggtaaaactattcatgtgacactgctttatcatctttcttattataactggcactttcatttgggagcatcattgactttctaaagatgctatttaatgttgaaattgttatgactgttagtctaagttgcaggatgattttataagggggaccttattttctatgaaagtcactacttcaaccacatgtgatagctcttcttaatttttttttccatttctactctttccccctgaaaactacatggaaaggcttacattttcagctggtaaccttcttctttttaaaatcactgaaatctcagctgttcctagacagttttcacttttaataagaaaataaagttgagtttcttggacctttctgttactatttcaacaactttagccaatccctgaaagagcaaaagactattctttcattggccttaatttgctgctgttgtacatgtcactgcggtagaatggcagcaatggtgcttagaggggatccatagagaacataatagaactgtgtggtgcttgtagtgtcaaagtcaatatgggaatgaggcttatgtgccctgatatttttaaccctttttgcaataggttagatggagagatttggtggatagaagctttgggtgaaacattacttttgctttctaaattgtgcaatttccaaaaaaggtatttcaagaaatgtaagcactaatgtgttcttcaagcaacatgattacatcattgagttttttgattatactactgtcttgaagccatctctcaacttcacagccactgtaagttggtatatttatttccagtccatagcagtaagttcagcttaatgaaatagaatgggaaataatattttcaatgcttttctaaaaatttaagccaaaaagtatataactttgtctctttagacatgtggggcatgtagatttgtttccaaatgacctttgtctattttttctaaaaaagtagttttcaacaacagtaaaaatattttttgaaaatgtatttttttttacaagcaatttaagttagatgtggattgctacactgaagcttttattaattaattgaaataattacatggtagtcaaaaatttcaatttcaatttggtttttataatgattaattgtagttctaacaaagatgttacattaaaaaattatcaacaaatctcattttttggagataatgttgaattaacacattaattgtgtgaatgttgacaatgagcaaggcatggtgttgggcagggctatagagccaagaagggctcagtccctaccctcaggttcacagtttttgtgatggtgacaggaaaatatatacttaactataactcatggcagattaagattagtgttacaaattaatttctgtagggatttaaagcaggggttgccaaactttttctgcaaagggctagagagtaaatattttcagctttgtggccatgtggtctttgtcacagctactcagccctgccattgtagtgtgaaagcagccatagccaatttataaataagtgagagtggaatgtgttccagtaaaactttgtttacaaacatactggattggcctatcagcttgcagtagttggctgatctctgatttaaagaaaggaggagtcaggcctggcgtggtggctcatgcctgtaatcccagcactttgggaggccgaggcaggcggatcacctgaggttaggagttcgagaccagcctgaccaacatggagaaaccccatctccactaaaaacacaaaaaattaggcgggcgtggtggcacatgcctgtaattccaactactcgggaggctgaggcagaagaatcgcttgaacccgggaggcggaggttgtggtgagtggagattgcgccattgcactccagcctgggcaacaaaagtggaactccatttcaaaaaaaaaaaaaaaaacaactaaaacgacaaagaaagaatggaggggtcacttgttgaatgggtcggtagacgtttcaggtagtttctgatttcaatgatcttaatttaggcagtttgaaaaggtcagtttaaaaaattgaggctgcaaatggtaatttctgaatgagtagagtgaaaccatgggcgttcagagggggagagttggctgtgggataggcatgggggttactatgggatgctttatggaagaggatggtctaaactgttttttaaaagattagttgtgttttaaatgcttgggcatgggagtgggaggtggggagcaagataagaagtgagacaagaaggagaggtctggctggcaatttcggggtgcactcaggtagtgtttgagggagactgctcaggctgtgtgcaggaggatggctgtagcccaggagtgaggggatgggcagccttgtggtggtagctgtgggagtggagaggatggaagagagaagcataggactttggtgttcattggatttggggggtggggagagggagggaaaattcagaaatgtaaagtcttgcaaaatcctgatgttagcgaaaataggaaaatgacatggttgctcattttagatatgtgaattcaaggtgtccaggatatccaagtagaaatttcctgcaggtaactggaggtgttgggcatgaactccagagaagggcaaggatgggaattttggctttagggtacaatcacagagatgacaagctaagcaataggattgctgagaccttggaggaagggagaagaacagatgtccaaaattcccacatttagggagaggaaagaggaactgaaaaggacactgagaggatgcagaaagagaggtaggaagtgaagggggttattttggagccgcggaagttaagggaatccataatattaacaagggagtaatcagtgatgttggatgcctcctagagagaggccactgaatctggatattatagtgctaccaataatttttgagagttcaatttcatgagaatactacagacggaaactgtatttaatcgtacttaaatcctttgtgtttttaagatgttttgttttgaccattaggtgaaggtaactcgtgctgatggcaaccaactgactcttgaagaaagaagaaataatgtagtcataacagtgacacagagaaactatactgagtactggagcggatctaacagtggaaatcagaaaatggaagctgttcagaaaataaattatactgtcccccaaagtggaacttttaagattgaattcccaatcctggaggattccagtgagctacagttgaaggtgccgtctgtttcccatcattgtgtcactgcaacaacaccattacagttgtaatcttgtggtaggcactcaacctgtcagaactgaaagtacataggaagtggacacatgtttctgtttcacaaaatggaggagagtggtaaaagtaatttaatgacagtggctgaaggttagactaaatttttccttgtaagttcattttgaagagcacagtaaaatatagcaatgtgaagatatgatggagaatttcctcaaatccccagtctagtgtttgaggtcagtcatttgaaattttattttggcaggaattgaaatgaaagataaatatatatacatatatatatatacacacatatatatgtatatgtatatgtgtgtgtgtatatatatatatatatattcctttcctgtgttttaaagctatcagtttctttacagtataagctataaaatgatgaaaatccaatgtctggtgaatgtatttttcttgcattaaaaaaaagtgaagatgaatttggtctttaataattgttcagcaggtaacatcttttctcaaggtgttactaaagatgaacatttacagctcctttttcttttattataggcctatttccttggtagtaaaagtagcatggcagttcatagtctgtttaagtctcctagtaagacatacatccaactaaaaacaagagatgaaaatataaaggtaatgcttacaattcacttgagaattacaatataattggactatcttgcttttgatatgtacatattaattaaggtttttttctaggtgggatcgccttttgagttggtggttagtggcaacaaacgattgaaggagttaagctatatggtaatctcttatagaatctaaatttatgatctattatagaatcatctttttattcacttttaagttctggggtacatgtgcaggtttgttatatagctaaactcatgtcatgggggtttgttgtacagattatttcatcacccaggtattaatcctagtacccattagttatttttcctgatcctctccctcctcctaccctccaccctcccataggacccagtgtcggttgttcccctctatgtgtccatgtgttctcatcatttagtgtgcacttataagtgagaatatgtggtatttggttttctgttcctgcattagtttgctaaggatagtggcctccagctccatccatcttcctgcaaaggacatgattttattcttttttatagctgcatagtattccgtagtatatgtgcaccacgttttctttatccagtccacctttgatgggcatttaggttgagaatcatcttaaatggtgtctaccttatatctacactaaacttattaatttttataactcagcctcatacctttttagcctgtcgttttaacattgcaatcaaataaaaaaccttttttatttcttcacctatgtttgcatttccaatttaaaatagataactaaaccttgttttatgtttaaaatttaaatttctatttgataagcaatatatattcattatagaaaattatacaatgtaagaaaaatgaaaaagaaaataaaaagtgctatccatgaccatgtaattttgctaattcagctttcccaatggtgccctgtattccttgcaatcaaaaatagattcttgactatgaagctactgttgctccaatcagtattactttaatcttaccattttctttttctcagtgtatgctctctgtgtttgtcagagaggttttcctgcattccaaaagactttgaagctggtttaattcaagaatttgtgtgtatgcatacaattatgattaatgtagcttcagatgagatcctactaggtttggcaataaattaaacccatctactgttcttttcaggtagtatccaggggacagttggtggctgtaggaaaacaaaattcaacaatgttctctttaacaccagaaaattcttggactccaaaagcctgtgtaattgtgtattatattgaagatgatggggaaattataagtgatgttctaaaaattcctgttcagcttgtttttaaaaataaggtaagatttaaggtaatgatgtttaaaagaaaactttatattagtgaagtctgagaaatgtaatatttctttagaaaagtatcattaagccaaactggttagaaatttatattattctatttctagaactatggttaaacttttcaatatttagcattacatatatattttcacttgtggagttattggggattgatagtctatttgtaaactatggttctggtctttaagactttaactcagatcatgaagctgtttcagaatttttcaaatggttgaattaatattgctcaggagtgaaattccagtggtaacatagaattcgtgtggtgttgatattgcagtgatataaagggtgtgggaaggagtgaggccatctaggataatacgtagaagcagagctgggctttcattaatctatttaattagctggaaactagcccagggcaacttttcaatgagcttgattaccctgcatgtgaggatctgtgtggagtctggtctccaagtgcaccagtgccttttatagcctaatgtgaccagcttgagtaggaaatccctttggaccccagctctgaaaagaaacctgattgcatcatctcatgtatataagaatgagccattgctgtcactaacaactgctgccctctccatcctcttatgtaaaaaattattcctttaaatagtgatttgcagagtatggttcttctatttctttttcctttatttgttaaaatatttgagaagtaacagtagtgatctgtactttcttttttaaaatttgtatttatttattttttgagactaggttataagactggctaatttttgtattttcggtagagagagggttttgccatgttgctcaggttggtctctaactcctgggctcaggagatccacctgcctcagcctctcaaggtgccgggattacagacatgagccactgcgccaggcctgtactttagttttgttgagaattattactctcactttgattgttaaaatataatacaaacttctatttttttagcttatgaaaagagttcttaaaaagataacactgtgttttgttagtggtagagcacaatttttgacttctagtgatcctagtaattgcctgacaaaggtgaattttaaagtagaagttatggaggcttaaacatctcagaaagcaagaccaggttctaaagctggttacagtgtaaaagatcacggactttgaggaagcagagtatatgaatagcatactttcaagtaatttcttagctataagagaaaggaattagtttcagttccatttgcaactatatatctttctcttatttttatgtgataatcatcacatttataaagaaacaacataattagaaatggaatatgtctaagcaagagaaagttagtgaatgtttattaatgttcagtaatgactatggaataaataaatgagctgtaggagcggcaagagatttaggtttagaaatcatccacacagaagtgcaagttgaagccatggggctctgaggtacagagtatagagtgagaagtacaaggtctaagactgagtcttgaacatgctgctccctctcccccatgctttagtgtgtaggtggagaaagtaacctacatacagtggtttgagaggcatgtcagaaaaaaattgcactggacaagttaaacaggtaaggaaggctttattcaagactattgcaatggaagagagagatgataaactcaactctgctgaaacaaaaggcagaagagtttttaagcactggggtgaactcatatgaaagtgctggaggacattgttgggaggttggtcaatgtgattaggtagtctgtgtttgctaattagtacttactgaagttaggtttccaccctcccacagagaatgagatcaattgggactctgtctttcttgatgattatacttcaaagggatggctctgaggaccttgagaaagacatgcctgggttgtaaaactggccagaggctgggaaaagatttacatctcagagagttggagaaagaatttataattgcatgttttctaaaataaatgctctaagaaaagggagctcaggggcttatagttaggaagaagcctgtctttgtaaagtcgagtcaacttgaggggaatgttaaggccctctttgtcaggcattataatcattggcacagcagaatgttgagatagagctagatgaaatgaaaattcccagcaggggccaggcactgtggctcacgcctgtaatcccagcactttgggaggccgaggcaggcggatcacctgtggtcaggagttggagaccagcctggccaacatggtgaaaccctgtctctactaaaaaagttacaaaaattagccgggcatggtggtgggcgcctgtaatcccagctacttgggaggctgaggcaggagaatcacttgaacccaggaggcaaaggttgcagtgagccgagattgtgccattgtactctagcctgggcaacaagagggaaactccatctcaaaaacaaaaaaagaaaatgcccagtaggcaggggcctcatgacaaggccatttggttcagcggtgatgcgatgactgatgctctttataaagtattagtaggtccaggaccatgctttcctgagggtaatggtgaggaagtttagaaggccatggagacagagcatgagggcctggactagtgtttgctctaaccacccactcccttggaaaggccacccctcaccctcagtccaggtggttctatgagtccccgaggagctctccttgccttcacttctgagctcacgtcaccaacatgtaggcatctgcagtttctttcctctttcgtgtttctcctttgtatactggccttccccaggtatgctagacactgctgtcttctactcagcactggctgggatctgaggggtgggagatggagaaagggaagaaaaaagatacagaattaaagaataattcagatttataccactgtgagaagttctatgacttgcaaacactctgggcgtagtttttttattcctttccctagagaagggttatgcatggttttagaattatccctgagccaaggcaaatgtagaatctttgctatattttgggccagtggtctgctattggcaagaatatgctttaaaaaagtgaaataagttaatcctcctttctctcttttttagataaagctatattggagtaaagtgaaagctgaaccatctgagaaagtctctcttaggatctctgtgacacagcctgactccatagttgggattgtagctgttgacaaaagtgtgaatctgatgaatgcctctaatgatattacaatggaaaatgtgagtttagctattttttcattatgaaaatatgtattacaagagaaagaggaaactttattgtactactttaaatatttaaagaaaatttcattagttcctttgcatgtgtaagtcaataattagtaaagcttccaaataaaaagtaatctgtatcgtaatggttatgttggaaagaagcttatgtggttaagtgtccttgctctctttcatcagaattatcacctaaagttagatgggaggaataaattctggaggtctgtggtacaacatggtgactgtagttaatgataatgtaccatatacttgaaaattgctaaaagagtagatttgaaatgttctcaccacaaaaaatgatcagtatgtagtatgcaaggtgatagatatgttaattagcttaatcattccacagtatatacaaatatcaatatatcacattgtactccataaatatatacaataaaaaatgccacctatagttttccttcattttcatactacattttaagacactcaagaaagtactaggaaattgacttgcattattctgggttacgttggataagagagctttagataatttttgaagtgtgttctattttggttatacgtaatggttataaataattacagcacccattgtgccgggcatggtggctcatgcctgtaatctcagcactttgggaggccgaggtgggtggatcacgaggtcaggagatcgagaccatcttggctaacatggtgaaaccccatctctactaaaaatacaaaaaattagccgggcgcggtggcgggcgcctgtagtcccagctactcgggaggctgaggcaggagaatggcatgaatcccaggaggcggagcttgcagcgagcagaaatcgtgccactgcactccagcctgggtgacagagcaagactctgtctcaaaaaaaaaaaaaaaaaaaaaaaaaattacagttcccattgaatcaggaatttgcagtatattgagggggtgaaatcatgttactctgcagaaaacttacaaaccaagaagtatatgaacatctagcattgcataaatgaatcccatattcattagtcatttgatttttatttatatgtccttgatatattcttgtgtaccattgtcagttgtaaactagattgtataatttttgtctattaatggattcagtagagtgattaacaatacatcagaaagaaaactatatatagttgggttgatttggtcattatattgtaaaatttagtttacttcagaaaaattgtacattttcttcagtctacacaactatttttttcaatcttttcttttttttgaaaactttttttttttttacaaccagttggtcatttttctctctttgacttttcctgagttcacattagttttatgtctgtagatcttgcatttgatttaactgaatataatagtatctgttattttatagctttctaaatatcttggtaaatatattatttcattttttcttccctacatatgtgtatgtttgtgtgtgtgtacgtgtgtgtatacatacacatacacacacaccccatctgatgtgctgcgcactctgtctctatttacatttatatatgtgtgttatatatatatttacaagtatatttatatatacatataatatatgcatatttacattaaattttttcagatattaagattgctttctcagctttcttttggttcatattttcctggtgtatttttacatttcttattttcaaccttttgtaaatttttaagtgtatcttttaaatagcatattattatattttgcttttgtttttcaatctaatctaatagtcaacatcttttaataagcttttatttaatccacttataagtatggtaattctttttctaatggggactttttctgcttttctatattttaaaatttagattctttttctgtgggctagattaaatttgttttggtgatttagaagttatgcgttctatttttgttactctgttagttacaaactgtacccatattaatttatccttgttggtttctaaaggttatcagtatttgaatcttctagtatgctcctattccttttggtctctacctctgtctcctccttctcctcctaccctggccagatcattttgctcgtgtacagattttagttccatgttaccatagattgcctttcaatctatgctgttctttcattttttaattgtggaaatctgtgttgattatttcttcctccccattttattttttctctccttctgggacttttgtttttcagaaattggcacttttacttcctccttcacatctcttagttttttgttttatgttttctatctctttgtcttttctgtgtttctctgggagagttcttcagtcaactcagcttagtgatcattcattagctgtattttaatctactttaatctctcatattgtgtcttttatgtcagctattatgtttttaatatctattatttttcctttgtttcttgtttttgttttatattctgatatttcccattatctccttgtgtatatttgctatgcgtattttaaattattgaattgaacttttcagtaattttgtgtcacatgctatatgttgtttagttggatgtcttacttctacagtatttatactctttggaagtctagctgatttggcagattctctgataatatctattggtgaaggctgagcataaggttccaatctgtgtaagtcctatgaccccttcctgaggggagaggactagcctcatggactagagaattactcttgatcctttctgctgcttgcctctattcagtgtttcactcttaaacccttggggaaaagagcaccagtaagagaaggtctcctgaggttatgattcaccagccctgagggtttggaggaggaggagaggaagagtgaatgcttgagggacatgggtctgtctgcatgttttcttcactttcccacactagaaaggcagaggtgctgacctggtattatcttttggatacttgcacaggccgtctgagctgctactgctgttgattttggcagtgaagttgccaggactgtgtgtcctgaggctaccccctggagggaaataagagcaaagtttaaaaacctctcctccagcctgttttctgactatgtctgctgctgtgtctagctgccttctgccttagactgaagcgatgcactttctcccaagaccaattcaagatgtccaccactcttcctggggttttctcactgtgcacgttaaagttttctgcttgcttttattgtttctccagttggttctggaggagggagtgaggatcctgtcatagtagactgttttggcttcttttttttttttgttaaatttatatgtgttgtcaatagcctcttgaatgccttggaataatttcagtatgtggatcttccaggaataatttttagacattagccttcttttcgttagcctatgctgcagctttgacagtgctttgtttattgagccagtgagaacaggaagatttctttatgagatagatgctgcttttgctttttttaaaataaattttattgtgtatatttgaggttatgtgtatatatatatatgtatatatgtatatatgaggttattgtgtatacatgaggttatggaatacatataggcagtatagtggttactgtagtgaagcaaatttacatatttatcattttgcatagctacatttttgtgacaaaagcagttaaaatcgatttaatgaaaactccaaatatcatacaattctattaactatatctctcatgttgtagattatataactgcattttttcatcccacatatctgctattttgtatcccttgacctctatattcccattttcttcttcctttccccctcgcccctgtaaccatcatttgtttatctatttatttatttaaacttttaagttcagggatacatgtgcaggtttgttacataggcaaccttgtgtcataggggtttgttgtacagattatttcattactcaggtatgaagtccagtacccattagttatttttcatgatcctctccctgctcccaccctccaccctctgacaatctctactgtgtgttgttcctctctatgtgtccatgtgttctcatcatttaactcccacttataagtgagaacatgtggtatttggttttctgttcctgagttagtttgctaaggataatgacctccagctccatccatgtacctgcaaaggagatgatcttgttctttatggctgcctagtattccatggtgtacatgtaccacattttctttatccagtctatcattgacggacatttagtttgattccatgtctttgcttttgtgaatagtgctgcaatgaacatatgcatgcatacatctttataatagaattatttatattcctttggatatatacccaataatgggattgctagtattgaaatggtgtttctgtcttaagtctttgaggaattgtcacattgtcttgtacaatggctgaattaattaacactcctaccgacagtgtataagtgttccttttctctacaacctctccagcatctgttatttttttgacttttaaatgatagctatccattctgactggtgtgagatggtatctcattgtgtgtttttattttattttatttttttgagacaaagtctcactctgttgctcaggctggagtgcggtggtgtgatctcggctcactgcaacctctgcctcccaggttcaagtaattctcctgcctcagcctcccgagtagctgggactacaggtgtcgcctccacgcctggctaatttttaaacttttttagtagagaaggggtttcaccatattggcctggctggtctcaaactcctgaccttgtgatccatctcccttggcctcctaaagtgctgggattgcaggcgtgagccaccacgcaagtggccttttttttttttttttttttttttgacagagccttgatctgtcacccaggctggagtgcagtgatgtgatcttggctcactgcaaccttcaccttctgggttcaagcgattctcctgcctcagcctcccaagtagctgggattacaggcgtgagccaccatacccagctgatttttgtattttaatagagacagggtttcaccatgttggacaggctggtttcaaactcctgatctcaagtgatcctcctgctttggcctcccaaagtgctggggttacagatgtgagcactgtgcctggtggcctccttatggttctgatttgcatttctctaatgatcagtgatgttgagctttttctcatatgattgtaggcctcatgtatgcatgtatgacttcttttgaaaagtgtctgttcatatcctttgcccattttttttttttttttttttgagatggagtttcccccttgttgcccacgctgcaacctccacctcccgggttcaagtgattctcctgcctcagcctcccaagtagctggaattacaggcatgtgccaccatgcttgctaattttgtattttttagtagagatggggtttcttcatgttggtcagcctggtcttgaaaactcttgatctcaggtgatctgcctaccttggcctcccaaagtgctgggattagaggcatgagccactgtgcctggcccctttgctggcttaaaaaaattttttttttcttgtaaatttgtttaagttccttatagatgctggatattagatctttgtcagatgcacagtttgcaaaaattttctcccattttgtaggttgtttactgtgttgatagtttctttcactgtgcagatctctttcatttaatttgatcccatttgtcaatttttgcttttgttgcaattgcttttgaaatctttggctgttcctatgtcctgaatggtattgcctaggttgtcttccagggtttttatagttttgggttttacatttaagtttttaatccatcttgcgttaatttttgtatatgatgtaaggaaggggtccagtttcagtcttctgcatatggctagccagctctcccagcaccatttattgaataggaaatcctttccccattgcttgtttttgtcagatttgttgaagatcagatagttgtagatgtgcagtcttatttctgggttctctattctgttccattggtctatgtgtctgtttttgtgtgagtaccatgctgttttggttactatagccttgtagtacagtttgaactctggtagcatgatgcctccagctttgttctttttgcttaggattgtcttggctatacgagctcttttttggttccatatgaattttaaaatagttttctctcgttctgtgaagaatgtagtagtttaataggaatagcattgaatacataaattgctttgggcagtatggccattttaacaatattgattcttcctatccatgagcatgggatgttttcccattggtttgttcatctatgatttctttgagcagtggtttgtagttctccttgtagagatcttttacctccctagttagctgtgttcttaagtattttatctttctgtggcagttgtgaatgggagtttatttctgatttggctctctgctgtgtgttgttggtgtataggaatgctagtgattttcacacatcgaggctttcctgaagttgtttatcggtttaagaaacttttgggctgagactatggggttttctagatataggattatgtcatctgcaaacagggatagtttgacttcctctcttcctatttggatgccctttgtttcattttttctttctttcttttgcctgattgccctggccagaacttctagtactatgttgaataggagcggtgagagagggcattcttgtcttgtgctgataaccactgtttagatgctgcttcttgaattggtgtctgagcaagagcggaagcaaatcttcatgtatttgtattttgtgacagagttggagtcatggaactctagccttttctctccaatatgcctggctctggctcccttgtctacttgccagcttcttgacacattatgaattttttttcttattttattgttttcttattggtgcctgttctagttgcttactgtggatgcagccatggttgttgttagcattgcagaaatagtggttgcaggtgtttaattttatgctggaatgtgtttttttcaacttagttttttttgtgatttttaattctgactttgtcttaatttgtctggttaatgtaaatttttagggagaagtatcaaaattattacctaaaaaggtataatacttgatttttattgaagttaaatatatataaaataaaatttgctatttttaccattttaagtgtttaattcagtgacattaattacttcacaatgttatatgaccatcaccaatatttccaaaacattttcatggctccagacagagactctgtaatcattaagcaataactactcatttccccttccccacagcccttgggaacctctagtctacttctgtctttatgaatttacctattctagatatttcacatttatataatatttgcccttttgtgtctggcttatttcacttagcacagtgttttcaagattattccatgttgtagcatgtatcagagcttcatttctttttttttttttgagacggagtcttgctctgttacccaggctggagtgcaatagcgcgatcttggctcactgcaacctccgcctcccgggttcaaaccattctccttcctcagcctcccgagtagttgggactacaggcgcccaccactacgcccagctaattttttgtatttttagtagagacggggtttcactatgttggccaggctggtctcaaactcctgacctcatgatccgcccgcctcggcctcccaaagtgctgggattacaggtgtgagccaccgcacctagcctcagagcttcatttctttttacggctgaataatattgtgttgtgtatatataccacattttgtttatccatttttctgctgatggacacaggttgtttccacctttttggctactgtaactattgctgcaatgaacggtggcatacaagtatctcttggagttactgttttcagtccttttgtgtatatacctaggagtggaattactgggtcatattctaattctatgttaattttttgaggatctgacagttttactcttttagcctgttttacccactcttgctagcaatgagtactattcttaaaaaaaaaagcattgctaatggggtacatgcaaatatctcatttttgaaaaaaccgtatctcatggatgttgtcatttgcattgctttgattatcagtgaggctgaatatttttctgcttattaataacttatatttctacttttgtaaattgcctcttcagtttttttttttttttgtaaatttaggtcactgacattttgcagagtttatattaatctccaacaaagttaacctgtttatctctgccaaaagtgtgggtcatttttaatgctgacctctgcctgttggtcttgtcaaacagtaacatctaagttctccctggcagaactcaagtccagacttcttaggaactctggattgctgaacagagtaaaagtaagtataagtaagaaatgtaattttggctgactgactgatgaacaaagcaattacatattcattactcctcaatttgtgtcattttcaaagttgataaacatgaaggctaagtcttatctaagttacttggaagaagtgtggaaagttgttatgaatccattcattccgtatattttatcttctccttaggtggtccatgagttggaactttataacacaggatattatttaggcatgttcatgaattcttttgcagtctttcaggtatgttttgcttgctatattgaaaagatattaatattttttactattgcaaaccctttcagaattagtgatcattttgagatgttaaccttttattaaatgtgtcactgaagcgcagtcttatgtaagtataatttgaaatgaaaagataatcatctttccagttttaaaagcaaatggaacaattcaccattggaaaagttaatggaattgacattaaacaaataatttttcctttatgtatatgtatttccatataaatatattttatgtatatatataatatataaatatattttatgtatatatataatatataaatatattttatgtatatatataatatataaatatattttatatatatttccttgactttgaaatggctcttatgcatgagtgtgtgtgtgtatgtgtgtgtgtgtaagactgatgtgtgtatgtgtgtttgagaaagatcaatgtgtatgtgcgtgtgtgtgtttgagagagatcgatatgtgtatgcgtgtgtgtgtgtgtttgagtgagaaagatcgatgggcatgtgcgtgtgtgtgtttgagtgagaaaggttgatgtgtgtgtgcgtgtgtgtgtgtgtttgagtgagagacagaaaggttgaatgtatgtgcatgtgtatacctaagctctcaaaaatgagcacaagtgagtcaggagtttgggagactgccttgataatttagtgagcatttaaacttgtgttttaaaagaaaataaaaattattctttttaggaatgtggactctgggtattgacagatgcaaacctcacgaaggattatattgatggtgtttgtaagtaatacatggcgacatgcttgtatttgtctttcacatgatattcaacattacttatataggtatggatattttttagatgaacatagagattgtgattttcatttttttgttctcttctcccaaaatgtttggagtttaatgtacttagaagtacatctgggccatgacatggaacgttcatctgagctcctgtctagatatttgaagtgaatgctttcctttttctctaggtaattctttgaggtagttatatttttggacatactggaagctgcttttcttctgatttgtatcacaaactgtcctattcttttggaattatttaaaaggagcaacatatttttgaaatatttttgtactccagctttttgatgttcacctagaaaatagcccagatgccatcatgccataaagtattctggtacacacacacacacacacacacagacacagacacacacacacacacagagacatagacacacacgcagacacacacacacacacacacccctcatcaatctgagttcttcctgggcaggattatatctgtacatcctagtttcttgaattgccaaatagagttggctgccaagtaataataagtgtgcttttagaaagacgtcataaaatgttttgactgaaggaaatgtataaatgtataaattagaatggttaaccttagaagggattctcttttctttcaggttaagttatactaacatcatcttgaaaggcttctagaactgtcacagtttctatccctgaattccctgtttctgtcaggtattctgttattccatgctaagcccccgggtctctttgggctggttcttgccccaacaggacacctgatcctctgtcctgaaacttaaaagataattttatagtcttgaagatgttatgaagtcattatgatctctctacattaaagacatgtggtacatttagctttcctcatataaccctctatcctagacaatcttccaagcatgtttttccatttttcagtctcagtttttacccatctttgttcttttttacatattgaatccttcttgatgcccactgtaatacccattctattgccagtaatacttactgtatcctttaccttttctgaaaaccttcttctaccttttcactgtaacagaaggaacctgatactctcactggcacattatgtctctggacacctcaagtgattgacattcattttgtatgtggagtttacaattcattctctccagtgactgtttttctaactactgtcaatgtttatttatagatgacaatgcagaatatgctgagaggtttatggaggaaaatgaaggacatattgtagatattcatgacttttctttgggtagcagtccacatgtccgaaagcattttccagagacttggatttggctagacaccaacatggggtaaaaatttataaagttctttgcccatacatattttgtttagtgtttgttttaaataagctttgcccgctttctaatgtttaagtacaaacatagtgtaactaagaactaagtagaccaaaaggattttttaggaaatgatatttattgaatctaaatacagtttttgataaagccacacataaattatggcaggaaggtctcatcaatgagaagataggcctttttttttttttttaactgaagggtgattttgacttccttgaagtctcatgattcttgttgaagaaaaattgctgggagtacatttgttgtcacaggatgggaagcactcatgattacctcctgtgacccctggcagtgctgctaactgaaccctgctcctcacaaagcattcccaggagtcacagggagaaggggcatgggtggtggaaagaattcagcttggctgataaaccccgtaccacctggcctgataattgagcaggtaaatcatgaaatccacatagtattttatagtcagctgtttaaagatacttgagttaacacatgagtgaaatctcaaggaaacaaataacagcattgacagggatacagagaaaaacttctgcaaatttagagaaaaaattggagttaagtttgaaaatgtgtatttattatctataaaaaatttgtgaaaaaataatgtttattctgaagatgtaaattttgcaggaagattttattagaatatggatcaatatgcagtattatgaccttatgatgacctattctttgaaaagttgggatttactgttttatacttaaaccttttaaatggttttaaattcagatatgtaaacaataggaaaaattgaaattcttccaaaaatagtttagattattttggcttatttcaaaatgtatcagttcttggttttgtgatgtttatatttattatcttgacttcagttacaggatttaccaagaatttgaagtaactgtacctgattctatcacttcttgggtggctactggttttgtgatctctgaggacctgggtcttggactaacaactactccagtggaggtattgtattaaagagctgcttatcagtattacggtgacattaagctaatacagcgtcagctcctcaattttttttttaaatgactgcttataatgtttatcacagtttagagattccttggctttgtcttttggtttttatctgttttatatttaagaatgtgagctatatatagctatataaactgctaaatgtgcaaagtccgtattaagatttgggtagaaaagtttattattgacctgaactaaccatctccaaaggccagaagagagagaaagaaaaagagagagagagaaagaggagaggagagagagagtgagtctttctatttgtcctcttcaagaatgaacagaacttctcaagatgttccctagccaatattccatcatgtcttttggtcaaattgcatcatatattgtttcctaagccagtcactggcaggaggaatataatgaccatgagtggcctgaattttctcatttgaaattgaaatgtaattttgatttacaaaataatcgtattcatgaaaaatacagtgtagattgaaaaatgctttgggtttatatagaaattggaattagattgtaagctcaggccactataaacagacaattcagcaacatgaatgtctgaagggacattcaagaatcattaggaacatggggcaatttttcattgtctggggctgtcctgagtattgcagactgtcacccactaactacctatagcaccttcgagtcatggtgacaatctaagacaccttcacaaatgtgcagataaactctagagggagttactgctgccagcaaaaccactggcctaaactaacccaggtttagctttagatgcaggtgtggggcttggccttttctgtaggacttggccaacaatatcagaattgggtcactgaggaggaagcacatgtattcagatgtcccacacattttctcatctgtatgtaaaaataaatcatatatatgttttagaaataatttccaatttcctctttaaatttagtcaggaagcacatgtattcagatgtcccacactagaacaggggctgttggatttggcagggcttttaaagcagattggtggagtcaatacagcatgaaagaagagcaaattgcttcgggattagacaggctgggttctagttctggctcctctacttgccagcaatatgaatttgtactagttacaaaaatctcaaaaatttaattttcttttctataaactaggagactaacagtaaccttatggggttgtaataaccaaacaaaataatttatgtgaagtgtttggttgctataaggcacttaataaagtatagcaattattatgttaagtaacataaatcaagtcaatttgccgtcattcatttgtgataagttgctgtttgctttctgttgatagcaagttgacatttctagctgaagttaaaagcttcacaggttttataaagattgcatttaattgcataaaatgtgaagaattttgacctgaataaaaatatgtactcgttgtgttctttccagctccaagccttccaaccatttttcatttttttgaatcttccctactctgttatcagaggtgaagaatttgctttggaaataactatattcaattatttgaaagatgccactgaggtaatgtattcaagcttttgttgatcatttacactacaggagaaaatcgaggtagcatgaagggattgggttgtgtagtatctgggcatttgggattctaagcactttatacatttctgggaggctgaaaatagacatgtcttgtcagtgatttcttctgctaattaatgtctaaggatatcattagcatgtctacacattacctccttgaattttcagcataggttttcctcttttgccatgtaagttccttattgtctctgaagcatgcctttctctttcctgcctccctgcattttcccctggttttccccacatctagaatgtgtggccacttctcactgccactctgtgcccactgcagttttgttttttttttttttttgagacatggtcttgctctgttgcccaggctggagtgcagtggcgtgatctcggctcactgcaacttccacctcacaggttcaagtgattctcctgcctcagcctccgagtagctgggattacaagggtgcaccaccacgcccagctaatttttgtgtttttcatagagacagagtgttgccatgttggccaggcgggtcttgatcaagtgatctgcctgccttggcctcccaaagtactggcattacaggcatgagccactgcacccgactgccattctgttatgtttgtagcccccatgtttatattttcaaggattcactattggattcacgtaccttgtatctccttagctagtcatttccctgggaggcaggaattaggttcactgtttgtgttgtgcccacagtatgttaaccaagtgactgatttttcttcccccagaataagtagagatacgtaattaactttattacatggatgaaaatgacatgatatctttaggcactttatatataattttttttaatttaaattttaattttttaagctttttattggcatatttggctgacaaagtgggaagtaaatttttttaattttttaattttactttttaacccagttatcatggtaccatgtaacaagcaaaagatctagagtctaaaggcctggattcaatgtttaatctaccacttaccagcaaatgacttaattgtagtttcctcatctgtaaaatggaaatgataattattacacccacaaaagataccttgagaagcagatgaaaacatcaatgggaaaataattcgtaaaatttaaggcctagataaatgctattattgatgagacttgcaactttgggataatttacaaaagtttacaaccaagatatgcttcaggactaatatctgagttgtactttccttggaataattttttacagagacttcaataaattcaataacatttcatgctgttttcttcaaatttttctaatgagaccagtatgttccaggatgacatagttaaatcaagctagtagtaatatcaatatatgaaatagcctttctgtgcacatccttaccatctaatggcttcagatagtgagagaatttaaagttagtgggaggactagaaatggggctgaagatcgccctttccccatgtatcttgaggttgaggggaaagcatagaaaagaaatgaagtttttcctgatactctttgatactctccttggtcgaatctttgtctcatcttttcgctagtcttgagcctcacagatttgacagtacttaaactggtgataaaagagcactttactgaagagcatctattatacaaaagctttgatttatttttttctttcaggttaaggtaatcattgagaaaagtgacaaatttgatattctaatgacttcaaatgaaataaatgccacaggccaccagcagacccttctggttcccagtgaggatggggcaactgttctttttcccatcaggccaacacatctgggagaaattcctatcacagtcacagctctttcacccactgcttctgatgctgtcacccagatgattttagtaaaggtaaatatttgatgtctgaaagaagtgaaatggaaatacgtaattaaaaaggaagcagaaggttttttctcttggttaaatttgttggcatatctagtaggtcatcttctttacccttctggtaatgcctaatcactttctaatctgtatgtaaaaataaatcatttagaaataattgccaatttcctctttaaatttagtcaggaatgtgactataaagactaacgtctcttttgcagtcatttacatctacagactgtgttacgttgggatcctattaaaacacggccaagtcgatttgtagatttggaattttggaatttttcagtgaaggtaaaagagattttgtagatttttttcagagaagacattctagacagaaagataatgagcatagtggataaacacttgtattctggggtgaggcaaccctgtgtttgaatctcagctctgccaagtgtgattttgaacaaggcatttaatctccccctctttggataataataggaactacctcaaagggtttttttccttacattttcatcttcttctttgtcattttcaaggccagtccatgagatgccttatctaatttaactagaacttactaacagatttgaagtaagaataacaaaatggtacgcgctgtgtcatcagatttaattcatttccatggaaatggtgcctattaagtgtaaatcctgtgaaaaacacgtttcagttaatgaagggtactgtacttagtgagaaaatctaaaaggaaaaattaatcaaagttatggtaattttttgtaatacagactaacaagaaaaactcattctgtgaaatgaatgtattacttgtttaaataaaatatctaatttaaagaaaacaaaataagaaatgataggtgcctatttagccacacacaaacctcagacacaacaggtcagatgtttgtgctcatatctgcgtatagttctctgtaaacatgtgagtagagaccatgttaattgtgttcatttttttcaacaggctgaaggaatagaaaaatcatattcacaatccatcttattagacttgactgacaataggctacagagtaccctgaaaactttgagtttctcatttcctcctaatacagtgactggcagtgaaagagttcagatcactgcaattggtaagaatagagtatatcaccatctattggtttaattgtatatgatcatatatgttgttcttgtaattatagatgtattttcttattgagtcctaataagaagagatggcaaaagtttttaattgggcccaacaactcattataatatgaaggctagcaaattatgtatcctaaaactgttctcaatcttcaatgtgcataagaatcacctgggagtcttgtttaaaagcaaattctggtttagtaggtctggagtggggcctgggaatctgcttttctaacaagctcctaggtgttggtgatactctggcttgtagaccacactttgagtagcaaggcctcactatactgtggctggagggagggttcattcctaagagcagagcttctggcttctgtggctctccaccactggtgaatatcagaatcttctaaggagctctgtaaatacagaaacatctgggttctactctaaacctactaagtcagaatcacttgggctatggcttggtcatgtatatcttaaaaaactttcacaagtgattctgataaccaaaggttgagtactcctccctagggcagttctctgtgcaggatatgagtaacctttacagtaggaagttactcactgacttcggtcgcctctcctagttactttccataacataatagtacttttcaaaaattggcaatttataattgtataaatttatgggatacaaagtgatattatggcttatgaatgcaatgtggaataactaaatcaagctggttgacgtagtcatcacctcaaatacttaactgtttttttttgtgggaagaacatttgaaatttgctgtctgagcattttttttttttgaggcggagtctcgctctgtcgcccaggctggagtgcagtggcgcgaacttggctcactgcaagctctgcttcccaggttcacgccattctcctgccacagcctcccgagtagttgggactacaggcacccgccaccatgcctggctaattttttttttttgtattttttagtagagatggggtttcaccatgttagccaggatggtctccatctcctgacttcatgatccacccgcctcggcctcccaaagtgctgggattacaggcgtgagccaccgcgcccggccttttttttttttttttcctttgatggagtcttgctctgtctcccaggctagagtgcagtggcaggatcttggctcactgcaacctctgcctcttggggttcaagcgagtctcctgcctcagcctcctgagtagctggaattacaggcacgtgccaccacacccagctagtttttgtagttttagtagagatgggcttttgcagtgttggccaggctggtctcgaacccctgacccaaagtgctgggattacaggtgtgagccatggcactcagcctttcttagcaatttggaaatttacaatactcttgttattaactatatttaccatgctaaaagtcctttttttttttttgagatggagtctttctctgtagcccaggctggagtgcagtggtgtgatctcggtttactgcaaccaccgcctactgggtttaagcagttcttataccttggcctcctatgaagatgggactacaggtgtgtgccaccatgcccggctaattttttatattttcattagagacgggatttcaccatgttggccaggctggtctcaaacccctcacctcaggtgatccacccacctcagcctcccaaagtgccaggattacaggcatgagccattgcgcccagcctaaaagtactttttaattagacatagtgtctacacaaatggtgttattaatcagggttctccagagaaacagaaccaataggagatacttacatataaaaggaggtttattatgaggaattggctcatgcaattatggaggctgagaagtcccatgatctgcttttgcaagctggagacccaggaaggctggtagtactcactctgagtccaaaggcctgtgaagcagaggagctgatgatgtaaatcccagtcctaggagaagatgagatgagatgtctgagctcaagtagtgaggcaggaaaaaaagggtgaattcctccttcctcaagcttttgtctattcaggctgtcagtggattggatggtgcccacacacactggggaggacaatctgctggacccagtccaccaattcaaatgctaatgtcatctagaaacaccctcacagacactcagaagcaatgcttaatctaggcaccccttggctcacttaagttgacacacaaaattaaccctcacaaatggtttgtttatatagttgtgctttacaaataactgaagaaaacattttaattaatgacgtctaatagtttgttaggtctttaaggtgttaggacctaaaacacttttttacagttcttttatatttcactggcagatgtaatgcttttttattataacccaaacaaccaatccataggcagaggactttatttggaggcatatatatacatacatacatatatatatatatatatatatatatatatatatatatatacacacacacacatacatatatatatatatatatatatatatataatttttttttggaagaggcagggtttagctatgtttgccccaggctggacttgaacttcttgaacttctgagctcatgtagtcctcctgcatcagccccttgcataactgggactacaggcatgcaccactgtgcctggccaattcttaaaaggaattaaataagctctctatttaggggaataaattctgcttaagcatctcccacagtgttgggatgctgggacatgcactacacatacatacacacacacacacgcacacatatacacagagggaaggagcctggctttttaaatatgtaccatcactccttacacaggaacgattctgtctgtcgtcatggaaataccccgaggcttaatttttatgcttgatgtaaagaacggattttcaagttttatttttacatgttgttttaaagacctattttgtatcccagaatgtacgaattttagcttgcttctttgaactttgatagtctatgttcactttgaatattaaacacatagcctgcagtggtaataatataacctctatttttttctttgtattcacatctaaaatttaagctatactggcacagcttcagtctattcatatgaaacaggttcttttcccttttttttcatattcattttttaggatcacattttcactaggtatttggacttggcttggtataagttttcaaaataaactacatagaaagatactcttatctatacagcttaacaatggcagggccaaaattagaactcagccttcatatttctgaatttttgctactttccctgagttgtggtgttaggatgtgggtagagggtttttaaagcatgtgttttgtttaattttagattactaactttgaatccttttccgaatccttttgatcctgagattaattgaaatacttatatttgttgttactaaacagaacacattggaaaacactacaatcctcattgtttttttaaaaaattatttctcaagttcttgtggtgtattagtaagaaataagaataaaattttgtttgcgtgaagaatactactaataaaatgtttctttcctgtatgagtgatggtgtcttactgatcaattctgtacatttttttaaagatttttaaaaaaattattttttgcctaatttatgccacatgcttaactgagctaaaaatgtcctctttcatcacatgacctacaagttcagtaccagagcattgttatttttttgaccagtcttcctaaaagcttggtgtaaagttccaactctgttccaagcagtggaataatgatattgcttcaatgacagaggtcttctgtactcagtggaagtctctttgccaccagcaggtcgttgttactgagtatttactgaatgaactgcatgtcttgtgcttccaggagatgttcttggtccttccatcaatggcttagcctcattgattcggatgccttatggctgtggtgaacagaacatgataaattttgctccaaatatttacattttggattatctgactaaaaagaaacaactgacagataatttgaaagaaaaagctctttcatttatgaggcaaggtaagcattttagagacctacatttgttcgtagaaaaaaatttgtttttttcaggtagggttcattctagaccatatttcaaaatagatggattttgtcttaggtaagtgatgggctacacggtgttgacatgtgcaggaagtgcaaacaatggaaagctgttacaattcttcctttagctaactattcactcactgctgctaagatgtttgttagtaatggcttaagagttagtttctggtgggagggcttgtattatactaagggatctgaaatttaaaactctttaaaaatctagaggaatactcattcattttattacacatttgtggagtgtttcttagtgccaggatcagttttcgtgccaagaaaacagagggagccagggcctctcttcttaaagaattcgctgctcaatggagaagatggtttgtatgtaaacagacgcagtgccgtgtggtggggaccttctcagtgcatttaaaatgatgaggaggacctgctggaccctgaggaaatgtcatgctagtgaagactgtatcttgacaccttgaaaggtggaaactgttcacaggcaccagatcctttgttttggcaacttgttaataacagcagctaacatgagttgagtgtgcactgtgtggcaggcagtgttctaaacactttacatctcatacctcattggaggctcaccattagtattagcatgatcctgtttttttgcagatgaggaagctgaggtgcagaaaagttagctaacttgcccagggtcatatagtcatacagagcttggatttggactctgagcctatgctcttgacatccatacctaggaggaaacaaagagatgtgagacacgtttgatgttaatagtaaagtaaatttgattttctgaaatactccaaaagcttaccaaagttacagtctcagttttgtatttccttattagtggacctctagcacaaactctctgtggctgagtttttctctctgcacaatttgtatgatatatcccgtccatcaaattcatgaggattagaagggttcacacaaactgattaaaaatgaggattcactataatctaagagaaatatagttcaacgctttccaactcctaatttttttagtttttgtttttttataacaaatggattaaatgaaaagtaggggcaagctattaattgattgctaatttctccataggagaattagaaaactcagtaaagacgccattgtaatgtgagcttttcttccaggttccattattgtgtagagacctttttattctaaaataattctatgtaatgacagtaataacaactaaatctgtttaaatgttttatgcatgtttcatttaattctcacaaaaactctaggagcaggtgtcctattattatttccattttatagatgaggaaattgaggcacagagaggttaaagaatttacccatggtggtctggttccaccgtccaccttgttcctcagaacactatgggataattctgatttgcagtgttctctggtttttagaagcaatgggatatgatggaaaggaccaggaatcgggacgggatgcttgagtatcaccaccgtgtaagaagtctcactggaggttttgataaaatattaagggaggaacatggggtcctgtagagaaggaggcaccaaaggttagaatatggggtgtcagattagactgaagtaagctgtaaggtgttgggtttaagtagatgagctggagtcagtgtctgaagatctccctgggataggatggcacagacaaaggctgaatgggagtcaaggggttgcgggtcaggaacaatgcagtgtatgagatcacaaaccaagggagaccagcgtttagaatcagggctgagtgtggcctgagttctgatgtggggaaggacatagtggtatattttcccttagattagggccagaccatgcagacactatgaccggccctgaaatgcaccaggtggttaggtcctctgggatatttgtagagatccagcagttggaatttcctgtatagagttgcccatgtaaaattatccctgtttatacacatttgtggattggtcgtgttttaaatatatacgtacaagaataacctttgtcaaattttattataattaaatttaagtggtaccaagtggtctctgtggtatattgtgtgccatgctaaacacaaatgagatctttacacattggcataatggctgtaattaaggagataagtaaaaattatttgggattgtcatgaatgaacgttgtaccttttgaaaacaagattccaatttgtattcaagatttagtttttctggtttgaagtagaaactacataggaattagactggtgctttataatttggcatacattctttgggatagtttaatacaataaggtaaaatatttcagaaaatgccacatataactccatatttacatatgctttatgaactgtaactctggctaatcaaatattaatgctttaaacagaaagcataaattttacatagcttgaaacatgtaagcagtgaaaactcattccttaaaaataatttcagtgtgtgtcctagatacttttgctgtggatcagcagccatcctacaaaatgaatggtccaaatcctcagtatctgttgttttcccaaagcagtgctagtccctcaggtcagtcctggccaggtaggtggccaaactgtagtttttcaaagtaacccagaaagggtctctactgattgcctgagatccctcctcacatctgttggttgttagaaggtacagtggccagagtgcaaaaaaagcatcgttctctaaatgccaagcagtcctcccaagtgttaagaatgccgagcgctacagctgtaagtaggcgagtttccccttgggaggaagtagatcaggggcatagcactggctgtggaggcagtcacacctgggcttgagtcctggctcccaccttggaacaggttcttagtgtctccaaggcttggtttttttacctggaagacagggataataaaagaacccacttagtagggttgtttttaggagtcagtgagaataaaagaacccacgtggtagggttgttttgaggagtcagtgagatatgtcagcattttattcaacacgtagtaagtgcacaacaaatatcagctatagtagaattagttgcctctaactagggagttagaggttaatgtcagtgaataaatcctgtccattctccctctaaaaacctgtatgaaattcttctatttctctccatcccactgctaattgttccattcaagcatgattgctcactggactactccaatggcttcctgactagtctttttctgtgttccctccccttcagtacattctccatataatgtaaaagggatcttagaaaatgtcaagatcctgtcacttgcttgcttatgatcctgcagtgggctcccggcttcagcctacagggtcctgggtgatctggcttggcctccctccttagcctcatctggagctggctgtatctggaacactctgagctccatgctgcttcagggacatgatgctttcccttccttctcctggcatgctttctccctaggtctttccatggctgctccttttcatcattcagttgatgtttgcctcagagtggccttccctgaccacttagtaactccaatttgtactactccattttatttcttcacttaacattatttaaaattatcctgtccatcatctgtcctggcatttagagggggagctctgtgacagtggggaccatgtttgaccttcattgtatagctatgtgggcatctcaataaatccagaatgggcgaataacttattaccaatattaacttttttggactttaggtccatcatcctaatgtggatattgggtccagaagatctctgaagcgcccccacagtgtagcatgatgatctctgggcactaatgggtcgacctggccttggcattctcttagcagctaagaaatgtgaccagtgatttccatgtgctactctttttgctggtgaagtggctggtgaaagtcagcacagtggagctccccaaaaatacatcatctcacatgctgaactaaattatatttcaaagtagtggacgaaagcttcttggtataatgcttttcattatgaaatcagtgtctcatttcagagactctcttttttcttttgccatcacatttgccaaagtcctttaaatatactgtagttgtttttattttttaagaaattaaagtggcttcagcgtgagtcatgattatattatttaaatgtcatttatgaacagcgcattgttttacttaaatcatgcttggcccaggagttctgtaacctaacccattattttgttggtcgaaacaacagctacatcattgaatgaagtgtctttgctgtcaaaaaggaaaatggaaccataacatgaaaactgtttttgataaggactggaaatccagaacctgttaattgaagatttaaaccatttgttttcctcagatatttaaatcacagtgcctgcagttcaactttttaaagtgagactcaaaccaagtctaattgaaaacactatatttcctccattgaaataaaaataagagaaagaaagcttcagaattcggatgttgccattggtcacaagtatggctagtggccatagcttaaaaaactgtgatgttctcactttccatttcatgtcatagaaagtaaacaaattcataatgatttcttttaatacagagaattttttttatcagcttattaccagagaaaattcttgaaatagtcacaaatagccgactttacttcctttatttacatgtaattttgacacgaggtaaatcaagtcaaactatgtttagaatgttactttttcattgtttgtgagcagtatttttatctttattataataaattttgaaattttacattcatggttatcccaaagaaaattcataacccagaacaggatgtgtcttatctggccctttatgaagggctcatgggtttttatcttaagagcctctggacttcttctgcatttcaattttcagaagtctatgaaagttctgagcatatgattacttttcaaactcaagagccattcctaaaatgattaacttaaaggtgatactcttatttcttcataaacttttggagagagctatgtttttgcttttctcgtctgttgtgatgcagagtctccaacttagatgactgcactggaaggctgagttgttgtattaacaacaaacattatagacgagtcatcagtgtcctgtttttttttttttcttttttttgtgatggagtcttgctctgtcatccagtctggagtacagtggtacgatcttggctcactgcaacctccgcctcccaggttcaagcgattctcctgcttcagcctcccgagtagctgggactacaggtgtgtgccaccacacctggctaatttttgtatttttagtagagacgggactttgccgtattagccaggctggtttcaaactcctgtcctcaggtaatccacccgccttggcctcccaaagtgctgggattataggcatgagccactgtgcctggacctgttttgtttatgggatggaaattaggcctgatacactgggacgatttggaggacagtcgagggatccacaatgatgcatgctggtttaagatgaggaaggaaacctcacaggacactcccacaaggagacatacaagttctctccaggttgtgaatttctcccttacttttccatgggagcagagctggtggtttggattctcggtgttatctaagcctgctcaagaaattcactgcaaaactggtgattgtctgtggcctcttctctgggctggctgcctctagtggaagaatggtgctagttgttgatattaatggggaatgttggaggtcagccacctgaggggtaatttccataagcactgatctcatctccatgatctcatctctgaccagtttcacaatatctcatacttggaactgccgttgttgatgtgatctgggggtgtggaggcacatggtcctctccttgaggttggtctttctgtcacaaaagtacctcaaacttttgtgtagggattgagggcctcaaacctattttcttttttcttcttccctctcctctccttctcctctcttctttccttttcctcttttttcttaggttgaattcatagttaaactctggctatctggagaatttagtccttaattaaaaattgagggagaatgtcctggactctcttgctgatttcttttgtgaacattaattattttgcttctctgtgatccctggctgcctctgaaaaatgagataatatactgcatttttaaaggttatgtgaaaactaaaacatgataaaatgaactagagtagtgataatgcctatcatttaattttttccacttttgtttatgttttgaacatgtagaacattaacatgcttccaaaagacaaacttattttgagacatatattcaaaagagtgtctcttcttccccttttctttctccctacctctcattggtaactatatcaggcggttcttgcattactataaagaaatacccgagtctgggtaatttataaggaaagaggtttaattggctcatgattctgcaggctgtgcaggaagcatagcgccggcatttccccctaggaaggtctcaggaagcttacagttgtggcagaaggtgaactgggaacaggcacatcacatggtgaaaacaaggacaagagagacagtgttgtggggaggtgccacacacttttaaatgaccagatcttacagtaactcactcactatctcaaggacagcaccaagatccagtcacctcccactgggccttacctccaacattgaggattataattgaacatgagatttgtgtggggacaaatataccagtaacctacttcattagtttctagactatcttttctctctctccttttgcgaaaataaatgtatacatatatgttttctcatttccccccgcaattttttttttttttttaaagacagggtgtctccctttgcccaggctgaagtgcagtggcacaatcatggttcactgcagccttgacttactgggctcaggtgattctcccacttcagccttctgaacaggtgggactacaggcacatgccaccatgctaattttttttttgtatttttgtagagacaggcttttgtcatattgcccaggctggtctcaaactcctaggctcaagtgatccactcgccttggcctcccaaagtgctgaaccttggcaataggtgaaccttggcgcctcgcctcatttcccctttttattacacagaagatagtatatctatatgtactattttacactttgcctttttcacttaacagtgtatccaggaaattactctgcatcagttgaccgagatattcctcattctttttttcacagctgcatagtactctgtcatgtgtgtgtaccatagtttattcaatcagtctccagttttggggcactaagattgtttccattatgttacaattacagatgatgctgtaatgaataatcttctgcgtatatatttttgtattgttggaggtgtgtcttcagggtagattcctagaactgggattactgggtcagagggtaaagacattcccctacatggtagttgtaaccttctgaattccctctagcagtggatcagtgctggtttccatacagctcacccaacagaaggagtggtcagacttttgaatcttaatgctaatctgatgggagagaaatggtatctcagtgtaggtttaatttgagtttctcctgttatgaagttgaatgtgttttctatattaagatttgtttttatatttttgtcttttgtgacttatctgttttgccttttattcacttatttttatagggctttttatcctttaccctaatttttaaaaagttctttatatatgtatatacatgtaagtaataacatataaacatacataaaaattacataatattgatattagctatttatccatcttatatgttgcaaatatggtcttaatttatttgttaagccttgcttatagtgtgttttgccttggaatagtttaaaaactttaaaaaaatagtcaaatgcatcaatttttttattttattgcatctggtgcttagagagccttcctctacacctatattagagagtaatgcctattgtttgttgagagctttccatgtaaaaggtacttactgaagtcttttcttcttttatcatgtttaataattggaagagctctgtgatttactaggagacagtggccttggagccagtgagaagttggctaatgcccaacttcactccctgttagctgtgtgaccatgcacatggtatctgtatccgctgagtttcagtctcattatctgtgaaaaagggaataataatagcatctatgtcagagggtccttgtgagaattacctgagttaatcctgcaaaggactttgaacagttcctgacatctaatgagtgctgaatgaaagttggctaattactgcaccgtaataattcaattagaaacataaaggtataatctaatgttcccttcttcaaattaagcaaagttttgtcatcactttaaatgtttatatttcttttaccataagtcttaaatatcaccatgaaacagagtatcttgttttctttttttctttatgcattttgttttattgtaacttaaatcagatacttaaatctagggtttcttttggaaagactcagggagaagtttgggacttgaaggtgagaacacattaactgtaatttcaaaccattaatgcttatgtttagttagtcaaatatgaattttacttatagttctggatgcttctaaaatatggtatttataaagtcaaatttgtttattactgcagtttactggggaaggatttcaacatttcacaaatgagtatggttgccaagctttgccatgtctgtgctgcaaagctcaggctgtgtgcccttacctgctaatagtggaattgtgtaatttttagaaggatttttggctatcagagagtcattgataatgccttgagctggaaatgttcctgctaatggttgcttttttagtgataagttattgctatcttaactttatttttagcaagttagtttattgtgggttgtttggtgaatcctttagagtttggcctatgttttctcacactggactttgagatctgtatgcattgctttgcaaatatgaaacatctgctttttatttgcaatcatttatttgtaactatttgcaaatatgacacatttgcttttattaatgcagatacgtgagccttaaccccctacctaatttacttctgtaatgatacttcattgccaccactgagcatgtgtatatgtgtgtgtgtgtgtgtgtgtgtgtgtgtgtgtatatatatatatatatttttttttttttttttttttttagtggaaacatggaattggtgggtaggcaggcaagttggttttttggtttacctcagttggccatggccccaaaatttaagagccaccgttttagattattttgcatttcctcctctgtattgctggtctaagaatagaaaatacttttattttctactttggccttaaacttaaattaatgataccctacatagtatttttcttctgtagcaaacagtgctagatgactaagcattgaaacaattttgatgaacactagacagttaaataaaagtctgcctttaatttgcttgcttttgaatggctagaagcatcatctatgccatagtgatcttttaatttactttaatttgggatatcacaggcttttttttttttttttgagatggaattttatttgctcttgttgcctaggctggagtgcaatgacgcaaatctcggctcactgcaacctctgcttcctgggttcaagcgattctcctgcctcagcctcccgagtagctgggattacaggcatgtgccaccatgcccggctactttttgtatttttaatagagatggggtttcaccatgttggtcaagctggtctcgaagtcctgacttcaggcaatccacctgcttcggcctcccaaagcgctgggattataggtgtgagccactgcacccggctgggatatcacagtttttattttatgcattctgcttgaaaagtcctggcattttaaatttccaacagcacttaaaatagaaagtactcaatattagctcttggtttgatttcttctttccccctctctgcccatatttcctcaatatgaagtcataattagaaactttcatccattgcaagttaatgattactttgactatctgtctatttttgtgtctaggttaccagagagaacttctctatcagagggaagatggctctttcagtgcttttgggaattatgacccttctgggagcacttggtaagtgtttttgccaactgaacaaatccgtgtcatggaatgggctttcactaggtcacaatagccacgaaattgacagatatatctctatatattatagactgggaggttgctttttccttagtctggctttaaaaagagattttctttttaaagtattattttcaaacattaacaaatcaaatgtttttatttagaaaagataagttataaatcctcttttttgtgtgtgtgtgtgtgtggaagaaacagaaataaaaatactttatgatgatggtgcagctgataaaatcagttagaaatgccgggtgattgtgacacagcttttttcttaatcttccatgactaatacaagtgttgtgttactgcggtcaatgtagtttaagtttgtgtgtgtgtgtgtgtgtgtgtaaaaaacaaacactgatggaattaagtatgaacagccagtttcctccagactggaatctctatgtggctgattttggatgattaatctctcttcttgtacttatcttcaatttcctattttactcagaaaaagattgactcactgtatcaatcaggatagactaggagtgctgtggtaacaaccaacctactcaatctaagcaattaacaatgaagggatttcttgttcatgctgcctttccattgtggtcagcagggggctctgcagctgccaacgtgtccacgtctccaaaaggaggctccaggtttgccacagcccggaaagaggaggctggagtctatagcactggcaattagagacttcatcctgagtgtatcatttctactcattttatccttctcagagctggtcacatggctatgcctaacctcactggggcaggaaagtgaattcctctgtgtaaccaaagtaaaagagaactgtatattattgaatatcagtaatgtttaccacatgaaactttatataacatgaaacagatctgcttcaaattttctcttggccttggcctgactcaggatatagaaccataaatagaaagctttcagtgttgtattgcttgatgttctttttacctcagtatagtgttttgtagcagggactggcaaacttttctgtaaagggtcagattgtaagtatttcaggcattttgggccacgtagagtctccgttgcatgctcttccctcttcctccctctcctccctctttctttcctgtctttgtctttttcttttccttcttttaaaacccgctcttaggtggtggtgttggcaggtgggggctgggttggtgcacacataaacaggccctgggctgtatttggcttgagggccagatttgggccaggcagatttggcccacaggctgcagttggctaactcccattctataggagagaaaaatatcattttccccacctgaagtactacagtttgcttgtgaactatgtatgcattcttcacccacagctttggggctgactgaagcgtgttggagctcttctttggtaaatatctagaacagtccaaagatgaataaccttggacaatttatttaactctcttggactgtcagattctcagtctacaaaataatggagctggattcaacagacacattaaaaaatgctcatcatcactggccatcagagaaatgtaaatcaaaaccacaatgagataccatctcacaccagttagaatggcgatcattataaaggcaggaaacaacaggtgctggagaagttgtggagagataggaatacttttacactgttggtgggaccataaactagttcaaccattgtggaagacagtgtggtgattcctcaaggatctagaactagaaataccatttgacccagctgtggggaaaagaaagagagatcagattgttaccgtgtctgtgtagaaagaagtagacataggagactccattttgttctgtactaagaaaaattcttctgccttgagatgctgttaatctgtaaccttacccccaaccctgagctctctgaaacatgtgctgtgtcaactcagggttaaagaattaagtgctgtgctttagatatgcatacacataaacatctcaatgccttaaagagcagtattgctgcccgcatgtcccacctccagccctaaggcaggtttcccctatctcagtagatggaacatacaatcggattttataccgagacattccattgcccagggacgggcaggagacagatgccttcctcttatctcaactgcaaagaggtgttccttctgcttatactaatcctcctcagcacagaccctttacgggtgttgggctgggggacggtcaggtctttcccttcccacgaggccacatttcagactatcacatggggagaaaccttggacaatacctggctttcctaggcagaggtccctgcagccttccgcagtgtttgtgtccctgggtacttgagattagggagtggtgatgactcttaacgagcatgctgccttcaagcatctgtttaacaaggcacatcttacacagcccttaatccatttaaccctgagttgacacagcacatgtttcagagggcacggggttgggggtaaggttacagattaacagcatctcaaggcagaagaatttttcttagtacagaacaaaatggagtctcctatgtctacttctttctacacagacacagcaacaatctgatctctctttcttttccccacacccagccatcccattactgggtatatacccaaaggattataaatcatgctgctataaagacacatgcacacgtatgtttattgcggcactattcacaatagcaaagacttggaaccagcccaaatgtccatcaatgatagactggattaagaaaatgtggcacatatacaccatggaatactatgcagccataaaaaaggatgagttcatgtcctttgtagggacatggatgaagctggaaaccatcattctcagcaaactatcgcaaggacaaaaaaccaaacaccacatgttctcactcataggtggaaattgaacaatgagaacacttggacacaggaaggggaacattacacaccgggacctgttgtggggtggggggatgggggagggatagcattaggagacatacctaatgtaaatgacgcgttaatgggtgcagcacaccaacatggcacatgtacacatatgtaacaaacctgcacgttgtgcacatgtaccctagaacttaaagtataataaataaaaataaaataaaataaaattaaattaaaaaatggagctggattagatcatttctaaattccttttcaaatctgagatttttgagtcaacatggaatttcttggccaagcccaagtggaaaccagttctgcctgctagagagagttgcttaccttgtccctttgctctttcttttttgatttttttctttccttgttccatcttcagtgattccccctttgatgaatcttctgatgggggaaaaagtgttgttgttttgcttgctcggtgttatttggtggaccttataaagtgtattttatgtaatttttttctcttttcataaaggttgtcagcttttgttttaagatgtttccttgaagccgatccttacatagatattgatcagaatgtgttacacagaacatacacttggcttaaaggacatcagaaatccaacggtgaattttgggatccaggaagagtgattcatagtgagcttcaaggtggcaataaaagtccagtaacacttacagcctatattgtaacttctctcctgggatatagaaagtatcaggtatttcgtatttaatttaataaatgatagatgggaaattcaaggaaggtaggtcttaatgggtcaaatatgtgtgtggaaacttaacaagttgcagctttacaacacatgtgaaatctgaatttgagtactcttttgctttgcatttgtggccatgttccaaaatctgagaataaaacattaacccactctttcagaataactaagagaattctaaaaatgctttttaatgtatgtattgtacttgctattggtaagataagtcaatacatgttttatcattgaaaaagttaatttctgaggggaaagaaaattatttaaaatttgacaatgtgttctagcaagtttagattgcaaaggattttttacttataaaacatcatggaacagttacaatatctgttaatttagtgctgagaacacccataccctgaaaagtgtgttgaatgggcatggatgcctggatgagaatgagtatgcgtggaaaggtatgctgacgagcttaagtttgctccagagctgtcctggtgattgatgcctttactgcttactgcctgctgcattttatgagatcaccaaagtcattctctttattaggacccccactattttcctattctactagtaggcatgtagaagctaaacttttcctgaacttagggccgcactaccagagatcaaatataagaaatatatttctcaagcagtttgctgttcttatctgagtctgtgatactgagtgggagaaacactggcaaaagctctacttttttcttctagggaatgacacctgtttacttataaaaactgaaaaacagtattattttcaaaatgcatggcaaatctttaaatgtcaatgatcatggttgattttcattgcctagtacaaggtcaggtacttcatagacactgaacttattttttaaatgcatgtttcaaagtactttttgtttcagtatgtggtaatgggttactctgaatagtaaaaaacatactttttttcttccaagcctaacattgatgtgcaagagtctatccattttttggagtctgaattcagtagaggaatttcagacaattatactctagcccttataacttatgcattgtcatcagtggggagtcctaaagcgaaggaagctttgaatatgctgacttggagagcagaacaagaaggtaatgtgctgggcccacttgaggttgttatgctttatgaaatatataacttacatgagaaaaatttttagccaggtttgaaattgattacatctgcatcttttggtgaaaagtaaaacacataatgagatcaggatgggcctgacatggccacaatgcttctggtctccccagggtttttttaacagaatttcagggctcagatgtctctttattttagatatgtgatgccctatgtcagttaccctttgagtgtctgataccatttctggtttggcagccttgtagtttgctaagaccttgggttctggagagacaggttaaatcctggttctgcaacttacctttatgaggttcagttttcccaactgtaaagtggaaatggcacctattttagagtggctgtgaggattaaatgagataatgtgcaatgctgctgcatagcaaatgctcaaaaataggaactaaaatgctaattaaaagaaatgtgaataccaaaaaatgctgctagtaagtggattatgtgtattttatatacaaagtggacagggatccaggtgtgtgtgtgtgtgtgtgtgtgtgtgtgtgtgtgtgtgtaagaaagagagagatggaagtgggtgggcagaaataagataatgtccagtgattcaaatttctgttaattcaagaagggataattgtagaaagtgtttagaggcttctgagactgtaaaaaatattgtattaacagtaacttagattgaatccttgttacctattccaaagctggggactagaagattattgcttagaggctgaagcagtctttgacattttttcttcttgtctgcttggctaagtggtagttatttaatattccacctaagtgtagtcatcatctaaatagtaatatttgagagagggcaccaagtaggatggtcctctggggactagagcagaattatctctaggaaggttgttgagttagctggggatcctaattttttttcctgggggcttgaacctcaaactgttaacaccaaccttcagataatgaaaatgggagagtccagaatattagagctagttaatggcagattcccagaataggattcttttcaatgtgtcttaaaatattttggggatggcctggtcagataagtaacgtgcattatcttctgaaatatattattttttccttttatttatagctatttagtcttgagccaaatatagggaatttctcataggaaaactattttcatcaaaacatcctcaaatgtaggcaatgggaaaaggatgaaaatttacaagtatataggttgagcatccctaatccaaaaatctgaaatccaaaatgctccaatgagcatttcctttgagtatcatgtcagtgctcaaaaagtttcagatttttgagcatttcagattttggaatgtatgtatgctgtaaatattccaaaaattcaaacaaaacagaaattggacacacttctggccccaagctttttggataagatatacttaacttgtaatagtttaaaaaagcaacatataggcacatgtatacatatgtatgtgtgtgtgtgtttgtgataaataaatacatgtctggcataaataaatacatgtatacaagtatatattttttatgctagttaagtacaacttggaccagagtaggaatatttaatggtataaaatgtaatttttctggataattgatcagaatgttattacttttctcttgcaggtggcatgcaattctgggtgtcatcagagtccaaactttctgactcctggcagccacgctccctggatattgaagttgcagcctatgcactgctctcacacttcttacaatttcagacttctgagggaatcccaattatgaggtggctaagcaggcaaagaaatagcttgggtggttttgcatctactcaggtgagagatgatagttttttccctttaaactataatatataatatagatttatttattatatatattttatatataatatatagtatatattatataaatcatatgttatatataaaaaatatttttaaaagtatgtggttgcattttttccttgatttcataaaattacttggcagtttcaaagtttagtaacttaagcaaaaggctaaattgattaattttaatttttttctcctcattactatgaatttcatgtctgatatttatatggaataaatcagttccccatctgccacttttcatctgtggtaactgatataagagtagatgttcagttccatatagcaaatattttctattttcttttcttctttttggaaaggaaaaactgaaacttttaatttactgataatatgatcatttatgagcaaaattcagtgggatctacagaaatgctactaggattaacaagtgaatttagtaacattgtggtataaaaaaatcattgtacagaaatccattgtatctatatataagcaatggaaatgcaaatcaaaatagcataacatgtgaactgtttagggattaatctgacaaaaatcaataagatctatacactaaaaactataaaacatttcagcaaacattttctgagcctctcctgtactctaggccaaggacattcaaatatgaatcagggagcattccactctgaaggaaggtgtatgaatatccagcaacaaaatactactaaatttactctttgaagaccttgatatatacttatatgtacaaatgtttttcttccctcaacaggataccactgtggctttaaaggctctgtctgaatttgcagccctaatgaatacagaaaggacaaatatccaagtgaccgtgacggggcctagctcaccaagtcctgtaaagtttctgattgacacacacaaccgcttactccttcagacagcagaggtgtgggcaaggggcagttatttaaaaatcagtgtagacaattctttatgctggaatactgctttatttgtaatctgttgatttcaacaaagacttgtttccagagctctgtagagtaatagggagaagtggtgcccttctttggctgaatttgctgagctctggaagggtggggaactttggaaagattggtggaacaggagaaaacacgggcactgtaacattgggaaaggaatgaacatagaaacctgttgagtttctattcttcataggcactgtgctagattactttacgtatgctattgcatttacttctcaaagtaactctaagaagtaagtacatctatccccatcatacatatgataaaactgaggctgagagaggttaagtgacttgctcaagatcatgttactagtaacagtagagctaggatttgaactcatcattccaaagaccatgttcttttcagctgctccacatgtcttcttaggagaattgatagaaccggtagtagaattcttactttaactggaataaaagaatatgctagttcttaactttattcccttgatcagttttgtgggaggttaagtacaatggatgaaggtgataacttcaattctggaaagtgatgaagagtggtgatgataaaggaaaagaaagaataaagttaggcacagtcatagacaatgtgttattgtgttcctgggagagccaggggattaggccctgattaagggaggtcaggggagggagatgactttagtttcttgtcttttatgcttcagccccaacaggaggacagacttgtcagtagcactaatgtgccctcccatgggcagaggtgagggagcttggggagtgtgcacttttcctttcatcaccttgcttacctatttgacccatagcttttgatatgtgagacagtgcaaagctcttagtgctgtcagtgattgcttcttttcctaatgagggataggactgatctgttttgctacttggaaggatctagatatggaaaattagcctcattcactttttttcaccttgattcagcttgctgtggtacagccaacggcagttaatatttccgcaaatggttttggatttgctatttgtcaggtatgtaacgatgcttatttttttaagttaaatatgactttttataataattatttggtttggggctttattaaatcttagataacttgaatataattcctaatgatttacatctgtgacttaagcaagatatatcactgtctttaataaaaagtattagagatgttgccagccaatgagtagagagcaattatatagattgattttctgtttgaaaagtatttcttggagattatttgctttttgaagtgagggttaatgtcctccaatatgattcctgataacaaatacatgttttacttataatttcttaatattcatctgagaatatagaagtcaacatggtagaattattgacttgaggggaggaattgtgtccctttaatgtaaaatcttatataatagtaaatataacaattaaagcataattatgctcaatcctctggtacagaaagaaaatataaagaaatctacatttttatagtgcagcaggagctacaataaaatggaagtagtatgaaaaatactgaataagataatcatcacactaaatagcacataaaaatattgaggactttaagatcatctcattagaacccaaagataatttgtgataggaccaaaaatggttgtgtgcttgggtagaaacaaacgaccaaacaaaaacactgaaacacttactgagaaagtaaaaaaatggctattatggagaaaaagtacttttttcaaaaaccagaacaaccattctagtagagagaacaggtatacttgctgagtagggcgggtataccttgctaagcaggtataatttagaaacttccaaataattctaagattattttgcattattctcagaatacagtaaacattttttaaaactaaaggaaaaatgatcacaatgaaaaggaatggtattcagtgaaaaggtcagaaagagaaagtaaacaatttatttcgatctttactaacaaagaccatgggaaaattctcattctcaagctgtcttgcaatccaagagtaaatccagaagtaaatatgctgtgtattctagataaaattgaaaaattatatgtacttaaatcaccaagttggttggaattcatttttgataaaccaaatgtagagaaattagtgaaaccaagtgattttttacttgaggcttgaagcacacaggggtaaatgtggtacctgttggtcagaatgttcatcaatcccatgttggtctcaaaatgcagaaaacaaaaggaagggactagtaggcacctctcatgagggaggaaaaaaattacaaagaagttggtcactcagaaatgggtattgagatttcttacagttgatatttttataagtgctctggaacaaggaaggcaaagaaaactcatgagtttgcagtaaatattaccctcttttgggtaataaaatactaaactgttggaaataaactgcaagatcttttgaagttccctgagtgagcagttgagctttattgccaaatgtaaaccctaatacttagttatggatgggcattaattatgacccaagagacagagtagtccttgtttgtttcctgatagcattggtctctaaagggacaattgcatgccaccgaaaggggaatgatggaaataatagaaagcatttctccaattttatgggactgttcttcagctgcttcaggaatactctataatttaatcattatactttaagaaagagttgacaaatgtccaaagtaagataaccagaaggatcgagtggatgaaggattatatcagtttggattgtggcagaataagaagattgcaacttttagatttgaaaaatgaaggcttagaggatttttttaagtatgcaaatccatgaggggtattagatgttagttctgaggggtgtgtgtgtatgtgtgtgcacgtgtgcatatgtgtatgtgttttaggtataagttaaaagaaaacacaaccttacaaagcaacagtaagctttatgaaccttgttataaaaaaggggatgaggtaaaacatttaaatagattccagtccatctggagatgagattggactaggaagttttgagtacattcctaagcttctgatttaggatccctttgagaaagacacgggtcctcaggctctcccagtgtgtctacggcacctttaacatcagtggtgggtacagagtagatgctgagaaagacccagttgacttgaaagctatcatttattttatcatctttgacaaacattgtatgaacaaaaataatcctcagcaatgttcaactaaggctcttcaatgttttagttttgatttatcctactgagagtttttgaagttctagttagagaaaaatgactccatttgggcaacaggccttagaaattagtgaatgctgtcaagaaagttttttcctcaagggaatgtttgccctctttggcaaaccagtatccccaaatatcacccatagtgttttattgtacttgttttggaatattacttaacagttttctccatttaaacataattttataaattttaatactatttgttaatagtgatactttgtgagcaagttaaattaatttttttctagtttgaagattaaaaatcttactgggtccatccaaagagaatcattacctaatacagtatttagaaggcaaaatgatggaaatttatgtccaacttgttatttatgctagtttattttttacagctcaatgttgtatataatgtgaaggcttctgggtcttctagaagacgaagatctatccaaaatcaagaagcctttgatttagatgttgctgtaaaagaaaataaagatgatctcaatcatgtggatttgaatgtgtgtacaaggtaagtgtctgcttaggtctctcttctttttttcctttaaaaaatagacttgaaggtttaattatgtatagttgtctatatcaatctaagagttatattgaacaaagaattcagttatgcactacacttcagattacaaactagaaatactactaattatattcaagcatttattagatgcatacaaaaattatttacaagttttccctggacgtataacacatttgaatagaacagtgtatatataaattaataaaactcaggtctaatttaggaaaactttaaattaagtcaatcatatttaatacttcaaaaatggttatttcttgttatgagttatttttctgtagaaataattatttcgggttcatattgaaccttaactgttaggcatttaacccaaaactttatgataacatgcaaattcaatcaagaggatagtttttttttaggtacattgaaaatattccagtttaggagtttctcatcttggataattagacacttaacagctgaagcttctgggagtactttttaagttgactcttaaattgttctatatccaaagcatctgagaatcttgttaataaggaaaaagggttctacctcaaccctaccgacttcacagtaagactgggaagctgcagatttttggtcattccactgcacacaggagtttgagcacactccttgtgaaagttcttgaatagaggcttcacttttttttctttcatgaatctattctcttacatctgcattatttggtattgagctgggacttaaagatgcactggcattgaggagctgctgtttgctcttctaaaatatcccatagtgtcctggtttaacttagcttattgttgggctagaagtccctgatgtggtcctgagctggcagatgttcttcaaggtcatctctgggtgttcatccaggtggccagccttcacagctattgcctggacccagtctccgccattactgctactgtctgtctttatctttggaccttcttgttcatgattttggggtaaaatgtcccacatttgtcccattcttcaccttcttgggcattttgggccagcagcaaggggatatcagaatctctgtgttggcatccttttcagctgagaaaaaagtaggactccaattattttttacgaatcttccaattggtctctgttatagaataacaacttcaagttattactattattataaacaataacaacctacagtaattacaaacttatatgccaggcacggcaccaaacacttttacatgcatatcatttaattcttacaggaactctgtaaagtatgtgttattattatccctgcttttctcaaattcctggcctcaagtgatcctcctgcgttggcctcccaaagtgctgggcttacaggcatgagccactgtgccccacctattcttgcttttctcattcatttatttattacctcagcaagtattgcttgagtctctactatgtgccaggcatggtgtctgtgtgtcaggcgtgggaatataattgtaagccaaaaaagatgcaagctctgccttgtggaacttgctgtttgagggggatacagaaattaatggaaacaatcccctaagcaagcctaaaattctaattttgaaaagggcaaagaagaagaccctcatggagttaagagagggagatttttacctcttaagagacatcaagtgaagaaacatgagaaaaatgaggtggggaaccagaattcagactgtgaagaggcctcagcatttataggggttcatgtaaggatgcaggatttccagttttaagcatcaggaatcaggtggacaacagagtttgggtggtaggtagagcctgaagaagggaaatgttttcatgtgagggtaggggttttataaaaaggatttaaatgctgctactagttttctgagaaaaatctggtatgaaggacaaataacatttaggtgtattttttcagcagcacattataaaaagattacttgatatttcagacactttattcctatcaacagggtccaagaccttacctcataccaggctttctgtgtggcatgatagtttaacaaattctgccccaggcttggtagagagatcagatcaaccaaggtacttttaatatgtactgaaagtataatctgcattgtgcttttaaatgtttaagcatattgactgtttttttctcatctatatttttttctcatatgcaaacacctccagtttcaatccatttattagttctcaataacatctattttggctcagtgcattgccagcagatggtttcagttgcttcggattgcagagccagctgaagtcagcagttgtactgtatggcttttcaatcagtcatgtttggcaggcatgggttttctgctctaaaaagcaagatttttctaacagcccttctttcttttgccatttaaaaggacaacaggtactaacttaatgctatgattcttttaaattggaggcaaacatctttccataatcacttagctgggctccttctgctctaaatgaaaagctcttgccatcaattcattttataaaaatatttatttccaagcagacaaaatgttgttggtgcccagatcctttgatgctcacatcacgtacaacagggcgagagaaggatgcagtgtaataaaactttccaaggcttgtaattccctcacggtggccaaagaaaacagaaaatgttcttcaagtatatgggagaacaaagggatggcttctccctactggtctgatgtttctaaaatcagacgaataaaaggttgctaagaccttggatgggacctatcactaagtggtcaagtgatgcctccttctcagggcctggctttcagcttgcaagtttggtgctgtcaacttttgagaaaaggaaaagtgcaattctgtttggaatcaccactagaaaatagagttgtctgttgaaaggatgtatattgtactgtcacagtttataagcacaagcctcttacattgcaggcattgcatactatctgtcagatgaacacattgttgtatcattattccttatatggttaaaagtttcaagattaccacacatgtaagaaaatgataacctgttttaaaattctctatagggaattttatttgaactcaaacagtgtttggaataacatttggtgggtttgatttttgtatgaagataatttgataacagctatgggtttttcctgaggagttttcattcatcctccctctttgatttagcttttcgggcccgggtaggagtggcatggctcttatggaagttaacctattaagtggctttatggtgccttcagaagcaatttctctgagcgagacagtgaagaaagtggaatatgatcatggaaaactcaacctctatttagattctgtaagtagtaaaacataaggtaactgttgacaaagccactgtgttttgtattcaggtgtctaccttactttaagtatgttttctttaattcattgttatttcaactttttgtgtccctgaatgagtttaacatggcaaccatatatttatctccattagaaagacataagtataaaatcagtttagagagttgctattaatgacgagcctgatgaaggccttcataactaaatctttatgtcatgccagatgtgaaatacatagaaatttccatagtataattaaaaaaattcaacccagatctgaatttgagagcctgtctgcctcagggagttgcctaggctggagtgcagtggtgcaatcgtagctcactgcaacctccaactcctgggctcaagtgatcctcccactgcaggctcctgagtagctgggaccacaggtgcatgccaccatgcctagataatttcatgagaattttttgtggagatagggtctcattttgttgcccaggctggttttgaactcctgacttcaagtgatcttctggtcttggcttctcaaagtgttgggattacaggtgtgagccgccatgcctggctggaagacgaaagtcttaattacacattttaaaattcttccatgaagcttttaacaaaccttaaaggggttcatattcttaccttgaaaaattaggtgctaaatcatggaagactattctcagagatttgatcttgacatagaattgttacataaccttcgtgtactctttccattgctgaatgagtactactgattcaagaataaagtgcacacagtgtgtagggaaaggtaagtgaagaattccagactctacttggtatgtctcaaaactggtggtatgtaagtttattcttttcttatagcatctatatgagcttcataggttgaactgatattgagttcatagtattctaaggatttcatattacatgggtgggtttgtatgtttcttatttcagttttgctaagtatgaatcagaaatatattgctgaatatatgcttttaaaaagatagttcttgaatggtctcatttgttttagcacttcaaaaccactttggttaaagccctgttaaaactaattcagtctcaatataatgttaaataacagtctctagattcttttgctgtttaggaattatcttacattaagcactgctcaaaagccattggcctgatttggaacttgaaaaacaaatttaaggttttagtttagttgtgtaattggttttggagtttgtcttctatctttgatgctcttgttaatggttattttaatttgagtgtccagtactctttgagtggtcagtttgacttttcatttactactattggcaaataagggaatgtttgaaggaatttactaatttctgattctttaatgagtgttgttgccaaattggccaaatctgtctctcttggagacagataggctggctttgtgtaccccatctcagtcattggctgtggagtacagccccatcttagttacctcacctactgggtgacatggcttgcatgagctgagggtgattcccaggagaaggaggctgctgtgagccattagtagtaactgggggtggggtgcaccatctgagacaggagatctaggtgcatgcaccctaaggcttgctgcttacttaaccactctaattctcagtatcccattcagcaaaatggaactaagaacaatacctttttgtaagcattgttttacaagttaaataagatagttcataggaagtacttactgcagtatctgacatgtagaaatcctccctaagtgttagtctctgtttcctaaaatgatgagaaaaaagaaatgtctttacttaatcatgaacacacagtgtgccaaccccttaagactcttttgtatttctcaaggtaaatgaaacccagttttgtgttaatattcctgctgtgagaaactttaaagtttcaaatacccaagatgcttcagtgtccatagtggattactatgagccaagtaagtatgctctggagttcttaatactttagaaattaagccaggcattcattcatttattggaagtgaccacacattggattggttctgtggcgagggcaggatttggtaggagaaagtacaggaggcgggggggcaggaagtgagcattccatttcagtgaagactattgataatttacaagggtcacatgataggttttagcatatgaaatttaaaaatgatatattttttcataatgttttagatttttaactcacaattataaactttacaaagaaaaattggcatatagagtccagttatgtctcatttgcaaggtattgcttctattccaagcacccattccatctgtttccagctttaaaatgggagttcttaatgtggcacatatacaccatggaatactatgcagctataaaaaaggatgagttcatgtcctttgtagggacatggatgaagctggaaaccattctcagcaaactatcgcaaggacaaaaaaccaaatgctgcatgttcgcactcataggtgggaattgaccaatgagaacacttggacacagagaggggatcatcacataccagggcctgtcatggggttgggggaggagggagggatagcattaggagttatacctaatgtaaatgacgagttaacgggtgcagcacaccaacatggcacatgtatacatatgtaacaaacctgcacgttgtgcacatgtaccctagcacttaaagtataataaaaaaaatgggagttcttctaaaagtagagctaccatttgaccccacaatcccgttactggatatatacccaaaggacaataaattgttctaccaaaaagccacctgcactcatatgtttatcacagcactaattcacaatagcaaagacaggtcatcgacctaggtgcccatcaactgtgaattggataaataaaatgtggtagatgtacatcatggaatactatacagccataaaaaagaactaaattatgtcttttgcagcaacatggatgcagctgaaggtcattattctaagtgaattaatgaagaaacagaaaaccaaatatcacatgttctcacttataagtgggagctgaacattgggtacacatggacataaaaatgggaacagtagacactggggacttcaaaagtggggagggagagggggaataagggctgaaacactctctattgggtacaatgttcattatttgggtgacaggatcaatagaagcccaaacctcagcatcacataatatacccttgtaacaaacttgtacatgtacccctgaacagtaaataaataagtaacaaataataaataaaatgggacttcttcaggagagaacatgaacagcatctgcaatgcagtagcatcggctggagttagctgtttctttattgagggagtctggtgttttgccgaaacttacacttgataacagatttttaaaagtgtgtcaagaaaaatatagcctgtttggaaaataacttgagcttctttgattttactggtaacttcttatttgtataaaatgatggaaatttaggacgtaaaaatagagagctggtctggagatggcccaagtttaataggatgaatccatgttcagtcagagggactttttgtatgtgatctctggcctaatgtttctaattgtaatatgtcttattttgaataagcgcttgtggtggctcagaatcatcacaatgcatgagaagcatgaacatttttgtgtgttaggtagttgtagataattacactttaaatgagatgaacttgttcccattgttgttagtgtttcatagagtattcaagactaaggtgtagcaccagttctctcctcatagtcaacaagccagttcctcttgtcttttgcctcctgttggcactgaggaagagggttcacccaggttcttgtgggacatttgggaaacactggtgtggcactttttttctagtggagggatggaatacactgtggagacccaatgacagtaactagttgtccaactttattactttacttagttgcaaaattccccaatttatatttcttgggtgcatggggaacattttaaacatttaagaaacaaaaataattcagtctatgcaaatttttatggaaaacaacttacttatgaggaggccattttaaaatgaaaatatgggaaagatcagaataagctaaaccttccaacccattttaaaaaatctcaaaacacatttacgatatggaaaacagtctctgaaaaatcatgaataaatttgaaaatagaatatgttttttaaaaattataaaactcatttatgttttacagtgagttcactgtggtgttataggtactttaagtagtttttgtcatgttcgcttttagatactatatgctgagtgttttaaaacaaggtattgcataggcagtcttcccttcatatttatgtatttttttactagagcccagggtaaatgccccttttcagtagttctaatgattagaatttgatttgagtgcatagagaatgtcatcctaaaataaactcttgaggaggttaaacagtccttaactgaaaattctccctgatgcagtaagagattaaagtggtgcttgtgattgcagtgtgcagctgcagtctatttgctctcttttaatgctggccaactgctggtggtagacagattggactgagcgcattgtttctctcttggatttggttagtactttggaccactcttggacatttcagttgtttcttgtaaagaaaaataaaattaggttaagaatggaaaactcagaaaagtgtatcgaaatgctcttatattttggcaaagtcaatgtttctaaacaagggagccgtgtgaactgatgtctgcttctttgaacagggagacaggcggtgagaagttacaactctgaagtgaagctgtcctcctgtgacctttgcagtgatgtccagggctgccgtccttgtgaggatggagcttcaggctcccatcatcactcttcagtcatttttattttctgtttcaagcttctgtactttatggaactttggctgtgatttatttttaaaggactctgtgtaacactaacatttccagtagtcacatgtgattgttttgttttcgtagaagaatactgcttctattttgaaaaaagagttttttttctttctatggggttgcagggatggtgtacaacaggtcctagcatgtatagctgcatagatttcttcacctgatctttgtgtggaagatcagaatgaatgcagttgtgtgtctatattttcccctctcaaaatcttttagaatttttttggaggtgtttgttttctccagaataaaggtattactttagaataggtattctcctcattttgtgaaagaaatgaacctagattcttaagcattattacacatccatgtttgcttaaagatggatttccctgggaatgggagaaaacagccagcaggaggagcttcatctgttcccttcccacctccaacctagccctactgcccaccccaccccaacccaccccatgcccagtggtctcagtagatacttcttaactggaaattctttcttttcagaatctaggtggtgaattttttttaagtggcacggtctttttctgcttgaaatctgatcacaccccccagccattgccctccctctctttttcctctgtagagaaatgtgaggggcagtacatttactgtgcttttcacaccatctcagaggttgaggagcatactgaaaattgccctggggggtgctgggtgtgctgtctccttcccacatcctcagccccacaccagctctatttcaggggtgagagtcagagagcactgcaatatgtgcttcatgggatttcgattcgaagatcctagaccagggagacactgtgagccagggatacaacaaaatactaggtaagtcactgcagaccgacctccctgcagtttgggaaagaagctgggtttgtggagaatcagagcatcttgacatgactgctgacctaaagatccctggcattggccagggatcctgtggaacctcttctagttcaggggtgtgagcattagactgccagttgtctagtgacatctgatgcttgctgtgaacttttaagatccccgaatcctgagcacctcaatctttaattgccctgtattccgaagggtaatataatttatctggatggaaattttaaagatgaatcccccttttttcttttcttctctcttttctttccttctccctttcttctttgccttctaaatatactgaaatgatttagatatgtgtcaacaattaatgatcttttattcaatctaagaaatggtttagtttttctctttagctctatggcatttcactcaagtggacaggggaaaaagtaattgccatgggctccaaagaatttgctttatgtttttagctatttaaaaataaatccatcaaaaataaagtatgcaaatgtatcttttaaagttaatttttaaaaatgctcttattttagtgaattttcagaaattatagtggaatggatgctcatatattgcttatggatattttggataccaaagtaggaataactgacattcagtattttaaagctggcaaacctgtacatagaaaatagatccccagacagtggtctatgaagagggcagttaagtatcaaatacttaattttcttgcctttttttcttaagtggggaaaagtttctagatctcttacacctctgacacaatctgttctaaaacaggcacttgtaatgttggggcctccttgtaaacgtgtttttgccctttactctctgggagttctttaaaggtgaaatcatcttacaaagaaattgggggagggtcttggcaaaggactttcccctcctctttcctggcctgggaaccttatactgacaatcaatactttatattttaaagtatataatttatagttaacttctagtgtaatatattaggaaacactagaatggaaaggccattggaagacaggttgtatcttttttagaccatatttccttgtttaaaaactatcatttgaatacttttttggtgaagaactccatgttttcaagttaaaggtcacctcgtaggccaggcgcagtggctcatgcctgtaatcccagcactctgggaggctgaggcgggtgaatcacaaggttaggagtttgagaccagcctggccaatatggtgaaaccccgtccctactaaaaatacaaaatttagccaggcgtggtggcatgcacctgtagtcccacctactcgggaggctgaggcaggagaatcacttgaacctgagagacagaggttgcagtgagccgagatcacgccactgcactccagcctgggggacagagtgagattctgtctcaaaaaacaaaaaacaaaaaagtcaccttgtaactcatctctttttattgtaagtttattaaaaatgaagaggacaacaatgagaaggaacataaagggttagctagcactgtctcctggtgcatggggctgtgcagatgtcccggccacttcttccttcatacttcccttagagaacttgctctgctacaagcagtgggcttggactaaaagtgattaaaataccacaggcataaggagaaaaggagtatatgtagtagtaataattactagtataaattattttcttcacatgctatgagtaataatattaaaaaactcattttaccattaagattccttatgctgaagctcttccatttagaatactgtcaatgtcatttactggtatgaactaaagtcccccttcttttccactcactgggaaccttagtaaaacaccagcatatcttacctctctttctgactggccgatgcttccagagactgaatgttgggaaaacctagtagccaaacaattctaggacagaataacatttttatatttggttccaccatcttattacatttagttatagttttaaaaaagaaattcaagcccattaaaatatgtctggtcaatgaaatgcttccttttattgtgttgtgctattgtactttgtttttcaaaacattgtaaaaatagtatctttggtttagtattttggattatatattataatctgaggagtgttttgcttatgtagaatccagatatatttctgttacctaggagatgttacttacatatgtaatactgtatcctgcacgtggaaatattcagaattgtagatagcataactctccctgctcctattcttttgagcctaggtataattttttttttttttttagaaaaagacatatttagctttaatttctatttatgctaaacatatttataagtagtctgtcaatataataccaactatttttatttttacataattcaattatttcatttgacatgtctggcagactcaagacattaagtaaaaaattggaactatgatttttctttgtcattttttaaaaaagaattattttattaacctgctggcatataatctggagttcttttcacaaccttactttttctgatttgctttattgaatgattgaatactcatttctttctaaaaatatgttgtaaattctcccttggcaagatttctccctatgagggtagttattatttgagtctgccaagtggttaccatggggcaaggtgccatgatgtattcttgggtgcattggttttttgcgcattgtaaatttaagacacttatagtaagtggactcattcatagatgagtttcagaaccttttacgttctcggtagaggcttctgtcggacaggcagaagagtgtattcctcacttttttttttgtcttcaaattccagtaaggcatagcacttttaagaaattagaatttttctatcatctatgcaaatgatatttatgttaatattaaatatcttatgttacactgggagtaatttgaggtgcaattatttttattactactttgaatagaggaccattatccttctttcttcagaaaactaagaagtaagtgtaacttttaaagtaagtatatatcagtgagagtaggcttgttttacaactatttctagccagtgagttgtgttttcatgtctcatcaaaagacaataccacattgcatcattttacaaaatatgttgtcattttcatttcagttgtaacataggaaaatagatatttcctagatgatttctgagtttcttactgcaaagaacagttataaattggtatacatgtgtctctgtaatagggataatattgatatatctgttgctacatatttaagaatcattctatcttatgttgtcttgaggccaagatttaccacgtttgcccagtgtattgaattggtggtagaaggtagttccatgttccatttgtagatctttaagattttatctttgataactttaatagaatgtggctcagttctggtccttcaagcctgtatggtttggattttcagtaggggacagttgatgtggagtcaatctctttggtacacaggaagctttataaaatttcattcacgaatctcttattttgggaagctgttttgcatatgagaagaacactgttgaaataaggaactaaagctttatatattgatcaaggtgattctgaaagttttaatttttaatgttgtaatgttatgttattgttaattgtactttattatgtattcaatagaaaatcatgatttattaataaaagcttaaattctcatctattt

SEQ ID NO: 2 - RNA, 9401 nt, Homo sapiens
  source 1..9401: mol_type = mRNA
atagttgctatgaatttctcctcacaaggatgggcctgtttactcaccctgggcatcgttggtagagcgctagtgtaaacagcctgaggaacccggtgggccggggaagtgggcgcgctctgttctccgcggccagctgggacgccgggccaggtggggccgcctgcgtttagcaactgctttctcaccccctggatttgcgatgtttgccacagcagcgagaagcgccattgtaatggggatgggaggggtggagcctccaagtcctgtctcaatttagatctctcactctgctgttaggcgcgcccatttcagattactaaactcgaattaagagggaaaaaaaatcagggaggaggtggcaagccacaccccacggtgcccgcgaacttccccggcagcggactgtagcccaggcagacgccgtcgagatgcagggcccaccgctcctgaccgccgcccacctcctctgcgtgtgcaccgccgcgctggccgtggctcccgggcctcggtttctggtgacagccccagggatcatcaggcccggaggaaatgtgactattggggtggagcttctggaacactgcccttcacaggtgactgtgaaggcggagctgctcaagacagcatcaaacctcactgtctctgtcctggaagcagaaggagtctttgaaaaaggctcttttaagacacttactcttccatcactacctctgaacagtgcagatgagatttatgagctacgtgtaaccggacgtacccaggatgagattttattctctaatagtacccgcttatcatttgagaccaagagaatatctgtcttcattcaaacagacaaggccttatacaagccaaagcaagaagtgaagtttcgcattgttacactcttctcagattttaagccttacaaaacctctttaaacattctcattaaggaccccaaatcaaatttgatccaacagtggttgtcacaacaaagtgatcttggagtcatttccaaaacttttcagctatcttcccatccaatacttggtgactggtctattcaagttcaagtgaatgaccagacatactatcaatcatttcaggtttcagaatatgtattaccaaaatttgaagtgactttgcagacaccattatattgttctatgaattctaagcatttaaatggtaccatcacggcaaagtatacatatgggaagccagtgaaaggagacgtaacgcttacatttttacctttatccttttggggaaagaagaaaaatattacaaaaacatttaagataaatggatctgcaaacttctcttttaatgatgaagagatgaaaaatgtaatggattcttcaaatggactttctgaatacctggatctatcttcccctggaccagtagaaattttaaccacagtgacagaatcagttacaggtatttcaagaaatgtaagcactaatgtgttcttcaagcaacatgattacatcattgagttttttgattatactactgtcttgaagccatctctcaacttcacagccactgtgaaggtaactcgtgctgatggcaaccaactgactcttgaagaaagaagaaataatgtagtcataacagtgacacagagaaactatactgagtactggagcggatctaacagtggaaatcagaaaatggaagctgttcagaaaataaattatactgtcccccaaagtggaacttttaagattgaattcccaatcctggaggattccagtgagctacagttgaaggcctatttccttggtagtaaaagtagcatggcagttcatagtctgtttaagtctcctagtaagacatacatccaactaaaaacaagagatgaaaatataaaggtgggatcgccttttgagttggtggttagtggcaacaaacgattgaaggagttaagctatatggtagtatccaggggacagttggtggctgtaggaaaacaaaattcaacaatgttctctttaacaccagaaaattcttggactccaaaagcctgtgtaattgtgtattatattgaagatgatggggaaattataagtgatgttctaaaaattcctgttcagcttgtttttaaaaataagataaagctatattggagtaaagtgaaagctgaaccatctgagaaagtctctcttaggatctctgtgacacagcctgactccatagttgggattgtagctgttgacaaaagtgtgaatctgatgaatgcctctaatgatattacaatggaaaatgtggtccatgagttggaactttataacacaggatattatttaggcatgttcatgaattcttttgcagtctttcaggaatgtggactctgggtattgacagatgcaaacctcacgaaggattatattgatggtgtttatgacaatgcagaatatgctgagaggtttatggaggaaaatgaaggacatattgtagatattcatgacttttctttgggtagcagtccacatgtccgaaagcattttccagagacttggatttggctagacaccaacatgggttacaggatttaccaagaatttgaagtaactgtacctgattctatcacttcttgggtggctactggttttgtgatctctgaggacctgggtcttggactaacaactactccagtggagctccaagccttccaaccatttttcatttttttgaatcttccctactctgttatcagaggtgaagaatttgctttggaaataactatattcaattatttgaaagatgccactgaggttaaggtaatcattgagaaaagtgacaaatttgatattctaatgacttcaaatgaaataaatgccacaggccaccagcagacccttctggttcccagtgaggatggggcaactgttctttttcccatcaggccaacacatctgggagaaattcctatcacagtcacagctctttcacccactgcttctgatgctgtcacccagatgattttagtaaaggctgaaggaatagaaaaatcatattcacaatccatcttattagacttgactgacaataggctacagagtaccctgaaaactttgagtttctcatttcctcctaatacagtgactggcagtgaaagagttcagatcactgcaattggagatgttcttggtccttccatcaatggcttagcctcattgattcggatgccttatggctgtggtgaacagaacatgataaattttgctccaaatatttacattttggattatctgactaaaaagaaacaactgacagataatttgaaagaaaaagctctttcatttatgaggcaaggttaccagagagaacttctctatcagagggaagatggctctttcagtgcttttgggaattatgacccttctgggagcacttggttgtcagcttttgttttaagatgtttccttgaagccgatccttacatagatattgatcagaatgtgttacacagaacatacacttggcttaaaggacatcagaaatccaacggtgaattttgggatccaggaagagtgattcatagtgagcttcaaggtggcaataaaagtccagtaacacttacagcctatattgtaacttctctcctgggatatagaaagtatcagcctaacattgatgtgcaagagtctatccattttttggagtctgaattcagtagaggaatttcagacaattatactctagcccttataacttatgcattgtcatcagtggggagtcctaaagcgaaggaagctttgaatatgctgacttggagagcagaacaagaaggtggcatgcaattctgggtgtcatcagagtccaaactttctgactcctggcagccacgctccctggatattgaagttgcagcctatgcactgctctcacacttcttacaatttcagacttctgagggaatcccaattatgaggtggctaagcaggcaaagaaatagcttgggtggttttgcatctactcaggataccactgtggctttaaaggctctgtctgaatttgcagccctaatgaatacagaaaggacaaatatccaagtgaccgtgacggggcctagctcaccaagtcctcttgctgtggtacagccaacggcagttaatatttccgcaaatggttttggatttgctatttgtcagctcaatgttgtatataatgtgaaggcttctgggtcttctagaagacgaagatctatccaaaatcaagaagcctttgatttagatgttgctgtaaaagaaaataaagatgatctcaatcatgtggatttgaatgtgtgtacaagcttttcgggcccgggtaggagtggcatggctcttatggaagttaacctattaagtggctttatggtgccttcagaagcaatttctctgagcgagacagtgaagaaagtggaatatgatcatggaaaactcaacctctatttagattctgtaaatgaaacccagttttgtgttaatattcctgctgtgagaaactttaaagtttcaaatacccaagatgcttcagtgtccatagtggattactatgagccaaggagacaggcggtgagaagttacaactctgaagtgaagctgtcctcctgtgacctttgcagtgatgtccagggctgccgtccttgtgaggatggagcttcaggctcccatcatcactcttcagtcatttttattttctgtttcaagcttctgtactttatggaactttggctgtgatttatttttaaaggactctgtgtaacactaacatttccagtagtcacatgtgattgttttgttttcgtagaagaatactgcttctattttgaaaaaagagttttttttctttctatggggttgcagggatggtgtacaacaggtcctagcatgtatagctgcatagatttcttcacctgatctttgtgtggaagatcagaatgaatgcagttgtgtgtctatattttcccctctcaaaatcttttagaatttttttggaggtgtttgttttctccagaataaaggtattactttagaataggtattctcctcattttgtgaaagaaatgaacctagattcttaagcattattacacatccatgtttgcttaaagatggatttccctgggaatgggagaaaacagccagcaggaggagcttcatctgttcccttcccacctccaacctagccctactgcccaccccaccccaacccaccccatgcccagtggtctcagtagatacttcttaactggaaattctttcttttcagaatctaggtggtgaattttttttaagtggcacggtctttttctgcttgaaatctgatcacaccccccagccattgccctccctctctttttcctctgtagagaaatgtgaggggcagtacatttactgtgcttttcacaccatctcagaggttgaggagcatactgaaaattgccctggggggtgctgggtgtgctgtctccttcccacatcctcagccccacaccagctctatttcaggggtgagagtcagagagcactgcaatatgtgcttcatgggatttcgattcgaagatcctagaccagggagacactgtgagccagggatacaacaaaatactaggtaagtcactgcagaccgacctccctgcagtttgggaaagaagctgggtttgtggagaatcagagcatcttgacatgactgctgacctaaagatccctggcattggccagggatcctgtggaacctcttctagttcaggggtgtgagcattagactgccagttgtctagtgacatctgatgcttgctgtgaacttttaagatccccgaatcctgagcacctcaatctttaattgccctgtattccgaagggtaatataatttatctggatggaaattttaaagatgaatcccccttttttcttttcttctctcttttctttccttctccctttcttctttgccttctaaatatactgaaatgatttagatatgtgtcaacaattaatgatcttttattcaatctaagaaatggtttagtttttctctttagctctatggcatttcactcaagtggacaggggaaaaagtaattgccatgggctccaaagaatttgctttatgtttttagctatttaaaaataaatccatcaaaaataaagtatgcaaatgtatcttttaaagttaatttttaaaaatgctcttattttagtgaattttcagaaattatagtggaatggatgctcatatattgcttatggatattttggataccaaagtaggaataactgacattcagtattttaaagctggcaaacctgtacatagaaaatagatccccagacagtggtctatgaagagggcagttaagtatcaaatacttaattttcttgcctttttttcttaagtggggaaaagtttctagatctcttacacctctgacacaatctgttctaaaacaggcacttgtaatgttggggcctccttgtaaacgtgtttttgccctttactctctgggagttctttaaaggtgaaatcatcttacaaagaaattgggggagggtcttggcaaaggactttcccctcctctttcctggcctgggaaccttatactgacaatcaatactttatattttaaagtatataatttatagttaacttctagtgtaatatattaggaaacactagaatggaaaggccattggaagacaggttgtatcttttttagaccatatttccttgtttaaaaactatcatttgaatacttttttggtgaagaactccatgttttcaagttaaaggtcacctcgtaggccaggcgcagtggctcatgcctgtaatcccagcactctgggaggctgaggcgggtgaatcacaaggttaggagtttgagaccagcctggccaatatggtgaaaccccgtccctactaaaaatacaaaatttagccaggcgtggtggcatgcacctgtagtcccacctactcgggaggctgaggcaggagaatcacttgaacctgagagacagaggttgcagtgagccgagatcacgccactgcactccagcctgggggacagagtgagattctgtctcaaaaaacaaaaaacaaaaaagtcaccttgtaactcatctctttttattgtaagtttattaaaaatgaagaggacaacaatgagaaggaacataaagggttagctagcactgtctcctggtgcatggggctgtgcagatgtcccggccacttcttccttcatacttcccttagagaacttgctctgctacaagcagtgggcttggactaaaagtgattaaaataccacaggcataaggagaaaaggagtatatgtagtagtaataattactagtataaattattttcttcacatgctatgagtaataatattaaaaaactcattttaccattaagattccttatgctgaagctcttccatttagaatactgtcaatgtcatttactggtatgaactaaagtcccccttcttttccactcactgggaaccttagtaaaacaccagcatatcttacctctctttctgactggccgatgcttccagagactgaatgttgggaaaacctagtagccaaacaattctaggacagaataacatttttatatttggttccaccatcttattacatttagttatagttttaaaaaagaaattcaagcccattaaaatatgtctggtcaatgaaatgcttccttttattgtgttgtgctattgtactttgtttttcaaaacattgtaaaaatagtatctttggtttagtattttggattatatattataatctgaggagtgttttgcttatgtagaatccagatatatttctgttacctaggagatgttacttacatatgtaatactgtatcctgcacgtggaaatattcagaattgtagatagcataactctccctgctcctattcttttgagcctaggtataattttttttttttttttagaaaaagacatatttagctttaatttctatttatgctaaacatatttataagtagtctgtcaatataataccaactatttttatttttacataattcaattatttcatttgacatgtctggcagactcaagacattaagtaaaaaattggaactatgatttttctttgtcattttttaaaaaagaattattttattaacctgctggcatataatctggagttcttttcacaaccttactttttctgatttgctttattgaatgattgaatactcatttctttctaaaaatatgttgtaaattctcccttggcaagatttctccctatgagggtagttattatttgagtctgccaagtggttaccatggggcaaggtgccatgatgtattcttgggtgcattggttttttgcgcattgtaaatttaagacacttatagtaagtggactcattcatagatgagtttcagaaccttttacgttctcggtagaggcttctgtcggacaggcagaagagtgtattcctcacttttttttttgtcttcaaattccagtaaggcatagcacttttaagaaattagaatttttctatcatctatgcaaatgatatttatgttaatattaaatatcttatgttacactgggagtaatttgaggtgcaattatttttattactactttgaatagaggaccattatccttctttcttcagaaaactaagaagtaagtgtaacttttaaagtaagtatatatcagtgagagtaggcttgttttacaactatttctagccagtgagttgtgttttcatgtctcatcaaaagacaataccacattgcatcattttacaaaatatgttgtcattttcatttcagttgtaacataggaaaatagatatttcctagatgatttctgagtttcttactgcaaagaacagttataaattggtatacatgtgtctctgtaatagggataatattgatatatctgttgctacatatttaagaatcattctatcttatgttgtcttgaggccaagatttaccacgtttgcccagtgtattgaattggtggtagaaggtagttccatgttccatttgtagatctttaagattttatctttgataactttaatagaatgtggctcagttctggtccttcaagcctgtatggtttggattttcagtaggggacagttgatgtggagtcaatctctttggtacacaggaagctttataaaatttcattcacgaatctcttattttgggaagctgttttgcatatgagaagaacactgttgaaataaggaactaaagctttatatattgatcaaggtgattctgaaagttttaatttttaatgttgtaatgttatgttattgttaattgtactttattatgtattcaatagaaaatcatgatttattaataaaagcttaaattctcatctattt

SEQ ID NO: 3 - RNA, 9221 nt, Homo sapiens
  source 1..9221: mol_type = mRNA
atagttgctatgaatttctcctcacaaggatgggcctgtttactcaccctgggcatcgttggtagagcgctagtgtaaacagcctgaggaacccggtgggccggggaagtgggcgcgctctgttctccgcggccagctgggacgccgggccaggtggggccgcctgcgtttagcaactgctttctcaccccctggatttgcgatgtttgccacagcagcgagaagcgccattgtaatggggatgggaggggtggagcctccaagtcctgtctcaatttagatctctcactctgctgttaggcgcgcccatttcagattactaaactcgaattaagagggaaaaaaaatcagggaggaggtggcaagccacaccccacggtgcccgcgaacttccccggcagcggactgtagcccaggcagacgccgtcgagatgcagggcccaccgctcctgaccgccgcccacctcctctgcgtgtgcaccgccgcgctggccgtggctcccgggcctcggtttctggtgacagccccagggatcatcaggcccggaggaaatgtgactattggggtggagcttctggaacactgcccttcacaggtgactgtgaaggcggagctgctcaagacagcatcaaacctcactgtctctgtcctggaagcagaaggagtctttgaaaaaggctcttttaagacacttactcttccatcagaccccaaatcaaatttgatccaacagtggttgtcacaacaaagtgatcttggagtcatttccaaaacttttcagctatcttcccatccaatacttggtgactggtctattcaagttcaagtgaatgaccagacatactatcaatcatttcaggtttcagaatatgtattaccaaaatttgaagtgactttgcagacaccattatattgttctatgaattctaagcatttaaatggtaccatcacggcaaagtatacatatgggaagccagtgaaaggagacgtaacgcttacatttttacctttatccttttggggaaagaagaaaaatattacaaaaacatttaagataaatggatctgcaaacttctcttttaatgatgaagagatgaaaaatgtaatggattcttcaaatggactttctgaatacctggatctatcttcccctggaccagtagaaattttaaccacagtgacagaatcagttacaggtatttcaagaaatgtaagcactaatgtgttcttcaagcaacatgattacatcattgagttttttgattatactactgtcttgaagccatctctcaacttcacagccactgtgaaggtaactcgtgctgatggcaaccaactgactcttgaagaaagaagaaataatgtagtcataacagtgacacagagaaactatactgagtactggagcggatctaacagtggaaatcagaaaatggaagctgttcagaaaataaattatactgtcccccaaagtggaacttttaagattgaattcccaatcctggaggattccagtgagctacagttgaaggcctatttccttggtagtaaaagtagcatggcagttcatagtctgtttaagtctcctagtaagacatacatccaactaaaaacaagagatgaaaatataaaggtgggatcgccttttgagttggtggttagtggcaacaaacgattgaaggagttaagctatatggtagtatccaggggacagttggtggctgtaggaaaacaaaattcaacaatgttctctttaacaccagaaaattcttggactccaaaagcctgtgtaattgtgtattatattgaagatgatggggaaattataagtgatgttctaaaaattcctgttcagcttgtttttaaaaataagataaagctatattggagtaaagtgaaagctgaaccatctgagaaagtctctcttaggatctctgtgacacagcctgactccatagttgggattgtagctgttgacaaaagtgtgaatctgatgaatgcctctaatgatattacaatggaaaatgtggtccatgagttggaactttataacacaggatattatttaggcatgttcatgaattcttttgcagtctttcaggaatgtggactctgggtattgacagatgcaaacctcacgaaggattatattgatggtgtttatgacaatgcagaatatgctgagaggtttatggaggaaaatgaaggacatattgtagatattcatgacttttctttgggtagcagtccacatgtccgaaagcattttccagagacttggatttggctagacaccaacatgggttacaggatttaccaagaatttgaagtaactgtacctgattctatcacttcttgggtggctactggttttgtgatctctgaggacctgggtcttggactaacaactactccagtggagctccaagccttccaaccatttttcatttttttgaatcttccctactctgttatcagaggtgaagaatttgctttggaaataactatattcaattatttgaaagatgccactgaggttaaggtaatcattgagaaaagtgacaaatttgatattctaatgacttcaaatgaaataaatgccacaggccaccagcagacccttctggttcccagtgaggatggggcaactgttctttttcccatcaggccaacacatctgggagaaattcctatcacagtcacagctctttcacccactgcttctgatgctgtcacccagatgattttagtaaaggctgaaggaatagaaaaatcatattcacaatccatcttattagacttgactgacaataggctacagagtaccctgaaaactttgagtttctcatttcctcctaatacagtgactggcagtgaaagagttcagatcactgcaattggagatgttcttggtccttccatcaatggcttagcctcattgattcggatgccttatggctgtggtgaacagaacatgataaattttgctccaaatatttacattttggattatctgactaaaaagaaacaactgacagataatttgaaagaaaaagctctttcatttatgaggcaaggttaccagagagaacttctctatcagagggaagatggctctttcagtgcttttgggaattatgacccttctgggagcacttggttgtcagcttttgttttaagatgtttccttgaagccgatccttacatagatattgatcagaatgtgttacacagaacatacacttggcttaaaggacatcagaaatccaacggtgaattttgggatccaggaagagtgattcatagtgagcttcaaggtggcaataaaagtccagtaacacttacagcctatattgtaacttctctcctgggatatagaaagtatcagcctaacattgatgtgcaagagtctatccattttttggagtctgaattcagtagaggaatttcagacaattatactctagcccttataacttatgcattgtcatcagtggggagtcctaaagcgaaggaagctttgaatatgctgacttggagagcagaacaagaaggtggcatgcaattctgggtgtcatcagagtccaaactttctgactcctggcagccacgctccctggatattgaagttgcagcctatgcactgctctcacacttcttacaatttcagacttctgagggaatcccaattatgaggtggctaagcaggcaaagaaatagcttgggtggttttgcatctactcaggataccactgtggctttaaaggctctgtctgaatttgcagccctaatgaatacagaaaggacaaatatccaagtgaccgtgacggggcctagctcaccaagtcctgtaaagtttctgattgacacacacaaccgcttactccttcagacagcagagcttgctgtggtacagccaacggcagttaatatttccgcaaatggttttggatttgctatttgtcagctcaatgttgtatataatgtgaaggcttctgggtcttctagaagacgaagatctatccaaaatcaagaagcctttgatttagatgttgctgtaaaagaaaataaagatgatctcaatcatgtggatttgaatgtgtgtacaagcttttcgggcccgggtaggagtggcatggctcttatggaagttaacctattaagtggctttatggtgccttcagaagcaatttctctgagcgagacagtgaagaaagtggaatatgatcatggaaaactcaacctctatttagattctgtaaatgaaacccagttttgtgttaatattcctgctgtgagaaactttaaagtttcaaatacccaagatgcttcagtgtccatagtggattactatgagccaaggagacaggcggtgagaagttacaactctgaagtgaagctgtcctcctgtgacctttgcagtgatgtccagggctgccgtccttgtgaggatggagcttcaggctcccatcatcactcttcagtcatttttattttctgtttcaagcttctgtactttatggaactttggctgtgatttatttttaaaggactctgtgtaacactaacatttccagtagtcacatgtgattgttttgttttcgtagaagaatactgcttctattttgaaaaaagagttttttttctttctatggggttgcagggatggtgtacaacaggtcctagcatgtatagctgcatagatttcttcacctgatctttgtgtggaagatcagaatgaatgcagttgtgtgtctatattttcccctctcaaaatcttttagaatttttttggaggtgtttgttttctccagaataaaggtattactttagaataggtattctcctcattttgtgaaagaaatgaacctagattcttaagcattattacacatccatgtttgcttaaagatggatttccctgggaatgggagaaaacagccagcaggaggagcttcatctgttcccttcccacctccaacctagccctactgcccaccccaccccaacccaccccatgcccagtggtctcagtagatacttcttaactggaaattctttcttttcagaatctaggtggtgaattttttttaagtggcacggtctttttctgcttgaaatctgatcacaccccccagccattgccctccctctctttttcctctgtagagaaatgtgaggggcagtacatttactgtgcttttcacaccatctcagaggttgaggagcatactgaaaattgccctggggggtgctgggtgtgctgtctccttcccacatcctcagccccacaccagctctatttcaggggtgagagtcagagagcactgcaatatgtgcttcatgggatttcgattcgaagatcctagaccagggagacactgtgagccagggatacaacaaaatactaggtaagtcactgcagaccgacctccctgcagtttgggaaagaagctgggtttgtggagaatcagagcatcttgacatgactgctgacctaaagatccctggcattggccagggatcctgtggaacctcttctagttcaggggtgtgagcattagactgccagttgtctagtgacatctgatgcttgctgtgaacttttaagatccccgaatcctgagcacctcaatctttaattgccctgtattccgaagggtaatataatttatctggatggaaattttaaagatgaatcccccttttttcttttcttctctcttttctttccttctccctttcttctttgccttctaaatatactgaaatgatttagatatgtgtcaacaattaatgatcttttattcaatctaagaaatggtttagtttttctctttagctctatggcatttcactcaagtggacaggggaaaaagtaattgccatgggctccaaagaatttgctttatgtttttagctatttaaaaataaatccatcaaaaataaagtatgcaaatgtatcttttaaagttaatttttaaaaatgctcttattttagtgaattttcagaaattatagtggaatggatgctcatatattgcttatggatattttggataccaaagtaggaataactgacattcagtattttaaagctggcaaacctgtacatagaaaatagatccccagacagtggtctatgaagagggcagttaagtatcaaatacttaattttcttgcctttttttcttaagtggggaaaagtttctagatctcttacacctctgacacaatctgttctaaaacaggcacttgtaatgttggggcctccttgtaaacgtgtttttgccctttactctctgggagttctttaaaggtgaaatcatcttacaaagaaattgggggagggtcttggcaaaggactttcccctcctctttcctggcctgggaaccttatactgacaatcaatactttatattttaaagtatataatttatagttaacttctagtgtaatatattaggaaacactagaatggaaaggccattggaagacaggttgtatcttttttagaccatatttccttgtttaaaaactatcatttgaatacttttttggtgaagaactccatgttttcaagttaaaggtcacctcgtaggccaggcgcagtggctcatgcctgtaatcccagcactctgggaggctgaggcgggtgaatcacaaggttaggagtttgagaccagcctggccaatatggtgaaaccccgtccctactaaaaatacaaaatttagccaggcgtggtggcatgcacctgtagtcccacctactcgggaggctgaggcaggagaatcacttgaacctgagagacagaggttgcagtgagccgagatcacgccactgcactccagcctgggggacagagtgagattctgtctcaaaaaacaaaaaacaaaaaagtcaccttgtaactcatctctttttattgtaagtttattaaaaatgaagaggacaacaatgagaaggaacataaagggttagctagcactgtctcctggtgcatggggctgtgcagatgtcccggccacttcttccttcatacttcccttagagaacttgctctgctacaagcagtgggcttggactaaaagtgattaaaataccacaggcataaggagaaaaggagtatatgtagtagtaataattactagtataaattattttcttcacatgctatgagtaataatattaaaaaactcattttaccattaagattccttatgctgaagctcttccatttagaatactgtcaatgtcatttactggtatgaactaaagtcccccttcttttccactcactgggaaccttagtaaaacaccagcatatcttacctctctttctgactggccgatgcttccagagactgaatgttgggaaaacctagtagccaaacaattctaggacagaataacatttttatatttggttccaccatcttattacatttagttatagttttaaaaaagaaattcaagcccattaaaatatgtctggtcaatgaaatgcttccttttattgtgttgtgctattgtactttgtttttcaaaacattgtaaaaatagtatctttggtttagtattttggattatatattataatctgaggagtgttttgcttatgtagaatccagatatatttctgttacctaggagatgttacttacatatgtaatactgtatcctgcacgtggaaatattcagaattgtagatagcataactctccctgctcctattcttttgagcctaggtataattttttttttttttttagaaaaagacatatttagctttaatttctatttatgctaaacatatttataagtagtctgtcaatataataccaactatttttatttttacataattcaattatttcatttgacatgtctggcagactcaagacattaagtaaaaaattggaactatgatttttctttgtcattttttaaaaaagaattattttattaacctgctggcatataatctggagttcttttcacaaccttactttttctgatttgctttattgaatgattgaatactcatttctttctaaaaatatgttgtaaattctcccttggcaagatttctccctatgagggtagttattatttgagtctgccaagtggttaccatggggcaaggtgccatgatgtattcttgggtgcattggttttttgcgcattgtaaatttaagacacttatagtaagtggactcattcatagatgagtttcagaaccttttacgttctcggtagaggcttctgtcggacaggcagaagagtgtattcctcacttttttttttgtcttcaaattccagtaaggcatagcacttttaagaaattagaatttttctatcatctatgcaaatgatatttatgttaatattaaatatcttatgttacactgggagtaatttgaggtgcaattatttttattactactttgaatagaggaccattatccttctttcttcagaaaactaagaagtaagtgtaacttttaaagtaagtatatatcagtgagagtaggcttgttttacaactatttctagccagtgagttgtgttttcatgtctcatcaaaagacaataccacattgcatcattttacaaaatatgttgtcattttcatttcagttgtaacataggaaaatagatatttcctagatgatttctgagtttcttactgcaaagaacagttataaattggtatacatgtgtctctgtaatagggataatattgatatatctgttgctacatatttaagaatcattctatcttatgttgtcttgaggccaagatttaccacgtttgcccagtgtattgaattggtggtagaaggtagttccatgttccatttgtagatctttaagattttatctttgataactttaatagaatgtggctcagttctggtccttcaagcctgtatggtttggattttcagtaggggacagttgatgtggagtcaatctctttggtacacaggaagctttataaaatttcattcacgaatctcttattttgggaagctgttttgcatatgagaagaacactgttgaaataaggaactaaagctttatatattgatcaaggtgattctgaaagttttaatttttaatgttgtaatgttatgttattgttaattgtactttattatgtattcaatagaaaatcatgatttattaataaaagcttaaattctcatctattt

SEQ ID NO: 4 - RNA, 9031 nt, Homo sapiens
  source 1..9031: mol_type = mRNA
agacgccgtcgagatgcagggcccaccgctcctgaccgccgcccacctcctctgcgtgtgcaccgccgcgctggccgtggctcccgggcctcggtttctggtgacagccccagggatcatcaggcccggaggaaatgtgactattggggtggagcttctggaacactgcccttcacaggtgactgtgaaggcggagctgctcaagacagcatcaaacctcactgtctctgtcctggaagcagaaggagtctttgaaaaaggctcttttaagacacttactcttccatcactacctctgaacagtgcagatgagatttatgagctacgtgtaaccggacgtacccaggatgagattttattctctaatagtacccgcttatcatttgagaccaagagaatatctgtcttcattcaaacagacaaggccttatacaagccaaagcaagaagtgaagtttcgcattgttacactcttctcagattttaagccttacaaaacctctttaaacattctcattaaggaccccaaatcaaatttgatccaacagtggttgtcacaacaaagtgatcttggagtcatttccaaaacttttcagctatcttcccatccaatacttggtgactggtctattcaagttcaagtgaatgaccagacatactatcaatcatttcaggtttcagaatatgtattaccaaaatttgaagtgactttgcagacaccattatattgttctatgaattctaagcatttaaatggtaccatcacggcaaagtatacatatgggaagccagtgaaaggagacgtaacgcttacatttttacctttatccttttggggaaagaagaaaaatattacaaaaacatttaagataaatggatctgcaaacttctcttttaatgatgaagagatgaaaaatgtaatggattcttcaaatggactttctgaatacctggatctatcttcccctggaccagtagaaattttaaccacagtgacagaatcagttacaggtatttcaagaaatgtaagcactaatgtgttcttcaagcaacatgattacatcattgagttttttgattatactactgtcttgaagccatctctcaacttcacagccactgtgaaggtaactcgtgctgatggcaaccaactgactcttgaagaaagaagaaataatgtagtcataacagtgacacagagaaactatactgagtactggagcggatctaacagtggaaatcagaaaatggaagctgttcagaaaataaattatactgtcccccaaagtggaacttttaagattgaattcccaatcctggaggattccagtgagctacagttgaaggcctatttccttggtagtaaaagtagcatggcagttcatagtctgtttaagtctcctagtaagacatacatccaactaaaaacaagagatgaaaatataaaggtgggatcgccttttgagttggtggttagtggcaacaaacgattgaaggagttaagctatatggtagtatccaggggacagttggtggctgtaggaaaacaaaattcaacaatgttctctttaacaccagaaaattcttggactccaaaagcctgtgtaattgtgtattatattgaagatgatggggaaattataagtgatgttctaaaaattcctgttcagcttgtttttaaaaataagataaagctatattggagtaaagtgaaagctgaaccatctgagaaagtctctcttaggatctctgtgacacagcctgactccatagttgggattgtagctgttgacaaaagtgtgaatctgatgaatgcctctaatgatattacaatggaaaatgtggtccatgagttggaactttataacacaggatattatttaggcatgttcatgaattcttttgcagtctttcaggaatgtggactctgggtattgacagatgcaaacctcacgaaggattatattgatggtgtttatgacaatgcagaatatgctgagaggtttatggaggaaaatgaaggacatattgtagatattcatgacttttctttgggtagcagtccacatgtccgaaagcattttccagagacttggatttggctagacaccaacatgggttacaggatttaccaagaatttgaagtaactgtacctgattctatcacttcttgggtggctactggttttgtgatctctgaggacctgggtcttggactaacaactactccagtggagctccaagccttccaaccatttttcatttttttgaatcttccctactctgttatcagaggtgaagaatttgctttggaaataactatattcaattatttgaaagatgccactgaggttaaggtaatcattgagaaaagtgacaaatttgatattctaatgacttcaaatgaaataaatgccacaggccaccagcagacccttctggttcccagtgaggatggggcaactgttctttttcccatcaggccaacacatctgggagaaattcctatcacagtcacagctctttcacccactgcttctgatgctgtcacccagatgattttagtaaaggctgaaggaatagaaaaatcatattcacaatccatcttattagacttgactgacaataggctacagagtaccctgaaaactttgagtttctcatttcctcctaatacagtgactggcagtgaaagagttcagatcactgcaattggagatgttcttggtccttccatcaatggcttagcctcattgattcggatgccttatggctgtggtgaacagaacatgataaattttgctccaaatatttacattttggattatctgactaaaaagaaacaactgacagataatttgaaagaaaaagctctttcatttatgaggcaaggttaccagagagaacttctctatcagagggaagatggctctttcagtgcttttgggaattatgacccttctgggagcacttggttgtcagcttttgttttaagatgtttccttgaagccgatccttacatagatattgatcagaatgtgttacacagaacatacacttggcttaaaggacatcagaaatccaacggtgaattttgggatccaggaagagtgattcatagtgagcttcaaggtggcaataaaagtccagtaacacttacagcctatattgtaacttctctcctgggatatagaaagtatcagcctaacattgatgtgcaagagtctatccattttttggagtctgaattcagtagaggaatttcagacaattatactctagcccttataacttatgcattgtcatcagtggggagtcctaaagcgaaggaagctttgaatatgctgacttggagagcagaacaagaaggtggcatgcaattctgggtgtcatcagagtccaaactttctgactcctggcagccacgctccctggatattgaagttgcagcctatgcactgctctcacacttcttacaatttcagacttctgagggaatcccaattatgaggtggctaagcaggcaaagaaatagcttgggtggttttgcatctactcaggataccactgtggctttaaaggctctgtctgaatttgcagccctaatgaatacagaaaggacaaatatccaagtgaccgtgacggggcctagctcaccaagtcctgtaaagtttctgattgacacacacaaccgcttactccttcagacagcagagcttgctgtggtacagccaacggcagttaatatttccgcaaatggttttggatttgctatttgtcagctcaatgttgtatataatgtgaaggcttctgggtcttctagaagacgaagatctatccaaaatcaagaagcctttgatttagatgttgctgtaaaagaaaataaagatgatctcaatcatgtggatttgaatgtgtgtacaagcttttcgggcccgggtaggagtggcatggctcttatggaagttaacctattaagtggctttatggtgccttcagaagcaatttctctgagcgagacagtgaagaaagtggaatatgatcatggaaaactcaacctctatttagattctgtaaatgaaacccagttttgtgttaatattcctgctgtgagaaactttaaagtttcaaatacccaagatgcttcagtgtccatagtggattactatgagccaaggagacaggcggtgagaagttacaactctgaagtgaagctgtcctcctgtgacctttgcagtgatgtccagggctgccgtccttgtgaggatggagcttcaggctcccatcatcactcttcagtcatttttattttctgtttcaagcttctgtactttatggaactttggctgtgatttatttttaaaggactctgtgtaacactaacatttccagtagtcacatgtgattgttttgttttcgtagaagaatactgcttctattttgaaaaaagagttttttttctttctatggggttgcagggatggtgtacaacaggtcctagcatgtatagctgcatagatttcttcacctgatctttgtgtggaagatcagaatgaatgcagttgtgtgtctatattttcccctctcaaaatcttttagaatttttttggaggtgtttgttttctccagaataaaggtattactttagaataggtattctcctcattttgtgaaagaaatgaacctagattcttaagcattattacacatccatgtttgcttaaagatggatttccctgggaatgggagaaaacagccagcaggaggagcttcatctgttcccttcccacctccaacctagccctactgcccaccccaccccaacccaccccatgcccagtggtctcagtagatacttcttaactggaaattctttcttttcagaatctaggtggtgaattttttttaagtggcacggtctttttctgcttgaaatctgatcacaccccccagccattgccctccctctctttttcctctgtagagaaatgtgaggggcagtacatttactgtgcttttcacaccatctcagaggttgaggagcatactgaaaattgccctggggggtgctgggtgtgctgtctccttcccacatcctcagccccacaccagctctatttcaggggtgagagtcagagagcactgcaatatgtgcttcatgggatttcgattcgaagatcctagaccagggagacactgtgagccagggatacaacaaaatactaggtaagtcactgcagaccgacctccctgcagtttgggaaagaagctgggtttgtggagaatcagagcatcttgacatgactgctgacctaaagatccctggcattggccagggatcctgtggaacctcttctagttcaggggtgtgagcattagactgccagttgtctagtgacatctgatgcttgctgtgaacttttaagatccccgaatcctgagcacctcaatctttaattgccctgtattccgaagggtaatataatttatctggatggaaattttaaagatgaatcccccttttttcttttcttctctcttttctttccttctccctttcttctttgccttctaaatatactgaaatgatttagatatgtgtcaacaattaatgatcttttattcaatctaagaaatggtttagtttttctctttagctctatggcatttcactcaagtggacaggggaaaaagtaattgccatgggctccaaagaatttgctttatgtttttagctatttaaaaataaatccatcaaaaataaagtatgcaaatgtatcttttaaagttaatttttaaaaatgctcttattttagtgaattttcagaaattatagtggaatggatgctcatatattgcttatggatattttggataccaaagtaggaataactgacattcagtattttaaagctggcaaacctgtacatagaaaatagatccccagacagtggtctatgaagagggcagttaagtatcaaatacttaattttcttgcctttttttcttaagtggggaaaagtttctagatctcttacacctctgacacaatctgttctaaaacaggcacttgtaatgttggggcctccttgtaaacgtgtttttgccctttactctctgggagttctttaaaggtgaaatcatcttacaaagaaattgggggagggtcttggcaaaggactttcccctcctctttcctggcctgggaaccttatactgacaatcaatactttatattttaaagtatataatttatagttaacttctagtgtaatatattaggaaacactagaatggaaaggccattggaagacaggttgtatcttttttagaccatatttccttgtttaaaaactatcatttgaatacttttttggtgaagaactccatgttttcaagttaaaggtcacctcgtaggccaggcgcagtggctcatgcctgtaatcccagcactctgggaggctgaggcgggtgaatcacaaggttaggagtttgagaccagcctggccaatatggtgaaaccccgtccctactaaaaatacaaaatttagccaggcgtggtggcatgcacctgtagtcccacctactcgggaggctgaggcaggagaatcacttgaacctgagagacagaggttgcagtgagccgagatcacgccactgcactccagcctgggggacagagtgagattctgtctcaaaaaacaaaaaacaaaaaagtcaccttgtaactcatctctttttattgtaagtttattaaaaatgaagaggacaacaatgagaaggaacataaagggttagctagcactgtctcctggtgcatggggctgtgcagatgtcccggccacttcttccttcatacttcccttagagaacttgctctgctacaagcagtgggcttggactaaaagtgattaaaataccacaggcataaggagaaaaggagtatatgtagtagtaataattactagtataaattattttcttcacatgctatgagtaataatattaaaaaactcattttaccattaagattccttatgctgaagctcttccatttagaatactgtcaatgtcatttactggtatgaactaaagtcccccttcttttccactcactgggaaccttagtaaaacaccagcatatcttacctctctttctgactggccgatgcttccagagactgaatgttgggaaaacctagtagccaaacaattctaggacagaataacatttttatatttggttccaccatcttattacatttagttatagttttaaaaaagaaattcaagcccattaaaatatgtctggtcaatgaaatgcttccttttattgtgttgtgctattgtactttgtttttcaaaacattgtaaaaatagtatctttggtttagtattttggattatatattataatctgaggagtgttttgcttatgtagaatccagatatatttctgttacctaggagatgttacttacatatgtaatactgtatcctgcacgtggaaatattcagaattgtagatagcataactctccctgctcctattcttttgagcctaggtataattttttttttttttttagaaaaagacatatttagctttaatttctatttatgctaaacatatttataagtagtctgtcaatataataccaactatttttatttttacataattcaattatttcatttgacatgtctggcagactcaagacattaagtaaaaaattggaactatgatttttctttgtcattttttaaaaaagaattattttattaacctgctggcatataatctggagttcttttcacaaccttactttttctgatttgctttattgaatgattgaatactcatttctttctaaaaatatgttgtaaattctcccttggcaagatttctccctatgagggtagttattatttgagtctgccaagtggttaccatggggcaaggtgccatgatgtattcttgggtgcattggttttttgcgcattgtaaatttaagacacttatagtaagtggactcattcatagatgagtttcagaaccttttacgttctcggtagaggcttctgtcggacaggcagaagagtgtattcctcacttttttttttgtcttcaaattccagtaaggcatagcacttttaagaaattagaatttttctatcatctatgcaaatgatatttatgttaatattaaatatcttatgttacactgggagtaatttgaggtgcaattatttttattactactttgaatagaggaccattatccttctttcttcagaaaactaagaagtaagtgtaacttttaaagtaagtatatatcagtgagagtaggcttgttttacaactatttctagccagtgagttgtgttttcatgtctcatcaaaagacaataccacattgcatcattttacaaaatatgttgtcattttcatttcagttgtaacataggaaaatagatatttcctagatgatttctgagtttcttactgcaaagaacagttataaattggtatacatgtgtctctgtaatagggataatattgatatatctgttgctacatatttaagaatcattctatcttatgttgtcttgaggccaagatttaccacgtttgcccagtgtattgaattggtggtagaaggtagttccatgttccatttgtagatctttaagattttatctttgataactttaatagaatgtggctcagttctggtccttcaagcctgtatggtttggattttcagtaggggacagttgatgtggagtcaatctctttggtacacaggaagctttataaaatttcattcacgaatctcttattttgggaagctgttttgcatatgagaagaacactgttgaaataaggaactaaagctttatatattgatcaaggtgattctgaaagttttaatttttaatgttgtaatgttatgttattgttaattgtactttattatgtattcaatagaaaatcatgatttattaataaaagcttaaattctcatcta

SEQ ID NO: 5 - RNA, 5883 nt, Homo sapiens
  source 1..5883: mol_type = mRNA
ctaaactcgaattaagagggaaaaaaaatcagggaggaggtggcaagccacaccccacggtgcccgcgaacttccccggcagcggactgtagcccaggcagacgccgtcgagatgcagggcccaccgctcctgaccgccgcccacctcctctgcgtgtgcaccgccgcgctggccgtggctcccgggcctcggtttctggtgacagccccagggatcatcaggcccggaggaaatgtgactattggggtggagcttctggaacactgcccttcacaggtgactgtgaaggcggagctgctcaagacagcatcaaacctcactgtctctgtcctggaagcagaaggagtctttgaaaaaggctcttttaagacacttactcttccatcactacctctgaacagtgcagatgagatttatgagctacgtgtaaccggacgtacccaggatgagattttattctctaatagtacccgcttatcatttgagaccaagagaatatctgtcttcattcaaacagacaaggccttatacaagccaaagcaagaagtgaagtttcgcattgttacactcttctcagattttaagccttacaaaacctctttaaacattctcattaaggaccccaaatcaaatttgatccaacagtggttgtcacaacaaagtgatcttggagtcatttccaaaacttttcagctatcttcccatccaatacttggtgactggtctattcaagttcaagtgaatgaccagacatattatcaatcatttcaggtttcagaatatgtattaccaaaatttgaagtgactttgcagacaccattatattgttctatgaattctaagcatttaaatggtaccatcacggcaaagtatacatatgggaagccagtgaaaggagacgtaacgcttacatttttacctttatccttttggggaaagaagaaaaatattacaaaaacatttaagataaatggatctgcaaacttctcttttaatgatgaagagatgaaaaatgtaatggattcttcaaatggactttctgaatacctggatctatcttcccctggaccagtagaaattttaaccacagtgacagaatcagttacaggtatttcaagaaatgtaagcactaatgtgttcttcaagcaacatgattacatcattgagttttttgattatactactgtcttgaagccatctctcaacttcacagccactgtgaaggtaactcgtgctgatggcaaccaactgactcttgaagaaagaagaaataatgtagtcataacagtgacacagagaaactatactgagtactggagcggatctaacagtggaaatcagaaaatggaagctgttcagaaaataaattatactgtcccccaaagtggaacttttaagattgaattcccaatcctggaggattccagtgagctacagttgaaggcctatttccttggtagtaaaagtagcatggcagttcatagtctgtttaagtctcctagtaagacatacatccaactaaaaacaagagatgaaaatataaaggtgggatcgccttttgagttggtggttagtggcaacaaacgattgaaggagttaagctatatggtagtatccaggggacagttggtggctgtaggaaaacaaaattcaacaatgttctctttaacaccagaaaattcttggactccaaaagcctgtgtaattgtgtattatattgaagatgatggggaaattataagtgatgttctaaaaattcctgttcagcttgtttttaaaaataagataaagctatattggagtaaagtgaaagctgaaccatctgagaaagtctctcttaggatctctgtgacacagcctgactccatagttgggattgtagctgttgacaaaagtgtgaatctgatgaatgcctctaatgatattacaatggaaaatgtggtccatgagttggaactttataacacaggatattatttaggcatgttcatgaattcttttgcagtctttcaggaatgtggactctgggtattgacagatgcaaacctcacgaaggattatattgatggtgtttatgacaatgcagaatatgctgagaggtttatggaggaaaatgaaggacatattgtagatattcatgacttttctttgggtagcagtccacatgtccgaaagcattttccagagacttggatttggctagacaccaacatgggttacaggatttaccaagaatttgaagtaactgtacctgattctatcacttcttgggtggctactggttttgtgatctctgaggacctgggtcttggactaacaactactccagtggagctccaagccttccaaccatttttcatttttttgaatcttccctactctgttatcagaggtgaagaatttgctttggaaataactatattcaattatttgaaagatgccactgaggttaaggtaatcattgagaaaagtgacaaatttgatattctaatgacttcaaatgaaataaatgccacaggccaccagcagacccttctggttcccagtgaggatggggcaactgttctttttcccatcaggccaacacatctgggagaaattcctatcacagtcacagctctttcacccactgcttctgatgctgtcacccagatgattttagtaaaggctgaaggaatagaaaaatcatattcacaatccatcttattagacttgactgacaataggctacagagtaccctgaaaactttgagtttctcatttcctcctaatacagtgactggcagtgaaagagttcagatcactgcaattggagatgttcttggtccttccatcaatggcttagcctcattgattcggatgccttatggctgtggtgaacagaacatgataaattttgctccaaatatttacattttggattatctgactaaaaagaaacaactgacagataatttgaaagaaaaagctctttcatttatgaggcaaggttaccagagagaacttctctatcagagggaagatggctctttcagtgcttttgggaattatgacccttctgggagcacttggttgtcagcttttgttttaagatgtttccttgaagccgatccttacatagatattgatcagaatgtgttacacagaacatacacttggcttaaaggacatcagaaatccaacggtgaattttgggatccaggaagagtgattcatagtgagcttcaaggtggcaataaaagtccagtaacacttacagcctatattgtaacttctctcctgggatatagaaagtatcagcctaacattgatgtgcaagagtctatccattttttggagtctgaattcagtagaggaatttcagacaattatactctagcccttataacttatgcattgtcatcagtggggagtcctaaagcgaaggaagctttgaatatgctgacttggagagcagaacaagaaggtggcatgcaattctgggtgtcatcagagtccaaactttctgactcctggcagccacgctccctggatattgaagttgcagcctatgcactgctctcacacttcttacaatttcagacttctgagggaatcccaattatgaggtggctaagcaggcaaagaaatagcttgggtggttttgcatctactcaggataccactgtggctttaaaggctctgtctgaatttgcagccctaatgaatacagaaaggacaaatatccaagtgaccgtgacggggcctagctcaccaagtcctgtaaagtttctgattgacacacacaaccgcttactccttcagacagcagagcttgctgtggtacagccaatggcagttaatatttccgcaaatggttttggatttgctatttgtcagctcaatgttgtatataatgtgaaggcttctgggtcttctagaagacgaagatctatccaaaatcaagaagcctttgatttagatgttgctgtaaaagaaaataaagatgatctcaatcatgtggatttgaatgtgtgtacaagcttttcgggcccgggtaggagtggcatggctcttatggaagttaacctattaagtggctttatggtgccttcagaagcaatttctctgagcgagacagtgaagaaagtggaatatgatcatggaaaactcaacctctatttagattctgtaaatgaaacccagttttgtgttaatattcctgctgtgagaaactttaaagtttcaaatacccaagatgcttcagtgtccatagtggattactatgagccaaggagacaggcggtgagaagttacaactctgaagtgaagctgtcctcctgtgacctttgcagtgatgtccagggctgccgtccttgtgaggatggagcttcaggctcccatcatcactcttcagtcatttttattttctgtttcaagcttctgtactttatggaactttggctgtgatttatttttaaaggactctgtgtaacactaacatttccagtagtcacatgtgattgttttgttttcgtagaagaatactgcttctattttgaaaaaagagttttttttctttctatggggttgcagggatggtgtacaacaggtcctagcatgtatagctgcatagatttcttcacctgatctttgtgtggaagatcagaatgaatgcagttgtgtgtctatattttcccctcacaaaatcttttagaatttttttggaggtgtttgttttctccagaataaaggtattactttagaaataggtattctcctcattttgtgaaagaaatgaacctagattcttaagcattattacacatccatgtttgcttaaagatggatttccctgggaatgggagaaaacagccagcaggaggagcttcatctgttcccttcccacctccaacctagccctactgcccaccccaccccaacccaccccatgcccagtggtctcagtagatacttcttaactggaaattctttcttttcagaatctaggtggtgaattttttttaagtggcacggtctttttctgcttgaaatctgatcacaccccccagccattgccctccctctctttttcctctgtagagaaatgtgaggggcagtacatttactgtgcttttcacaccatctcagaggttgaggagcatactgaaaattgccctggggggtgctgggtgtgctgtctccttcccacatcctcagccccacaccagctctatttcaggggtgagagtcagagagcactgcaatatgtgcttcatgggatttcgattcgaagatcctagaccagggagacactgtgagccagggatacaacaaaatactaggtaagtcactgcagaccgacctccctgcagtttgggaaagaagctgggtttgtggagaatcagagcatcttgacatgactgctgacctaaagatccctggcattggccagggatcctgtggaacctcttctagttcaggggtgtgagcattagactgccagttgtctagtgacatctgatgcttgctgtgaacttttaagatccccgaatcctgagcacctcaatctttaattgccctgtattccgaagggtaatataatttatctggatggaaattttaaagatgaatcccccttttttcttttcttctctcttttctttccttctccctttcttctttgccttctaaatatactgaaatgatttagatatgtgtcaacaattaatgatcttttattcaatctaagaaatggtttagtttttctctttagctctatggcatttcactcaagtggacaggggaaaaagtaattgccatgggctccaaagaatttgctttatgtttttagctatttaaaaataaatccatcaaaaataaagtatgcaaatgtatctttt

SEQ ID NO: 6 - RNA, 4449 nt, Homo sapiens
  source 1..4449: mol_type = mRNA
aaaactcgaattaagagggaaagaaaatcagggaggaggtggcaagccacaccccacggtgcccgcgaacttccccggcagcggactgtagcccaggcagacgccgtcgagatgcagggcccaccgctcctgaccgccgcccacctcctctgcgtgtgcaccgccgcgctggccgtggctcccgggcctcggtttctggtgacagccccagggatcatcaggcccggaggaaatgtgactattggggtggagcttctggaacactgcccttcacaggtgactgtgaaggcggagctgctcaagacagcatcaaacctcactgtctctgtcctggaagcagaaggagtctttgaaaaaggctcttttaagacacttactcttccatcactacctctgaacagtgcagatgagatttatgagctacgtgtaaccggacgtacccaggatgagattttattctctaatagtacccgcttatcatttgagaccaagagaatatctgtcttcattcaaacagacaaggccttatacaagccaaagcaagaagtgaagtttcgcattgttacactcttctcagattttaagccttacaaaacctctttaaacattctcattaaggaccccaaatcaaatttgatccaacagtggttgtcacaacaaagtgatcttggagtcatttccaaaacttttcagctatcttcccatccaatacttggtgactggtctattcaagttcaagtgaatgaccagacatattatcaatcatttcaggtttcagaatatgtattaccaaaatttgaagtgactttgcagacaccattatattgttctatgaattctaagcatttaaatggtaccatcacggcaaagtatacatatgggaagccagtgaaaggagacgtaacgcttacatttttacctttatccttttggggaaagaagaaaaatattacaaaaacatttaagataaatggatctgcaaacttctcttttaatgatgaagagatgaaaaatgtaatggattcttcaaatggactttctgaatacctggatctatcttcccctggaccagtagaaattttaaccacagtgacagaatcagttacaggtatttcaagaaatgtaagcactaatgtgttcttcaagcaacatgattacatcattgagttttttgattatactactgtcttgaagccatctctcaacttcacagccactgtgaaggtaactcgtgctgatggcaaccaactgactcttgaagaaagaagaaataatgtagtcataacagtgacacagagaaactatactgagtactggagcggatctaacagtggaaatcagaaaatggaagctgttcagaaaataaattatactgtcccccaaagtggaacttttaagattgaattcccaatcctggaggattccagtgagctacagttgaaggcctatttccttggtagtaaaagtagcatggcagttcatagtctgtttaagtctcctagtaagacatacatccaactaaaaacaagagatgaaaatataaaggtgggatcgccttttgagttggtggttagtggcaacaaacgattgaaggagttaagctatatggtagtatccaggggacagttggtggctgtaggaaaacaaaattcaacaatgttctctttaacaccagaaaattcttggactccaaaagcctgtgtaattgtgtattatattgaagatgatggggaaattataagtgatgttctaaaaattcctgttcagcttgtttttaaaaataagataaagctatattggagtaaagtgaaagctgaaccatctgagaaagtctctcttaggatctctgtgacacagcctgactccatagttgggattgtagctgttgacaaaagtgtgaatctgatgaatgcctctaatgatattacaatggaaaatgtggtccatgagttggaactttataacacaggatattatttaggcatgttcatgaattcttttgcagtctttcaggaatgtggactctgggtattgacagatgcaaacctcacgaaggattatattgatggtgtttatgacaatgcagaatatgctgagaggtttatggaggaaaatgaaggacatattgtagatattcatgacttttctttgggtagcagtccacatgtccgaaagcattttccagagacttggatttggctagacaccaacatgggttccaggatttaccaagaatttgaagtaactgtacctgattctatcacttcttgggtggctactggttttgtgatctctgaggacctgggtcttggactaacaactactccagtggagctccaagccttccaaccatttttcatttttttgaatcttccctactctgttatcagaggtgaagaatttgctttggaaataactatattcaattatttgaaagatgccactgaggttaaggtaatcattgagaaaagtgacgaatttgatattctaatgacttcaaatgaaataaatgccacaggccaccagcagacccttctggttcccagtgaggatggggcaactgttctttttcccatcaggccaacacatctgggagaaattcctatcacagtcacagctctttcacccactgcttctgatgctgtcacccagatgattttagtaaaggctgaaggaatagaaaaatcatattcacaatccatcttattagacttgactgacaataggctacagagtaccctgaaaactttgagtttctcatttcctcctaatacagtgactggcagtgaaagagttcagatcactgcaattggagatgttcttggtccttccatcaatggcttagcctcattgattcggatgccttatggctgtggtgaacagaacatgataaattttgctccaaatatttacattttggattatctgactaaaaagaaacaactgacagataatttgaaagaaaaagctctttcatttatgaggcaaggttaccagagagaacttctctatcagagggaagatggctctttcagtgcttttgggaattatgacccttctgggagcacttggttgtcagcttttgttttaagatgtttccttgaagccgatccttacatagatattgatcagaatgtgttacacagaacatacacttggcttaaaggacatcagaaatccaacggtgaattttgggatccaggaagagtgattcatagtgagcttcaaggtggcaataaaagtccagtaacacttacagcctatattgtaacttctctcctgggatatagaaagtatcagcctaacattgatgtgcaagagtctatccattttttggagtctgaattcagtagaggaatttcagacaattatactctagcccttataacttatgcattgtcatcagtggggagtcctaaagcgaaggaagctttgaatatgctgacttggagagcagaacaagaaggtggcatgcaattctgggtgtcatcagagtccaaactttctgactcctggcagccacgctccctggatattgaagttgcagcctatgcactgctctcacacttcttacaatttcagacttctgagggaatcccaattatgaggtggctaagcaggcaaagaaatagcttgggtggttttgcatctactcaggataccactgtggctttaaaggctctgtctgaatttgcagccctaatgaatacagaaaggacaaatatccaggtgaccgtgacggggcctagctcaccaagtcctgtaaagtttctgattgacacacacaaccgcttactccttcagacagcagagcttgctgtggtacagccaacggcagttaatatttccgcaaatggttttggatttgctatttgtcagctcaatgttgtatataatgtgaaggcttctgggtcttctagaagacgaagatctatccaaaatcaagaagcctttgatttagatgttgctgtaaaagaaaataaagatgatctcaatcatgtggatttgaatgtgtgtacaagcttttcgggcccgggtaggagtggcatggctcttatggaagttaacctattaagtggctttatggtgccttcagaagcaatttctctgagcgagacagtgaagaaagtggaatatgatcatggaaaactcaacctctatttagattctgtaaatgaaacccagttttgtgttaatattcctgctgtgagaaactttaaagtttcaaatacccaagatgcttcagtgtccatagtggattactatgagccaaggagacaggcggtgagaagttacaactctgaagtgaagctgtcctcctgtgacctttgcagtgatgtccagggctgccgtccttgtgaggatggagcttcaggctcccatcatcactcttcagtcatttttattttctgtttcaagcttctgtactttatggaactttggctgtga

SEQ ID NO: 7 - RNA, 2273 nt, Homo sapiens
  source 1..2273: mol_type = mRNA
acacaccccacggtgcccgcgaacttccccggcagcggactgtagcccaggcagacgccgtcgagatgcagggcccaccgctcctgaccgccgcccacctcctctgcgtgtgcaccgccgcgctggccgtggctcccgggcctcggtttctggtgacagccccagggatcatcaggcccggaggaaatgtgactattggggtggagcttctggaacactgcccttcacaggtgactgtgaaggcggagctgctcaagacagcatcaaacctcactgtctctgtcctggaagcagaaggagtctttgaaaaaggctcttttaagacacttactcttccatcactacctctgaacagtgcagatgagatttatgagctacgtgtaaccggacgtacccaggatgagattttattctctaatagtacccgcttatcatttgagaccaagagaatatctgtcttcattcaaacagacaaggccttatacaagccaaagcaagaagtgaagtttcgcattgttacactcttctcagattttaagccttacaaaacctctttaaacattctcattaaggaccccaaatcaaatttgatccaacagtggttgtcacaacaaagtgatcttggagtcatttccaaaacttttcagctatcttcccatccaatacttggtgactggtctattcaagttcaagtgaatgaccagacatactatcaatcatttcaggtttcagaatatgtattaccaaaatttgaagtgactttgcagacaccattatattgttctatgaattctaagcatttaaatggtaccatcacggcaaagtatacatatgggaagccagtgaaaggagacgtaacgcttacatttttacctttatccttttggggaaagaagaaaaatattacaaaaacatttaagataaatggatctgcaaacttctcttttaatgatgaagagatgaaaaatgtaatggattcttcaaatggactttctgaatacctggatctatcttcccctggaccagtagaaattttaaccacagtgacagaatcagttacaggtatttcaagaaatgtaagcactaatgtgttcttcaagcaacatgattacatcattgagttttttgattatactactgtcttgaagccatctctcaacttcacagccactgtgaaggtaactcgtgctgatggcaaccaactgactcttgaagaaagaagaaataatgtagtcataacagtgacacagagaaactatactgagtactggagcggatctaacagtggaaatcagaaaatggaagctgttcagaaaataaattatactgtcccccaaagtggaacttttaagattgaattcccaatcctggaggattccagtgagctacagttgaaggcctatttccttggtagtaaaagtagcatggcagttcatagtctgtttaagtctcctagtaagacatacatccaactaaaaacaagagatgaaaatataaaggtgggatcgccttttgagttggtggttagtggcaacaaacgattgaaggagttaagctatatggtagtatccaggggacagttggtggctgtaggaaaacaaaattcaacaatgttctctttaacaccagaaaattcttggactccaaaagcctgtgtaattgtgtattatattgaagatgatggggaaattataagtgatgttctaaaaattcctgttcagcttgtttttaaaaataagataaagctatattggagtaaagtgaaagctgaaccatctgagaaagtctctcttaggatctctgtgacacagcctgactccatagttgggattgtagctgttgacaaaagtgtgaatctgatgaatgcctctaatgatattacaatggaaaatgtggtccatgagttggaactttataacacaggatattatttaggcatgttcatgaattcttttgcagtctttcaggaatgtggactctgggtattgacagatgcaaacctcacgaaggattatattgatggtgtttatgacaatctctttggtacacaggaagctttataaaatttcattcacgaatctcttattttgggaagctgttttgcatatgagaagaacactgttgaaataaggaactaaagctttatatattgatcaaggtgattctgaaagttttaatttttaatgttgtaatgttatgttattgttaattgtactttattatgtattcaatagaaaatcatgatttattaataaaagcttaaattctcatct

SEQ ID NO: 8 - RNA, 4688 nt, Homo sapiens
  source 1..4688: mol_type = mRNA
gggactgtagcccaggcagacgccgtcgagatgcagggcccaccgctcctgaccgccgcccacctcctctgcgtgtgcaccgccgcgctggccgtggctcccgggcctcggtttctggtgacagccccagggatcatcaggcccggaggaaatgtgactattggggtggagcttctggaacactgcccttcacaggtgactgtgaaggcggagctgctcaagacagcatcaaacctcactgtctctgtcctggaagcagaaggagtctttgaaaaaggctcttttaagacacttactcttccatcactacctctgaacagtgcagatgagatttatgagctacgtgtaaccggacgtacccaggatgagattttattctctaatagtacccgcttatcatttgagaccaagagaatatctgtcttcattcaaacagacaaggccttatacaagccaaagcaagaagtgaagtttcgcattgttacactcttctcagattttaagccttacaaaacctctttaaacattctcattaaggaccccaaatcaaatttgatccaacagtggttgtcacaacaaagtgatcttggagtcatttccaaaacttttcagctatcttcccatccaatacttggtgactggtctattcaagttcaagtgaatgaccagacatattatcaatcatttcaggtttcagaatatgtattaccaaaatttgaagtgactttgcagacaccattatattgttctatgaattctaagcatttaaatggtaccatcacggcaaagtatacatatgggaagccagtgaaaggagacgtaacgcttacatttttacctttatccttttggggaaagaagaaaaatattacaaaaacatttaagataaatggatctgcaaacttctcttttaatgatgaagagatgaaaaatgtaatggattcttcaaatggactttctgaatacctggatctatcttcccctggaccagtagaaattttaaccacagtgacagaatcagttacaggtatttcaagaaatgtaagcactaatgtgttcttcaagcaacatgattacatcattgagttttttgattataccactgtcttgaagccatctctcaacttcacagccactgtgaaggtaactcgtgctgatggcaaccaactgactcttgaagaaagaagaaataatgtagtcataacagtgacacagagaaactatactgagtactggagcggatctaacagtggaaatcagaaaatggaagctgttcagaaaataaattatactgtcccccaaagtggaacttttaagattgaattcccaatcctggaggattccagtgagctacagttgaaggcctatttccttggtagtaaaagtagcatggcagttcatagtctgtttaagtctcctagtaagacatacatccaactaaaaacaagagatgaaaatataaaggtgggatcgccttttgagttggtggttagtggcaacaaacgattgaaggagttaagctatatggtagtatccaggggacagttggtggctgtaggaaaacaaaattcaacaatgttctctttaacaccagaaaattcttggactccaaaagcctgtgtaattgtgtattatattgaagatgatggggaaattataagtgatgttctaaaaattcctgttcagcttgtttttaaaaataagataaagctatattggagtaaagtgaaagctgaaccatctgagaaagtctctcttaggatctctgtgacacagcctgactccatagttgggattgtagctgttgacaaaagtgtgaatctgatgaatgcctctaatgatattacaatggaaaatgtggtccatgagttggaactttataacacaggatattatttaggcatgttcatgaattcttttgcagtctttcaggaatgtggactctgggtattgacagatgcaaacctcacgaaggattatattgatggtgtttatgacaatgcagaatatgctgagaggtttatggaggaaaatgaaggacatattgtagatattcatgacttttctttgggtagcagtccacatgtccgaaagcattttccagagacttggatttggctagacaccaacatgggttccaggatttaccaagaatttgaagtaactgtacctgattctatcacttcttgggtggctactggttttgtgatctctgaggacctgggtcttggactaacaactactccagtggagctccaagccttccaaccatttttcatttttttgaatcttccctactctgttatcagaggtgaagaatttgctttggaaataactatattcaattatttgaaagatgccactgaggttaaggtaatcattgagaaaagtgacaaatttgatattctaatgacttcaagtgaaataaatgccacaggccaccagcagacccttctggttcccagtgaggatggggcaactgttctttttcccatcaggccaacacatctgggagaaattcctatcacagtcacagctctttcacccactgcttctgatgctatcacccagatgattttagtaaaggctgaaggaatagaaaaatcatattcacaatccatcttattagacttgactgacaataggctacagagtaccctgaaaactttgagtttctcatttcctcctaatacagtgactggcagtgaaagagttcagatcactgcaattggagatgttcttggtccttccatcaatggcttagcctcattgattcggatgccttatggctgtggtgaacagaacatgataaattttgctccaaatatttacattttggattatctgactaaaaagaaacaactgacagataatttgaaagaaaaagctctttcatttatgaggcaaggttaccagagagaacttctctatcagagggaagatggctctttcagtgcttttgggaattatgacccttctgggagcacttggttgtcagcttttgttttaagatgtttccttgaagccgatccttacatagatattgatcagaatgtgttacacagaacatacacttggcttaaaggacatcagaaatccaacggtgaattttgggatccaggaagagtgattcatagtgagcttcaaggtggcaataaaagtccagcaacacttacagcctatattgtaacttctctcctgggatatagaaagtatcagcctaacattgatgtgcaagagtctatccattttttggagtctgaattcagtagaggaatttcagacaattatactctagcccttataacttatgcattgtcatcagtggggagtcctaaagcgaaggaagctttgaatatgctgacttggagagcagaacaagaaggtggcatgcaattctgggtgtcatcagagtccaaactttctgactcctggcagccacgctccctggatattgaagttgcagcctatgcactgctctcacacttcttacaatttcagacttctgagggaatcccaattatgaggtggctaagcaggcaaagaaatagcttgggtggttttgcatctactcaggataccactgtggctttaaaggctctgtctgaatttgcagccctaatgaatacagaaaggacaaatatccaagtgaccgtgacggggcctagctcaccaagtcctgtaaagtttctgattgacacacacaaccgcttactccttcagacagcagagcttgctgtggtacagccaatggcagttaatatttccgcaaatggttttggatttgctatttgtcagctcaatgttgtatataatgtgaaggcttctgggtcttctagaagacgaagatctatccaaaatcaagaagcctttgatttagatgttgctgtaaaagaaaataaagatgatctcaatcatgtggatttgaatgtgtgtacaagcttttcgggcccgggtaggagtggcatggctcttatggaagttaacctattaagtggctttatggtgccttcagaagcaatttctctgagcgagacagtgaagaaagtggaatatgatcatggaaaactcaacctctatttagattctgtaaatgaaacccagttttgtgttaatattcctgctgtgagaaactttaaagtttcaaatacccaagatgcttcagtgtccatagtggattactatgagccaaggagacaggcggtgagaagttacaactctgaagtgaagctgtcctcctgtgacctttgcagtgatgtccagggctgccgtccttgtgaggatggagcttcaggctcccatcatcactcttcagtcatttttattttctgtttcaagcttctgtactttatggaactttggctgtgatttatttttaaaggactctgtgtaacactaacatttccagtagtcacatgtgattgttttgttttcgtagaagaatactgcttctattttgaaaaaagagttttttttctttctatggggttgcagggatggtgtacaacaggtcctagcatgtatagctgcatagatttcttcacctgatctttgtgtggaagatcagaatgaatgcagttgtgtgtctatattttcccctctcaaaatcttttagaatttttttggaggtgtttgttttctccagaataaaggtattactttagaaaacaaaaaaaaaaaaaaaaaaa

SEQ ID NO: 9 - RNA, 4369 nt, Homo sapiens
  source 1..4369: mol_type = mRNA
tgtagcccaggcagacgccgtcgagatgcagggcccaccgctcctgaccgccgcccacctcctctgcgtgtgcaccgccgcgctggccgtggctcccgggcctcggtttctggtgacagccccagggatcatcaggcccggaggaaatgtgactattggggtggagcttctggaacactgcccttcacaggtgactgtgaaggcggagctgctcaagacagcatcaaacctcactgtctctgtcctggaagcagaaggagtctttgaaaaaggctcttttaagacacttactcttccatcactacctctgaacagtgcagatgagatttatgagctacgtgtaaccggacgtacccaggatgagattttattctctaatagtacccgcttatcatttgagaccaagagaatatctgtcttcattcaaacagacaaggccttatacaagccaaagcaagaagtgaagtttcgcattgttacactcttctcagattttaagccttacaaaacctctttaaacattctcattaaggaccccaaatcaaatttgatccaacagtggttgtcacaacaaagtgatcttggagtcatttccaaaacttttcagctatcttcccatccaatacttggtgactggtctattcaagttcaagtgaatgaccagacatattatcaatcatttcaggtttcagaatatgtattaccaaaatttgaagtgactttgcagacaccattatattgttctatgaattctaagcatttaaatggtaccatcacggcaaagtatacatatgggaagccagtgaaaggagacgtaacgcttacatttttacctttatccttttggggaaagaagaaaaatattacaaaaacatttaagataaatggatctgcaaacttctcttttaatgatgaagagatgaaaaatgtaatggattcttcaaatggactttctgaatacctggatctatcttcccctggaccagtagaaattttaaccacagtgacagaatcagttacaggtatttcaagaaatgtaagcactaatgtgttcttcaagcaacatgattacatcattgagttttttgattatactactgtcttgaagccatctctcaacttcacagccactgtgaaggtaactcgtgctgatggcaaccaactgactcttgaagaaagaagaaataatgtagtcataacagtgacacagagaaactatactgagtactggagcggatctaacagtggaaatcagaaaatggaagctgttcagaaaataaattatactgtcccccaaagtggaacttttaagattgaattcccaatcctggaggattccagtgagctacagttgaaggcctatttccttggtagtaaaagtagcatggcagttcatagtctgtttaagtctcctagtaagacatacatccaactaaaaacaagagatgaaaatataaaggtgggatcgccttttgagttggtggttagtggcaacaaacgattgaaggagttaagctatatggtagtatccaggggacagttggtggctgtaggaaaacaaaattcaacaatgttctctttaacaccagaaaattcttggactccaaaagcctgtgtaattgtgtattatattgaagatgatggggaaattataagtgatgttctaaaaattcctgttcagcttgtttttaaaaataagataaagctatattggagtaaagtgaaagctgaaccatctgagaaagtctctcttaggatctctgtgacacagcctgactccatagttgggattgtagctgttgacaaaagtgtgaatctgatgaatgcctctaatgatattacaatggaaaatgtggtccatgagttggaactttataacacaggatattatttaggcatgttcatgaattcttttgcagtctttcaggaatgtggactctgggtattgacagatgcaaacctcacgaaggattatattgatggtgtttatgacaatgcagaatatgctgagaggtttatggaggaaaatgaaggacatattgtagatattcatgacttttctttgggtagcagtccacatgtccgaaagcattttccagagacttggatttggctagacaccaacatgggttccaggatttaccaagaatttgaagtaactgtacctgattctatcacttcttgggtggctactggttttgtgatctctgaggacctgggtcttggactaacaactactccagtggagctccaagccttccaaccatttttcatttttttgaatcttccctactctgttatcagaggtgaagaatttgctttggaaataactatattcaattatttgaaagatgccactgaggttaaggtaatcattgagaaaagtgacaaatttgatattctaatgacttcaagtgaaataaatgccacaggccaccagcagacccttctggttcccagtgaggatggggcaactgttctttttcccatcaggccaacacatctgggagaaattcctatcacagtcacagctctttcacccactgcttctgatgctatcacccagatgattttagtaaaggctgaaggaatagaaaaatcatattcacaatccatcttattagacttgactgacaataggctacagagtaccctgaaaactttgagtttctcatttcctcctaatacagtgactggcagtgaaagagttcagatcactgcaattggagatgttcttggtccttccatcaatggcttagcctcattgattcggatgccttatggctgtggtgaacagaacatgataaattttgctccaaatatttacattttggattatctgactaaaaagaaacaactgacagataatttgaaagaaaaagctctttcatttatgaggcaaggttaccagagagaacttctctatcagagggaagatggctctttcagtgcttttgggaattatgacccttctgggagcacttggttgtcagcttttgttttaagatgtttccttgaagccgatccttacatagatattgatcagaatgtgttacacagaacatacacttggcttaaaggacatcagaaatccaacggtgaattttgggatccaggaagagtgattcatagtgagcttcaaggtggcaataaaagtccagtaacacttacagcctatattgtaacttctctcctgggatatagaaagtatcagcctaacattgatgtgcaagagtctatccattttttggagtctgaattcagtagaggaatttcagacaattatactctagcccttataacttatgcattgtcatcagtggggagtcctaaagcgaaggaagctttgaatatgctgacttggagagcagaacaagaaggtggcatgcaattctgggtgtcatcagagtccaaactttctgactcctggcagccacgctccctggatattgaagttgcagcctatgcactgctctcacacttcttacaatttcagacttctgagggaatcccaattatgaggtggctaagcaggcaaagaaatagcttgggtggttttgcatctactcaggataccactgtggctttaaaggctctgtctgaatttgcagccctaatgaatacagaaaggacaaatatccaagtgaccgtgacggggcctagctcaccaagtcctcttgctgtggtacagccaacggcagttaatatttccgcaaatggttttggatttgctatttgtcagctcaatgttgtatataatgtgaaggcttctgggtcttctagaagacgaagatctatccaaaatcaagaagcctttgatttagatgttgctgtaaaagaaaataaagatgatctcaatcatgtggatttgaatgtgtgtacaagcttttcgggcccgggtaggagtggcatggctcttatggaagttaacctattaagtggctttatggtgccttcagaagcaatttctctgagcgagacagtgaagaaagtggaatatgatcatggaaaactcaacctctatttagattctgtaaatgaaacccagttttgtgttaatattcctgctgtgagaaactttaaagtttcaaatacccaagatgcttcagtgtccatagtggattactatgagccaaggagacaggcggtgagaagttacaactctgaagtgaagctgtcctcctgtgacctttgcagtgatgtccagggctgccgtccttgtgaggatggagcttcaggctcccatcatcactcttcagtcatttttattttctgtttcaagcttctgtactttatggaactttggctgtgatttatttttaaaggactctgtgtaacactaacatttccagtagtcacatgtgattgt

SEQ ID NO: 10 - RNA, 4237 nt, Homo sapiens
  source 1..4237: mol_type = mRNA
gaggcagacgccgtcgagatgcagggcccaccgctcctgaccgccgcccacctcctctgcgtgtgcaccgccgcgctggccgtggctcccgggcctcggtttctggtgacagccccagggatcatcaggcccggaggaaatgtgactattggggtggagcttctggaacactgcccttcacaggtgactgtgaaggcggagctgctcaagacagcatcaaacctcactgtctctgtcctggaagcagaaggagtctttgaaaaaggctcttttaagacacttactcttccatcagaccccaaatcaaatttgatccaacagtggttgtcacaacaaagtgatcttggagtcatttccaaaacttttcagctatcttcccatccaatacttggtgactggtctattcaagttcaagtgaatgaccagacatactatcaatcatttcaggtttcagaatatgtattaccaaaatttgaagtgactttgcagacaccattatattgttctatgaattctaagcatttaaatggtaccatcacggcaaagtatacatatgggaagccagtgaaaggagacgtaacgcttacatttttacctttatccttttggggaaagaagaaaaatattacaaaaacatttaagataaatggatctgcaaacttctcttttaatgatgaagagatgaaaaatgtaatggattcttcaaatggactttctgaatacctggatctatcttcccctggaccagtagaaattttaaccacagtgacagaatcagttacaggtatttcaagaaatgtaagcactaatgtgttcttcaagcaacatgattacatcattgagttttttgattatactactgtcttgaagccatctctcaacttcacagccactgtgaaggtaactcgtgctgatggcaaccaactgactcttgaagaaagaagaaataatgtagtcataacagtgacacagagaaactatactgagtactggagcggatctaacagtggaaatcagaaaatggaagctgttcagaaaataaattatactgtcccccaaagtggaacttttaagattgaattcccaatcctggaggattccagtgagctacagttgaaggcctatttccttggtagtaaaagtagcatggcagttcatagtctgtttaagtctcctagtaagacatacatccaactaaaaacaagagatgaaaatataaaggtgggatcgccttttgagttggtggttagtggcaacaaacgattgaaggagttaagctatatggtagtatccaggggacagttggtggctgtaggaaaacaaaattcaacaatgttctctttaacaccagaaaattcttggactccaaaagcctgtgtaattgtgtattatattgaagatgatggggaaattataagtgatgttctaaaaattcctgttcagcttgtttttaaaaataagataaagctatattggagtaaagtgaaagctgaaccatctgagaaagtctctcttaggatctctgtgacacagcctgactccatagttgggattgtagctgttgacaaaagtgtgaatctgatgaatgcctctaatgatattacaatggaaaatgtggtccatgagttggaactttataacacaggatattatttaggcatgttcatgaattcttttgcagtctttcaggaatgtggactctgggtattgacagatgcaaacctcacgaaggattatattgatggtgtttatgacaatgcagaatatgctgagaggtttatggaggaaaatgaaggacatattgtagatattcatgacttttctttgggtagcagtccacatgtccgaaagcattttccagagacttggatttggctagacaccaacatgggttacaggatttaccaagaatttgaagtaactgtacctgattctatcacttcttgggtggctactggttttgtgatctctgaggacctgggtcttggactaacaactactccagtggagctccaagccttccaaccatttttcatttttttgaatcttccctactctgttatcagaggtgaagaatttgctttggaaataactatattcaattatttgaaagatgccactgaggttaaggtaatcattgagaaaagtgacaaatttgatattctaatgacttcaaatgaaataaatgccacaggccaccagcagacccttctggttcccagtgaggatggggcaactgttctttttcccatcaggccaacacatctgggagaaattcctatcacagtcacagctctttcacccactgcttctgatgctgtcacccagatgattttagtaaaggctgaaggaatagaaaaatcatattcacaatccatcttattagacttgactgacaataggctacagagtaccctgaaaactttgagtttctcatttcctcctaatacagtgactggcagtgaaagagttcagatcactgcaattggagatgttcttggtccttccatcaatggcttagcctcattgattcggatgccttatggctgtggtgaacagaacatgataaattttgctccaaatatttacattttggattatctgactaaaaagaaacaactgacagataatttgaaagaaaaagctctttcatttatgaggcaaggttaccagagagaacttctctatcagagggaagatggctctttcagtgcttttgggaattatgacccttctgggagcacttggttgtcagcctttgttttaagatgtttccttgaagccgatccttacatagatattgatcagaatgtgttacacagaacatacacttggcttaaaggacatcagaaatccaacggtgaattttgggatccaggaagagtgattcatagtgagcttcaaggtggcaataaaagtccagtaacacttacagcctatattgtaacttctctcctgggatatagaaagtatcagcctaacattgatgtgcaagagtctatccattttttggagtctgaattcagtagaggaatttcagacaattatactctagcccttataacttatgcattgtcatcagtggggagtcctaaagcgaaggaagctttgaatatgctgacttggagagcagaacaagaaggtggcatgcaattctgggtgtcatcagagtccaaactttctgactcctggcagccacgctccctggatattgaagttgcagcctatgcactgctctcacacttcttacaatttcagacttctgagggaatcccaattatgaggtggctaagcaggcaaagaaatagcttgggtggttttgcatctactcaggataccactgtggctttaaaggctctgtctgaatttgcagccctaatgaatacagaaaggacaaatatccaagtgaccgtgacggggcctagctcaccaagtcctgtaaagtttctgattgacacacacaaccgcttactccttcagacagcagagcttgctgtggtacagccaatggcagttaatatttccgcaaatggttttggatttgctatttgtcagctcaatgttgtatataatgtgaaggcttctgggtcttctagaagacgaagatctatccaaaatcaagaagccttttgatttagatgttgctgtaaaagaaaataaagatgatctcaatcatgtggatttgaatgtgtgtacaagcttttcgggcccgggtaggagtggcatggctcttatggaagttaacctattaagtggctttatggtgccttcagaagcaatttctctgagcgagacagtgaagaaagtggaatatgatcatggaaaactcaacctctatttagattctgtaaatgaaacccagttttgtgttaatattcctgctgtgagaaactttaaagtttcaaatacccaagatgcttcagtgtccatagtggattactatgagccaaggagacaggcggtgagaagttacaactctgaagtgaagctgtcctcctgtgacctttgcagtgatgtccagggctgccgtccttgtgaggatggagcttcaggctcccatcatcactcttcagtcatttttattttctgtttcaagcttctgtactttatggaactttggctgtgatttatttttaaaggactctgtgtaacactaacatttccagtagtcacatgtgattgttttgttttcgtagaagaatactgcttctattttgaaaaaaaaaaaaaaaaaaca

SEQ ID NO: 11 - RNA, 4338 nt, Homo sapiens
  source 1..4338: mol_type = mRNA
atgcagggcccaccgctcctgaccgccgcccacctcctctgcgtgtgcaccgccgcgctggccgtggctcccgggcctcggtttctggtgacagccccagggatcatcaggcccggaggaaatgtgactattggggtggagcttctggaacactgcccttcacaggtgactgtgaaggcggagctgctcaagacagcatcaaacctcactgtctctgtcctggaagcagaaggagtctttgaaaaaggctcttttaagacacttactcttccatcactacctctgaacagtgcagatgagatttatgagctacgtgtaaccggacgtacccaggatgagattttattctctaatagtacccgcttatcatttgagaccaagagaatatctgtcttcattcaaacagacaaggccttatacaagccaaagcaagaagtgaagtttcgcattgttacactcttctcagattttaagccttacaaaacctctttaaacattctcattaaggaccccaaatcaaatttgatccaacagtggttgtcacaacaaagtgatcttggagtcatttccaaaacttttcagctatcttcccatccaatacttggtgactggtctattcaagttcaagtgaatgaccagacatattatcaatcatttcaggtttcagaatatgtattaccaaaatttgaagtgactttgcagacaccattatattgttctatgaattctaagcatttaaatggtaccatcacggcaaagtatacatatgggaagccagtgaaaggagacgtaacgcttacatttttacctttatccttttggggaaagaagaaaaatattacaaaaacatttaagataaatggatctgcaaacttctcttttaatgatgaagagatgaaaaatgtaatggattcttcaaatggactttctgaatacctggatctatcttcccctggaccagtagaaattttaaccacagtgacagaatcagttacaggtatttcaagaaatgtaagcactaatgtgttcttcaagcaacatgattacatcattgagttttttgattatactactgtcttgaagccatctctcaacttcacagccactgtgaaggtaactcgtgctgatggcaaccaactgactcttgaagaaagaagaaataatgtagtcataacagtgacacagagaaactatactgagtactggagcggatctaacagtggaaatcagaaaatggaagctgttcagaaaataaattatactgtcccccaaagtggaacttttaagattgaattcccaatcctggaggattccagtgagctacagttgaaggcctatttccttggtagtaaaagtagcatggcagttcatagtctgtttaagtctcctagtaagacatacatccaactaaaaacaagagatgaaaatataaaggtgggatcgccttttgagttggtggttagtggcaacaaacgattgaaggagttaagctatatggtagtatccaggggacagttggtggctgtaggaaaacaaaattcaacaatgttctctttaacaccagaaaattcttggactccaaaagcctgtgtaattgtgtattatattgaagatgatggggaaattataagtgatgttctaaaaattcctgttcagcttgtttttaaaaataagataaagctatattggagtaaagtgaaagctgaaccatctgagaaagtctctcttaggatctctgtgacacagcctgactccatagttgggattgtagctgttgacaaaagtgtgaatctgatgaatgcctctaatgatattacaatggaaaatgtggtccatgagttggaactttataacacaggatattatttaggcatgttcataaattcttttgcagtctttcaggaatgtggactctgggtattgacagatgcaaacctcacgaaggattatattgatggtgtttatgacaatgcagaatatgctgagaggtttatggaggaaaatgaaggacatattgtagatattcatgacttttctttgggtagcagtccacatgtccgaaagcattttccagagacttggatttggctagacaccaacatgggttccaggatttaccaagaatttgaagtaactgtacctgattctatcacttcttgggtggctactggttttgtgatctctgaggacctgggtcttggactaacaactactccagtggagctccaagccttccaaccatttttcatttttttgaatcttccctactctgttatcagaggtgaagaatttgctttggaaataactatattcaattatttgaaagatgccactgaggttaaggtaatcattgagaaaagtgacaaatttgatattctaatgacttcaagtgaaataaatgccacaagccaccagcagacccttctggttcccagtgaggatggggcaactgttctttttcccatcaggccaacacatctgggagaaattcctatcacagtcacagctctttcacccactgcttctgatgctatcacccagatgattttagtaaaggctgaaggaatagaaaaatcatattcacaatccatcttattagacttgactgacaataggctacagagtaccctgaaaactttgagtttctcatttcctcctaatacagtgactggcagtgaaagagttcagatcactgcaattggagatgttcttggtccttccatcaatggcttagcctcattgattcggatgccttatggctgtggtgaacagaacatgataaattttgctccaaatatttacattttggattatctgactaaaaagaaacaactgacagataatttgaaagaaaaagctctttcatttatgaggcaaggttaccagagagaacttctctatcagagggaagatggctctttcagtgcttttgggaattatgacccttctgggagcacttggttgtcagcttttgttttaagatgtttccttgaagccgatccttacatagatattgatcagaatgtgttacacagaacatacacttggcttaaaggacatcagaaatccaacggtgaattttgggatccaggaagagtgattcatagtgagcttcaaggtggcaataaaagtccagtaacacttacagcctatattgtaacttctctcctgggatatagaaagtatcagcctaacattgatgtgcaagagtctatccattttttggagtctgaattcagtagaggaatttcagacaattatactctagcccttataacttatgcattgtcatcagtggggagtcctaaagcgaaggaagctttgaatatgctgacttggagagcagaacaagaaggtggcatgcaattctgggtgtcatcagagtccaaactttctgactcctggcagccacgctccctggatattgaagttgcagcctatgcactgctctcacacttcttacaatttcagacttctgagggaatcccaattatgaggtggctaagcaggcaaagaaatagcttgggtggttttgcatctactcaggataccactgtggctttaaaggctctgtctgaatttgcagccctaatgaatacagaaaggacaaatatccaagtgaccgtgacggggcctagctcaccaagtcctgtaaagtttctgattgacacacacaaccgcttactccttcagacagcagagcttgctgtggtacagccaacggcagttaatatttccgcaaatggttttggatttgctatttgtcagctcaatgttgtatataatgtgaaggcttctgggtcttctagaagacgaagatctatccaaaatcaagaagcctttgatttagatgttgctgtaaaagaaaataaagatgatctcaatcatgtggatttgaatgtgtgtacaagcttttcgggcccgggtaggagtggcatggctcttatggaagttaacctattaagtggctttatggtgccttcagaagcaatttctctgagcgagacagtgaagaaagtggaatatgatcatggaaaactcaacctctatttagattctgtaaatgaaacccagttttgtgttaatattcctgctgtgagaaactttaaagtttcaaatacccaagatgcttcagtgtctatagtggattactatgagccaaggagacaggcggtgagaagttacaactctgaagtgaagctgtcctcctgtgacctttgcagtgatgtccagggctgccgtccttgtgaggatggagcttcaggctcccatcatcactcttcagtcatttttattttctgtttcaagcttctgtactttatggaactttggctgtga

SEQ ID NO: 12 - RNA, 2938 nt, Homo sapiens
  source 1..2938: mol_type = mRNA
gttgacaaaagtgtgaatctgatgaatgcctctaatgatattacaatggaaaatgtggtccatgagttggaactttataacacaggatattatttaggcatgttcatgaattcttttgcagtctttcaggaatgtggactctgggtattgacagatgcaaacctcacgaaggattatattgatggtgtttatgacaatgcagaatatgctgagaggtttatggaggaaaatgaaggacatattgtagatattcatgacttttctttgggtagcagtccacatgtccgaaagcattttccagagacttggatttggctagacaccaacatgggttccaggatttaccaagaatttgaagtaactgtacctgattctatcacttcttgggtggctactggttttgtgatctctgaggacctgggtcttggactaacaactactccagtggagctccaagccttccaaccatttttcatttttttgaatcttccctactctgttatcagaggtgaagaatttgctttggaaataactatattcaattatttgaaagatgccactgaggttaaggtaatcattgagaaaagtgacaaatttgatattctaatgacttcaagtgaaataaatgccacaggccaccagcagacccttctggttcccagtgaggatggggcaactgttctttttcccatcaggccaacacatctgggagaaattcctatcacagtcacagctctttcacccactgcttctgatgctatcacccagatgattttagtaaaggctgaaggaatagaaaaatcatattcacaatccatcttattagacttgactgacaataggctacagagtaccctgaaaactttgagtttctcatttcctcctaatacagtgactggcagtgaaagagttcagatcactgcaattggagatgttcttggtccttccatcaatggcttagcctcattgattcggatgccttatggctgtggtgaacagaacatgataaattttgctccaaatatttacattttggattatctgactaaaaagaaacaactgacagataatttgaaagaaaaagctctttcatttatgaggcaaggttaccagagagaacttctctatcagagggaagatggctctttcagtgcttttgggaattatgacccttctgggagcacttggttgtcagcttttgttttaagatgtttccttgaagccgatccttacatagatattgatcagaatgtgttacacagaacatacacttggcttaaaggacatcagaaatccaacggtgaattttgggatccaggaagagtgattcatagtgagcttcaaggtggcaataaaagtccagtaacacttacagcctatattgtaacttctctcctgggatatagaaagtatcagcctaacattgatgtgcaagagtctatccattttttggagtctgaattcagtagaggaatttcagacaattatactctagcccttataacttatgcattgtcatcagtggggagtcctaaagcgaaggaagctttgaatatgctgacttggagagcagaacaagaaggtggcatgcaattctgggtgtcatcagagtccaaactttctgactcctggcagccacgctccctggatattgaagttgcagcctatgcactgctctcacacttcttacaatttcagacttctgagggaatcccaattatgaggtggctaagcaggcaaagaaatagcttgggtggttttgcatctactcaggataccactgtggctttaaaggctctgtctgaatttgcagccctaatgaatacagaaaggacaaatatccaagtgaccgtgacggggcctagctcaccaagtcctgtaaagtttctgattgacacacacaaccgcttactccttcagacagcagagcttgctgtggtacagccaacggcagttaatatttccgcaaatggttttggatttgctatttgtcagctcaatgttgtatataatgtgaaggcttctgggtcttctagaagacgaagatctatccaaaatcaagaagcctttgatttagatgttgctgtaaaagaaaataaagatgatctcaatcatgtggatttgaatgtgtgtacaagcttttcgggcccgggtaggagtggcatggctcttatggaagttaacctattaagtggctttatggtgccttcagaagcaatttctctgagcgagacagtgaagaaagtggaatatgatcatggaaaactcaacctctatttagattctgtaaatgaaacccagttttgtgttaatattcctgctgtgagaaactttaaagtttcaaatacccaagatgcttcagtgtccatagtggattactatgagccaaggagacaggcggtgagaagttacaactctgaagtgaagctgtcctcctgtgacctttgcagtgatgtccagggctgccgtccttgtgaggatggagcttcaggctcccatcatcactcttcagtcatttttattttctgtttcaagcttctgtactttatggaactttggctgtgatttatttttaaaggactctgtgtaacactaacatttccagtagtcacatgtgattgttttgttttcgtagaagaatactgcttctattttgaaaaaagagttttttttctttctatggggttgcagggatggtgtacaacaggtcctagcatgtatagctgcatagatttcttcacctgatctttgtgtggaagatcagaatgaatgcagttgtgtgtctatattttcccctctcaaaatcttttagaatttttttggaggtgtttgttttctccagaataaaggtattactttagaataaaaaaaaaaaaaaaaaaaaaaaaaaaaaaaaaaaaaaaaaaaaaaaaaaaaaaaaaaaaaaaaaaaaaaaaaa

SEQ ID NO: 13 - RNA, 2843 nt, Homo sapiens
  source 1..2843: mol_type = mRNA
ctgatgaatgcctctaatgatattacaatggaaaatgtggtccatgagttggaactttataacacaggatattatttaggcatgttcatgaattcttttgcagtctttcaggaatgtggactctgggtattgacagatgcaaacctcacgaaggattatattgatggtgtttatgacaatgcagaatatgctgagaggtttatggaggaaaatgaaggacatattgtagatattcatgacttttctttgggtagcagtccacatgtccgaaagcattttccagagacttggatttggctagacaccaacatgggttccaggatttaccaagaatttgaagtaactgtacctgattctatcacttcttgggtggctactggttttgtgatctctgaggacctgggtcttggactaacaactactccagtggagctccaagccttccaaccatttttcatttttttgaatcttccctactctgttatcagaggtgaagaatttgctttggaaataactatattcaattatttgaaagatgccactgaggttaaggtaatcattgagaaaagtgacaaatttgatattctaatgacttcaagtgaaataaatgccacaggccaccagcagacccttctggttcccagtgaggatggggcaactgttctttttcccatcaggccaacacatctgggagaaattcctatcacagtcacagctctttcacccactgcttctgatgctatcacccagatgattttagtaaaggctgaaggaatagaaaaatcatattcacaatccatcttattagacttgactgacaataggctacagagtaccctgaaaactttgagtttctcatttcctcctaatacagtgactggcagtgaaagagttcagatcactgcaattggagatgttcttggtccttccatcaatggcttagcctcattgattcggatgccttatggctgtggtgaacagaacatgataaattttgctccaaatatttacattttggattatctgactaaaaagaaacaactgacagataatttgaaagaaaaagctctttcatttatgaggcaaggttaccagagagaacttctctatcagagggaagatggctctttcagtgcttttgggaattatgacccttctgggagcacttggttgtcagcttttgttttaagatgtttccttgaagccgatccttacatagatattgatcagaatgtgttacacagaacatacacttggcttaaaggacatcagaaatccaacggtgaattttgggatccaggaagagtgattcatagtgagcttcaaggtggcaataaaagtccagtaacacttacagcctatattgtaacttctctcctgggatatagaaagtatcagcctaacattgatgtgcaagagtctatccattttttggagtctgaattcagtagaggaatttcagacaattatactctagcccttataacttatgcattgtcatcagtggggagtcctaaagcgaaggaagctttgaatatgctgacttggagagcagaacaagaaggtggcatgcaattctgggtgtcatcagagtccaaactttctgactcctggcagccacgctccctggatattgaagttgcagcctatgcactgctctcacacttcttacaatttcagacttctgagggaatcccaattatgaggtggctaagcaggcaaagaaatagcttgggtggttttgcatctactcaggataccactgtggctttaaaggctctgtctgaatttgcagccctaatgaatacagaaaggacaaatatccaagtgaccgtgacggggcctagctcaccaagtcctgtaaagtttctgattgacacacacaaccgcttactccttcagacagcagagcttgctgtggtacagccaacggcagttaatatttccgcaaatggttttggatttgctatttgtcagctcaatgttgtatataatgtgaaggcttctgggtcttctagaagacgaagatctatccaaaatcaagaagcctttgatttagatgttgctgtaaaagaaaataaagatgatctcaatcatgtggatttgaatgtgtgtacaagcttttcgggcccgggtaggagtggcatggctcttatggaagttaacctattaagtggctttatggtgccttcagaagcaatttctctgagcgagacagtgaagaaagtggaatatgatcatggaaaactcaacctctatttagattctgtaaatgaaacccagttttgtgttaatattcctgctgtgagaaactttaaagtttcaaatacccaagatgcttcagtgtccatagtggattactatgagccaaggagacaggcggtgagaagttacaactctgaagtgaagctgtcctcctgtgacctttgcagtgatgtccagggctgccgtccttgtgagaatggagcttcaggctcccatcatcactcttcagtcatttttattttctgtttcaagcttctgtactttatggaactttggctgtgatttatttttaaaggactctgtgtaacactaacatttccagtagtcacatgtgattgttttgttttcgtagaagaatactgcttctattttgaaaaaagagttttttttctttctatggggttgcagggatggtgtacaacaggtcctagcatgtatagctgcatagatttcttcacctgatctttgtgtggaagatcagaatgaatgcagttgtgtgtctatattttcccctctcaaaatcttttagaatttttttggaggtgtttgttttctccagaataaaggtattactttag

SEQ ID NO: 14 - RNA, 8800 nt, Homo sapiens
  source 1..8800: mol_type = mRNA
agacgccgtcgagatgcagggcccaccgctcctgaccgccgcccacctcctctgcgtgtgcaccgccgcgctggccgtggctcccgggcctcggtttctggtgacagccccagggatcatcaggcccggaggaaatgtgactattggggtggagcttctggaacactgcccttcacaggtgactgtgaaggcggagctgctcaagacagcatcaaacctcactgtctctgtcctggaagcagaaggagtctttgaaaaaggctcttttaagacacttactcttccatcagaccccaaatcaaatttgatccaacagtggttgtcacaacaaagtgatcttggagtcatttccaaaacttttcagctatcttcccatccaatacttggtgactggtctattcaagttcaagtgaatgaccagacatactatcaatcatttcaggtttcagaatatgtattaccaaaatttgaagtgactttgcagacaccattatattgttctatgaattctaagcatttaaatggtaccatcacggcaaagtatacatatgggaagccagtgaaaggagacgtaacgcttacatttttacctttatccttttggggaaagaagaaaaatattacaaaaacatttaagataaatggatctgcaaacttctcttttaatgatgaagagatgaaaaatgtaatggattcttcaaatggactttctgaatacctggatctatcttcccctggaccagtagaaattttaaccacagtgacagaatcagttacaggtatttcaagaaatgtaagcactaatgtgttcttcaagcaacatgattacatcattgagttttttgattatactactgtcttgaagccatctctcaacttcacagccactgtgaaggtaactcgtgctgatggcaaccaactgactcttgaagaaagaagaaataatgtagtcataacagtgacacagagaaactatactgagtactggagcggatctaacagtggaaatcagaaaatggaagctgttcagaaaataaattatactgtcccccaaagtggaacttttaagattgaattcccaatcctggaggattccagtgagctacagttgaaggcctatttccttggtagtaaaagtagcatggcagttcatagtctgtttaagtctcctagtaagacatacatccaactaaaaacaagagatgaaaatataaaggtgggatcgccttttgagttggtggttagtggcaacaaacgattgaaggagttaagctatatggtagtatccaggggacagttggtggctgtaggaaaacaaaattcaacaatgttctctttaacaccagaaaattcttggactccaaaagcctgtgtaattgtgtattatattgaagatgatggggaaattataagtgatgttctaaaaattcctgttcagcttgtttttaaaaataagataaagctatattggagtaaagtgaaagctgaaccatctgagaaagtctctcttaggatctctgtgacacagcctgactccatagttgggattgtagctgttgacaaaagtgtgaatctgatgaatgcctctaatgatattacaatggaaaatgtggtccatgagttggaactttataacacaggatattatttaggcatgttcatgaattcttttgcagtctttcaggaatgtggactctgggtattgacagatgcaaacctcacgaaggattatattgatggtgtttatgacaatgcagaatatgctgagaggtttatggaggaaaatgaaggacatattgtagatattcatgacttttctttgggtagcagtccacatgtccgaaagcattttccagagacttggatttggctagacaccaacatgggttacaggatttaccaagaatttgaagtaactgtacctgattctatcacttcttgggtggctactggttttgtgatctctgaggacctgggtcttggactaacaactactccagtggagctccaagccttccaaccatttttcatttttttgaatcttccctactctgttatcagaggtgaagaatttgctttggaaataactatattcaattatttgaaagatgccactgaggttaaggtaatcattgagaaaagtgacaaatttgatattctaatgacttcaaatgaaataaatgccacaggccaccagcagacccttctggttcccagtgaggatggggcaactgttctttttcccatcaggccaacacatctgggagaaattcctatcacagtcacagctctttcacccactgcttctgatgctgtcacccagatgattttagtaaaggctgaaggaatagaaaaatcatattcacaatccatcttattagacttgactgacaataggctacagagtaccctgaaaactttgagtttctcatttcctcctaatacagtgactggcagtgaaagagttcagatcactgcaattggagatgttcttggtccttccatcaatggcttagcctcattgattcggatgccttatggctgtggtgaacagaacatgataaattttgctccaaatatttacattttggattatctgactaaaaagaaacaactgacagataatttgaaagaaaaagctctttcatttatgaggcaaggttaccagagagaacttctctatcagagggaagatggctctttcagtgcttttgggaattatgacccttctgggagcacttggttgtcagcttttgttttaagatgtttccttgaagccgatccttacatagatattgatcagaatgtgttacacagaacatacacttggcttaaaggacatcagaaatccaacggtgaattttgggatccaggaagagtgattcatagtgagcttcaaggtggcaataaaagtccagtaacacttacagcctatattgtaacttctctcctgggatatagaaagtatcagcctaacattgatgtgcaagagtctatccattttttggagtctgaattcagtagaggaatttcagacaattatactctagcccttataacttatgcattgtcatcagtggggagtcctaaagcgaaggaagctttgaatatgctgacttggagagcagaacaagaaggtggcatgcaattctgggtgtcatcagagtccaaactttctgactcctggcagccacgctccctggatattgaagttgcagcctatgcactgctctcacacttcttacaatttcagacttctgagggaatcccaattatgaggtggctaagcaggcaaagaaatagcttgggtggttttgcatctactcaggataccactgtggctttaaaggctctgtctgaatttgcagccctaatgaatacagaaaggacaaatatccaagtgaccgtgacggggcctagctcaccaagtcctgtaaagtttctgattgacacacacaaccgcttactccttcagacagcagagcttgctgtggtacagccaacggcagttaatatttccgcaaatggttttggatttgctatttgtcagctcaatgttgtatataatgtgaaggcttctgggtcttctagaagacgaagatctatccaaaatcaagaagcctttgatttagatgttgctgtaaaagaaaataaagatgatctcaatcatgtggatttgaatgtgtgtacaagcttttcgggcccgggtaggagtggcatggctcttatggaagttaacctattaagtggctttatggtgccttcagaagcaatttctctgagcgagacagtgaagaaagtggaatatgatcatggaaaactcaacctctatttagattctgtaaatgaaacccagttttgtgttaatattcctgctgtgagaaactttaaagtttcaaatacccaagatgcttcagtgtccatagtggattactatgagccaaggagacaggcggtgagaagttacaactctgaagtgaagctgtcctcctgtgacctttgcagtgatgtccagggctgccgtccttgtgaggatggagcttcaggctcccatcatcactcttcagtcatttttattttctgtttcaagcttctgtactttatggaactttggctgtgatttatttttaaaggactctgtgtaacactaacatttccagtagtcacatgtgattgttttgttttcgtagaagaatactgcttctattttgaaaaaagagttttttttctttctatggggttgcagggatggtgtacaacaggtcctagcatgtatagctgcatagatttcttcacctgatctttgtgtggaagatcagaatgaatgcagttgtgtgtctatattttcccctctcaaaatcttttagaatttttttggaggtgtttgttttctccagaataaaggtattactttagaataggtattctcctcattttgtgaaagaaatgaacctagattcttaagcattattacacatccatgtttgcttaaagatggatttccctgggaatgggagaaaacagccagcaggaggagcttcatctgttcccttcccacctccaacctagccctactgcccaccccaccccaacccaccccatgcccagtggtctcagtagatacttcttaactggaaattctttcttttcagaatctaggtggtgaattttttttaagtggcacggtctttttctgcttgaaatctgatcacaccccccagccattgccctccctctctttttcctctgtagagaaatgtgaggggcagtacatttactgtgcttttcacaccatctcagaggttgaggagcatactgaaaattgccctggggggtgctgggtgtgctgtctccttcccacatcctcagccccacaccagctctatttcaggggtgagagtcagagagcactgcaatatgtgcttcatgggatttcgattcgaagatcctagaccagggagacactgtgagccagggatacaacaaaatactaggtaagtcactgcagaccgacctccctgcagtttgggaaagaagctgggtttgtggagaatcagagcatcttgacatgactgctgacctaaagatccctggcattggccagggatcctgtggaacctcttctagttcaggggtgtgagcattagactgccagttgtctagtgacatctgatgcttgctgtgaacttttaagatccccgaatcctgagcacctcaatctttaattgccctgtattccgaagggtaatataatttatctggatggaaattttaaagatgaatcccccttttttcttttcttctctcttttctttccttctccctttcttctttgccttctaaatatactgaaatgatttagatatgtgtcaacaattaatgatcttttattcaatctaagaaatggtttagtttttctctttagctctatggcatttcactcaagtggacaggggaaaaagtaattgccatgggctccaaagaatttgctttatgtttttagctatttaaaaataaatccatcaaaaataaagtatgcaaatgtatcttttaaagttaatttttaaaaatgctcttattttagtgaattttcagaaattatagtggaatggatgctcatatattgcttatggatattttggataccaaagtaggaataactgacattcagtattttaaagctggcaaacctgtacatagaaaatagatccccagacagtggtctatgaagagggcagttaagtatcaaatacttaattttcttgcctttttttcttaagtggggaaaagtttctagatctcttacacctctgacacaatctgttctaaaacaggcacttgtaatgttggggcctccttgtaaacgtgtttttgccctttactctctgggagttctttaaaggtgaaatcatcttacaaagaaattgggggagggtcttggcaaaggactttcccctcctctttcctggcctgggaaccttatactgacaatcaatactttatattttaaagtatataatttatagttaacttctagtgtaatatattaggaaacactagaatggaaaggccattggaagacaggttgtatcttttttagaccatatttccttgtttaaaaactatcatttgaatacttttttggtgaagaactccatgttttcaagttaaaggtcacctcgtaggccaggcgcagtggctcatgcctgtaatcccagcactctgggaggctgaggcgggtgaatcacaaggttaggagtttgagaccagcctggccaatatggtgaaaccccgtccctactaaaaatacaaaatttagccaggcgtggtggcatgcacctgtagtcccacctactcgggaggctgaggcaggagaatcacttgaacctgagagacagaggttgcagtgagccgagatcacgccactgcactccagcctgggggacagagtgagattctgtctcaaaaaacaaaaaacaaaaaagtcaccttgtaactcatctctttttattgtaagtttattaaaaatgaagaggacaacaatgagaaggaacataaagggttagctagcactgtctcctggtgcatggggctgtgcagatgtcccggccacttcttccttcatacttcccttagagaacttgctctgctacaagcagtgggcttggactaaaagtgattaaaataccacaggcataaggagaaaaggagtatatgtagtagtaataattactagtataaattattttcttcacatgctatgagtaataatattaaaaaactcattttaccattaagattccttatgctgaagctcttccatttagaatactgtcaatgtcatttactggtatgaactaaagtcccccttcttttccactcactgggaaccttagtaaaacaccagcatatcttacctctctttctgactggccgatgcttccagagactgaatgttgggaaaacctagtagccaaacaattctaggacagaataacatttttatatttggttccaccatcttattacatttagttatagttttaaaaaagaaattcaagcccattaaaatatgtctggtcaatgaaatgcttccttttattgtgttgtgctattgtactttgtttttcaaaacattgtaaaaatagtatctttggtttagtattttggattatatattataatctgaggagtgttttgcttatgtagaatccagatatatttctgttacctaggagatgttacttacatatgtaatactgtatcctgcacgtggaaatattcagaattgtagatagcataactctccctgctcctattcttttgagcctaggtataattttttttttttttttagaaaaagacatatttagctttaatttctatttatgctaaacatatttataagtagtctgtcaatataataccaactatttttatttttacataattcaattatttcatttgacatgtctggcagactcaagacattaagtaaaaaattggaactatgatttttctttgtcattttttaaaaaagaattattttattaacctgctggcatataatctggagttcttttcacaaccttactttttctgatttgctttattgaatgattgaatactcatttctttctaaaaatatgttgtaaattctcccttggcaagatttctccctatgagggtagttattatttgagtctgccaagtggttaccatggggcaaggtgccatgatgtattcttgggtgcattggttttttgcgcattgtaaatttaagacacttatagtaagtggactcattcatagatgagtttcagaaccttttacgttctcggtagaggcttctgtcggacaggcagaagagtgtattcctcacttttttttttgtcttcaaattccagtaaggcatagcacttttaagaaattagaatttttctatcatctatgcaaatgatatttatgttaatattaaatatcttatgttacactgggagtaatttgaggtgcaattatttttattactactttgaatagaggaccattatccttctttcttcagaaaactaagaagtaagtgtaacttttaaagtaagtatatatcagtgagagtaggcttgttttacaactatttctagccagtgagttgtgttttcatgtctcatcaaaagacaataccacattgcatcattttacaaaatatgttgtcattttcatttcagttgtaacataggaaaatagatatttcctagatgatttctgagtttcttactgcaaagaacagttataaattggtatacatgtgtctctgtaatagggataatattgatatatctgttgctacatatttaagaatcattctatcttatgttgtcttgaggccaagatttaccacgtttgcccagtgtattgaattggtggtagaaggtagttccatgttccatttgtagatctttaagattttatctttgataactttaatagaatgtggctcagttctggtccttcaagcctgtatggtttggattttcagtaggggacagttgatgtggagtcaatctctttggtacacaggaagctttataaaatttcattcacgaatctcttattttgggaagctgttttgcatatgagaagaacactgttgaaataaggaactaaagctttatatattgatcaaggtgattctgaaagttttaatttttaatgttgtaatgttatgttattgttaattgtactttattatgtattcaatagaaaatcatgatttattaataaaagcttaaattctcatcta

SEQ ID NO: 15 - RNA, 8980 nt, Homo sapiens
  source 1..8980: mol_type = mRNA
agacgccgtcgagatgcagggcccaccgctcctgaccgccgcccacctcctctgcgtgtgcaccgccgcgctggccgtggctcccgggcctcggtttctggtgacagccccagggatcatcaggcccggaggaaatgtgactattggggtggagcttctggaacactgcccttcacaggtgactgtgaaggcggagctgctcaagacagcatcaaacctcactgtctctgtcctggaagcagaaggagtctttgaaaaaggctcttttaagacacttactcttccatcactacctctgaacagtgcagatgagatttatgagctacgtgtaaccggacgtacccaggatgagattttattctctaatagtacccgcttatcatttgagaccaagagaatatctgtcttcattcaaacagacaaggccttatacaagccaaagcaagaagtgaagtttcgcattgttacactcttctcagattttaagccttacaaaacctctttaaacattctcattaaggaccccaaatcaaatttgatccaacagtggttgtcacaacaaagtgatcttggagtcatttccaaaacttttcagctatcttcccatccaatacttggtgactggtctattcaagttcaagtgaatgaccagacatactatcaatcatttcaggtttcagaatatgtattaccaaaatttgaagtgactttgcagacaccattatattgttctatgaattctaagcatttaaatggtaccatcacggcaaagtatacatatgggaagccagtgaaaggagacgtaacgcttacatttttacctttatccttttggggaaagaagaaaaatattacaaaaacatttaagataaatggatctgcaaacttctcttttaatgatgaagagatgaaaaatgtaatggattcttcaaatggactttctgaatacctggatctatcttcccctggaccagtagaaattttaaccacagtgacagaatcagttacaggtatttcaagaaatgtaagcactaatgtgttcttcaagcaacatgattacatcattgagttttttgattatactactgtcttgaagccatctctcaacttcacagccactgtgaaggtaactcgtgctgatggcaaccaactgactcttgaagaaagaagaaataatgtagtcataacagtgacacagagaaactatactgagtactggagcggatctaacagtggaaatcagaaaatggaagctgttcagaaaataaattatactgtcccccaaagtggaacttttaagattgaattcccaatcctggaggattccagtgagctacagttgaaggcctatttccttggtagtaaaagtagcatggcagttcatagtctgtttaagtctcctagtaagacatacatccaactaaaaacaagagatgaaaatataaaggtgggatcgccttttgagttggtggttagtggcaacaaacgattgaaggagttaagctatatggtagtatccaggggacagttggtggctgtaggaaaacaaaattcaacaatgttctctttaacaccagaaaattcttggactccaaaagcctgtgtaattgtgtattatattgaagatgatggggaaattataagtgatgttctaaaaattcctgttcagcttgtttttaaaaataagataaagctatattggagtaaagtgaaagctgaaccatctgagaaagtctctcttaggatctctgtgacacagcctgactccatagttgggattgtagctgttgacaaaagtgtgaatctgatgaatgcctctaatgatattacaatggaaaatgtggtccatgagttggaactttataacacaggatattatttaggcatgttcatgaattcttttgcagtctttcaggaatgtggactctgggtattgacagatgcaaacctcacgaaggattatattgatggtgtttatgacaatgcagaatatgctgagaggtttatggaggaaaatgaaggacatattgtagatattcatgacttttctttgggtagcagtccacatgtccgaaagcattttccagagacttggatttggctagacaccaacatgggttacaggatttaccaagaatttgaagtaactgtacctgattctatcacttcttgggtggctactggttttgtgatctctgaggacctgggtcttggactaacaactactccagtggagctccaagccttccaaccatttttcatttttttgaatcttccctactctgttatcagaggtgaagaatttgctttggaaataactatattcaattatttgaaagatgccactgaggttaaggtaatcattgagaaaagtgacaaatttgatattctaatgacttcaaatgaaataaatgccacaggccaccagcagacccttctggttcccagtgaggatggggcaactgttctttttcccatcaggccaacacatctgggagaaattcctatcacagtcacagctctttcacccactgcttctgatgctgtcacccagatgattttagtaaaggctgaaggaatagaaaaatcatattcacaatccatcttattagacttgactgacaataggctacagagtaccctgaaaactttgagtttctcatttcctcctaatacagtgactggcagtgaaagagttcagatcactgcaattggagatgttcttggtccttccatcaatggcttagcctcattgattcggatgccttatggctgtggtgaacagaacatgataaattttgctccaaatatttacattttggattatctgactaaaaagaaacaactgacagataatttgaaagaaaaagctctttcatttatgaggcaaggttaccagagagaacttctctatcagagggaagatggctctttcagtgcttttgggaattatgacccttctgggagcacttggttgtcagcttttgttttaagatgtttccttgaagccgatccttacatagatattgatcagaatgtgttacacagaacatacacttggcttaaaggacatcagaaatccaacggtgaattttgggatccaggaagagtgattcatagtgagcttcaaggtggcaataaaagtccagtaacacttacagcctatattgtaacttctctcctgggatatagaaagtatcagcctaacattgatgtgcaagagtctatccattttttggagtctgaattcagtagaggaatttcagacaattatactctagcccttataacttatgcattgtcatcagtggggagtcctaaagcgaaggaagctttgaatatgctgacttggagagcagaacaagaaggtggcatgcaattctgggtgtcatcagagtccaaactttctgactcctggcagccacgctccctggatattgaagttgcagcctatgcactgctctcacacttcttacaatttcagacttctgagggaatcccaattatgaggtggctaagcaggcaaagaaatagcttgggtggttttgcatctactcaggataccactgtggctttaaaggctctgtctgaatttgcagccctaatgaatacagaaaggacaaatatccaagtgaccgtgacggggcctagctcaccaagtcctcttgctgtggtacagccaacggcagttaatatttccgcaaatggttttggatttgctatttgtcagctcaatgttgtatataatgtgaaggcttctgggtcttctagaagacgaagatctatccaaaatcaagaagcctttgatttagatgttgctgtaaaagaaaataaagatgatctcaatcatgtggatttgaatgtgtgtacaagcttttcgggcccgggtaggagtggcatggctcttatggaagttaacctattaagtggctttatggtgccttcagaagcaatttctctgagcgagacagtgaagaaagtggaatatgatcatggaaaactcaacctctatttagattctgtaaatgaaacccagttttgtgttaatattcctgctgtgagaaactttaaagtttcaaatacccaagatgcttcagtgtccatagtggattactatgagccaaggagacaggcggtgagaagttacaactctgaagtgaagctgtcctcctgtgacctttgcagtgatgtccagggctgccgtccttgtgaggatggagcttcaggctcccatcatcactcttcagtcatttttattttctgtttcaagcttctgtactttatggaactttggctgtgatttatttttaaaggactctgtgtaacactaacatttccagtagtcacatgtgattgttttgttttcgtagaagaatactgcttctattttgaaaaaagagttttttttctttctatggggttgcagggatggtgtacaacaggtcctagcatgtatagctgcatagatttcttcacctgatctttgtgtggaagatcagaatgaatgcagttgtgtgtctatattttcccctctcaaaatcttttagaatttttttggaggtgtttgttttctccagaataaaggtattactttagaataggtattctcctcattttgtgaaagaaatgaacctagattcttaagcattattacacatccatgtttgcttaaagatggatttccctgggaatgggagaaaacagccagcaggaggagcttcatctgttcccttcccacctccaacctagccctactgcccaccccaccccaacccaccccatgcccagtggtctcagtagatacttcttaactggaaattctttcttttcagaatctaggtggtgaattttttttaagtggcacggtctttttctgcttgaaatctgatcacaccccccagccattgccctccctctctttttcctctgtagagaaatgtgaggggcagtacatttactgtgcttttcacaccatctcagaggttgaggagcatactgaaaattgccctggggggtgctgggtgtgctgtctccttcccacatcctcagccccacaccagctctatttcaggggtgagagtcagagagcactgcaatatgtgcttcatgggatttcgattcgaagatcctagaccagggagacactgtgagccagggatacaacaaaatactaggtaagtcactgcagaccgacctccctgcagtttgggaaagaagctgggtttgtggagaatcagagcatcttgacatgactgctgacctaaagatccctggcattggccagggatcctgtggaacctcttctagttcaggggtgtgagcattagactgccagttgtctagtgacatctgatgcttgctgtgaacttttaagatccccgaatcctgagcacctcaatctttaattgccctgtattccgaagggtaatataatttatctggatggaaattttaaagatgaatcccccttttttcttttcttctctcttttctttccttctccctttcttctttgccttctaaatatactgaaatgatttagatatgtgtcaacaattaatgatcttttattcaatctaagaaatggtttagtttttctctttagctctatggcatttcactcaagtggacaggggaaaaagtaattgccatgggctccaaagaatttgctttatgtttttagctatttaaaaataaatccatcaaaaataaagtatgcaaatgtatcttttaaagttaatttttaaaaatgctcttattttagtgaattttcagaaattatagtggaatggatgctcatatattgcttatggatattttggataccaaagtaggaataactgacattcagtattttaaagctggcaaacctgtacatagaaaatagatccccagacagtggtctatgaagagggcagttaagtatcaaatacttaattttcttgcctttttttcttaagtggggaaaagtttctagatctcttacacctctgacacaatctgttctaaaacaggcacttgtaatgttggggcctccttgtaaacgtgtttttgccctttactctctgggagttctttaaaggtgaaatcatcttacaaagaaattgggggagggtcttggcaaaggactttcccctcctctttcctggcctgggaaccttatactgacaatcaatactttatattttaaagtatataatttatagttaacttctagtgtaatatattaggaaacactagaatggaaaggccattggaagacaggttgtatcttttttagaccatatttccttgtttaaaaactatcatttgaatacttttttggtgaagaactccatgttttcaagttaaaggtcacctcgtaggccaggcgcagtggctcatgcctgtaatcccagcactctgggaggctgaggcgggtgaatcacaaggttaggagtttgagaccagcctggccaatatggtgaaaccccgtccctactaaaaatacaaaatttagccaggcgtggtggcatgcacctgtagtcccacctactcgggaggctgaggcaggagaatcacttgaacctgagagacagaggttgcagtgagccgagatcacgccactgcactccagcctgggggacagagtgagattctgtctcaaaaaacaaaaaacaaaaaagtcaccttgtaactcatctctttttattgtaagtttattaaaaatgaagaggacaacaatgagaaggaacataaagggttagctagcactgtctcctggtgcatggggctgtgcagatgtcccggccacttcttccttcatacttcccttagagaacttgctctgctacaagcagtgggcttggactaaaagtgattaaaataccacaggcataaggagaaaaggagtatatgtagtagtaataattactagtataaattattttcttcacatgctatgagtaataatattaaaaaactcattttaccattaagattccttatgctgaagctcttccatttagaatactgtcaatgtcatttactggtatgaactaaagtcccccttcttttccactcactgggaaccttagtaaaacaccagcatatcttacctctctttctgactggccgatgcttccagagactgaatgttgggaaaacctagtagccaaacaattctaggacagaataacatttttatatttggttccaccatcttattacatttagttatagttttaaaaaagaaattcaagcccattaaaatatgtctggtcaatgaaatgcttccttttattgtgttgtgctattgtactttgtttttcaaaacattgtaaaaatagtatctttggtttagtattttggattatatattataatctgaggagtgttttgcttatgtagaatccagatatatttctgttacctaggagatgttacttacatatgtaatactgtatcctgcacgtggaaatattcagaattgtagatagcataactctccctgctcctattcttttgagcctaggtataattttttttttttttttagaaaaagacatatttagctttaatttctatttatgctaaacatatttataagtagtctgtcaatataataccaactatttttatttttacataattcaattatttcatttgacatgtctggcagactcaagacattaagtaaaaaattggaactatgatttttctttgtcattttttaaaaaagaattattttattaacctgctggcatataatctggagttcttttcacaaccttactttttctgatttgctttattgaatgattgaatactcatttctttctaaaaatatgttgtaaattctcccttggcaagatttctccctatgagggtagttattatttgagtctgccaagtggttaccatggggcaaggtgccatgatgtattcttgggtgcattggttttttgcgcattgtaaatttaagacacttatagtaagtggactcattcatagatgagtttcagaaccttttacgttctcggtagaggcttctgtcggacaggcagaagagtgtattcctcacttttttttttgtcttcaaattccagtaaggcatagcacttttaagaaattagaatttttctatcatctatgcaaatgatatttatgttaatattaaatatcttatgttacactgggagtaatttgaggtgcaattatttttattactactttgaatagaggaccattatccttctttcttcagaaaactaagaagtaagtgtaacttttaaagtaagtatatatcagtgagagtaggcttgttttacaactatttctagccagtgagttgtgttttcatgtctcatcaaaagacaataccacattgcatcattttacaaaatatgttgtcattttcatttcagttgtaacataggaaaatagatatttcctagatgatttctgagtttcttactgcaaagaacagttataaattggtatacatgtgtctctgtaatagggataatattgatatatctgttgctacatatttaagaatcattctatcttatgttgtcttgaggccaagatttaccacgtttgcccagtgtattgaattggtggtagaaggtagttccatgttccatttgtagatctttaagattttatctttgataactttaatagaatgtggctcagttctggtccttcaagcctgtatggtttggattttcagtaggggacagttgatgtggagtcaatctctttggtacacaggaagctttataaaatttcattcacgaatctcttattttgggaagctgttttgcatatgagaagaacactgttgaaataaggaactaaagctttatatattgatcaaggtgattctgaaagttttaatttttaatgttgtaatgttatgttattgttaattgtactttattatgtattcaatagaaaatcatgatttattaataaaagcttaaattctcatcta

SEQ ID NO: 16 - DNA, 9401 bp, Homo sapiens
  source 1..9401: mol_type = genomic DNA
atagttgctatgaatttctcctcacaaggatgggcctgtttactcaccctgggcatcgttggtagagcgctagtgtaaacagcctgaggaacccggtgggccggggaagtgggcgcgctctgttctccgcggccagctgggacgccgggccaggtggggccgcctgcgtttagcaactgctttctcaccccctggatttgcgatgtttgccacagcagcgagaagcgccattgtaatggggatgggaggggtggagcctccaagtcctgtctcaatttagatctctcactctgctgttaggcgcgcccatttcagattactaaactcgaattaagagggaaaaaaaatcagggaggaggtggcaagccacaccccacggtgcccgcgaacttccccggcagcggactgtagcccaggcagacgccgtcgagatgcagggcccaccgctcctgaccgccgcccacctcctctgcgtgtgcaccgccgcgctggccgtggctcccgggcctcggtttctggtgacagccccagggatcatcaggcccggaggaaatgtgactattggggtggagcttctggaacactgcccttcacaggtgactgtgaaggcggagctgctcaagacagcatcaaacctcactgtctctgtcctggaagcagaaggagtctttgaaaaaggctcttttaagacacttactcttccatcactacctctgaacagtgcagatgagatttatgagctacgtgtaaccggacgtacccaggatgagattttattctctaatagtacccgcttatcatttgagaccaagagaatatctgtcttcattcaaacagacaaggccttatacaagccaaagcaagaagtgaagtttcgcattgttacactcttctcagattttaagccttacaaaacctctttaaacattctcattaaggaccccaaatcaaatttgatccaacagtggttgtcacaacaaagtgatcttggagtcatttccaaaacttttcagctatcttcccatccaatacttggtgactggtctattcaagttcaagtgaatgaccagacatactatcaatcatttcaggtttcagaatatgtattaccaaaatttgaagtgactttgcagacaccattatattgttctatgaattctaagcatttaaatggtaccatcacggcaaagtatacatatgggaagccagtgaaaggagacgtaacgcttacatttttacctttatccttttggggaaagaagaaaaatattacaaaaacatttaagataaatggatctgcaaacttctcttttaatgatgaagagatgaaaaatgtaatggattcttcaaatggactttctgaatacctggatctatcttcccctggaccagtagaaattttaaccacagtgacagaatcagttacaggtatttcaagaaatgtaagcactaatgtgttcttcaagcaacatgattacatcattgagttttttgattatactactgtcttgaagccatctctcaacttcacagccactgtgaaggtaactcgtgctgatggcaaccaactgactcttgaagaaagaagaaataatgtagtcataacagtgacacagagaaactatactgagtactggagcggatctaacagtggaaatcagaaaatggaagctgttcagaaaataaattatactgtcccccaaagtggaacttttaagattgaattcccaatcctggaggattccagtgagctacagttgaaggcctatttccttggtagtaaaagtagcatggcagttcatagtctgtttaagtctcctagtaagacatacatccaactaaaaacaagagatgaaaatataaaggtgggatcgccttttgagttggtggttagtggcaacaaacgattgaaggagttaagctatatggtagtatccaggggacagttggtggctgtaggaaaacaaaattcaacaatgttctctttaacaccagaaaattcttggactccaaaagcctgtgtaattgtgtattatattgaagatgatggggaaattataagtgatgttctaaaaattcctgttcagcttgtttttaaaaataagataaagctatattggagtaaagtgaaagctgaaccatctgagaaagtctctcttaggatctctgtgacacagcctgactccatagttgggattgtagctgttgacaaaagtgtgaatctgatgaatgcctctaatgatattacaatggaaaatgtggtccatgagttggaactttataacacaggatattatttaggcatgttcatgaattcttttgcagtctttcaggaatgtggactctgggtattgacagatgcaaacctcacgaaggattatattgatggtgtttatgacaatgcagaatatgctgagaggtttatggaggaaaatgaaggacatattgtagatattcatgacttttctttgggtagcagtccacatgtccgaaagcattttccagagacttggatttggctagacaccaacatgggttacaggatttaccaagaatttgaagtaactgtacctgattctatcacttcttgggtggctactggttttgtgatctctgaggacctgggtcttggactaacaactactccagtggagctccaagccttccaaccatttttcatttttttgaatcttccctactctgttatcagaggtgaagaatttgctttggaaataactatattcaattatttgaaagatgccactgaggttaaggtaatcattgagaaaagtgacaaatttgatattctaatgacttcaaatgaaataaatgccacaggccaccagcagacccttctggttcccagtgaggatggggcaactgttctttttcccatcaggccaacacatctgggagaaattcctatcacagtcacagctctttcacccactgcttctgatgctgtcacccagatgattttagtaaaggctgaaggaatagaaaaatcatattcacaatccatcttattagacttgactgacaataggctacagagtaccctgaaaactttgagtttctcatttcctcctaatacagtgactggcagtgaaagagttcagatcactgcaattggagatgttcttggtccttccatcaatggcttagcctcattgattcggatgccttatggctgtggtgaacagaacatgataaattttgctccaaatatttacattttggattatctgactaaaaagaaacaactgacagataatttgaaagaaaaagctctttcatttatgaggcaaggttaccagagagaacttctctatcagagggaagatggctctttcagtgcttttgggaattatgacccttctgggagcacttggttgtcagcttttgttttaagatgtttccttgaagccgatccttacatagatattgatcagaatgtgttacacagaacatacacttggcttaaaggacatcagaaatccaacggtgaattttgggatccaggaagagtgattcatagtgagcttcaaggtggcaataaaagtccagtaacacttacagcctatattgtaacttctctcctgggatatagaaagtatcagcctaacattgatgtgcaagagtctatccattttttggagtctgaattcagtagaggaatttcagacaattatactctagcccttataacttatgcattgtcatcagtggggagtcctaaagcgaaggaagctttgaatatgctgacttggagagcagaacaagaaggtggcatgcaattctgggtgtcatcagagtccaaactttctgactcctggcagccacgctccctggatattgaagttgcagcctatgcactgctctcacacttcttacaatttcagacttctgagggaatcccaattatgaggtggctaagcaggcaaagaaatagcttgggtggttttgcatctactcaggataccactgtggctttaaaggctctgtctgaatttgcagccctaatgaatacagaaaggacaaatatccaagtgaccgtgacggggcctagctcaccaagtcctcttgctgtggtacagccaacggcagttaatatttccgcaaatggttttggatttgctatttgtcagctcaatgttgtatataatgtgaaggcttctgggtcttctagaagacgaagatctatccaaaatcaagaagcctttgatttagatgttgctgtaaaagaaaataaagatgatctcaatcatgtggatttgaatgtgtgtacaagcttttcgggcccgggtaggagtggcatggctcttatggaagttaacctattaagtggctttatggtgccttcagaagcaatttctctgagcgagacagtgaagaaagtggaatatgatcatggaaaactcaacctctatttagattctgtaaatgaaacccagttttgtgttaatattcctgctgtgagaaactttaaagtttcaaatacccaagatgcttcagtgtccatagtggattactatgagccaaggagacaggcggtgagaagttacaactctgaagtgaagctgtcctcctgtgacctttgcagtgatgtccagggctgccgtccttgtgaggatggagcttcaggctcccatcatcactcttcagtcatttttattttctgtttcaagcttctgtactttatggaactttggctgtgatttatttttaaaggactctgtgtaacactaacatttccagtagtcacatgtgattgttttgttttcgtagaagaatactgcttctattttgaaaaaagagttttttttctttctatggggttgcagggatggtgtacaacaggtcctagcatgtatagctgcatagatttcttcacctgatctttgtgtggaagatcagaatgaatgcagttgtgtgtctatattttcccctctcaaaatcttttagaatttttttggaggtgtttgttttctccagaataaaggtattactttagaataggtattctcctcattttgtgaaagaaatgaacctagattcttaagcattattacacatccatgtttgcttaaagatggatttccctgggaatgggagaaaacagccagcaggaggagcttcatctgttcccttcccacctccaacctagccctactgcccaccccaccccaacccaccccatgcccagtggtctcagtagatacttcttaactggaaattctttcttttcagaatctaggtggtgaattttttttaagtggcacggtctttttctgcttgaaatctgatcacaccccccagccattgccctccctctctttttcctctgtagagaaatgtgaggggcagtacatttactgtgcttttcacaccatctcagaggttgaggagcatactgaaaattgccctggggggtgctgggtgtgctgtctccttcccacatcctcagccccacaccagctctatttcaggggtgagagtcagagagcactgcaatatgtgcttcatgggatttcgattcgaagatcctagaccagggagacactgtgagccagggatacaacaaaatactaggtaagtcactgcagaccgacctccctgcagtttgggaaagaagctgggtttgtggagaatcagagcatcttgacatgactgctgacctaaagatccctggcattggccagggatcctgtggaacctcttctagttcaggggtgtgagcattagactgccagttgtctagtgacatctgatgcttgctgtgaacttttaagatccccgaatcctgagcacctcaatctttaattgccctgtattccgaagggtaatataatttatctggatggaaattttaaagatgaatcccccttttttcttttcttctctcttttctttccttctccctttcttctttgccttctaaatatactgaaatgatttagatatgtgtcaacaattaatgatcttttattcaatctaagaaatggtttagtttttctctttagctctatggcatttcactcaagtggacaggggaaaaagtaattgccatgggctccaaagaatttgctttatgtttttagctatttaaaaataaatccatcaaaaataaagtatgcaaatgtatcttttaaagttaatttttaaaaatgctcttattttagtgaattttcagaaattatagtggaatggatgctcatatattgcttatggatattttggataccaaagtaggaataactgacattcagtattttaaagctggcaaacctgtacatagaaaatagatccccagacagtggtctatgaagagggcagttaagtatcaaatacttaattttcttgcctttttttcttaagtggggaaaagtttctagatctcttacacctctgacacaatctgttctaaaacaggcacttgtaatgttggggcctccttgtaaacgtgtttttgccctttactctctgggagttctttaaaggtgaaatcatcttacaaagaaattgggggagggtcttggcaaaggactttcccctcctctttcctggcctgggaaccttatactgacaatcaatactttatattttaaagtatataatttatagttaacttctagtgtaatatattaggaaacactagaatggaaaggccattggaagacaggttgtatcttttttagaccatatttccttgtttaaaaactatcatttgaatacttttttggtgaagaactccatgttttcaagttaaaggtcacctcgtaggccaggcgcagtggctcatgcctgtaatcccagcactctgggaggctgaggcgggtgaatcacaaggttaggagtttgagaccagcctggccaatatggtgaaaccccgtccctactaaaaatacaaaatttagccaggcgtggtggcatgcacctgtagtcccacctactcgggaggctgaggcaggagaatcacttgaacctgagagacagaggttgcagtgagccgagatcacgccactgcactccagcctgggggacagagtgagattctgtctcaaaaaacaaaaaacaaaaaagtcaccttgtaactcatctctttttattgtaagtttattaaaaatgaagaggacaacaatgagaaggaacataaagggttagctagcactgtctcctggtgcatggggctgtgcagatgtcccggccacttcttccttcatacttcccttagagaacttgctctgctacaagcagtgggcttggactaaaagtgattaaaataccacaggcataaggagaaaaggagtatatgtagtagtaataattactagtataaattattttcttcacatgctatgagtaataatattaaaaaactcattttaccattaagattccttatgctgaagctcttccatttagaatactgtcaatgtcatttactggtatgaactaaagtcccccttcttttccactcactgggaaccttagtaaaacaccagcatatcttacctctctttctgactggccgatgcttccagagactgaatgttgggaaaacctagtagccaaacaattctaggacagaataacatttttatatttggttccaccatcttattacatttagttatagttttaaaaaagaaattcaagcccattaaaatatgtctggtcaatgaaatgcttccttttattgtgttgtgctattgtactttgtttttcaaaacattgtaaaaatagtatctttggtttagtattttggattatatattataatctgaggagtgttttgcttatgtagaatccagatatatttctgttacctaggagatgttacttacatatgtaatactgtatcctgcacgtggaaatattcagaattgtagatagcataactctccctgctcctattcttttgagcctaggtataattttttttttttttttagaaaaagacatatttagctttaatttctatttatgctaaacatatttataagtagtctgtcaatataataccaactatttttatttttacataattcaattatttcatttgacatgtctggcagactcaagacattaagtaaaaaattggaactatgatttttctttgtcattttttaaaaaagaattattttattaacctgctggcatataatctggagttcttttcacaaccttactttttctgatttgctttattgaatgattgaatactcatttctttctaaaaatatgttgtaaattctcccttggcaagatttctccctatgagggtagttattatttgagtctgccaagtggttaccatggggcaaggtgccatgatgtattcttgggtgcattggttttttgcgcattgtaaatttaagacacttatagtaagtggactcattcatagatgagtttcagaaccttttacgttctcggtagaggcttctgtcggacaggcagaagagtgtattcctcacttttttttttgtcttcaaattccagtaaggcatagcacttttaagaaattagaatttttctatcatctatgcaaatgatatttatgttaatattaaatatcttatgttacactgggagtaatttgaggtgcaattatttttattactactttgaatagaggaccattatccttctttcttcagaaaactaagaagtaagtgtaacttttaaagtaagtatatatcagtgagagtaggcttgttttacaactatttctagccagtgagttgtgttttcatgtctcatcaaaagacaataccacattgcatcattttacaaaatatgttgtcattttcatttcagttgtaacataggaaaatagatatttcctagatgatttctgagtttcttactgcaaagaacagttataaattggtatacatgtgtctctgtaatagggataatattgatatatctgttgctacatatttaagaatcattctatcttatgttgtcttgaggccaagatttaccacgtttgcccagtgtattgaattggtggtagaaggtagttccatgttccatttgtagatctttaagattttatctttgataactttaatagaatgtggctcagttctggtccttcaagcctgtatggtttggattttcagtaggggacagttgatgtggagtcaatctctttggtacacaggaagctttataaaatttcattcacgaatctcttattttgggaagctgttttgcatatgagaagaacactgttgaaataaggaactaaagctttatatattgatcaaggtgattctgaaagttttaatttttaatgttgtaatgttatgttattgttaattgtactttattatgtattcaatagaaaatcatgatttattaataaaagcttaaattctcatctattt

SEQ ID NO: 17 - DNA, 9221 bp, Homo sapiens
  source 1..9221: mol_type = genomic DNA
atagttgctatgaatttctcctcacaaggatgggcctgtttactcaccctgggcatcgttggtagagcgctagtgtaaacagcctgaggaacccggtgggccggggaagtgggcgcgctctgttctccgcggccagctgggacgccgggccaggtggggccgcctgcgtttagcaactgctttctcaccccctggatttgcgatgtttgccacagcagcgagaagcgccattgtaatggggatgggaggggtggagcctccaagtcctgtctcaatttagatctctcactctgctgttaggcgcgcccatttcagattactaaactcgaattaagagggaaaaaaaatcagggaggaggtggcaagccacaccccacggtgcccgcgaacttccccggcagcggactgtagcccaggcagacgccgtcgagatgcagggcccaccgctcctgaccgccgcccacctcctctgcgtgtgcaccgccgcgctggccgtggctcccgggcctcggtttctggtgacagccccagggatcatcaggcccggaggaaatgtgactattggggtggagcttctggaacactgcccttcacaggtgactgtgaaggcggagctgctcaagacagcatcaaacctcactgtctctgtcctggaagcagaaggagtctttgaaaaaggctcttttaagacacttactcttccatcagaccccaaatcaaatttgatccaacagtggttgtcacaacaaagtgatcttggagtcatttccaaaacttttcagctatcttcccatccaatacttggtgactggtctattcaagttcaagtgaatgaccagacatactatcaatcatttcaggtttcagaatatgtattaccaaaatttgaagtgactttgcagacaccattatattgttctatgaattctaagcatttaaatggtaccatcacggcaaagtatacatatgggaagccagtgaaaggagacgtaacgcttacatttttacctttatccttttggggaaagaagaaaaatattacaaaaacatttaagataaatggatctgcaaacttctcttttaatgatgaagagatgaaaaatgtaatggattcttcaaatggactttctgaatacctggatctatcttcccctggaccagtagaaattttaaccacagtgacagaatcagttacaggtatttcaagaaatgtaagcactaatgtgttcttcaagcaacatgattacatcattgagttttttgattatactactgtcttgaagccatctctcaacttcacagccactgtgaaggtaactcgtgctgatggcaaccaactgactcttgaagaaagaagaaataatgtagtcataacagtgacacagagaaactatactgagtactggagcggatctaacagtggaaatcagaaaatggaagctgttcagaaaataaattatactgtcccccaaagtggaacttttaagattgaattcccaatcctggaggattccagtgagctacagttgaaggcctatttccttggtagtaaaagtagcatggcagttcatagtctgtttaagtctcctagtaagacatacatccaactaaaaacaagagatgaaaatataaaggtgggatcgccttttgagttggtggttagtggcaacaaacgattgaaggagttaagctatatggtagtatccaggggacagttggtggctgtaggaaaacaaaattcaacaatgttctctttaacaccagaaaattcttggactccaaaagcctgtgtaattgtgtattatattgaagatgatggggaaattataagtgatgttctaaaaattcctgttcagcttgtttttaaaaataagataaagctatattggagtaaagtgaaagctgaaccatctgagaaagtctctcttaggatctctgtgacacagcctgactccatagttgggattgtagctgttgacaaaagtgtgaatctgatgaatgcctctaatgatattacaatggaaaatgtggtccatgagttggaactttataacacaggatattatttaggcatgttcatgaattcttttgcagtctttcaggaatgtggactctgggtattgacagatgcaaacctcacgaaggattatattgatggtgtttatgacaatgcagaatatgctgagaggtttatggaggaaaatgaaggacatattgtagatattcatgacttttctttgggtagcagtccacatgtccgaaagcattttccagagacttggatttggctagacaccaacatgggttacaggatttaccaagaatttgaagtaactgtacctgattctatcacttcttgggtggctactggttttgtgatctctgaggacctgggtcttggactaacaactactccagtggagctccaagccttccaaccatttttcatttttttgaatcttccctactctgttatcagaggtgaagaatttgctttggaaataactatattcaattatttgaaagatgccactgaggttaaggtaatcattgagaaaagtgacaaatttgatattctaatgacttcaaatgaaataaatgccacaggccaccagcagacccttctggttcccagtgaggatggggcaactgttctttttcccatcaggccaacacatctgggagaaattcctatcacagtcacagctctttcacccactgcttctgatgctgtcacccagatgattttagtaaaggctgaaggaatagaaaaatcatattcacaatccatcttattagacttgactgacaataggctacagagtaccctgaaaactttgagtttctcatttcctcctaatacagtgactggcagtgaaagagttcagatcactgcaattggagatgttcttggtccttccatcaatggcttagcctcattgattcggatgccttatggctgtggtgaacagaacatgataaattttgctccaaatatttacattttggattatctgactaaaaagaaacaactgacagataatttgaaagaaaaagctctttcatttatgaggcaaggttaccagagagaacttctctatcagagggaagatggctctttcagtgcttttgggaattatgacccttctgggagcacttggttgtcagcttttgttttaagatgtttccttgaagccgatccttacatagatattgatcagaatgtgttacacagaacatacacttggcttaaaggacatcagaaatccaacggtgaattttgggatccaggaagagtgattcatagtgagcttcaaggtggcaataaaagtccagtaacacttacagcctatattgtaacttctctcctgggatatagaaagtatcagcctaacattgatgtgcaagagtctatccattttttggagtctgaattcagtagaggaatttcagacaattatactctagcccttataacttatgcattgtcatcagtggggagtcctaaagcgaaggaagctttgaatatgctgacttggagagcagaacaagaaggtggcatgcaattctgggtgtcatcagagtccaaactttctgactcctggcagccacgctccctggatattgaagttgcagcctatgcactgctctcacacttcttacaatttcagacttctgagggaatcccaattatgaggtggctaagcaggcaaagaaatagcttgggtggttttgcatctactcaggataccactgtggctttaaaggctctgtctgaatttgcagccctaatgaatacagaaaggacaaatatccaagtgaccgtgacggggcctagctcaccaagtcctgtaaagtttctgattgacacacacaaccgcttactccttcagacagcagagcttgctgtggtacagccaacggcagttaatatttccgcaaatggttttggatttgctatttgtcagctcaatgttgtatataatgtgaaggcttctgggtcttctagaagacgaagatctatccaaaatcaagaagcctttgatttagatgttgctgtaaaagaaaataaagatgatctcaatcatgtggatttgaatgtgtgtacaagcttttcgggcccgggtaggagtggcatggctcttatggaagttaacctattaagtggctttatggtgccttcagaagcaatttctctgagcgagacagtgaagaaagtggaatatgatcatggaaaactcaacctctatttagattctgtaaatgaaacccagttttgtgttaatattcctgctgtgagaaactttaaagtttcaaatacccaagatgcttcagtgtccatagtggattactatgagccaaggagacaggcggtgagaagttacaactctgaagtgaagctgtcctcctgtgacctttgcagtgatgtccagggctgccgtccttgtgaggatggagcttcaggctcccatcatcactcttcagtcatttttattttctgtttcaagcttctgtactttatggaactttggctgtgatttatttttaaaggactctgtgtaacactaacatttccagtagtcacatgtgattgttttgttttcgtagaagaatactgcttctattttgaaaaaagagttttttttctttctatggggttgcagggatggtgtacaacaggtcctagcatgtatagctgcatagatttcttcacctgatctttgtgtggaagatcagaatgaatgcagttgtgtgtctatattttcccctctcaaaatcttttagaatttttttggaggtgtttgttttctccagaataaaggtattactttagaataggtattctcctcattttgtgaaagaaatgaacctagattcttaagcattattacacatccatgtttgcttaaagatggatttccctgggaatgggagaaaacagccagcaggaggagcttcatctgttcccttcccacctccaacctagccctactgcccaccccaccccaacccaccccatgcccagtggtctcagtagatacttcttaactggaaattctttcttttcagaatctaggtggtgaattttttttaagtggcacggtctttttctgcttgaaatctgatcacaccccccagccattgccctccctctctttttcctctgtagagaaatgtgaggggcagtacatttactgtgcttttcacaccatctcagaggttgaggagcatactgaaaattgccctggggggtgctgggtgtgctgtctccttcccacatcctcagccccacaccagctctatttcaggggtgagagtcagagagcactgcaatatgtgcttcatgggatttcgattcgaagatcctagaccagggagacactgtgagccagggatacaacaaaatactaggtaagtcactgcagaccgacctccctgcagtttgggaaagaagctgggtttgtggagaatcagagcatcttgacatgactgctgacctaaagatccctggcattggccagggatcctgtggaacctcttctagttcaggggtgtgagcattagactgccagttgtctagtgacatctgatgcttgctgtgaacttttaagatccccgaatcctgagcacctcaatctttaattgccctgtattccgaagggtaatataatttatctggatggaaattttaaagatgaatcccccttttttcttttcttctctcttttctttccttctccctttcttctttgccttctaaatatactgaaatgatttagatatgtgtcaacaattaatgatcttttattcaatctaagaaatggtttagtttttctctttagctctatggcatttcactcaagtggacaggggaaaaagtaattgccatgggctccaaagaatttgctttatgtttttagctatttaaaaataaatccatcaaaaataaagtatgcaaatgtatcttttaaagttaatttttaaaaatgctcttattttagtgaattttcagaaattatagtggaatggatgctcatatattgcttatggatattttggataccaaagtaggaataactgacattcagtattttaaagctggcaaacctgtacatagaaaatagatccccagacagtggtctatgaagagggcagttaagtatcaaatacttaattttcttgcctttttttcttaagtggggaaaagtttctagatctcttacacctctgacacaatctgttctaaaacaggcacttgtaatgttggggcctccttgtaaacgtgtttttgccctttactctctgggagttctttaaaggtgaaatcatcttacaaagaaattgggggagggtcttggcaaaggactttcccctcctctttcctggcctgggaaccttatactgacaatcaatactttatattttaaagtatataatttatagttaacttctagtgtaatatattaggaaacactagaatggaaaggccattggaagacaggttgtatcttttttagaccatatttccttgtttaaaaactatcatttgaatacttttttggtgaagaactccatgttttcaagttaaaggtcacctcgtaggccaggcgcagtggctcatgcctgtaatcccagcactctgggaggctgaggcgggtgaatcacaaggttaggagtttgagaccagcctggccaatatggtgaaaccccgtccctactaaaaatacaaaatttagccaggcgtggtggcatgcacctgtagtcccacctactcgggaggctgaggcaggagaatcacttgaacctgagagacagaggttgcagtgagccgagatcacgccactgcactccagcctgggggacagagtgagattctgtctcaaaaaacaaaaaacaaaaaagtcaccttgtaactcatctctttttattgtaagtttattaaaaatgaagaggacaacaatgagaaggaacataaagggttagctagcactgtctcctggtgcatggggctgtgcagatgtcccggccacttcttccttcatacttcccttagagaacttgctctgctacaagcagtgggcttggactaaaagtgattaaaataccacaggcataaggagaaaaggagtatatgtagtagtaataattactagtataaattattttcttcacatgctatgagtaataatattaaaaaactcattttaccattaagattccttatgctgaagctcttccatttagaatactgtcaatgtcatttactggtatgaactaaagtcccccttcttttccactcactgggaaccttagtaaaacaccagcatatcttacctctctttctgactggccgatgcttccagagactgaatgttgggaaaacctagtagccaaacaattctaggacagaataacatttttatatttggttccaccatcttattacatttagttatagttttaaaaaagaaattcaagcccattaaaatatgtctggtcaatgaaatgcttccttttattgtgttgtgctattgtactttgtttttcaaaacattgtaaaaatagtatctttggtttagtattttggattatatattataatctgaggagtgttttgcttatgtagaatccagatatatttctgttacctaggagatgttacttacatatgtaatactgtatcctgcacgtggaaatattcagaattgtagatagcataactctccctgctcctattcttttgagcctaggtataattttttttttttttttagaaaaagacatatttagctttaatttctatttatgctaaacatatttataagtagtctgtcaatataataccaactatttttatttttacataattcaattatttcatttgacatgtctggcagactcaagacattaagtaaaaaattggaactatgatttttctttgtcattttttaaaaaagaattattttattaacctgctggcatataatctggagttcttttcacaaccttactttttctgatttgctttattgaatgattgaatactcatttctttctaaaaatatgttgtaaattctcccttggcaagatttctccctatgagggtagttattatttgagtctgccaagtggttaccatggggcaaggtgccatgatgtattcttgggtgcattggttttttgcgcattgtaaatttaagacacttatagtaagtggactcattcatagatgagtttcagaaccttttacgttctcggtagaggcttctgtcggacaggcagaagagtgtattcctcacttttttttttgtcttcaaattccagtaaggcatagcacttttaagaaattagaatttttctatcatctatgcaaatgatatttatgttaatattaaatatcttatgttacactgggagtaatttgaggtgcaattatttttattactactttgaatagaggaccattatccttctttcttcagaaaactaagaagtaagtgtaacttttaaagtaagtatatatcagtgagagtaggcttgttttacaactatttctagccagtgagttgtgttttcatgtctcatcaaaagacaataccacattgcatcattttacaaaatatgttgtcattttcatttcagttgtaacataggaaaatagatatttcctagatgatttctgagtttcttactgcaaagaacagttataaattggtatacatgtgtctctgtaatagggataatattgatatatctgttgctacatatttaagaatcattctatcttatgttgtcttgaggccaagatttaccacgtttgcccagtgtattgaattggtggtagaaggtagttccatgttccatttgtagatctttaagattttatctttgataactttaatagaatgtggctcagttctggtccttcaagcctgtatggtttggattttcagtaggggacagttgatgtggagtcaatctctttggtacacaggaagctttataaaatttcattcacgaatctcttattttgggaagctgttttgcatatgagaagaacactgttgaaataaggaactaaagctttatatattgatcaaggtgattctgaaagttttaatttttaatgttgtaatgttatgttattgttaattgtactttattatgtattcaatagaaaatcatgatttattaataaaagcttaaattctcatctattt

SEQ ID NO: 18 - DNA, 9031 bp, Homo sapiens
  source 1..9031: mol_type = genomic DNA
agacgccgtcgagatgcagggcccaccgctcctgaccgccgcccacctcctctgcgtgtgcaccgccgcgctggccgtggctcccgggcctcggtttctggtgacagccccagggatcatcaggcccggaggaaatgtgactattggggtggagcttctggaacactgcccttcacaggtgactgtgaaggcggagctgctcaagacagcatcaaacctcactgtctctgtcctggaagcagaaggagtctttgaaaaaggctcttttaagacacttactcttccatcactacctctgaacagtgcagatgagatttatgagctacgtgtaaccggacgtacccaggatgagattttattctctaatagtacccgcttatcatttgagaccaagagaatatctgtcttcattcaaacagacaaggccttatacaagccaaagcaagaagtgaagtttcgcattgttacactcttctcagattttaagccttacaaaacctctttaaacattctcattaaggaccccaaatcaaatttgatccaacagtggttgtcacaacaaagtgatcttggagtcatttccaaaacttttcagctatcttcccatccaatacttggtgactggtctattcaagttcaagtgaatgaccagacatactatcaatcatttcaggtttcagaatatgtattaccaaaatttgaagtgactttgcagacaccattatattgttctatgaattctaagcatttaaatggtaccatcacggcaaagtatacatatgggaagccagtgaaaggagacgtaacgcttacatttttacctttatccttttggggaaagaagaaaaatattacaaaaacatttaagataaatggatctgcaaacttctcttttaatgatgaagagatgaaaaatgtaatggattcttcaaatggactttctgaatacctggatctatcttcccctggaccagtagaaattttaaccacagtgacagaatcagttacaggtatttcaagaaatgtaagcactaatgtgttcttcaagcaacatgattacatcattgagttttttgattatactactgtcttgaagccatctctcaacttcacagccactgtgaaggtaactcgtgctgatggcaaccaactgactcttgaagaaagaagaaataatgtagtcataacagtgacacagagaaactatactgagtactggagcggatctaacagtggaaatcagaaaatggaagctgttcagaaaataaattatactgtcccccaaagtggaacttttaagattgaattcccaatcctggaggattccagtgagctacagttgaaggcctatttccttggtagtaaaagtagcatggcagttcatagtctgtttaagtctcctagtaagacatacatccaactaaaaacaagagatgaaaatataaaggtgggatcgccttttgagttggtggttagtggcaacaaacgattgaaggagttaagctatatggtagtatccaggggacagttggtggctgtaggaaaacaaaattcaacaatgttctctttaacaccagaaaattcttggactccaaaagcctgtgtaattgtgtattatattgaagatgatggggaaattataagtgatgttctaaaaattcctgttcagcttgtttttaaaaataagataaagctatattggagtaaagtgaaagctgaaccatctgagaaagtctctcttaggatctctgtgacacagcctgactccatagttgggattgtagctgttgacaaaagtgtgaatctgatgaatgcctctaatgatattacaatggaaaatgtggtccatgagttggaactttataacacaggatattatttaggcatgttcatgaattcttttgcagtctttcaggaatgtggactctgggtattgacagatgcaaacctcacgaaggattatattgatggtgtttatgacaatgcagaatatgctgagaggtttatggaggaaaatgaaggacatattgtagatattcatgacttttctttgggtagcagtccacatgtccgaaagcattttccagagacttggatttggctagacaccaacatgggttacaggatttaccaagaatttgaagtaactgtacctgattctatcacttcttgggtggctactggttttgtgatctctgaggacctgggtcttggactaacaactactccagtggagctccaagccttccaaccatttttcatttttttgaatcttccctactctgttatcagaggtgaagaatttgctttggaaataactatattcaattatttgaaagatgccactgaggttaaggtaatcattgagaaaagtgacaaatttgatattctaatgacttcaaatgaaataaatgccacaggccaccagcagacccttctggttcccagtgaggatggggcaactgttctttttcccatcaggccaacacatctgggagaaattcctatcacagtcacagctctttcacccactgcttctgatgctgtcacccagatgattttagtaaaggctgaaggaatagaaaaatcatattcacaatccatcttattagacttgactgacaataggctacagagtaccctgaaaactttgagtttctcatttcctcctaatacagtgactggcagtgaaagagttcagatcactgcaattggagatgttcttggtccttccatcaatggcttagcctcattgattcggatgccttatggctgtggtgaacagaacatgataaattttgctccaaatatttacattttggattatctgactaaaaagaaacaactgacagataatttgaaagaaaaagctctttcatttatgaggcaaggttaccagagagaacttctctatcagagggaagatggctctttcagtgcttttgggaattatgacccttctgggagcacttggttgtcagcttttgttttaagatgtttccttgaagccgatccttacatagatattgatcagaatgtgttacacagaacatacacttggcttaaaggacatcagaaatccaacggtgaattttgggatccaggaagagtgattcatagtgagcttcaaggtggcaataaaagtccagtaacacttacagcctatattgtaacttctctcctgggatatagaaagtatcagcctaacattgatgtgcaagagtctatccattttttggagtctgaattcagtagaggaatttcagacaattatactctagcccttataacttatgcattgtcatcagtggggagtcctaaagcgaaggaagctttgaatatgctgacttggagagcagaacaagaaggtggcatgcaattctgggtgtcatcagagtccaaactttctgactcctggcagccacgctccctggatattgaagttgcagcctatgcactgctctcacacttcttacaatttcagacttctgagggaatcccaattatgaggtggctaagcaggcaaagaaatagcttgggtggttttgcatctactcaggataccactgtggctttaaaggctctgtctgaatttgcagccctaatgaatacagaaaggacaaatatccaagtgaccgtgacggggcctagctcaccaagtcctgtaaagtttctgattgacacacacaaccgcttactccttcagacagcagagcttgctgtggtacagccaacggcagttaatatttccgcaaatggttttggatttgctatttgtcagctcaatgttgtatataatgtgaaggcttctgggtcttctagaagacgaagatctatccaaaatcaagaagcctttgatttagatgttgctgtaaaagaaaataaagatgatctcaatcatgtggatttgaatgtgtgtacaagcttttcgggcccgggtaggagtggcatggctcttatggaagttaacctattaagtggctttatggtgccttcagaagcaatttctctgagcgagacagtgaagaaagtggaatatgatcatggaaaactcaacctctatttagattctgtaaatgaaacccagttttgtgttaatattcctgctgtgagaaactttaaagtttcaaatacccaagatgcttcagtgtccatagtggattactatgagccaaggagacaggcggtgagaagttacaactctgaagtgaagctgtcctcctgtgacctttgcagtgatgtccagggctgccgtccttgtgaggatggagcttcaggctcccatcatcactcttcagtcatttttattttctgtttcaagcttctgtactttatggaactttggctgtgatttatttttaaaggactctgtgtaacactaacatttccagtagtcacatgtgattgttttgttttcgtagaagaatactgcttctattttgaaaaaagagttttttttctttctatggggttgcagggatggtgtacaacaggtcctagcatgtatagctgcatagatttcttcacctgatctttgtgtggaagatcagaatgaatgcagttgtgtgtctatattttcccctctcaaaatcttttagaatttttttggaggtgtttgttttctccagaataaaggtattactttagaataggtattctcctcattttgtgaaagaaatgaacctagattcttaagcattattacacatccatgtttgcttaaagatggatttccctgggaatgggagaaaacagccagcaggaggagcttcatctgttcccttcccacctccaacctagccctactgcccaccccaccccaacccaccccatgcccagtggtctcagtagatacttcttaactggaaattctttcttttcagaatctaggtggtgaattttttttaagtggcacggtctttttctgcttgaaatctgatcacaccccccagccattgccctccctctctttttcctctgtagagaaatgtgaggggcagtacatttactgtgcttttcacaccatctcagaggttgaggagcatactgaaaattgccctggggggtgctgggtgtgctgtctccttcccacatcctcagccccacaccagctctatttcaggggtgagagtcagagagcactgcaatatgtgcttcatgggatttcgattcgaagatcctagaccagggagacactgtgagccagggatacaacaaaatactaggtaagtcactgcagaccgacctccctgcagtttgggaaagaagctgggtttgtggagaatcagagcatcttgacatgactgctgacctaaagatccctggcattggccagggatcctgtggaacctcttctagttcaggggtgtgagcattagactgccagttgtctagtgacatctgatgcttgctgtgaacttttaagatccccgaatcctgagcacctcaatctttaattgccctgtattccgaagggtaatataatttatctggatggaaattttaaagatgaatcccccttttttcttttcttctctcttttctttccttctccctttcttctttgccttctaaatatactgaaatgatttagatatgtgtcaacaattaatgatcttttattcaatctaagaaatggtttagtttttctctttagctctatggcatttcactcaagtggacaggggaaaaagtaattgccatgggctccaaagaatttgctttatgtttttagctatttaaaaataaatccatcaaaaataaagtatgcaaatgtatcttttaaagttaatttttaaaaatgctcttattttagtgaattttcagaaattatagtggaatggatgctcatatattgcttatggatattttggataccaaagtaggaataactgacattcagtattttaaagctggcaaacctgtacatagaaaatagatccccagacagtggtctatgaagagggcagttaagtatcaaatacttaattttcttgcctttttttcttaagtggggaaaagtttctagatctcttacacctctgacacaatctgttctaaaacaggcacttgtaatgttggggcctccttgtaaacgtgtttttgccctttactctctgggagttctttaaaggtgaaatcatcttacaaagaaattgggggagggtcttggcaaaggactttcccctcctctttcctggcctgggaaccttatactgacaatcaatactttatattttaaagtatataatttatagttaacttctagtgtaatatattaggaaacactagaatggaaaggccattggaagacaggttgtatcttttttagaccatatttccttgtttaaaaactatcatttgaatacttttttggtgaagaactccatgttttcaagttaaaggtcacctcgtaggccaggcgcagtggctcatgcctgtaatcccagcactctgggaggctgaggcgggtgaatcacaaggttaggagtttgagaccagcctggccaatatggtgaaaccccgtccctactaaaaatacaaaatttagccaggcgtggtggcatgcacctgtagtcccacctactcgggaggctgaggcaggagaatcacttgaacctgagagacagaggttgcagtgagccgagatcacgccactgcactccagcctgggggacagagtgagattctgtctcaaaaaacaaaaaacaaaaaagtcaccttgtaactcatctctttttattgtaagtttattaaaaatgaagaggacaacaatgagaaggaacataaagggttagctagcactgtctcctggtgcatggggctgtgcagatgtcccggccacttcttccttcatacttcccttagagaacttgctctgctacaagcagtgggcttggactaaaagtgattaaaataccacaggcataaggagaaaaggagtatatgtagtagtaataattactagtataaattattttcttcacatgctatgagtaataatattaaaaaactcattttaccattaagattccttatgctgaagctcttccatttagaatactgtcaatgtcatttactggtatgaactaaagtcccccttcttttccactcactgggaaccttagtaaaacaccagcatatcttacctctctttctgactggccgatgcttccagagactgaatgttgggaaaacctagtagccaaacaattctaggacagaataacatttttatatttggttccaccatcttattacatttagttatagttttaaaaaagaaattcaagcccattaaaatatgtctggtcaatgaaatgcttccttttattgtgttgtgctattgtactttgtttttcaaaacattgtaaaaatagtatctttggtttagtattttggattatatattataatctgaggagtgttttgcttatgtagaatccagatatatttctgttacctaggagatgttacttacatatgtaatactgtatcctgcacgtggaaatattcagaattgtagatagcataactctccctgctcctattcttttgagcctaggtataattttttttttttttttagaaaaagacatatttagctttaatttctatttatgctaaacatatttataagtagtctgtcaatataataccaactatttttatttttacataattcaattatttcatttgacatgtctggcagactcaagacattaagtaaaaaattggaactatgatttttctttgtcattttttaaaaaagaattattttattaacctgctggcatataatctggagttcttttcacaaccttactttttctgatttgctttattgaatgattgaatactcatttctttctaaaaatatgttgtaaattctcccttggcaagatttctccctatgagggtagttattatttgagtctgccaagtggttaccatggggcaaggtgccatgatgtattcttgggtgcattggttttttgcgcattgtaaatttaagacacttatagtaagtggactcattcatagatgagtttcagaaccttttacgttctcggtagaggcttctgtcggacaggcagaagagtgtattcctcacttttttttttgtcttcaaattccagtaaggcatagcacttttaagaaattagaatttttctatcatctatgcaaatgatatttatgttaatattaaatatcttatgttacactgggagtaatttgaggtgcaattatttttattactactttgaatagaggaccattatccttctttcttcagaaaactaagaagtaagtgtaacttttaaagtaagtatatatcagtgagagtaggcttgttttacaactatttctagccagtgagttgtgttttcatgtctcatcaaaagacaataccacattgcatcattttacaaaatatgttgtcattttcatttcagttgtaacataggaaaatagatatttcctagatgatttctgagtttcttactgcaaagaacagttataaattggtatacatgtgtctctgtaatagggataatattgatatatctgttgctacatatttaagaatcattctatcttatgttgtcttgaggccaagatttaccacgtttgcccagtgtattgaattggtggtagaaggtagttccatgttccatttgtagatctttaagattttatctttgataactttaatagaatgtggctcagttctggtccttcaagcctgtatggtttggattttcagtaggggacagttgatgtggagtcaatctctttggtacacaggaagctttataaaatttcattcacgaatctcttattttgggaagctgttttgcatatgagaagaacactgttgaaataaggaactaaagctttatatattgatcaaggtgattctgaaagttttaatttttaatgttgtaatgttatgttattgttaattgtactttattatgtattcaatagaaaatcatgatttattaataaaagcttaaattctcatcta

SEQ ID NO: 19 - DNA, 5883 bp, Homo sapiens
  source 1..5883: mol_type = genomic DNA
ctaaactcgaattaagagggaaaaaaaatcagggaggaggtggcaagccacaccccacggtgcccgcgaacttccccggcagcggactgtagcccaggcagacgccgtcgagatgcagggcccaccgctcctgaccgccgcccacctcctctgcgtgtgcaccgccgcgctggccgtggctcccgggcctcggtttctggtgacagccccagggatcatcaggcccggaggaaatgtgactattggggtggagcttctggaacactgcccttcacaggtgactgtgaaggcggagctgctcaagacagcatcaaacctcactgtctctgtcctggaagcagaaggagtctttgaaaaaggctcttttaagacacttactcttccatcactacctctgaacagtgcagatgagatttatgagctacgtgtaaccggacgtacccaggatgagattttattctctaatagtacccgcttatcatttgagaccaagagaatatctgtcttcattcaaacagacaaggccttatacaagccaaagcaagaagtgaagtttcgcattgttacactcttctcagattttaagccttacaaaacctctttaaacattctcattaaggaccccaaatcaaatttgatccaacagtggttgtcacaacaaagtgatcttggagtcatttccaaaacttttcagctatcttcccatccaatacttggtgactggtctattcaagttcaagtgaatgaccagacatattatcaatcatttcaggtttcagaatatgtattaccaaaatttgaagtgactttgcagacaccattatattgttctatgaattctaagcatttaaatggtaccatcacggcaaagtatacatatgggaagccagtgaaaggagacgtaacgcttacatttttacctttatccttttggggaaagaagaaaaatattacaaaaacatttaagataaatggatctgcaaacttctcttttaatgatgaagagatgaaaaatgtaatggattcttcaaatggactttctgaatacctggatctatcttcccctggaccagtagaaattttaaccacagtgacagaatcagttacaggtatttcaagaaatgtaagcactaatgtgttcttcaagcaacatgattacatcattgagttttttgattatactactgtcttgaagccatctctcaacttcacagccactgtgaaggtaactcgtgctgatggcaaccaactgactcttgaagaaagaagaaataatgtagtcataacagtgacacagagaaactatactgagtactggagcggatctaacagtggaaatcagaaaatggaagctgttcagaaaataaattatactgtcccccaaagtggaacttttaagattgaattcccaatcctggaggattccagtgagctacagttgaaggcctatttccttggtagtaaaagtagcatggcagttcatagtctgtttaagtctcctagtaagacatacatccaactaaaaacaagagatgaaaatataaaggtgggatcgccttttgagttggtggttagtggcaacaaacgattgaaggagttaagctatatggtagtatccaggggacagttggtggctgtaggaaaacaaaattcaacaatgttctctttaacaccagaaaattcttggactccaaaagcctgtgtaattgtgtattatattgaagatgatggggaaattataagtgatgttctaaaaattcctgttcagcttgtttttaaaaataagataaagctatattggagtaaagtgaaagctgaaccatctgagaaagtctctcttaggatctctgtgacacagcctgactccatagttgggattgtagctgttgacaaaagtgtgaatctgatgaatgcctctaatgatattacaatggaaaatgtggtccatgagttggaactttataacacaggatattatttaggcatgttcatgaattcttttgcagtctttcaggaatgtggactctgggtattgacagatgcaaacctcacgaaggattatattgatggtgtttatgacaatgcagaatatgctgagaggtttatggaggaaaatgaaggacatattgtagatattcatgacttttctttgggtagcagtccacatgtccgaaagcattttccagagacttggatttggctagacaccaacatgggttacaggatttaccaagaatttgaagtaactgtacctgattctatcacttcttgggtggctactggttttgtgatctctgaggacctgggtcttggactaacaactactccagtggagctccaagccttccaaccatttttcatttttttgaatcttccctactctgttatcagaggtgaagaatttgctttggaaataactatattcaattatttgaaagatgccactgaggttaaggtaatcattgagaaaagtgacaaatttgatattctaatgacttcaaatgaaataaatgccacaggccaccagcagacccttctggttcccagtgaggatggggcaactgttctttttcccatcaggccaacacatctgggagaaattcctatcacagtcacagctctttcacccactgcttctgatgctgtcacccagatgattttagtaaaggctgaaggaatagaaaaatcatattcacaatccatcttattagacttgactgacaataggctacagagtaccctgaaaactttgagtttctcatttcctcctaatacagtgactggcagtgaaagagttcagatcactgcaattggagatgttcttggtccttccatcaatggcttagcctcattgattcggatgccttatggctgtggtgaacagaacatgataaattttgctccaaatatttacattttggattatctgactaaaaagaaacaactgacagataatttgaaagaaaaagctctttcatttatgaggcaaggttaccagagagaacttctctatcagagggaagatggctctttcagtgcttttgggaattatgacccttctgggagcacttggttgtcagcttttgttttaagatgtttccttgaagccgatccttacatagatattgatcagaatgtgttacacagaacatacacttggcttaaaggacatcagaaatccaacggtgaattttgggatccaggaagagtgattcatagtgagcttcaaggtggcaataaaagtccagtaacacttacagcctatattgtaacttctctcctgggatatagaaagtatcagcctaacattgatgtgcaagagtctatccattttttggagtctgaattcagtagaggaatttcagacaattatactctagcccttataacttatgcattgtcatcagtggggagtcctaaagcgaaggaagctttgaatatgctgacttggagagcagaacaagaaggtggcatgcaattctgggtgtcatcagagtccaaactttctgactcctggcagccacgctccctggatattgaagttgcagcctatgcactgctctcacacttcttacaatttcagacttctgagggaatcccaattatgaggtggctaagcaggcaaagaaatagcttgggtggttttgcatctactcaggataccactgtggctttaaaggctctgtctgaatttgcagccctaatgaatacagaaaggacaaatatccaagtgaccgtgacggggcctagctcaccaagtcctgtaaagtttctgattgacacacacaaccgcttactccttcagacagcagagcttgctgtggtacagccaatggcagttaatatttccgcaaatggttttggatttgctatttgtcagctcaatgttgtatataatgtgaaggcttctgggtcttctagaagacgaagatctatccaaaatcaagaagcctttgatttagatgttgctgtaaaagaaaataaagatgatctcaatcatgtggatttgaatgtgtgtacaagcttttcgggcccgggtaggagtggcatggctcttatggaagttaacctattaagtggctttatggtgccttcagaagcaatttctctgagcgagacagtgaagaaagtggaatatgatcatggaaaactcaacctctatttagattctgtaaatgaaacccagttttgtgttaatattcctgctgtgagaaactttaaagtttcaaatacccaagatgcttcagtgtccatagtggattactatgagccaaggagacaggcggtgagaagttacaactctgaagtgaagctgtcctcctgtgacctttgcagtgatgtccagggctgccgtccttgtgaggatggagcttcaggctcccatcatcactcttcagtcatttttattttctgtttcaagcttctgtactttatggaactttggctgtgatttatttttaaaggactctgtgtaacactaacatttccagtagtcacatgtgattgttttgttttcgtagaagaatactgcttctattttgaaaaaagagttttttttctttctatggggttgcagggatggtgtacaacaggtcctagcatgtatagctgcatagatttcttcacctgatctttgtgtggaagatcagaatgaatgcagttgtgtgtctatattttcccctcacaaaatcttttagaatttttttggaggtgtttgttttctccagaataaaggtattactttagaaataggtattctcctcattttgtgaaagaaatgaacctagattcttaagcattattacacatccatgtttgcttaaagatggatttccctgggaatgggagaaaacagccagcaggaggagcttcatctgttcccttcccacctccaacctagccctactgcccaccccaccccaacccaccccatgcccagtggtctcagtagatacttcttaactggaaattctttcttttcagaatctaggtggtgaattttttttaagtggcacggtctttttctgcttgaaatctgatcacaccccccagccattgccctccctctctttttcctctgtagagaaatgtgaggggcagtacatttactgtgcttttcacaccatctcagaggttgaggagcatactgaaaattgccctggggggtgctgggtgtgctgtctccttcccacatcctcagccccacaccagctctatttcaggggtgagagtcagagagcactgcaatatgtgcttcatgggatttcgattcgaagatcctagaccagggagacactgtgagccagggatacaacaaaatactaggtaagtcactgcagaccgacctccctgcagtttgggaaagaagctgggtttgtggagaatcagagcatcttgacatgactgctgacctaaagatccctggcattggccagggatcctgtggaacctcttctagttcaggggtgtgagcattagactgccagttgtctagtgacatctgatgcttgctgtgaacttttaagatccccgaatcctgagcacctcaatctttaattgccctgtattccgaagggtaatataatttatctggatggaaattttaaagatgaatcccccttttttcttttcttctctcttttctttccttctccctttcttctttgccttctaaatatactgaaatgatttagatatgtgtcaacaattaatgatcttttattcaatctaagaaatggtttagtttttctctttagctctatggcatttcactcaagtggacaggggaaaaagtaattgccatgggctccaaagaatttgctttatgtttttagctatttaaaaataaatccatcaaaaataaagtatgcaaatgtatctttt

SEQ ID NO: 20 - DNA, 4449 bp, Homo sapiens
  source 1..4449: mol_type = genomic DNA
aaaactcgaattaagagggaaagaaaatcagggaggaggtggcaagccacaccccacggtgcccgcgaacttccccggcagcggactgtagcccaggcagacgccgtcgagatgcagggcccaccgctcctgaccgccgcccacctcctctgcgtgtgcaccgccgcgctggccgtggctcccgggcctcggtttctggtgacagccccagggatcatcaggcccggaggaaatgtgactattggggtggagcttctggaacactgcccttcacaggtgactgtgaaggcggagctgctcaagacagcatcaaacctcactgtctctgtcctggaagcagaaggagtctttgaaaaaggctcttttaagacacttactcttccatcactacctctgaacagtgcagatgagatttatgagctacgtgtaaccggacgtacccaggatgagattttattctctaatagtacccgcttatcatttgagaccaagagaatatctgtcttcattcaaacagacaaggccttatacaagccaaagcaagaagtgaagtttcgcattgttacactcttctcagattttaagccttacaaaacctctttaaacattctcattaaggaccccaaatcaaatttgatccaacagtggttgtcacaacaaagtgatcttggagtcatttccaaaacttttcagctatcttcccatccaatacttggtgactggtctattcaagttcaagtgaatgaccagacatattatcaatcatttcaggtttcagaatatgtattaccaaaatttgaagtgactttgcagacaccattatattgttctatgaattctaagcatttaaatggtaccatcacggcaaagtatacatatgggaagccagtgaaaggagacgtaacgcttacatttttacctttatccttttggggaaagaagaaaaatattacaaaaacatttaagataaatggatctgcaaacttctcttttaatgatgaagagatgaaaaatgtaatggattcttcaaatggactttctgaatacctggatctatcttcccctggaccagtagaaattttaaccacagtgacagaatcagttacaggtatttcaagaaatgtaagcactaatgtgttcttcaagcaacatgattacatcattgagttttttgattatactactgtcttgaagccatctctcaacttcacagccactgtgaaggtaactcgtgctgatggcaaccaactgactcttgaagaaagaagaaataatgtagtcataacagtgacacagagaaactatactgagtactggagcggatctaacagtggaaatcagaaaatggaagctgttcagaaaataaattatactgtcccccaaagtggaacttttaagattgaattcccaatcctggaggattccagtgagctacagttgaaggcctatttccttggtagtaaaagtagcatggcagttcatagtctgtttaagtctcctagtaagacatacatccaactaaaaacaagagatgaaaatataaaggtgggatcgccttttgagttggtggttagtggcaacaaacgattgaaggagttaagctatatggtagtatccaggggacagttggtggctgtaggaaaacaaaattcaacaatgttctctttaacaccagaaaattcttggactccaaaagcctgtgtaattgtgtattatattgaagatgatggggaaattataagtgatgttctaaaaattcctgttcagcttgtttttaaaaataagataaagctatattggagtaaagtgaaagctgaaccatctgagaaagtctctcttaggatctctgtgacacagcctgactccatagttgggattgtagctgttgacaaaagtgtgaatctgatgaatgcctctaatgatattacaatggaaaatgtggtccatgagttggaactttataacacaggatattatttaggcatgttcatgaattcttttgcagtctttcaggaatgtggactctgggtattgacagatgcaaacctcacgaaggattatattgatggtgtttatgacaatgcagaatatgctgagaggtttatggaggaaaatgaaggacatattgtagatattcatgacttttctttgggtagcagtccacatgtccgaaagcattttccagagacttggatttggctagacaccaacatgggttccaggatttaccaagaatttgaagtaactgtacctgattctatcacttcttgggtggctactggttttgtgatctctgaggacctgggtcttggactaacaactactccagtggagctccaagccttccaaccatttttcatttttttgaatcttccctactctgttatcagaggtgaagaatttgctttggaaataactatattcaattatttgaaagatgccactgaggttaaggtaatcattgagaaaagtgacgaatttgatattctaatgacttcaaatgaaataaatgccacaggccaccagcagacccttctggttcccagtgaggatggggcaactgttctttttcccatcaggccaacacatctgggagaaattcctatcacagtcacagctctttcacccactgcttctgatgctgtcacccagatgattttagtaaaggctgaaggaatagaaaaatcatattcacaatccatcttattagacttgactgacaataggctacagagtaccctgaaaactttgagtttctcatttcctcctaatacagtgactggcagtgaaagagttcagatcactgcaattggagatgttcttggtccttccatcaatggcttagcctcattgattcggatgccttatggctgtggtgaacagaacatgataaattttgctccaaatatttacattttggattatctgactaaaaagaaacaactgacagataatttgaaagaaaaagctctttcatttatgaggcaaggttaccagagagaacttctctatcagagggaagatggctctttcagtgcttttgggaattatgacccttctgggagcacttggttgtcagcttttgttttaagatgtttccttgaagccgatccttacatagatattgatcagaatgtgttacacagaacatacacttggcttaaaggacatcagaaatccaacggtgaattttgggatccaggaagagtgattcatagtgagcttcaaggtggcaataaaagtccagtaacacttacagcctatattgtaacttctctcctgggatatagaaagtatcagcctaacattgatgtgcaagagtctatccattttttggagtctgaattcagtagaggaatttcagacaattatactctagcccttataacttatgcattgtcatcagtggggagtcctaaagcgaaggaagctttgaatatgctgacttggagagcagaacaagaaggtggcatgcaattctgggtgtcatcagagtccaaactttctgactcctggcagccacgctccctggatattgaagttgcagcctatgcactgctctcacacttcttacaatttcagacttctgagggaatcccaattatgaggtggctaagcaggcaaagaaatagcttgggtggttttgcatctactcaggataccactgtggctttaaaggctctgtctgaatttgcagccctaatgaatacagaaaggacaaatatccaggtgaccgtgacggggcctagctcaccaagtcctgtaaagtttctgattgacacacacaaccgcttactccttcagacagcagagcttgctgtggtacagccaacggcagttaatatttccgcaaatggttttggatttgctatttgtcagctcaatgttgtatataatgtgaaggcttctgggtcttctagaagacgaagatctatccaaaatcaagaagcctttgatttagatgttgctgtaaaagaaaataaagatgatctcaatcatgtggatttgaatgtgtgtacaagcttttcgggcccgggtaggagtggcatggctcttatggaagttaacctattaagtggctttatggtgccttcagaagcaatttctctgagcgagacagtgaagaaagtggaatatgatcatggaaaactcaacctctatttagattctgtaaatgaaacccagttttgtgttaatattcctgctgtgagaaactttaaagtttcaaatacccaagatgcttcagtgtccatagtggattactatgagccaaggagacaggcggtgagaagttacaactctgaagtgaagctgtcctcctgtgacctttgcagtgatgtccagggctgccgtccttgtgaggatggagcttcaggctcccatcatcactcttcagtcatttttattttctgtttcaagcttctgtactttatggaactttggctgtga

SEQ ID NO: 21 - DNA, 2273 bp, Homo sapiens
  source 1..2273: mol_type = genomic DNA
acacaccccacggtgcccgcgaacttccccggcagcggactgtagcccaggcagacgccgtcgagatgcagggcccaccgctcctgaccgccgcccacctcctctgcgtgtgcaccgccgcgctggccgtggctcccgggcctcggtttctggtgacagccccagggatcatcaggcccggaggaaatgtgactattggggtggagcttctggaacactgcccttcacaggtgactgtgaaggcggagctgctcaagacagcatcaaacctcactgtctctgtcctggaagcagaaggagtctttgaaaaaggctcttttaagacacttactcttccatcactacctctgaacagtgcagatgagatttatgagctacgtgtaaccggacgtacccaggatgagattttattctctaatagtacccgcttatcatttgagaccaagagaatatctgtcttcattcaaacagacaaggccttatacaagccaaagcaagaagtgaagtttcgcattgttacactcttctcagattttaagccttacaaaacctctttaaacattctcattaaggaccccaaatcaaatttgatccaacagtggttgtcacaacaaagtgatcttggagtcatttccaaaacttttcagctatcttcccatccaatacttggtgactggtctattcaagttcaagtgaatgaccagacatactatcaatcatttcaggtttcagaatatgtattaccaaaatttgaagtgactttgcagacaccattatattgttctatgaattctaagcatttaaatggtaccatcacggcaaagtatacatatgggaagccagtgaaaggagacgtaacgcttacatttttacctttatccttttggggaaagaagaaaaatattacaaaaacatttaagataaatggatctgcaaacttctcttttaatgatgaagagatgaaaaatgtaatggattcttcaaatggactttctgaatacctggatctatcttcccctggaccagtagaaattttaaccacagtgacagaatcagttacaggtatttcaagaaatgtaagcactaatgtgttcttcaagcaacatgattacatcattgagttttttgattatactactgtcttgaagccatctctcaacttcacagccactgtgaaggtaactcgtgctgatggcaaccaactgactcttgaagaaagaagaaataatgtagtcataacagtgacacagagaaactatactgagtactggagcggatctaacagtggaaatcagaaaatggaagctgttcagaaaataaattatactgtcccccaaagtggaacttttaagattgaattcccaatcctggaggattccagtgagctacagttgaaggcctatttccttggtagtaaaagtagcatggcagttcatagtctgtttaagtctcctagtaagacatacatccaactaaaaacaagagatgaaaatataaaggtgggatcgccttttgagttggtggttagtggcaacaaacgattgaaggagttaagctatatggtagtatccaggggacagttggtggctgtaggaaaacaaaattcaacaatgttctctttaacaccagaaaattcttggactccaaaagcctgtgtaattgtgtattatattgaagatgatggggaaattataagtgatgttctaaaaattcctgttcagcttgtttttaaaaataagataaagctatattggagtaaagtgaaagctgaaccatctgagaaagtctctcttaggatctctgtgacacagcctgactccatagttgggattgtagctgttgacaaaagtgtgaatctgatgaatgcctctaatgatattacaatggaaaatgtggtccatgagttggaactttataacacaggatattatttaggcatgttcatgaattcttttgcagtctttcaggaatgtggactctgggtattgacagatgcaaacctcacgaaggattatattgatggtgtttatgacaatctctttggtacacaggaagctttataaaatttcattcacgaatctcttattttgggaagctgttttgcatatgagaagaacactgttgaaataaggaactaaagctttatatattgatcaaggtgattctgaaagttttaatttttaatgttgtaatgttatgttattgttaattgtactttattatgtattcaatagaaaatcatgatttattaataaaagcttaaattctcatct

SEQ ID NO: 22 - DNA, 4688 bp, Homo sapiens
  source 1..4688: mol_type = genomic DNA
gggactgtagcccaggcagacgccgtcgagatgcagggcccaccgctcctgaccgccgcccacctcctctgcgtgtgcaccgccgcgctggccgtggctcccgggcctcggtttctggtgacagccccagggatcatcaggcccggaggaaatgtgactattggggtggagcttctggaacactgcccttcacaggtgactgtgaaggcggagctgctcaagacagcatcaaacctcactgtctctgtcctggaagcagaaggagtctttgaaaaaggctcttttaagacacttactcttccatcactacctctgaacagtgcagatgagatttatgagctacgtgtaaccggacgtacccaggatgagattttattctctaatagtacccgcttatcatttgagaccaagagaatatctgtcttcattcaaacagacaaggccttatacaagccaaagcaagaagtgaagtttcgcattgttacactcttctcagattttaagccttacaaaacctctttaaacattctcattaaggaccccaaatcaaatttgatccaacagtggttgtcacaacaaagtgatcttggagtcatttccaaaacttttcagctatcttcccatccaatacttggtgactggtctattcaagttcaagtgaatgaccagacatattatcaatcatttcaggtttcagaatatgtattaccaaaatttgaagtgactttgcagacaccattatattgttctatgaattctaagcatttaaatggtaccatcacggcaaagtatacatatgggaagccagtgaaaggagacgtaacgcttacatttttacctttatccttttggggaaagaagaaaaatattacaaaaacatttaagataaatggatctgcaaacttctcttttaatgatgaagagatgaaaaatgtaatggattcttcaaatggactttctgaatacctggatctatcttcccctggaccagtagaaattttaaccacagtgacagaatcagttacaggtatttcaagaaatgtaagcactaatgtgttcttcaagcaacatgattacatcattgagttttttgattataccactgtcttgaagccatctctcaacttcacagccactgtgaaggtaactcgtgctgatggcaaccaactgactcttgaagaaagaagaaataatgtagtcataacagtgacacagagaaactatactgagtactggagcggatctaacagtggaaatcagaaaatggaagctgttcagaaaataaattatactgtcccccaaagtggaacttttaagattgaattcccaatcctggaggattccagtgagctacagttgaaggcctatttccttggtagtaaaagtagcatggcagttcatagtctgtttaagtctcctagtaagacatacatccaactaaaaacaagagatgaaaatataaaggtgggatcgccttttgagttggtggttagtggcaacaaacgattgaaggagttaagctatatggtagtatccaggggacagttggtggctgtaggaaaacaaaattcaacaatgttctctttaacaccagaaaattcttggactccaaaagcctgtgtaattgtgtattatattgaagatgatggggaaattataagtgatgttctaaaaattcctgttcagcttgtttttaaaaataagataaagctatattggagtaaagtgaaagctgaaccatctgagaaagtctctcttaggatctctgtgacacagcctgactccatagttgggattgtagctgttgacaaaagtgtgaatctgatgaatgcctctaatgatattacaatggaaaatgtggtccatgagttggaactttataacacaggatattatttaggcatgttcatgaattcttttgcagtctttcaggaatgtggactctgggtattgacagatgcaaacctcacgaaggattatattgatggtgtttatgacaatgcagaatatgctgagaggtttatggaggaaaatgaaggacatattgtagatattcatgacttttctttgggtagcagtccacatgtccgaaagcattttccagagacttggatttggctagacaccaacatgggttccaggatttaccaagaatttgaagtaactgtacctgattctatcacttcttgggtggctactggttttgtgatctctgaggacctgggtcttggactaacaactactccagtggagctccaagccttccaaccatttttcatttttttgaatcttccctactctgttatcagaggtgaagaatttgctttggaaataactatattcaattatttgaaagatgccactgaggttaaggtaatcattgagaaaagtgacaaatttgatattctaatgacttcaagtgaaataaatgccacaggccaccagcagacccttctggttcccagtgaggatggggcaactgttctttttcccatcaggccaacacatctgggagaaattcctatcacagtcacagctctttcacccactgcttctgatgctatcacccagatgattttagtaaaggctgaaggaatagaaaaatcatattcacaatccatcttattagacttgactgacaataggctacagagtaccctgaaaactttgagtttctcatttcctcctaatacagtgactggcagtgaaagagttcagatcactgcaattggagatgttcttggtccttccatcaatggcttagcctcattgattcggatgccttatggctgtggtgaacagaacatgataaattttgctccaaatatttacattttggattatctgactaaaaagaaacaactgacagataatttgaaagaaaaagctctttcatttatgaggcaaggttaccagagagaacttctctatcagagggaagatggctctttcagtgcttttgggaattatgacccttctgggagcacttggttgtcagcttttgttttaagatgtttccttgaagccgatccttacatagatattgatcagaatgtgttacacagaacatacacttggcttaaaggacatcagaaatccaacggtgaattttgggatccaggaagagtgattcatagtgagcttcaaggtggcaataaaagtccagcaacacttacagcctatattgtaacttctctcctgggatatagaaagtatcagcctaacattgatgtgcaagagtctatccattttttggagtctgaattcagtagaggaatttcagacaattatactctagcccttataacttatgcattgtcatcagtggggagtcctaaagcgaaggaagctttgaatatgctgacttggagagcagaacaagaaggtggcatgcaattctgggtgtcatcagagtccaaactttctgactcctggcagccacgctccctggatattgaagttgcagcctatgcactgctctcacacttcttacaatttcagacttctgagggaatcccaattatgaggtggctaagcaggcaaagaaatagcttgggtggttttgcatctactcaggataccactgtggctttaaaggctctgtctgaatttgcagccctaatgaatacagaaaggacaaatatccaagtgaccgtgacggggcctagctcaccaagtcctgtaaagtttctgattgacacacacaaccgcttactccttcagacagcagagcttgctgtggtacagccaatggcagttaatatttccgcaaatggttttggatttgctatttgtcagctcaatgttgtatataatgtgaaggcttctgggtcttctagaagacgaagatctatccaaaatcaagaagcctttgatttagatgttgctgtaaaagaaaataaagatgatctcaatcatgtggatttgaatgtgtgtacaagcttttcgggcccgggtaggagtggcatggctcttatggaagttaacctattaagtggctttatggtgccttcagaagcaatttctctgagcgagacagtgaagaaagtggaatatgatcatggaaaactcaacctctatttagattctgtaaatgaaacccagttttgtgttaatattcctgctgtgagaaactttaaagtttcaaatacccaagatgcttcagtgtccatagtggattactatgagccaaggagacaggcggtgagaagttacaactctgaagtgaagctgtcctcctgtgacctttgcagtgatgtccagggctgccgtccttgtgaggatggagcttcaggctcccatcatcactcttcagtcatttttattttctgtttcaagcttctgtactttatggaactttggctgtgatttatttttaaaggactctgtgtaacactaacatttccagtagtcacatgtgattgttttgttttcgtagaagaatactgcttctattttgaaaaaagagttttttttctttctatggggttgcagggatggtgtacaacaggtcctagcatgtatagctgcatagatttcttcacctgatctttgtgtggaagatcagaatgaatgcagttgtgtgtctatattttcccctctcaaaatcttttagaatttttttggaggtgtttgttttctccagaataaaggtattactttagaaaacaaaaaaaaaaaaaaaaaaa

SEQ ID NO: 23 - DNA, 4369 bp, Homo sapiens
  source 1..4369: mol_type = genomic DNA
tgtagcccaggcagacgccgtcgagatgcagggcccaccgctcctgaccgccgcccacctcctctgcgtgtgcaccgccgcgctggccgtggctcccgggcctcggtttctggtgacagccccagggatcatcaggcccggaggaaatgtgactattggggtggagcttctggaacactgcccttcacaggtgactgtgaaggcggagctgctcaagacagcatcaaacctcactgtctctgtcctggaagcagaaggagtctttgaaaaaggctcttttaagacacttactcttccatcactacctctgaacagtgcagatgagatttatgagctacgtgtaaccggacgtacccaggatgagattttattctctaatagtacccgcttatcatttgagaccaagagaatatctgtcttcattcaaacagacaaggccttatacaagccaaagcaagaagtgaagtttcgcattgttacactcttctcagattttaagccttacaaaacctctttaaacattctcattaaggaccccaaatcaaatttgatccaacagtggttgtcacaacaaagtgatcttggagtcatttccaaaacttttcagctatcttcccatccaatacttggtgactggtctattcaagttcaagtgaatgaccagacatattatcaatcatttcaggtttcagaatatgtattaccaaaatttgaagtgactttgcagacaccattatattgttctatgaattctaagcatttaaatggtaccatcacggcaaagtatacatatgggaagccagtgaaaggagacgtaacgcttacatttttacctttatccttttggggaaagaagaaaaatattacaaaaacatttaagataaatggatctgcaaacttctcttttaatgatgaagagatgaaaaatgtaatggattcttcaaatggactttctgaatacctggatctatcttcccctggaccagtagaaattttaaccacagtgacagaatcagttacaggtatttcaagaaatgtaagcactaatgtgttcttcaagcaacatgattacatcattgagttttttgattatactactgtcttgaagccatctctcaacttcacagccactgtgaaggtaactcgtgctgatggcaaccaactgactcttgaagaaagaagaaataatgtagtcataacagtgacacagagaaactatactgagtactggagcggatctaacagtggaaatcagaaaatggaagctgttcagaaaataaattatactgtcccccaaagtggaacttttaagattgaattcccaatcctggaggattccagtgagctacagttgaaggcctatttccttggtagtaaaagtagcatggcagttcatagtctgtttaagtctcctagtaagacatacatccaactaaaaacaagagatgaaaatataaaggtgggatcgccttttgagttggtggttagtggcaacaaacgattgaaggagttaagctatatggtagtatccaggggacagttggtggctgtaggaaaacaaaattcaacaatgttctctttaacaccagaaaattcttggactccaaaagcctgtgtaattgtgtattatattgaagatgatggggaaattataagtgatgttctaaaaattcctgttcagcttgtttttaaaaataagataaagctatattggagtaaagtgaaagctgaaccatctgagaaagtctctcttaggatctctgtgacacagcctgactccatagttgggattgtagctgttgacaaaagtgtgaatctgatgaatgcctctaatgatattacaatggaaaatgtggtccatgagttggaactttataacacaggatattatttaggcatgttcatgaattcttttgcagtctttcaggaatgtggactctgggtattgacagatgcaaacctcacgaaggattatattgatggtgtttatgacaatgcagaatatgctgagaggtttatggaggaaaatgaaggacatattgtagatattcatgacttttctttgggtagcagtccacatgtccgaaagcattttccagagacttggatttggctagacaccaacatgggttccaggatttaccaagaatttgaagtaactgtacctgattctatcacttcttgggtggctactggttttgtgatctctgaggacctgggtcttggactaacaactactccagtggagctccaagccttccaaccatttttcatttttttgaatcttccctactctgttatcagaggtgaagaatttgctttggaaataactatattcaattatttgaaagatgccactgaggttaaggtaatcattgagaaaagtgacaaatttgatattctaatgacttcaagtgaaataaatgccacaggccaccagcagacccttctggttcccagtgaggatggggcaactgttctttttcccatcaggccaacacatctgggagaaattcctatcacagtcacagctctttcacccactgcttctgatgctatcacccagatgattttagtaaaggctgaaggaatagaaaaatcatattcacaatccatcttattagacttgactgacaataggctacagagtaccctgaaaactttgagtttctcatttcctcctaatacagtgactggcagtgaaagagttcagatcactgcaattggagatgttcttggtccttccatcaatggcttagcctcattgattcggatgccttatggctgtggtgaacagaacatgataaattttgctccaaatatttacattttggattatctgactaaaaagaaacaactgacagataatttgaaagaaaaagctctttcatttatgaggcaaggttaccagagagaacttctctatcagagggaagatggctctttcagtgcttttgggaattatgacccttctgggagcacttggttgtcagcttttgttttaagatgtttccttgaagccgatccttacatagatattgatcagaatgtgttacacagaacatacacttggcttaaaggacatcagaaatccaacggtgaattttgggatccaggaagagtgattcatagtgagcttcaaggtggcaataaaagtccagtaacacttacagcctatattgtaacttctctcctgggatatagaaagtatcagcctaacattgatgtgcaagagtctatccattttttggagtctgaattcagtagaggaatttcagacaattatactctagcccttataacttatgcattgtcatcagtggggagtcctaaagcgaaggaagctttgaatatgctgacttggagagcagaacaagaaggtggcatgcaattctgggtgtcatcagagtccaaactttctgactcctggcagccacgctccctggatattgaagttgcagcctatgcactgctctcacacttcttacaatttcagacttctgagggaatcccaattatgaggtggctaagcaggcaaagaaatagcttgggtggttttgcatctactcaggataccactgtggctttaaaggctctgtctgaatttgcagccctaatgaatacagaaaggacaaatatccaagtgaccgtgacggggcctagctcaccaagtcctcttgctgtggtacagccaacggcagttaatatttccgcaaatggttttggatttgctatttgtcagctcaatgttgtatataatgtgaaggcttctgggtcttctagaagacgaagatctatccaaaatcaagaagcctttgatttagatgttgctgtaaaagaaaataaagatgatctcaatcatgtggatttgaatgtgtgtacaagcttttcgggcccgggtaggagtggcatggctcttatggaagttaacctattaagtggctttatggtgccttcagaagcaatttctctgagcgagacagtgaagaaagtggaatatgatcatggaaaactcaacctctatttagattctgtaaatgaaacccagttttgtgttaatattcctgctgtgagaaactttaaagtttcaaatacccaagatgcttcagtgtccatagtggattactatgagccaaggagacaggcggtgagaagttacaactctgaagtgaagctgtcctcctgtgacctttgcagtgatgtccagggctgccgtccttgtgaggatggagcttcaggctcccatcatcactcttcagtcatttttattttctgtttcaagcttctgtactttatggaactttggctgtgatttatttttaaaggactctgtgtaacactaacatttccagtagtcacatgtgattgt

SEQ ID NO: 24 - DNA, 4237 bp, Homo sapiens
  source 1..4237: mol_type = genomic DNA
gaggcagacgccgtcgagatgcagggcccaccgctcctgaccgccgcccacctcctctgcgtgtgcaccgccgcgctggccgtggctcccgggcctcggtttctggtgacagccccagggatcatcaggcccggaggaaatgtgactattggggtggagcttctggaacactgcccttcacaggtgactgtgaaggcggagctgctcaagacagcatcaaacctcactgtctctgtcctggaagcagaaggagtctttgaaaaaggctcttttaagacacttactcttccatcagaccccaaatcaaatttgatccaacagtggttgtcacaacaaagtgatcttggagtcatttccaaaacttttcagctatcttcccatccaatacttggtgactggtctattcaagttcaagtgaatgaccagacatactatcaatcatttcaggtttcagaatatgtattaccaaaatttgaagtgactttgcagacaccattatattgttctatgaattctaagcatttaaatggtaccatcacggcaaagtatacatatgggaagccagtgaaaggagacgtaacgcttacatttttacctttatccttttggggaaagaagaaaaatattacaaaaacatttaagataaatggatctgcaaacttctcttttaatgatgaagagatgaaaaatgtaatggattcttcaaatggactttctgaatacctggatctatcttcccctggaccagtagaaattttaaccacagtgacagaatcagttacaggtatttcaagaaatgtaagcactaatgtgttcttcaagcaacatgattacatcattgagttttttgattatactactgtcttgaagccatctctcaacttcacagccactgtgaaggtaactcgtgctgatggcaaccaactgactcttgaagaaagaagaaataatgtagtcataacagtgacacagagaaactatactgagtactggagcggatctaacagtggaaatcagaaaatggaagctgttcagaaaataaattatactgtcccccaaagtggaacttttaagattgaattcccaatcctggaggattccagtgagctacagttgaaggcctatttccttggtagtaaaagtagcatggcagttcatagtctgtttaagtctcctagtaagacatacatccaactaaaaacaagagatgaaaatataaaggtgggatcgccttttgagttggtggttagtggcaacaaacgattgaaggagttaagctatatggtagtatccaggggacagttggtggctgtaggaaaacaaaattcaacaatgttctctttaacaccagaaaattcttggactccaaaagcctgtgtaattgtgtattatattgaagatgatggggaaattataagtgatgttctaaaaattcctgttcagcttgtttttaaaaataagataaagctatattggagtaaagtgaaagctgaaccatctgagaaagtctctcttaggatctctgtgacacagcctgactccatagttgggattgtagctgttgacaaaagtgtgaatctgatgaatgcctctaatgatattacaatggaaaatgtggtccatgagttggaactttataacacaggatattatttaggcatgttcatgaattcttttgcagtctttcaggaatgtggactctgggtattgacagatgcaaacctcacgaaggattatattgatggtgtttatgacaatgcagaatatgctgagaggtttatggaggaaaatgaaggacatattgtagatattcatgacttttctttgggtagcagtccacatgtccgaaagcattttccagagacttggatttggctagacaccaacatgggttacaggatttaccaagaatttgaagtaactgtacctgattctatcacttcttgggtggctactggttttgtgatctctgaggacctgggtcttggactaacaactactccagtggagctccaagccttccaaccatttttcatttttttgaatcttccctactctgttatcagaggtgaagaatttgctttggaaataactatattcaattatttgaaagatgccactgaggttaaggtaatcattgagaaaagtgacaaatttgatattctaatgacttcaaatgaaataaatgccacaggccaccagcagacccttctggttcccagtgaggatggggcaactgttctttttcccatcaggccaacacatctgggagaaattcctatcacagtcacagctctttcacccactgcttctgatgctgtcacccagatgattttagtaaaggctgaaggaatagaaaaatcatattcacaatccatcttattagacttgactgacaataggctacagagtaccctgaaaactttgagtttctcatttcctcctaatacagtgactggcagtgaaagagttcagatcactgcaattggagatgttcttggtccttccatcaatggcttagcctcattgattcggatgccttatggctgtggtgaacagaacatgataaattttgctccaaatatttacattttggattatctgactaaaaagaaacaactgacagataatttgaaagaaaaagctctttcatttatgaggcaaggttaccagagagaacttctctatcagagggaagatggctctttcagtgcttttgggaattatgacccttctgggagcacttggttgtcagcctttgttttaagatgtttccttgaagccgatccttacatagatattgatcagaatgtgttacacagaacatacacttggcttaaaggacatcagaaatccaacggtgaattttgggatccaggaagagtgattcatagtgagcttcaaggtggcaataaaagtccagtaacacttacagcctatattgtaacttctctcctgggatatagaaagtatcagcctaacattgatgtgcaagagtctatccattttttggagtctgaattcagtagaggaatttcagacaattatactctagcccttataacttatgcattgtcatcagtggggagtcctaaagcgaaggaagctttgaatatgctgacttggagagcagaacaagaaggtggcatgcaattctgggtgtcatcagagtccaaactttctgactcctggcagccacgctccctggatattgaagttgcagcctatgcactgctctcacacttcttacaatttcagacttctgagggaatcccaattatgaggtggctaagcaggcaaagaaatagcttgggtggttttgcatctactcaggataccactgtggctttaaaggctctgtctgaatttgcagccctaatgaatacagaaaggacaaatatccaagtgaccgtgacggggcctagctcaccaagtcctgtaaagtttctgattgacacacacaaccgcttactccttcagacagcagagcttgctgtggtacagccaatggcagttaatatttccgcaaatggttttggatttgctatttgtcagctcaatgttgtatataatgtgaaggcttctgggtcttctagaagacgaagatctatccaaaatcaagaagccttttgatttagatgttgctgtaaaagaaaataaagatgatctcaatcatgtggatttgaatgtgtgtacaagcttttcgggcccgggtaggagtggcatggctcttatggaagttaacctattaagtggctttatggtgccttcagaagcaatttctctgagcgagacagtgaagaaagtggaatatgatcatggaaaactcaacctctatttagattctgtaaatgaaacccagttttgtgttaatattcctgctgtgagaaactttaaagtttcaaatacccaagatgcttcagtgtccatagtggattactatgagccaaggagacaggcggtgagaagttacaactctgaagtgaagctgtcctcctgtgacctttgcagtgatgtccagggctgccgtccttgtgaggatggagcttcaggctcccatcatcactcttcagtcatttttattttctgtttcaagcttctgtactttatggaactttggctgtgatttatttttaaaggactctgtgtaacactaacatttccagtagtcacatgtgattgttttgttttcgtagaagaatactgcttctattttgaaaaaaaaaaaaaaaaaaca

SEQ ID NO: 25 - DNA, 4338 bp, Homo sapiens
  source 1..4338: mol_type = genomic DNA
atgcagggcccaccgctcctgaccgccgcccacctcctctgcgtgtgcaccgccgcgctggccgtggctcccgggcctcggtttctggtgacagccccagggatcatcaggcccggaggaaatgtgactattggggtggagcttctggaacactgcccttcacaggtgactgtgaaggcggagctgctcaagacagcatcaaacctcactgtctctgtcctggaagcagaaggagtctttgaaaaaggctcttttaagacacttactcttccatcactacctctgaacagtgcagatgagatttatgagctacgtgtaaccggacgtacccaggatgagattttattctctaatagtacccgcttatcatttgagaccaagagaatatctgtcttcattcaaacagacaaggccttatacaagccaaagcaagaagtgaagtttcgcattgttacactcttctcagattttaagccttacaaaacctctttaaacattctcattaaggaccccaaatcaaatttgatccaacagtggttgtcacaacaaagtgatcttggagtcatttccaaaacttttcagctatcttcccatccaatacttggtgactggtctattcaagttcaagtgaatgaccagacatattatcaatcatttcaggtttcagaatatgtattaccaaaatttgaagtgactttgcagacaccattatattgttctatgaattctaagcatttaaatggtaccatcacggcaaagtatacatatgggaagccagtgaaaggagacgtaacgcttacatttttacctttatccttttggggaaagaagaaaaatattacaaaaacatttaagataaatggatctgcaaacttctcttttaatgatgaagagatgaaaaatgtaatggattcttcaaatggactttctgaatacctggatctatcttcccctggaccagtagaaattttaaccacagtgacagaatcagttacaggtatttcaagaaatgtaagcactaatgtgttcttcaagcaacatgattacatcattgagttttttgattatactactgtcttgaagccatctctcaacttcacagccactgtgaaggtaactcgtgctgatggcaaccaactgactcttgaagaaagaagaaataatgtagtcataacagtgacacagagaaactatactgagtactggagcggatctaacagtggaaatcagaaaatggaagctgttcagaaaataaattatactgtcccccaaagtggaacttttaagattgaattcccaatcctggaggattccagtgagctacagttgaaggcctatttccttggtagtaaaagtagcatggcagttcatagtctgtttaagtctcctagtaagacatacatccaactaaaaacaagagatgaaaatataaaggtgggatcgccttttgagttggtggttagtggcaacaaacgattgaaggagttaagctatatggtagtatccaggggacagttggtggctgtaggaaaacaaaattcaacaatgttctctttaacaccagaaaattcttggactccaaaagcctgtgtaattgtgtattatattgaagatgatggggaaattataagtgatgttctaaaaattcctgttcagcttgtttttaaaaataagataaagctatattggagtaaagtgaaagctgaaccatctgagaaagtctctcttaggatctctgtgacacagcctgactccatagttgggattgtagctgttgacaaaagtgtgaatctgatgaatgcctctaatgatattacaatggaaaatgtggtccatgagttggaactttataacacaggatattatttaggcatgttcataaattcttttgcagtctttcaggaatgtggactctgggtattgacagatgcaaacctcacgaaggattatattgatggtgtttatgacaatgcagaatatgctgagaggtttatggaggaaaatgaaggacatattgtagatattcatgacttttctttgggtagcagtccacatgtccgaaagcattttccagagacttggatttggctagacaccaacatgggttccaggatttaccaagaatttgaagtaactgtacctgattctatcacttcttgggtggctactggttttgtgatctctgaggacctgggtcttggactaacaactactccagtggagctccaagccttccaaccatttttcatttttttgaatcttccctactctgttatcagaggtgaagaatttgctttggaaataactatattcaattatttgaaagatgccactgaggttaaggtaatcattgagaaaagtgacaaatttgatattctaatgacttcaagtgaaataaatgccacaagccaccagcagacccttctggttcccagtgaggatggggcaactgttctttttcccatcaggccaacacatctgggagaaattcctatcacagtcacagctctttcacccactgcttctgatgctatcacccagatgattttagtaaaggctgaaggaatagaaaaatcatattcacaatccatcttattagacttgactgacaataggctacagagtaccctgaaaactttgagtttctcatttcctcctaatacagtgactggcagtgaaagagttcagatcactgcaattggagatgttcttggtccttccatcaatggcttagcctcattgattcggatgccttatggctgtggtgaacagaacatgataaattttgctccaaatatttacattttggattatctgactaaaaagaaacaactgacagataatttgaaagaaaaagctctttcatttatgaggcaaggttaccagagagaacttctctatcagagggaagatggctctttcagtgcttttgggaattatgacccttctgggagcacttggttgtcagcttttgttttaagatgtttccttgaagccgatccttacatagatattgatcagaatgtgttacacagaacatacacttggcttaaaggacatcagaaatccaacggtgaattttgggatccaggaagagtgattcatagtgagcttcaaggtggcaataaaagtccagtaacacttacagcctatattgtaacttctctcctgggatatagaaagtatcagcctaacattgatgtgcaagagtctatccattttttggagtctgaattcagtagaggaatttcagacaattatactctagcccttataacttatgcattgtcatcagtggggagtcctaaagcgaaggaagctttgaatatgctgacttggagagcagaacaagaaggtggcatgcaattctgggtgtcatcagagtccaaactttctgactcctggcagccacgctccctggatattgaagttgcagcctatgcactgctctcacacttcttacaatttcagacttctgagggaatcccaattatgaggtggctaagcaggcaaagaaatagcttgggtggttttgcatctactcaggataccactgtggctttaaaggctctgtctgaatttgcagccctaatgaatacagaaaggacaaatatccaagtgaccgtgacggggcctagctcaccaagtcctgtaaagtttctgattgacacacacaaccgcttactccttcagacagcagagcttgctgtggtacagccaacggcagttaatatttccgcaaatggttttggatttgctatttgtcagctcaatgttgtatataatgtgaaggcttctgggtcttctagaagacgaagatctatccaaaatcaagaagcctttgatttagatgttgctgtaaaagaaaataaagatgatctcaatcatgtggatttgaatgtgtgtacaagcttttcgggcccgggtaggagtggcatggctcttatggaagttaacctattaagtggctttatggtgccttcagaagcaatttctctgagcgagacagtgaagaaagtggaatatgatcatggaaaactcaacctctatttagattctgtaaatgaaacccagttttgtgttaatattcctgctgtgagaaactttaaagtttcaaatacccaagatgcttcagtgtctatagtggattactatgagccaaggagacaggcggtgagaagttacaactctgaagtgaagctgtcctcctgtgacctttgcagtgatgtccagggctgccgtccttgtgaggatggagcttcaggctcccatcatcactcttcagtcatttttattttctgtttcaagcttctgtactttatggaactttggctgtga

SEQ ID NO: 26 - DNA, 2938 bp, Homo sapiens
  source 1..2938: mol_type = genomic DNA
gttgacaaaagtgtgaatctgatgaatgcctctaatgatattacaatggaaaatgtggtccatgagttggaactttataacacaggatattatttaggcatgttcatgaattcttttgcagtctttcaggaatgtggactctgggtattgacagatgcaaacctcacgaaggattatattgatggtgtttatgacaatgcagaatatgctgagaggtttatggaggaaaatgaaggacatattgtagatattcatgacttttctttgggtagcagtccacatgtccgaaagcattttccagagacttggatttggctagacaccaacatgggttccaggatttaccaagaatttgaagtaactgtacctgattctatcacttcttgggtggctactggttttgtgatctctgaggacctgggtcttggactaacaactactccagtggagctccaagccttccaaccatttttcatttttttgaatcttccctactctgttatcagaggtgaagaatttgctttggaaataactatattcaattatttgaaagatgccactgaggttaaggtaatcattgagaaaagtgacaaatttgatattctaatgacttcaagtgaaataaatgccacaggccaccagcagacccttctggttcccagtgaggatggggcaactgttctttttcccatcaggccaacacatctgggagaaattcctatcacagtcacagctctttcacccactgcttctgatgctatcacccagatgattttagtaaaggctgaaggaatagaaaaatcatattcacaatccatcttattagacttgactgacaataggctacagagtaccctgaaaactttgagtttctcatttcctcctaatacagtgactggcagtgaaagagttcagatcactgcaattggagatgttcttggtccttccatcaatggcttagcctcattgattcggatgccttatggctgtggtgaacagaacatgataaattttgctccaaatatttacattttggattatctgactaaaaagaaacaactgacagataatttgaaagaaaaagctctttcatttatgaggcaaggttaccagagagaacttctctatcagagggaagatggctctttcagtgcttttgggaattatgacccttctgggagcacttggttgtcagcttttgttttaagatgtttccttgaagccgatccttacatagatattgatcagaatgtgttacacagaacatacacttggcttaaaggacatcagaaatccaacggtgaattttgggatccaggaagagtgattcatagtgagcttcaaggtggcaataaaagtccagtaacacttacagcctatattgtaacttctctcctgggatatagaaagtatcagcctaacattgatgtgcaagagtctatccattttttggagtctgaattcagtagaggaatttcagacaattatactctagcccttataacttatgcattgtcatcagtggggagtcctaaagcgaaggaagctttgaatatgctgacttggagagcagaacaagaaggtggcatgcaattctgggtgtcatcagagtccaaactttctgactcctggcagccacgctccctggatattgaagttgcagcctatgcactgctctcacacttcttacaatttcagacttctgagggaatcccaattatgaggtggctaagcaggcaaagaaatagcttgggtggttttgcatctactcaggataccactgtggctttaaaggctctgtctgaatttgcagccctaatgaatacagaaaggacaaatatccaagtgaccgtgacggggcctagctcaccaagtcctgtaaagtttctgattgacacacacaaccgcttactccttcagacagcagagcttgctgtggtacagccaacggcagttaatatttccgcaaatggttttggatttgctatttgtcagctcaatgttgtatataatgtgaaggcttctgggtcttctagaagacgaagatctatccaaaatcaagaagcctttgatttagatgttgctgtaaaagaaaataaagatgatctcaatcatgtggatttgaatgtgtgtacaagcttttcgggcccgggtaggagtggcatggctcttatggaagttaacctattaagtggctttatggtgccttcagaagcaatttctctgagcgagacagtgaagaaagtggaatatgatcatggaaaactcaacctctatttagattctgtaaatgaaacccagttttgtgttaatattcctgctgtgagaaactttaaagtttcaaatacccaagatgcttcagtgtccatagtggattactatgagccaaggagacaggcggtgagaagttacaactctgaagtgaagctgtcctcctgtgacctttgcagtgatgtccagggctgccgtccttgtgaggatggagcttcaggctcccatcatcactcttcagtcatttttattttctgtttcaagcttctgtactttatggaactttggctgtgatttatttttaaaggactctgtgtaacactaacatttccagtagtcacatgtgattgttttgttttcgtagaagaatactgcttctattttgaaaaaagagttttttttctttctatggggttgcagggatggtgtacaacaggtcctagcatgtatagctgcatagatttcttcacctgatctttgtgtggaagatcagaatgaatgcagttgtgtgtctatattttcccctctcaaaatcttttagaatttttttggaggtgtttgttttctccagaataaaggtattactttagaataaaaaaaaaaaaaaaaaaaaaaaaaaaaaaaaaaaaaaaaaaaaaaaaaaaaaaaaaaaaaaaaaaaaaaaaaa

SEQ ID NO: 27 - DNA, 2843 bp, Homo sapiens
  source 1..2843: mol_type = genomic DNA
ctgatgaatgcctctaatgatattacaatggaaaatgtggtccatgagttggaactttataacacaggatattatttaggcatgttcatgaattcttttgcagtctttcaggaatgtggactctgggtattgacagatgcaaacctcacgaaggattatattgatggtgtttatgacaatgcagaatatgctgagaggtttatggaggaaaatgaaggacatattgtagatattcatgacttttctttgggtagcagtccacatgtccgaaagcattttccagagacttggatttggctagacaccaacatgggttccaggatttaccaagaatttgaagtaactgtacctgattctatcacttcttgggtggctactggttttgtgatctctgaggacctgggtcttggactaacaactactccagtggagctccaagccttccaaccatttttcatttttttgaatcttccctactctgttatcagaggtgaagaatttgctttggaaataactatattcaattatttgaaagatgccactgaggttaaggtaatcattgagaaaagtgacaaatttgatattctaatgacttcaagtgaaataaatgccacaggccaccagcagacccttctggttcccagtgaggatggggcaactgttctttttcccatcaggccaacacatctgggagaaattcctatcacagtcacagctctttcacccactgcttctgatgctatcacccagatgattttagtaaaggctgaaggaatagaaaaatcatattcacaatccatcttattagacttgactgacaataggctacagagtaccctgaaaactttgagtttctcatttcctcctaatacagtgactggcagtgaaagagttcagatcactgcaattggagatgttcttggtccttccatcaatggcttagcctcattgattcggatgccttatggctgtggtgaacagaacatgataaattttgctccaaatatttacattttggattatctgactaaaaagaaacaactgacagataatttgaaagaaaaagctctttcatttatgaggcaaggttaccagagagaacttctctatcagagggaagatggctctttcagtgcttttgggaattatgacccttctgggagcacttggttgtcagcttttgttttaagatgtttccttgaagccgatccttacatagatattgatcagaatgtgttacacagaacatacacttggcttaaaggacatcagaaatccaacggtgaattttgggatccaggaagagtgattcatagtgagcttcaaggtggcaataaaagtccagtaacacttacagcctatattgtaacttctctcctgggatatagaaagtatcagcctaacattgatgtgcaagagtctatccattttttggagtctgaattcagtagaggaatttcagacaattatactctagcccttataacttatgcattgtcatcagtggggagtcctaaagcgaaggaagctttgaatatgctgacttggagagcagaacaagaaggtggcatgcaattctgggtgtcatcagagtccaaactttctgactcctggcagccacgctccctggatattgaagttgcagcctatgcactgctctcacacttcttacaatttcagacttctgagggaatcccaattatgaggtggctaagcaggcaaagaaatagcttgggtggttttgcatctactcaggataccactgtggctttaaaggctctgtctgaatttgcagccctaatgaatacagaaaggacaaatatccaagtgaccgtgacggggcctagctcaccaagtcctgtaaagtttctgattgacacacacaaccgcttactccttcagacagcagagcttgctgtggtacagccaacggcagttaatatttccgcaaatggttttggatttgctatttgtcagctcaatgttgtatataatgtgaaggcttctgggtcttctagaagacgaagatctatccaaaatcaagaagcctttgatttagatgttgctgtaaaagaaaataaagatgatctcaatcatgtggatttgaatgtgtgtacaagcttttcgggcccgggtaggagtggcatggctcttatggaagttaacctattaagtggctttatggtgccttcagaagcaatttctctgagcgagacagtgaagaaagtggaatatgatcatggaaaactcaacctctatttagattctgtaaatgaaacccagttttgtgttaatattcctgctgtgagaaactttaaagtttcaaatacccaagatgcttcagtgtccatagtggattactatgagccaaggagacaggcggtgagaagttacaactctgaagtgaagctgtcctcctgtgacctttgcagtgatgtccagggctgccgtccttgtgagaatggagcttcaggctcccatcatcactcttcagtcatttttattttctgtttcaagcttctgtactttatggaactttggctgtgatttatttttaaaggactctgtgtaacactaacatttccagtagtcacatgtgattgttttgttttcgtagaagaatactgcttctattttgaaaaaagagttttttttctttctatggggttgcagggatggtgtacaacaggtcctagcatgtatagctgcatagatttcttcacctgatctttgtgtggaagatcagaatgaatgcagttgtgtgtctatattttcccctctcaaaatcttttagaatttttttggaggtgtttgttttctccagaataaaggtattactttag

SEQ ID NO: 28 - DNA, 8800 bp, Homo sapiens
  source 1..8800: mol_type = genomic DNA
agacgccgtcgagatgcagggcccaccgctcctgaccgccgcccacctcctctgcgtgtgcaccgccgcgctggccgtggctcccgggcctcggtttctggtgacagccccagggatcatcaggcccggaggaaatgtgactattggggtggagcttctggaacactgcccttcacaggtgactgtgaaggcggagctgctcaagacagcatcaaacctcactgtctctgtcctggaagcagaaggagtctttgaaaaaggctcttttaagacacttactcttccatcagaccccaaatcaaatttgatccaacagtggttgtcacaacaaagtgatcttggagtcatttccaaaacttttcagctatcttcccatccaatacttggtgactggtctattcaagttcaagtgaatgaccagacatactatcaatcatttcaggtttcagaatatgtattaccaaaatttgaagtgactttgcagacaccattatattgttctatgaattctaagcatttaaatggtaccatcacggcaaagtatacatatgggaagccagtgaaaggagacgtaacgcttacatttttacctttatccttttggggaaagaagaaaaatattacaaaaacatttaagataaatggatctgcaaacttctcttttaatgatgaagagatgaaaaatgtaatggattcttcaaatggactttctgaatacctggatctatcttcccctggaccagtagaaattttaaccacagtgacagaatcagttacaggtatttcaagaaatgtaagcactaatgtgttcttcaagcaacatgattacatcattgagttttttgattatactactgtcttgaagccatctctcaacttcacagccactgtgaaggtaactcgtgctgatggcaaccaactgactcttgaagaaagaagaaataatgtagtcataacagtgacacagagaaactatactgagtactggagcggatctaacagtggaaatcagaaaatggaagctgttcagaaaataaattatactgtcccccaaagtggaacttttaagattgaattcccaatcctggaggattccagtgagctacagttgaaggcctatttccttggtagtaaaagtagcatggcagttcatagtctgtttaagtctcctagtaagacatacatccaactaaaaacaagagatgaaaatataaaggtgggatcgccttttgagttggtggttagtggcaacaaacgattgaaggagttaagctatatggtagtatccaggggacagttggtggctgtaggaaaacaaaattcaacaatgttctctttaacaccagaaaattcttggactccaaaagcctgtgtaattgtgtattatattgaagatgatggggaaattataagtgatgttctaaaaattcctgttcagcttgtttttaaaaataagataaagctatattggagtaaagtgaaagctgaaccatctgagaaagtctctcttaggatctctgtgacacagcctgactccatagttgggattgtagctgttgacaaaagtgtgaatctgatgaatgcctctaatgatattacaatggaaaatgtggtccatgagttggaactttataacacaggatattatttaggcatgttcatgaattcttttgcagtctttcaggaatgtggactctgggtattgacagatgcaaacctcacgaaggattatattgatggtgtttatgacaatgcagaatatgctgagaggtttatggaggaaaatgaaggacatattgtagatattcatgacttttctttgggtagcagtccacatgtccgaaagcattttccagagacttggatttggctagacaccaacatgggttacaggatttaccaagaatttgaagtaactgtacctgattctatcacttcttgggtggctactggttttgtgatctctgaggacctgggtcttggactaacaactactccagtggagctccaagccttccaaccatttttcatttttttgaatcttccctactctgttatcagaggtgaagaatttgctttggaaataactatattcaattatttgaaagatgccactgaggttaaggtaatcattgagaaaagtgacaaatttgatattctaatgacttcaaatgaaataaatgccacaggccaccagcagacccttctggttcccagtgaggatggggcaactgttctttttcccatcaggccaacacatctgggagaaattcctatcacagtcacagctctttcacccactgcttctgatgctgtcacccagatgattttagtaaaggctgaaggaatagaaaaatcatattcacaatccatcttattagacttgactgacaataggctacagagtaccctgaaaactttgagtttctcatttcctcctaatacagtgactggcagtgaaagagttcagatcactgcaattggagatgttcttggtccttccatcaatggcttagcctcattgattcggatgccttatggctgtggtgaacagaacatgataaattttgctccaaatatttacattttggattatctgactaaaaagaaacaactgacagataatttgaaagaaaaagctctttcatttatgaggcaaggttaccagagagaacttctctatcagagggaagatggctctttcagtgcttttgggaattatgacccttctgggagcacttggttgtcagcttttgttttaagatgtttccttgaagccgatccttacatagatattgatcagaatgtgttacacagaacatacacttggcttaaaggacatcagaaatccaacggtgaattttgggatccaggaagagtgattcatagtgagcttcaaggtggcaataaaagtccagtaacacttacagcctatattgtaacttctctcctgggatatagaaagtatcagcctaacattgatgtgcaagagtctatccattttttggagtctgaattcagtagaggaatttcagacaattatactctagcccttataacttatgcattgtcatcagtggggagtcctaaagcgaaggaagctttgaatatgctgacttggagagcagaacaagaaggtggcatgcaattctgggtgtcatcagagtccaaactttctgactcctggcagccacgctccctggatattgaagttgcagcctatgcactgctctcacacttcttacaatttcagacttctgagggaatcccaattatgaggtggctaagcaggcaaagaaatagcttgggtggttttgcatctactcaggataccactgtggctttaaaggctctgtctgaatttgcagccctaatgaatacagaaaggacaaatatccaagtgaccgtgacggggcctagctcaccaagtcctgtaaagtttctgattgacacacacaaccgcttactccttcagacagcagagcttgctgtggtacagccaacggcagttaatatttccgcaaatggttttggatttgctatttgtcagctcaatgttgtatataatgtgaaggcttctgggtcttctagaagacgaagatctatccaaaatcaagaagcctttgatttagatgttgctgtaaaagaaaataaagatgatctcaatcatgtggatttgaatgtgtgtacaagcttttcgggcccgggtaggagtggcatggctcttatggaagttaacctattaagtggctttatggtgccttcagaagcaatttctctgagcgagacagtgaagaaagtggaatatgatcatggaaaactcaacctctatttagattctgtaaatgaaacccagttttgtgttaatattcctgctgtgagaaactttaaagtttcaaatacccaagatgcttcagtgtccatagtggattactatgagccaaggagacaggcggtgagaagttacaactctgaagtgaagctgtcctcctgtgacctttgcagtgatgtccagggctgccgtccttgtgaggatggagcttcaggctcccatcatcactcttcagtcatttttattttctgtttcaagcttctgtactttatggaactttggctgtgatttatttttaaaggactctgtgtaacactaacatttccagtagtcacatgtgattgttttgttttcgtagaagaatactgcttctattttgaaaaaagagttttttttctttctatggggttgcagggatggtgtacaacaggtcctagcatgtatagctgcatagatttcttcacctgatctttgtgtggaagatcagaatgaatgcagttgtgtgtctatattttcccctctcaaaatcttttagaatttttttggaggtgtttgttttctccagaataaaggtattactttagaataggtattctcctcattttgtgaaagaaatgaacctagattcttaagcattattacacatccatgtttgcttaaagatggatttccctgggaatgggagaaaacagccagcaggaggagcttcatctgttcccttcccacctccaacctagccctactgcccaccccaccccaacccaccccatgcccagtggtctcagtagatacttcttaactggaaattctttcttttcagaatctaggtggtgaattttttttaagtggcacggtctttttctgcttgaaatctgatcacaccccccagccattgccctccctctctttttcctctgtagagaaatgtgaggggcagtacatttactgtgcttttcacaccatctcagaggttgaggagcatactgaaaattgccctggggggtgctgggtgtgctgtctccttcccacatcctcagccccacaccagctctatttcaggggtgagagtcagagagcactgcaatatgtgcttcatgggatttcgattcgaagatcctagaccagggagacactgtgagccagggatacaacaaaatactaggtaagtcactgcagaccgacctccctgcagtttgggaaagaagctgggtttgtggagaatcagagcatcttgacatgactgctgacctaaagatccctggcattggccagggatcctgtggaacctcttctagttcaggggtgtgagcattagactgccagttgtctagtgacatctgatgcttgctgtgaacttttaagatccccgaatcctgagcacctcaatctttaattgccctgtattccgaagggtaatataatttatctggatggaaattttaaagatgaatcccccttttttcttttcttctctcttttctttccttctccctttcttctttgccttctaaatatactgaaatgatttagatatgtgtcaacaattaatgatcttttattcaatctaagaaatggtttagtttttctctttagctctatggcatttcactcaagtggacaggggaaaaagtaattgccatgggctccaaagaatttgctttatgtttttagctatttaaaaataaatccatcaaaaataaagtatgcaaatgtatcttttaaagttaatttttaaaaatgctcttattttagtgaattttcagaaattatagtggaatggatgctcatatattgcttatggatattttggataccaaagtaggaataactgacattcagtattttaaagctggcaaacctgtacatagaaaatagatccccagacagtggtctatgaagagggcagttaagtatcaaatacttaattttcttgcctttttttcttaagtggggaaaagtttctagatctcttacacctctgacacaatctgttctaaaacaggcacttgtaatgttggggcctccttgtaaacgtgtttttgccctttactctctgggagttctttaaaggtgaaatcatcttacaaagaaattgggggagggtcttggcaaaggactttcccctcctctttcctggcctgggaaccttatactgacaatcaatactttatattttaaagtatataatttatagttaacttctagtgtaatatattaggaaacactagaatggaaaggccattggaagacaggttgtatcttttttagaccatatttccttgtttaaaaactatcatttgaatacttttttggtgaagaactccatgttttcaagttaaaggtcacctcgtaggccaggcgcagtggctcatgcctgtaatcccagcactctgggaggctgaggcgggtgaatcacaaggttaggagtttgagaccagcctggccaatatggtgaaaccccgtccctactaaaaatacaaaatttagccaggcgtggtggcatgcacctgtagtcccacctactcgggaggctgaggcaggagaatcacttgaacctgagagacagaggttgcagtgagccgagatcacgccactgcactccagcctgggggacagagtgagattctgtctcaaaaaacaaaaaacaaaaaagtcaccttgtaactcatctctttttattgtaagtttattaaaaatgaagaggacaacaatgagaaggaacataaagggttagctagcactgtctcctggtgcatggggctgtgcagatgtcccggccacttcttccttcatacttcccttagagaacttgctctgctacaagcagtgggcttggactaaaagtgattaaaataccacaggcataaggagaaaaggagtatatgtagtagtaataattactagtataaattattttcttcacatgctatgagtaataatattaaaaaactcattttaccattaagattccttatgctgaagctcttccatttagaatactgtcaatgtcatttactggtatgaactaaagtcccccttcttttccactcactgggaaccttagtaaaacaccagcatatcttacctctctttctgactggccgatgcttccagagactgaatgttgggaaaacctagtagccaaacaattctaggacagaataacatttttatatttggttccaccatcttattacatttagttatagttttaaaaaagaaattcaagcccattaaaatatgtctggtcaatgaaatgcttccttttattgtgttgtgctattgtactttgtttttcaaaacattgtaaaaatagtatctttggtttagtattttggattatatattataatctgaggagtgttttgcttatgtagaatccagatatatttctgttacctaggagatgttacttacatatgtaatactgtatcctgcacgtggaaatattcagaattgtagatagcataactctccctgctcctattcttttgagcctaggtataattttttttttttttttagaaaaagacatatttagctttaatttctatttatgctaaacatatttataagtagtctgtcaatataataccaactatttttatttttacataattcaattatttcatttgacatgtctggcagactcaagacattaagtaaaaaattggaactatgatttttctttgtcattttttaaaaaagaattattttattaacctgctggcatataatctggagttcttttcacaaccttactttttctgatttgctttattgaatgattgaatactcatttctttctaaaaatatgttgtaaattctcccttggcaagatttctccctatgagggtagttattatttgagtctgccaagtggttaccatggggcaaggtgccatgatgtattcttgggtgcattggttttttgcgcattgtaaatttaagacacttatagtaagtggactcattcatagatgagtttcagaaccttttacgttctcggtagaggcttctgtcggacaggcagaagagtgtattcctcacttttttttttgtcttcaaattccagtaaggcatagcacttttaagaaattagaatttttctatcatctatgcaaatgatatttatgttaatattaaatatcttatgttacactgggagtaatttgaggtgcaattatttttattactactttgaatagaggaccattatccttctttcttcagaaaactaagaagtaagtgtaacttttaaagtaagtatatatcagtgagagtaggcttgttttacaactatttctagccagtgagttgtgttttcatgtctcatcaaaagacaataccacattgcatcattttacaaaatatgttgtcattttcatttcagttgtaacataggaaaatagatatttcctagatgatttctgagtttcttactgcaaagaacagttataaattggtatacatgtgtctctgtaatagggataatattgatatatctgttgctacatatttaagaatcattctatcttatgttgtcttgaggccaagatttaccacgtttgcccagtgtattgaattggtggtagaaggtagttccatgttccatttgtagatctttaagattttatctttgataactttaatagaatgtggctcagttctggtccttcaagcctgtatggtttggattttcagtaggggacagttgatgtggagtcaatctctttggtacacaggaagctttataaaatttcattcacgaatctcttattttgggaagctgttttgcatatgagaagaacactgttgaaataaggaactaaagctttatatattgatcaaggtgattctgaaagttttaatttttaatgttgtaatgttatgttattgttaattgtactttattatgtattcaatagaaaatcatgatttattaataaaagcttaaattctcatcta

SEQ ID NO: 29 - DNA, 8980 bp, Homo sapiens
  source 1..8980: mol_type = genomic DNA
agacgccgtcgagatgcagggcccaccgctcctgaccgccgcccacctcctctgcgtgtgcaccgccgcgctggccgtggctcccgggcctcggtttctggtgacagccccagggatcatcaggcccggaggaaatgtgactattggggtggagcttctggaacactgcccttcacaggtgactgtgaaggcggagctgctcaagacagcatcaaacctcactgtctctgtcctggaagcagaaggagtctttgaaaaaggctcttttaagacacttactcttccatcactacctctgaacagtgcagatgagatttatgagctacgtgtaaccggacgtacccaggatgagattttattctctaatagtacccgcttatcatttgagaccaagagaatatctgtcttcattcaaacagacaaggccttatacaagccaaagcaagaagtgaagtttcgcattgttacactcttctcagattttaagccttacaaaacctctttaaacattctcattaaggaccccaaatcaaatttgatccaacagtggttgtcacaacaaagtgatcttggagtcatttccaaaacttttcagctatcttcccatccaatacttggtgactggtctattcaagttcaagtgaatgaccagacatactatcaatcatttcaggtttcagaatatgtattaccaaaatttgaagtgactttgcagacaccattatattgttctatgaattctaagcatttaaatggtaccatcacggcaaagtatacatatgggaagccagtgaaaggagacgtaacgcttacatttttacctttatccttttggggaaagaagaaaaatattacaaaaacatttaagataaatggatctgcaaacttctcttttaatgatgaagagatgaaaaatgtaatggattcttcaaatggactttctgaatacctggatctatcttcccctggaccagtagaaattttaaccacagtgacagaatcagttacaggtatttcaagaaatgtaagcactaatgtgttcttcaagcaacatgattacatcattgagttttttgattatactactgtcttgaagccatctctcaacttcacagccactgtgaaggtaactcgtgctgatggcaaccaactgactcttgaagaaagaagaaataatgtagtcataacagtgacacagagaaactatactgagtactggagcggatctaacagtggaaatcagaaaatggaagctgttcagaaaataaattatactgtcccccaaagtggaacttttaagattgaattcccaatcctggaggattccagtgagctacagttgaaggcctatttccttggtagtaaaagtagcatggcagttcatagtctgtttaagtctcctagtaagacatacatccaactaaaaacaagagatgaaaatataaaggtgggatcgccttttgagttggtggttagtggcaacaaacgattgaaggagttaagctatatggtagtatccaggggacagttggtggctgtaggaaaacaaaattcaacaatgttctctttaacaccagaaaattcttggactccaaaagcctgtgtaattgtgtattatattgaagatgatggggaaattataagtgatgttctaaaaattcctgttcagcttgtttttaaaaataagataaagctatattggagtaaagtgaaagctgaaccatctgagaaagtctctcttaggatctctgtgacacagcctgactccatagttgggattgtagctgttgacaaaagtgtgaatctgatgaatgcctctaatgatattacaatggaaaatgtggtccatgagttggaactttataacacaggatattatttaggcatgttcatgaattcttttgcagtctttcaggaatgtggactctgggtattgacagatgcaaacctcacgaaggattatattgatggtgtttatgacaatgcagaatatgctgagaggtttatggaggaaaatgaaggacatattgtagatattcatgacttttctttgggtagcagtccacatgtccgaaagcattttccagagacttggatttggctagacaccaacatgggttacaggatttaccaagaatttgaagtaactgtacctgattctatcacttcttgggtggctactggttttgtgatctctgaggacctgggtcttggactaacaactactccagtggagctccaagccttccaaccatttttcatttttttgaatcttccctactctgttatcagaggtgaagaatttgctttggaaataactatattcaattatttgaaagatgccactgaggttaaggtaatcattgagaaaagtgacaaatttgatattctaatgacttcaaatgaaataaatgccacaggccaccagcagacccttctggttcccagtgaggatggggcaactgttctttttcccatcaggccaacacatctgggagaaattcctatcacagtcacagctctttcacccactgcttctgatgctgtcacccagatgattttagtaaaggctgaaggaatagaaaaatcatattcacaatccatcttattagacttgactgacaataggctacagagtaccctgaaaactttgagtttctcatttcctcctaatacagtgactggcagtgaaagagttcagatcactgcaattggagatgttcttggtccttccatcaatggcttagcctcattgattcggatgccttatggctgtggtgaacagaacatgataaattttgctccaaatatttacattttggattatctgactaaaaagaaacaactgacagataatttgaaagaaaaagctctttcatttatgaggcaaggttaccagagagaacttctctatcagagggaagatggctctttcagtgcttttgggaattatgacccttctgggagcacttggttgtcagcttttgttttaagatgtttccttgaagccgatccttacatagatattgatcagaatgtgttacacagaacatacacttggcttaaaggacatcagaaatccaacggtgaattttgggatccaggaagagtgattcatagtgagcttcaaggtggcaataaaagtccagtaacacttacagcctatattgtaacttctctcctgggatatagaaagtatcagcctaacattgatgtgcaagagtctatccattttttggagtctgaattcagtagaggaatttcagacaattatactctagcccttataacttatgcattgtcatcagtggggagtcctaaagcgaaggaagctttgaatatgctgacttggagagcagaacaagaaggtggcatgcaattctgggtgtcatcagagtccaaactttctgactcctggcagccacgctccctggatattgaagttgcagcctatgcactgctctcacacttcttacaatttcagacttctgagggaatcccaattatgaggtggctaagcaggcaaagaaatagcttgggtggttttgcatctactcaggataccactgtggctttaaaggctctgtctgaatttgcagccctaatgaatacagaaaggacaaatatccaagtgaccgtgacggggcctagctcaccaagtcctcttgctgtggtacagccaacggcagttaatatttccgcaaatggttttggatttgctatttgtcagctcaatgttgtatataatgtgaaggcttctgggtcttctagaagacgaagatctatccaaaatcaagaagcctttgatttagatgttgctgtaaaagaaaataaagatgatctcaatcatgtggatttgaatgtgtgtacaagcttttcgggcccgggtaggagtggcatggctcttatggaagttaacctattaagtggctttatggtgccttcagaagcaatttctctgagcgagacagtgaagaaagtggaatatgatcatggaaaactcaacctctatttagattctgtaaatgaaacccagttttgtgttaatattcctgctgtgagaaactttaaagtttcaaatacccaagatgcttcagtgtccatagtggattactatgagccaaggagacaggcggtgagaagttacaactctgaagtgaagctgtcctcctgtgacctttgcagtgatgtccagggctgccgtccttgtgaggatggagcttcaggctcccatcatcactcttcagtcatttttattttctgtttcaagcttctgtactttatggaactttggctgtgatttatttttaaaggactctgtgtaacactaacatttccagtagtcacatgtgattgttttgttttcgtagaagaatactgcttctattttgaaaaaagagttttttttctttctatggggttgcagggatggtgtacaacaggtcctagcatgtatagctgcatagatttcttcacctgatctttgtgtggaagatcagaatgaatgcagttgtgtgtctatattttcccctctcaaaatcttttagaatttttttggaggtgtttgttttctccagaataaaggtattactttagaataggtattctcctcattttgtgaaagaaatgaacctagattcttaagcattattacacatccatgtttgcttaaagatggatttccctgggaatgggagaaaacagccagcaggaggagcttcatctgttcccttcccacctccaacctagccctactgcccaccccaccccaacccaccccatgcccagtggtctcagtagatacttcttaactggaaattctttcttttcagaatctaggtggtgaattttttttaagtggcacggtctttttctgcttgaaatctgatcacaccccccagccattgccctccctctctttttcctctgtagagaaatgtgaggggcagtacatttactgtgcttttcacaccatctcagaggttgaggagcatactgaaaattgccctggggggtgctgggtgtgctgtctccttcccacatcctcagccccacaccagctctatttcaggggtgagagtcagagagcactgcaatatgtgcttcatgggatttcgattcgaagatcctagaccagggagacactgtgagccagggatacaacaaaatactaggtaagtcactgcagaccgacctccctgcagtttgggaaagaagctgggtttgtggagaatcagagcatcttgacatgactgctgacctaaagatccctggcattggccagggatcctgtggaacctcttctagttcaggggtgtgagcattagactgccagttgtctagtgacatctgatgcttgctgtgaacttttaagatccccgaatcctgagcacctcaatctttaattgccctgtattccgaagggtaatataatttatctggatggaaattttaaagatgaatcccccttttttcttttcttctctcttttctttccttctccctttcttctttgccttctaaatatactgaaatgatttagatatgtgtcaacaattaatgatcttttattcaatctaagaaatggtttagtttttctctttagctctatggcatttcactcaagtggacaggggaaaaagtaattgccatgggctccaaagaatttgctttatgtttttagctatttaaaaataaatccatcaaaaataaagtatgcaaatgtatcttttaaagttaatttttaaaaatgctcttattttagtgaattttcagaaattatagtggaatggatgctcatatattgcttatggatattttggataccaaagtaggaataactgacattcagtattttaaagctggcaaacctgtacatagaaaatagatccccagacagtggtctatgaagagggcagttaagtatcaaatacttaattttcttgcctttttttcttaagtggggaaaagtttctagatctcttacacctctgacacaatctgttctaaaacaggcacttgtaatgttggggcctccttgtaaacgtgtttttgccctttactctctgggagttctttaaaggtgaaatcatcttacaaagaaattgggggagggtcttggcaaaggactttcccctcctctttcctggcctgggaaccttatactgacaatcaatactttatattttaaagtatataatttatagttaacttctagtgtaatatattaggaaacactagaatggaaaggccattggaagacaggttgtatcttttttagaccatatttccttgtttaaaaactatcatttgaatacttttttggtgaagaactccatgttttcaagttaaaggtcacctcgtaggccaggcgcagtggctcatgcctgtaatcccagcactctgggaggctgaggcgggtgaatcacaaggttaggagtttgagaccagcctggccaatatggtgaaaccccgtccctactaaaaatacaaaatttagccaggcgtggtggcatgcacctgtagtcccacctactcgggaggctgaggcaggagaatcacttgaacctgagagacagaggttgcagtgagccgagatcacgccactgcactccagcctgggggacagagtgagattctgtctcaaaaaacaaaaaacaaaaaagtcaccttgtaactcatctctttttattgtaagtttattaaaaatgaagaggacaacaatgagaaggaacataaagggttagctagcactgtctcctggtgcatggggctgtgcagatgtcccggccacttcttccttcatacttcccttagagaacttgctctgctacaagcagtgggcttggactaaaagtgattaaaataccacaggcataaggagaaaaggagtatatgtagtagtaataattactagtataaattattttcttcacatgctatgagtaataatattaaaaaactcattttaccattaagattccttatgctgaagctcttccatttagaatactgtcaatgtcatttactggtatgaactaaagtcccccttcttttccactcactgggaaccttagtaaaacaccagcatatcttacctctctttctgactggccgatgcttccagagactgaatgttgggaaaacctagtagccaaacaattctaggacagaataacatttttatatttggttccaccatcttattacatttagttatagttttaaaaaagaaattcaagcccattaaaatatgtctggtcaatgaaatgcttccttttattgtgttgtgctattgtactttgtttttcaaaacattgtaaaaatagtatctttggtttagtattttggattatatattataatctgaggagtgttttgcttatgtagaatccagatatatttctgttacctaggagatgttacttacatatgtaatactgtatcctgcacgtggaaatattcagaattgtagatagcataactctccctgctcctattcttttgagcctaggtataattttttttttttttttagaaaaagacatatttagctttaatttctatttatgctaaacatatttataagtagtctgtcaatataataccaactatttttatttttacataattcaattatttcatttgacatgtctggcagactcaagacattaagtaaaaaattggaactatgatttttctttgtcattttttaaaaaagaattattttattaacctgctggcatataatctggagttcttttcacaaccttactttttctgatttgctttattgaatgattgaatactcatttctttctaaaaatatgttgtaaattctcccttggcaagatttctccctatgagggtagttattatttgagtctgccaagtggttaccatggggcaaggtgccatgatgtattcttgggtgcattggttttttgcgcattgtaaatttaagacacttatagtaagtggactcattcatagatgagtttcagaaccttttacgttctcggtagaggcttctgtcggacaggcagaagagtgtattcctcacttttttttttgtcttcaaattccagtaaggcatagcacttttaagaaattagaatttttctatcatctatgcaaatgatatttatgttaatattaaatatcttatgttacactgggagtaatttgaggtgcaattatttttattactactttgaatagaggaccattatccttctttcttcagaaaactaagaagtaagtgtaacttttaaagtaagtatatatcagtgagagtaggcttgttttacaactatttctagccagtgagttgtgttttcatgtctcatcaaaagacaataccacattgcatcattttacaaaatatgttgtcattttcatttcagttgtaacataggaaaatagatatttcctagatgatttctgagtttcttactgcaaagaacagttataaattggtatacatgtgtctctgtaatagggataatattgatatatctgttgctacatatttaagaatcattctatcttatgttgtcttgaggccaagatttaccacgtttgcccagtgtattgaattggtggtagaaggtagttccatgttccatttgtagatctttaagattttatctttgataactttaatagaatgtggctcagttctggtccttcaagcctgtatggtttggattttcagtaggggacagttgatgtggagtcaatctctttggtacacaggaagctttataaaatttcattcacgaatctcttattttgggaagctgttttgcatatgagaagaacactgttgaaataaggaactaaagctttatatattgatcaaggtgattctgaaagttttaatttttaatgttgtaatgttatgttattgttaattgtactttattatgtattcaatagaaaatcatgatttattaataaaagcttaaattctcatcta

SEQ ID NO: 30 - protein, 1428 aa, Homo sapiens
MQGPPLLTAAHLLCVCTAALAVAPGPRFLVTAPGIIRPGGNVTIGVELLEHCPSQVTVKAELLKTASNLTVSVLEAEGVFEKGSFKTLTLPSLPLNSADEIYELRVTGRTQDEILFSNSTRLSFETKRISVFIQTDKALYKPKQEVKFRIVTLFSDFKPYKTSLNILIKDPKSNLIQQWLSQQSDLGVISKTFQLSSHPILGDWSIQVQVNDQTYYQSFQVSEYVLPKFEVTLQTPLYCSMNSKHLNGTITAKYTYGKPVKGDVTLTFLPLSFWGKKKNITKTFKINGSANFSFNDEEMKNVMDSSNGLSEYLDLSSPGPVEILTTVTESVTGISRNVSTNVFFKQHDYIIEFFDYTTVLKPSLNFTATVKVTRADGNQLTLEERRNNVVITVTQRNYTEYWSGSNSGNQKMEAVQKINYTVPQSGTFKIEFPILEDSSELQLKAYFLGSKSSMAVHSLFKSPSKTYIQLKTRDENIKVGSPFELVVSGNKRLKELSYMVVSRGQLVAVGKQNSTMFSLTPENSWTPKACVIVYYIEDDGEIISDVLKIPVQLVFKNKIKLYWSKVKAEPSEKVSLRISVTQPDSIVGIVAVDKSVNLMNASNDITMENVVHELELYNTGYYLGMFMNSFAVFQECGLWVLTDANLTKDYIDGVYDNAEYAERFMEENEGHIVDIHDFSLGSSPHVRKHFPETWIWLDTNMGYRIYQEFEVTVPDSITSWVATGFVISEDLGLGLTTTPVELQAFQPFFIFLNLPYSVIRGEEFALEITIFNYLKDATEVKVIIEKSDKFDILMTSNEINATGHQQTLLVPSEDGATVLFPIRPTHLGEIPITVTALSPTASDAVTQMILVKAEGIEKSYSQSILLDLTDNRLQSTLKTLSFSFPPNTVTGSERVQITAIGDVLGPSINGLASLIRMPYGCGEQNMINFAPNIYILDYLTKKKQLTDNLKEKALSFMRQGYQRELLYQREDGSFSAFGNYDPSGSTWLSAFVLRCFLEADPYIDIDQNVLHRTYTWLKGHQKSNGEFWDPGRVIHSELQGGNKSPVTLTAYIVTSLLGYRKYQPNIDVQESIHFLESEFSRGISDNYTLALITYALSSVGSPKAKEALNMLTWRAEQEGGMQFWVSSESKLSDSWQPRSLDIEVAAYALLSHFLQFQTSEGIPIMRWLSRQRNSLGGFASTQDTTVALKALSEFAALMNTERTNIQVTVTGPSSPSPLAVVQPTAVNISANGFGFAICQLNVVYNVKASGSSRRRRSIQNQEAFDLDVAVKENKDDLNHVDLNVCTSFSGPGRSGMALMEVNLLSGFMVPSEAISLSETVKKVEYDHGKLNLYLDSVNETQFCVNIPAVRNFKVSNTQDASVSIVDYYEPRRQAVRSYNSEVKLSSCDLCSDVQGCRPCEDGASGSHHHSSVIFIFCFKLLYFMELWL

SEQ ID NO: 31 - protein, 1368 aa, Homo sapiens
MQGPPLLTAAHLLCVCTAALAVAPGPRFLVTAPGIIRPGGNVTIGVELLEHCPSQVTVKAELLKTASNLTVSVLEAEGVFEKGSFKTLTLPSDPKSNLIQQWLSQQSDLGVISKTFQLSSHPILGDWSIQVQVNDQTYYQSFQVSEYVLPKFEVTLQTPLYCSMNSKHLNGTITAKYTYGKPVKGDVTLTFLPLSFWGKKKNITKTFKINGSANFSFNDEEMKNVMDSSNGLSEYLDLSSPGPVEILTTVTESVTGISRNVSTNVFFKQHDYIIEFFDYTTVLKPSLNFTATVKVTRADGNQLTLEERRNNVVITVTQRNYTEYWSGSNSGNQKMEAVQKINYTVPQSGTFKIEFPILEDSSELQLKAYFLGSKSSMAVHSLFKSPSKTYIQLKTRDENIKVGSPFELVVSGNKRLKELSYMVVSRGQLVAVGKQNSTMFSLTPENSWTPKACVIVYYIEDDGEIISDVLKIPVQLVFKNKIKLYWSKVKAEPSEKVSLRISVTQPDSIVGIVAVDKSVNLMNASNDITMENVVHELELYNTGYYLGMFMNSFAVFQECGLWVLTDANLTKDYIDGVYDNAEYAERFMEENEGHIVDIHDFSLGSSPHVRKHFPETWIWLDTNMGYRIYQEFEVTVPDSITSWVATGFVISEDLGLGLTTTPVELQAFQPFFIFLNLPYSVIRGEEFALEITIFNYLKDATEVKVIIEKSDKFDILMTSNEINATGHQQTLLVPSEDGATVLFPIRPTHLGEIPITVTALSPTASDAVTQMILVKAEGIEKSYSQSILLDLTDNRLQSTLKTLSFSFPPNTVTGSERVQITAIGDVLGPSINGLASLIRMPYGCGEQNMINFAPNIYILDYLTKKKQLTDNLKEKALSFMRQGYQRELLYQREDGSFSAFGNYDPSGSTWLSAFVLRCFLEADPYIDIDQNVLHRTYTWLKGHQKSNGEFWDPGRVIHSELQGGNKSPVTLTAYIVTSLLGYRKYQPNIDVQESIHFLESEFSRGISDNYTLALITYALSSVGSPKAKEALNMLTWRAEQEGGMQFWVSSESKLSDSWQPRSLDIEVAAYALLSHFLQFQTSEGIPIMRWLSRQRNSLGGFASTQDTTVALKALSEFAALMNTERTNIQVTVTGPSSPSPVKFLIDTHNRLLLQTAELAVVQPTAVNISANGFGFAICQLNVVYNVKASGSSRRRRSIQNQEAFDLDVAVKENKDDLNHVDLNVCTSFSGPGRSGMALMEVNLLSGFMVPSEAISLSETVKKVEYDHGKLNLYLDSVNETQFCVNIPAVRNFKVSNTQDASVSIVDYYEPRRQAVRSYNSEVKLSSCDLCSDVQGCRPCEDGASGSHHHSSVIFIFCFKLLYFMELWL

SEQ ID NO: 32 - protein, 1445 aa, Homo sapiens
MQGPPLLTAAHLLCVCTAALAVAPGPRFLVTAPGIIRPGGNVTIGVELLEHCPSQVTVKAELLKTASNLTVSVLEAEGVFEKGSFKTLTLPSLPLNSADEIYELRVTGRTQDEILFSNSTRLSFETKRISVFIQTDKALYKPKQEVKFRIVTLFSDFKPYKTSLNILIKDPKSNLIQQWLSQQSDLGVISKTFQLSSHPILGDWSIQVQVNDQTYYQSFQVSEYVLPKFEVTLQTPLYCSMNSKHLNGTITAKYTYGKPVKGDVTLTFLPLSFWGKKKNITKTFKINGSANFSFNDEEMKNVMDSSNGLSEYLDLSSPGPVEILTTVTESVTGISRNVSTNVFFKQHDYIIEFFDYTTVLKPSLNFTATVKVTRADGNQLTLEERRNNVVITVTQRNYTEYWSGSNSGNQKMEAVQKINYTVPQSGTFKIEFPILEDSSELQLKAYFLGSKSSMAVHSLFKSPSKTYIQLKTRDENIKVGSPFELVVSGNKRLKELSYMVVSRGQLVAVGKQNSTMFSLTPENSWTPKACVIVYYIEDDGEIISDVLKIPVQLVFKNKIKLYWSKVKAEPSEKVSLRISVTQPDSIVGIVAVDKSVNLMNASNDITMENVVHELELYNTGYYLGMFMNSFAVFQECGLWVLTDANLTKDYIDGVYDNAEYAERFMEENEGHIVDIHDFSLGSSPHVRKHFPETWIWLDTNMGYRIYQEFEVTVPDSITSWVATGFVISEDLGLGLTTTPVELQAFQPFFIFLNLPYSVIRGEEFALEITIFNYLKDATEVKVIIEKSDKFDILMTSNEINATGHQQTLLVPSEDGATVLFPIRPTHLGEIPITVTALSPTASDAVTQMILVKAEGIEKSYSQSILLDLTDNRLQSTLKTLSFSFPPNTVTGSERVQITAIGDVLGPSINGLASLIRMPYGCGEQNMINFAPNIYILDYLTKKKQLTDNLKEKALSFMRQGYQRELLYQREDGSFSAFGNYDPSGSTWLSAFVLRCFLEADPYIDIDQNVLHRTYTWLKGHQKSNGEFWDPGRVIHSELQGGNKSPVTLTAYIVTSLLGYRKYQPNIDVQESIHFLESEFSRGISDNYTLALITYALSSVGSPKAKEALNMLTWRAEQEGGMQFWVSSESKLSDSWQPRSLDIEVAAYALLSHFLQFQTSEGIPIMRWLSRQRNSLGGFASTQDTTVALKALSEFAALMNTERTNIQVTVTGPSSPSPVKFLIDTHNRLLLQTAELAVVQPTAVNISANGFGFAICQLNVVYNVKASGSSRRRRSIQNQEAFDLDVAVKENKDDLNHVDLNVCTSFSGPGRSGMALMEVNLLSGFMVPSEAISLSETVKKVEYDHGKLNLYLDSVNETQFCVNIPAVRNFKVSNTQDASVSIVDYYEPRRQAVRSYNSEVKLSSCDLCSDVQGCRPCEDGASGSHHHSSVIFIFCFKLLYFMELWL

SEQ ID NO: 33 - protein, 665 aa, Homo sapiens
MQGPPLLTAAHLLCVCTAALAVAPGPRFLVTAPGIIRPGGNVTIGVELLEHCPSQVTVKAELLKTASNLTVSVLEAEGVFEKGSFKTLTLPSLPLNSADEIYELRVTGRTQDEILFSNSTRLSFETKRISVFIQTDKALYKPKQEVKFRIVTLFSDFKPYKTSLNILIKDPKSNLIQQWLSQQSDLGVISKTFQLSSHPILGDWSIQVQVNDQTYYQSFQVSEYVLPKFEVTLQTPLYCSMNSKHLNGTITAKYTYGKPVKGDVTLTFLPLSFWGKKKNITKTFKINGSANFSFNDEEMKNVMDSSNGLSEYLDLSSPGPVEILTTVTESVTGISRNVSTNVFFKQHDYIIEFFDYTTVLKPSLNFTATVKVTRADGNQLTLEERRNNVVITVTQRNYTEYWSGSNSGNQKMEAVQKINYTVPQSGTFKIEFPILEDSSELQLKAYFLGSKSSMAVHSLFKSPSKTYIQLKTRDENIKVGSPFELVVSGNKRLKELSYMVVSRGQLVAVGKQNSTMFSLTPENSWTPKACVIVYYIEDDGEIISDVLKIPVQLVFKNKIKLYWSKVKAEPSEKVSLRISVTQPDSIVGIVAVDKSVNLMNASNDITMENVVHELELYNTGYYLGMFMNSFAVFQECGLWVLTDANLTKDYIDGVYDNLFGTQEAL

SEQ ID NO: 34 - protein, 1374 aa, Homo sapiens
EADAVEMQGPPLLTAAHLLCVCTAALAVAPGPRFLVTAPGIIRPGGNVTIGVELLEHCPSQVTVKAELLKTASNLTVSVLEAEGVFEKGSFKTLTLPSDPKSNLIQQWLSQQSDLGVISKTFQLSSHPILGDWSIQVQVNDQTYYQSFQVSEYVLPKFEVTLQTPLYCSMNSKHLNGTITAKYTYGKPVKGDVTLTFLPLSFWGKKKNITKTFKINGSANFSFNDEEMKNVMDSSNGLSEYLDLSSPGPVEILTTVTESVTGISRNVSTNVFFKQHDYIIEFFDYTTVLKPSLNFTATVKVTRADGNQLTLEERRNNVVITVTQRNYTEYWSGSNSGNQKMEAVQKINYTVPQSGTFKIEFPILEDSSELQLKAYFLGSKSSMAVHSLFKSPSKTYIQLKTRDENIKVGSPFELVVSGNKRLKELSYMVVSRGQLVAVGKQNSTMFSLTPENSWTPKACVIVYYIEDDGEIISDVLKIPVQLVFKNKIKLYWSKVKAEPSEKVSLRISVTQPDSIVGIVAVDKSVNLMNASNDITMENVVHELELYNTGYYLGMFMNSFAVFQECGLWVLTDANLTKDYIDGVYDNAEYAERFMEENEGHIVDIHDFSLGSSPHVRKHFPETWIWLDTNMGYRIYQEFEVTVPDSITSWVATGFVISEDLGLGLTTTPVELQAFQPFFIFLNLPYSVIRGEEFALEITIFNYLKDATEVKVIIEKSDKFDILMTSNEINATGHQQTLLVPSEDGATVLFPIRPTHLGEIPITVTALSPTASDAVTQMILVKAEGIEKSYSQSILLDLTDNRLQSTLKTLSFSFPPNTVTGSERVQITAIGDVLGPSINGLASLIRMPYGCGEQNMINFAPNIYILDYLTKKKQLTDNLKEKALSFMRQGYQRELLYQREDGSFSAFGNYDPSGSTWLSAFVLRCFLEADPYIDIDQNVLHRTYTWLKGHQKSNGEFWDPGRVIHSELQGGNKSPVTLTAYIVTSLLGYRKYQPNIDVQESIHFLESEFSRGISDNYTLALITYALSSVGSPKAKEALNMLTWRAEQEGGMQFWVSSESKLSDSWQPRSLDIEVAAYALLSHFLQFQTSEGIPIMRWLSRQRNSLGGFASTQDTTVALKALSEFAALMNTERTNIQVTVTGPSSPSPVKFLIDTHNRLLLQTAELAVVQPMAVNISANGFGFAICQLNVVYNVKASGSSRRRRSIQNQEAFDLDVAVKENKDDLNHVDLNVCTSFSGPGRSGMALMEVNLLSGFMVPSEAISLSETVKKVEYDHGKLNLYLDSVNETQFCVNIPAVRNFKVSNTQDASVSIVDYYEPRRQAVRSYNSEVKLSSCDLCSDVQGCRPCEDGASGSHHHSSVIFIFCFKLLYFMELWL

SEQ ID NO: 35 - protein, 854 aa, Homo sapiens
VDKSVNLMNASNDITMENVVHELELYNTGYYLGMFMNSFAVFQECGLWVLTDANLTKDYIDGVYDNAEYAERFMEENEGHIVDIHDFSLGSSPHVRKHFPETWIWLDTNMGSRIYQEFEVTVPDSITSWVATGFVISEDLGLGLTTTPVELQAFQPFFIFLNLPYSVIRGEEFALEITIFNYLKDATEVKVIIEKSDKFDILMTSSEINATGHQQTLLVPSEDGATVLFPIRPTHLGEIPITVTALSPTASDAITQMILVKAEGIEKSYSQSILLDLTDNRLQSTLKTLSFSFPPNTVTGSERVQITAIGDVLGPSINGLASLIRMPYGCGEQNMINFAPNIYILDYLTKKKQLTDNLKEKALSFMRQGYQRELLYQREDGSFSAFGNYDPSGSTWLSAFVLRCFLEADPYIDIDQNVLHRTYTWLKGHQKSNGEFWDPGRVIHSELQGGNKSPVTLTAYIVTSLLGYRKYQPNIDVQESIHFLESEFSRGISDNYTLALITYALSSVGSPKAKEALNMLTWRAEQEGGMQFWVSSESKLSDSWQPRSLDIEVAAYALLSHFLQFQTSEGIPIMRWLSRQRNSLGGFASTQDTTVALKALSEFAALMNTERTNIQVTVTGPSSPSPVKFLIDTHNRLLLQTAELAVVQPTAVNISANGFGFAICQLNVVYNVKASGSSRRRRSIQNQEAFDLDVAVKENKDDLNHVDLNVCTSFSGPGRSGMALMEVNLLSGFMVPSEAISLSETVKKVEYDHGKLNLYLDSVNETQFCVNIPAVRNFKVSNTQDASVSIVDYYEPRRQAVRSYNSEVKLSSCDLCSDVQGCRPCEDGASGSHHHSSVIFIFCFKLLYFMELWL

SEQ ID NO: 36 - protein, 847 aa, Homo sapiens [sequence omitted]

SEQ ID NO: 37 - DNA, 21 bp, synthetic construct
  misc_feature 1..21: note = Synthetic sequence; gRNA Recognition Sequence
  source 1..21: mol_type = other DNA
gcctccaagtcctgtctcaat

SEQ ID NO: 38 - DNA, 21 bp, synthetic construct
  misc_feature 1..21: note = Synthetic sequence; gRNA Recognition Sequence
  source 1..21: mol_type = other DNA
ggtaccatcacggcaaagtat

SEQ ID NO: 39 - DNA, 21 bp, synthetic construct
  misc_feature 1..21: note = Synthetic sequence; gRNA Recognition Sequence
  source 1..21: mol_type = other DNA
gtaccatcacggcaaagtata

SEQ ID NO: 40 - DNA, 21 bp, synthetic construct
  misc_feature 1..21: note = Synthetic sequence; gRNA Recognition Sequence
  source 1..21: mol_type = other DNA
gctacagttgaaggcctattt

SEQ ID NO: 41 - DNA, 21 bp, synthetic construct
  misc_feature 1..21: note = Synthetic sequence; gRNA Recognition Sequence
  source 1..21: mol_type = other DNA
gattgaaggagttaagctata

SEQ ID NO: 42 - DNA, 21 bp, synthetic construct
  misc_feature 1..21: note = Synthetic sequence; gRNA Recognition Sequence
  source 1..21: mol_type = other DNA
ggtcttggactaacaactact

SEQ ID NO: 43 - DNA, 21 bp, synthetic construct
  misc_feature 1..21: note = Synthetic sequence; gRNA Recognition Sequence
  source 1..21: mol_type = other DNA
gaaagatgccactgaggttaa

SEQ ID NO: 44 - DNA, 21 bp, synthetic construct
  misc_feature 1..21: note = Synthetic sequence; gRNA Recognition Sequence
  source 1..21: mol_type = other DNA
gcatctactcaggataccact

SEQ ID NO: 45 - DNA, 21 bp, synthetic construct
  misc_feature 1..21: note = Synthetic sequence; gRNA Recognition Sequence
  source 1..21: mol_type = other DNA
ggtacagccaacggcagttaa

SEQ ID NO: 46 - DNA, 21 bp, synthetic construct
  misc_feature 1..21: note = Synthetic sequence; gRNA Recognition Sequence
  source 1..21: mol_type = other DNA
ggctcttatggaagttaacct

SEQ ID NO: 47 - DNA, 21 bp, synthetic construct
  misc_feature 1..21: note = Synthetic sequence; gRNA Recognition Sequence
  source 1..21: mol_type = other DNA
gacaggcggtgagaagttaca

SEQ ID NO: 48 - DNA, 21 bp, synthetic construct
  misc_feature 1..21: note = Synthetic sequence; gRNA Recognition Sequence
  source 1..21: mol_type = other DNA
gcccagtggtctcagtagata

SEQ ID NO: 49 - DNA, 19 bp, synthetic construct
  misc_feature 1..19: note = Synthetic sequence; gRNA Recognition Sequence
  source 1..19: mol_type = other DNA
gccgatccttacatagata

SEQ ID NO: 50 - DNA, 19 bp, synthetic construct
  misc_feature 1..19: note = Synthetic sequence; gRNA Recognition Sequence
  source 1..19: mol_type = other DNA
cctagattcttaagcatta

SEQ ID NO: 51 - DNA, 19 bp, synthetic construct
  misc_feature 1..19: note = Synthetic sequence; gRNA Recognition Sequence
  source 1..19: mol_type = other DNA
aagcctgtgtaattgtgta

SEQ ID NO: 52 - DNA, 19 bp, synthetic construct
  misc_feature 1..19: note = Synthetic sequence; gRNA Recognition Sequence
  source 1..19: mol_type = other DNA
agagttcagatcactgcaa

SEQ ID NO: 53 - DNA, 19 bp, synthetic construct
  misc_feature 1..19: note = Synthetic sequence; gRNA Recognition Sequence
  source 1..19: mol_type = other DNA
aggagacgtaacgcttaca

SEQ ID NO: 54 - DNA, 19 bp, synthetic construct
  misc_feature 1..19: note = Synthetic sequence; gRNA Recognition Sequence
  source 1..19: mol_type = other DNA
tgtaagcactaatgtgttc

SEQ ID NO: 55 - DNA, 19 bp, synthetic construct
  misc_feature 1..19: note = Synthetic sequence; gRNA Recognition Sequence
  source 1..19: mol_type = other DNA
tgcagaatatgctgagagg

SEQ ID NO: 56 - DNA, 19 bp, synthetic construct
  misc_feature 1..19: note = Synthetic sequence; gRNA Recognition Sequence
  source 1..19: mol_type = other DNA
actaagaagtaagtgtaac

SEQ ID NO: 57 - DNA, 19 bp, synthetic construct
  misc_feature 1..19: note = Synthetic sequence; gRNA Recognition Sequence
  source 1..19: mol_type = other DNA
tgcagaatatgctgagagg

SEQ ID NO: 58 - DNA, 19 bp, synthetic construct
  misc_feature 1..19: note = Synthetic sequence; gRNA Recognition Sequence
  source 1..19: mol_type = other DNA
ttggagatgttcttggtcc